(19) United States
(12) Patent Application Publication
(10) Pub. No.: US 2022/0002802 A1
(43) Pub. Date: Jan. 6, 2022

(54) COMPOSITIONS AND METHODS FOR IMMUNE REPERTOIRE SEQUENCING

(71) Applicant: Life Technologies Corporation, Carlsbad, CA (US)

(72) Inventors: Timothy LOONEY, Austin, TX (US); Geoffrey LOWMAN, Carlsbad, CA (US); Lifeng LIN, Dublin, CA (US); Chenchen YANG, Foster City, CA (US)

(21) Appl. No.: 17/250,408

(22) PCT Filed: Jul. 18, 2019
(86) PCT No.: PCT/US2019/042474

§ 371 Date: Jan. 18, 2021

Related U.S. Application Data
(60) Provisional application No. 62/700,168, filed on Jul. 18, 2018.
(60) Provisional application No. 62/839,505, filed on Apr. 26, 2019.

(51) Int. Cl. C12Q 1/6881; C12N 15/10

(52) U.S. Cl. CPC C12Q 1/6881; C12N 15/1072; C12Q 2600/16; C12Q 2600/158; C12Q 2600/156

(57) ABSTRACT

The present disclosure provides methods, compositions, kits, and systems useful in the determination and evaluation of the immune repertoire. In one aspect, target-specific primer panels provide for the effective amplification of sequences of B cell receptor chains with improved sequencing accuracy and resolution over the repertoire. Variable regions associated with the immune cell receptor are resolved to effectively portray clonal diversity of a biological sample and/or differences associated with the immune cell repertoire of a biological sample.

### CROSS-REFERENCE TO RELATED APPLICATIONS

This application claims priority to and the benefit of U.S. Provisional Application No. 62/839,505 filed Apr. 26, 2019 and U.S. Provisional Application No. 62/700,168 filed Jul. 18, 2018. The entire contents of each of the aforementioned applications are incorporated herein by reference.

### BACKGROUND

Adaptive immune response comprises selective response of B and T cells recognizing antigens. The immunoglobulin genes encoding antibody (Ab, in B cell) and T-cell receptor (TCR, in T cell) antigen receptors comprise complex loci wherein extensive diversity of receptors is produced as a result of recombination of the respective variable (V), diversity (D), and joining (J) gene segments, as well as subsequent somatic hypermutation events during early lymphoid differentiation. The recombination process occurs separately for both subunit chains of each receptor and subsequent heterodimeric pairing creates still greater combinatorial diversity. Calculations of the potential combinatorial and junctional possibilities that contribute to the human immune receptor repertoire have estimated that the number of possibilities greatly exceeds the total number of peripheral B or T cells in an individual. See, for example, Davis and Bjorkman (1988) Nature 334:395-402; Arstila et al. (1999) Science 286:958-961; van Dongen et al., In: Leukemia, Henderson et al. (eds) Philadelphia: WB Saunders Company, 2002, pp 85-129.

Extensive efforts have been made over years to improve analysis of the immune repertoire at high resolution. Means for specific detection and monitoring of expanded clones of lymphocytes would provide significant opportunities for characterization and analysis of normal and pathogenic immune reactions and responses. Despite efforts, effective high resolution analysis has provided challenges. Low throughput techniques such as Sanger sequencing may provide resolution, but are limited to provide efficient means to broadly capture the entire immune repertoire. Advances in next generation sequencing (NGS) have provided access to capturing the repertoire, however, due to the nature of the numerous related sequences and introduction of sequence errors as a result of the technology, efficient and effective reflection of the true repertoire has proven difficult. Thus, improved sequencing methodologies and workflows capable of resolving complex populations of highly variable immune cell receptor sequences are being developed. There remains a need for new methods for effective profiling of vast repertoires of immune cell receptors to better understand immune cell response, enhance diagnostic and treatment capabilities, and devise new therapeutics.

### SUMMARY OF THE INVENTION

In one aspect of the invention compositions are provided for a single stream determination of an immune repertoire in a sample. In some embodiments the composition comprises at least one set of primers i) and ii), wherein i) consists of a plurality of variable (V) gene primers directed to a majority of different variable regions of an immune receptor coding sequence; and ii) consists one or more constant (C) gene primers directed to at least a portion of the respective target constant region of the respective immune receptor coding sequence. In some embodiments the composition comprises at least one set of primers i) and ii), wherein i) consists of a plurality of variable (V) gene primers directed to a majority of different V genes of an immune receptor coding sequence; and ii) consists of a plurality of joining (J) gene primers directed to a majority of different J genes of the respective target immune receptor coding sequence. In some embodiments the composition for analysis of a B cell receptor (BCR) repertoire in a sample comprises at least one set of primers i) and ii), wherein i) consists of (a) a plurality of V gene primers directed to a majority of different V genes of at least one BCR coding sequence comprising at least a portion of framework region 1 (FR1) within the V gene, or (b) a plurality of V gene primers directed to a majority of different V genes of at least one BCR coding sequence comprising at least a portion of framework region 3 (FR3) within the V gene; and ii) consists of (a) one or more C gene primers directed to at least a portion of a C gene of the at least one BCR coding sequence, or (b) a plurality of J gene primers directed to at least a portion of a majority of different J genes of the at least one BCR coding sequence; wherein each set of i) and ii) primers directed to coding sequences of the same target BCR gene selected from IgH, IgL, and IgK; and wherein each set of i) and ii) primers directed to the same target BCR is configured to amplify the target BCR repertoire.

In some embodiments the V gene primers recognize at least a portion of FR1 within the V gene. In some embodiments the V gene primers recognize at least a portion of FR2 within the V gene. In some embodiments the V gene primers recognize at least a portion of FR3 within the V gene. Each set of i) and ii) primers are directed to the same target BCR sequence selected from the group consisting of IgH, IgL, and IgK, and configured such that resulting amplicons generated using such compositions represent the repertoire of sequences of the respective receptor in a sample. In particular embodiments, provided compositions include a plurality of primer pair reagents selected from Table 2 and Table 5. In particular embodiments, provided compositions include a plurality of primer pair reagents selected from Table 3 and Table 5. In particular embodiments, provided compositions include a plurality of primer pair reagents selected from Table 3 and from Tables 6-10. In particular embodiments, provided compositions include a plurality of primer pair reagents selected from Table 2 and from Tables 6-10. In some embodiments, provided compositions include a plurality of primer pair reagents selected from Table 4 and Table 5 or selected from Table 4 and Tables 6-10. In some embodiments a multiplex assay comprising compositions of the invention is provided. In some embodiments a test kit comprising compositions of the invention is provided.

In other aspects of the invention, methods are provided for determining immune repertoire activity in a biological sample. Such methods comprise performing multiplex amplification with primer set which target two different types of immune receptors, for example, multiplex amplification of TCR and BCR targets in a single reaction.

In some embodiments, the method for amplification of expression nucleic acid sequences of an immune receptor repertoire in a sample comprises performing a single multiplex amplification reaction to amplify expressed target immune receptor nucleic acid template molecules using at least one set of:
i) (a) a plurality of V gene primers directed to a majority of different V genes of at least one immune receptor coding sequence comprising at least a portion of FR1 within the V gene,
   (b) a plurality of V gene primers directed to a majority of different V genes of at least one immune receptor coding sequence comprising at least a portion of FR2 within the V gene, or
   (c) a plurality of V gene primers directed to a majority of different V genes of at least one immune receptor coding sequence comprising at least a portion of FR3 within the V gene; and
ii) (a) one or more C gene primers directed to at least a portion of a C gene of the at least one immune receptor coding sequence, or
   (b) a plurality of J gene primers directed to at least a portion of a majority of different J genes of the at least one immune receptor coding sequence;
wherein each set of i) and ii) primers is directed to coding sequences of the same target immune receptor gene selected from a T cell receptor gene or an antibody receptor gene and wherein performing the amplification using the at least one set of i) and ii) primers results in amplicon molecules representing the target immune receptor repertoire in the sample; thereby generating immune receptor amplicon molecules comprising the target immune receptor repertoire.

In certain embodiments at least a portion of the first framework region (FR1) of the V gene to at least a portion of the C gene of the immune receptor sequence is encompassed within amplified target immune receptor sequences. In certain embodiments at least a portion of the second framework region (FR2) of the V gene to at least a portion of the C gene of the immune receptor sequence is encompassed within amplified target immune receptor sequences. In certain embodiments at least a portion of the third framework region (FR3) of the V gene to at least a portion of the C gene of the immune receptor sequence is encompassed within amplified target immune receptor sequences. In other embodiments, methods comprise amplification of expression nucleic acid sequences of an immune receptor repertoire in a sample comprising performing a multiplex amplification reaction in the presence of a polymerase under amplification conditions to produce a plurality of amplified target expression sequences comprising one or more immune receptors of interest having a variable, diversity, and joining (VDJ) gene portion or one or more immune receptors of interest having a variable and joining (VJ) gene portion. In certain embodiments at least a portion of the first framework region (FR1) of the V gene to at least a portion of the joining (J) gene of the immune receptor sequence is encompassed within amplified target immune receptor sequences. In certain embodiments at least a portion of the second framework region (FR2) of the V gene to at least a portion of the joining (J) gene of the immune receptor sequence is encompassed within amplified target immune receptor sequences. In certain embodiments at least a portion of the third framework region (FR3) of the V gene to at least a portion of the joining (J) gene of the immune receptor sequence is encompassed within amplified target immune receptor sequences.

Methods of the invention further comprise preparing a BCR repertoire library using the amplified target immune receptor sequences through introducing adapter sequences to the termini of the amplified target sequences. In some embodiments, the adapter-modified immune receptor repertoire library is clonally amplified.

The methods further comprise detecting sequences of the immune repertoire of each of the immune receptors in the sample and/or expression of each of the plurality of target immune receptor sequences, wherein a change in the level of repertoire sequences and/or expression of one or more target immune receptor markers as compared with a second sample or a control sample determines a change in immune repertoire activity in the sample. In certain embodiments sequencing of the immune receptor amplicon molecules is carried out using next generation sequence analysis to determine sequence of the immune receptor amplicons. In particular embodiments determining the sequence of the immune receptor amplicon molecules includes obtaining initial sequence reads, aligning and identifying productive reads and correcting errors to generate rescued productive reads and determining the sequences of the resulting total productive reads, thereby providing sequence of the immune repertoire in the sample. Provided methods described herein utilize compositions of the invention provided herein. In still other aspects of the invention, particular analysis methodology for error correction is provided in order to generate comprehensive, effective sequence information from methods provided herein.

In another aspect, methods are provided for identifying or screening for a biomarker for a disease or condition in a subject. In some embodiments, such methods comprise performing a single multiplex amplification reaction to amplify target BCR nucleic acid template molecules obtained from a subject's sample using at least one set of:
i) (a) a plurality of V gene primers directed to a majority of different V genes of at least one BCR coding sequence comprising at least a portion of FR1 within the V gene,
   (b) a plurality of V gene primers directed to a majority of different V genes of at least one BCR coding sequence comprising at least a portion of FR2 within the V gene, or
   (c) a plurality of V gene primers directed to a majority of different V genes of at least one BCR coding sequence comprising at least a portion of FR3 within the V gene; and
ii) (a) one or more C gene primers directed to at least a portion of a C gene of the at least one BCR coding sequence, or
   (b) a plurality of J gene primers directed to at least a portion of a majority of different J genes of the at least one BCR coding sequence;
wherein each set of i) and ii) primers is directed to coding sequences of the same target BCR gene selected from an IgH, IgL, and IgK gene and wherein performing the amplification using the at least one set of i) and ii) primers results in amplicon molecules representing the target BCR repertoire in the sample; thereby generating target BCR amplicon molecules comprising the target BCR repertoire. The method further comprises performing sequencing of the target BCR amplicon molecules and determining the sequence of the molecules, wherein determining the sequence includes obtaining initial sequence reads, aligning the initial sequence read to a reference sequence, identifying productive reads, and correcting one or more indel errors to generate rescued productive sequence reads; identifying BCR repertoire clonal populations from the determined target BCR sequences; and identifying the sequence of at least one BCR clone for use as a biomarker for the disease or condition. In some embodiments, the disease or condition a biomarker is identified or screened is selected from cancer, autoimmune disease, infectious disease, allergy, response to vaccination, and response to an immunotherapy treatment.

### BRIEF DESCRIPTION OF THE DRAWINGS

FIG. 1 is a diagram of an exemplary workflow for removal of PCR or sequencing-derived errors using stepwise clustering of similar CDR3 nucleotides sequences with steps: (A) very fast heuristic clustering into groups based on similarity (cd-hit-est); (B) cluster representative chosen as most common sequence, randomly picked for ties; (C) merge reads into representatives; (D) compare representatives and if within allotted hamming distance, merge clusters.

FIG. 2 is a diagram of an exemplary workflow for removal of residual insertion/deletion (indel) error by comparing homopolymer collapsed CDR3 sequences using Levenshtein distance with the steps: (A) collapse homopolymers and calculate Levenshtein distances between cluster representatives; (B) merge reads that now cluster together, these represent complex indel errors; (C) report lineages to user.

FIG. 3 is a graph depicting results of number of reads and characterization of read quality (productive vs off target or unproductive) for a BCR assay alone, a BCR and TCR assay amplified in one pool, and a BCR and TCR assay amplified in two separate pools.

FIGS. 4A-4B depict (FIG. 4A) the total number of clones detected and (FIG. 4B) the population of BCR and TCR clones in the combined assays (by percentage of the total).

FIGS. 5A-5F are histograms depicting sequence read lengths of the IgH repertoire from RNA of various cell or tissue samples: (FIG. 5A) PBL, (FIG. 5B) CD19+ cells, (FIG. 5C) tonsil FFPE, (FIG. 5D) lung tumor FFPE, (FIG. 5E) bone marrow, (FIG. 5F) normal spleen, and (FIG. 5G) normal brain.

FIG. 6 depicts sequence read lengths obtained following multiplex amplification of PBL cDNA using exemplary IgH V gene FR1-C gene primer sets 1-7.

FIGS. 7A-7B bar graphs depict total isotype representation within a PBL sample as sequence reads per isotype (FIG. 7A) and clones detected per isotype (FIG. 7B) obtained from an assay using an exemplary IgH V gene FR1-C gene multiplex amplification reaction.

FIGS. 8A-8B depict histograms of IgH V gene mutation rates in a PBL sample for (FIG. 8A) all IgH isotypes and (FIG. 8B) IgD only.

FIG. 9 depict IgH clonal analysis results for the total productive reads from a sample (rightmost point on each graph) and for 8 downsampled data sets derived from the total productive reads.

FIG. 10 depicts a graph showing the linearity of plasmid detection in IgH library generated from a pool of 20 control plasmids at equimolar concentrations mixed with leukocyte cDNA. The plasmid associated with the plasmid ID number is shown in Table 15.

### DESCRIPTION OF THE INVENTION

We have developed a multiplex next generation sequencing workflow for effective detection and analysis of the immune repertoire in a sample. Provided methods, compositions, systems, and kits are for use in high accuracy amplification and sequencing of immune cell receptor sequences (e.g., T cell receptor (TCR), B cell receptor (BCR or Ab) targets) in monitoring and resolving complex immune cell repertoire(s) in a subject. The target immune cell receptor genes have undergone rearrangement (or recombination) of the VDJ or VJ gene segments, the gene segments depending on the particular receptor gene (e.g., IgH, IgK, TCR beta or TCR alpha). In certain embodiments, the present disclosure provides methods, compositions, and systems that use nucleic acid amplification, such as polymerase chain reaction (PCR), to enrich expressed variable regions of immune receptor target nucleic acid for subsequent sequencing. In certain embodiments, the present disclosure provides methods, compositions, and systems that use nucleic acid amplification, such as PCR, to enrich rearranged target immune cell receptor gene sequences from gDNA for subsequent sequencing. In certain embodiments, the present disclosure also provides methods and systems for effective identification and removal of amplification or sequencing-derived error(s) to improve read assignment accuracy and lower the false positive rate. In particular, provided methods described herein may improve accuracy and performance in sequencing applications with nucleotide sequences associated with genomic recombination and high variability. In some embodiments, methods, compositions, systems, and kits provided herein are for use in amplification and sequencing of the complementarity determining regions (CDRs) of an expressed immune receptor in a sample. In some embodiments, methods, compositions, systems, and kits provided herein are for use in amplification and sequencing of the CDRs of rearranged immune cell receptor gDNA in a sample. Thus, provided herein are multiplex immune cell receptor expression compositions and immune cell receptor gene-directed compositions for multiplex library preparation, used in conjunction with next generation sequencing technologies and workflow solutions (e.g., manual or automated), for effective detection and characterization of the immune repertoire in a sample.

The CDRs of a TCR or BCR result from genomic DNA undergoing recombination of the V(D)J gene segments as well as addition and/or deletion of nucleotides at the gene segment junctions. Recombination of the V(D)J gene segments and subsequent hypermutation events leads to extensive diversity of the expressed immune cell receptors. With the stochastic nature of V(D)J recombination, it is often the case that rearrangement of the T or B cell receptor genomic DNA will fail to produce a functional receptor, instead producing what is termed an “unproductive” rearrangement. Typically, unproductive rearrangements have out-of-frame Variable and Joining coding segments, and lead to the presence of premature stop codons and synthesis of irrelevant peptides. Unproductive TCR or BCR gene rearrangements are generally rare in cDNA-based repertoire sequencing for a number of biological or physiological reasons such as: 1) nonsense-mediated decay, which destroys mRNA containing premature stop codons, 2) B and T cell selection, where only B and T cells with a functional receptor survive, and 3) allelic exclusion, where only a single rearranged receptor allele is expressed in any given B or T cell.

Accordingly, in some embodiments, methods and compositions provided herein are used for amplifying the recombined, expressed variable regions of immune cell receptor mRNA, eg BCR and/or TCR mRNA. In some embodiments, RNA extracted from biological samples is converted to cDNA. Multiplex amplification is used to enrich for a portion of BCR or TCR cDNA which includes at least a portion of the variable region of the receptor. In some embodiments, the amplified cDNA includes one or more complementarity determining regions CDR1, CDR2, and/or CDR3 for the target receptor. In some embodiments, the amplified cDNA includes one or more complementarity determining regions CDR1, CDR2, and/or CDR3 for immunoglobulin heavy chain (IgH).

BCR and TCR sequences can also appear as unproductive rearrangements from errors introduced during amplification reactions or during sequencing processes. For example, an insertion or deletion (indel) error during a target amplification or sequencing reaction can cause a frameshift in the reading frame of the resulting coding sequence. Such a change may result in a target sequence read of a productive rearrangement being interpreted as an unproductive rearrangement and discarded from the group of identified clonotypes. Accordingly, in some embodiments, methods and systems provided herein include processes for identification and/or removing PCR or sequencing-derived error from the determined immune receptor sequence.

In some embodiments, methods and compositions provided are used for amplifying the rearranged variable regions of immune cell receptor gDNA, e.g., rearranged BCR and/or TCR gene DNA. Multiplex amplification is used to enrich for a portion of rearranged BCR or TCR gDNA which includes at least a portion of the variable region of the receptor. In some embodiments, the amplified gDNA includes one or more complementarity determining regions CDR1, CDR2, and/or CDR3 for the target receptor. In some embodiments, the amplified gDNA includes one or more complementarity determining regions CDR1, CDR2, and/or CDR3 for IgH. In some embodiments, the amplified gDNA includes primarily CDR3 for the target receptor, e.g., CDR3 for IgH.

As used herein, “immune cell receptor” and “immune receptor” are used interchangeably.

As used herein, the terms “complementarity determining region” and “CDR” refer to regions of a T cell receptor or an antibody (immunoglobulin) where the molecule complements an antigen's conformation, thereby determining the molecule's specificity and contact with a specific antigen. In the variable regions of T cell receptors and antibodies, the CDRs are interspersed with regions that are more conserved, termed framework regions (FR). Each variable region of a T cell receptor and an antibody contains 3 CDRs, designated CDR1, CDR2 and CDR3, and also contains 4 framework sub-regions, designated FR1, FR2, FR3 and FR4.

As used herein, the term “framework” or “framework region” or “FR” refers to the residues of the variable region other than the CDR residues as defined herein. There are four separate framework sub-regions that make up the framework: FR1, FR2, FR3, and FR4.

The particular designation in the art for the exact location of the CDRs and FRs within the receptor molecule (TCR or immunoglobulin) varies depending on what definition is employed. Unless specifically stated otherwise, the IMGT designations are used herein in describing the CDR and FR regions (see Brochet et al. (2008) Nucleic Acids Res. 36:W503-508, herein specifically incorporated by reference). As one example of CDR/FR amino acid designations, the residues that make up the FRs and CDRs of T cell receptor beta have been characterized by IMGT as follows: residues 1-26 (FR1), 27-38 (CDR1), 39-55 (FR2), 56-65 (CDR2), 66-104 (FR3), 105-117 (CDR3), and 118-128 (FR4).

Other well-known standard designations for describing the regions include those found in Kabat et al., (1991) Sequences of Proteins of Immunological Interest, 5th Ed. Public Health Service, National Institutes of Health, Bethesda, Md., and in Chothia and Lesk (1987) J. Mol. Biol. 196:901-917; herein specifically incorporated by reference. As one example of CDR designations, the residues that make up the six immunoglobulin CDRs have been characterized by Kabat as follows: residues 24-34 (CDRL1), 50-56 (CDRL2) and 89-97 (CDRL3) in the light chain variable region and 31-35 (CDRH1), 50-65 (CDRH2) and 95-102 (CDRH3) in the heavy chain variable region; and by Chothia as follows: residues 26-32 (CDRL1), 50-52 (CDRL2) and 91-96 (CDRL3) in the light chain variable region and 26-32 (CDRH1), 53-55 (CDRH2) and 96-101 (CDRH3) in the heavy chain variable region.

The term “T cell receptor” or “T cell antigen receptor” or “TCR,” as used herein, refers to the antigen/MHC binding heterodimeric protein product of a vertebrate, e.g. mammalian, TCR gene complex, including the human TCR alpha, beta, gamma and delta chains. For example, the complete sequence of the human TCR beta locus has been sequenced, see, for example, Rowen et al. (1996) Science 272:1755-1762; the human TCR alpha locus has been sequenced and resequenced, see, for example, Mackelprang et al. (2006) Hum Genet. 119:255-266; and see, for example, Arden (1995) Immunogenetics 42:455-500 for a general analysis of the T-cell receptor V gene segment families; each of which is herein specifically incorporated by reference for the sequence information provided and referenced in the publication.

The term “antibody” or immunoglobulin” or “B cell receptor” or “BCR,” as used herein, is intended to refer to immunoglobulin molecules comprised of four polypeptide chains, two heavy (H) chains and two light (L) chains (lambda or kappa) inter-connected by disulfide bonds. An antibody has a known specific antigen with which it binds. Each heavy chain of an antibody is comprised of a heavy chain variable region (abbreviated herein as HCVR, HV or VH) and a heavy chain constant region. The heavy chain constant region is comprised of three domains, CH1, CH2 and CH3. Each light chain is comprised of a light chain variable region (abbreviated herein as LCVR or VL or KV or LV to designate kappa or lambda light chains) and a light chain constant region. The light chain constant region is comprised of one domain, CL. The heavy chain determines the class or isotype to which the immunoglobulin belongs. In mammals, for example, the five main immunoglobulin isotypes are IgA, IgD, IgG, IgE and IgM and they are classed according to the alpha, delta, epsilon, gamma or mu heavy chain they contain, respectively.

As noted, the diversity of the TCR and BCR chain CDRs is created by recombination of germline variable (V), diversity (D), and joining (J) gene segments, as well as by independent addition and deletion of nucleotides at each of the gene segment junctions during the process of TCR and BCR gene rearrangement. In the rearranged nucleic acid encoding a BCR heavy chain, CDR1 and CDR2 are found in the V gene segment and CDR3 includes some of the V gene segment and the D and J gene segments. In the rearranged nucleic acid encoding a BCR light chain, CDR1 and CDR2 are found in the V gene segment and CDR3 includes some of the V gene segment and the J gene segment. In the rearranged nucleic acid encoding a TCR beta and a TCR delta, for example, CDR1 and CDR2 are found in the V gene segments and CDR3 includes some of the V gene segment, and the D and J gene segments. In the rearranged nucleic acid encoding a TCR alpha and a TCR gamma, CDR1 and CDR2 are found in the V gene segments and CDR3 includes some of the V gene segment and the J gene segment.

In some embodiments, a multiplex amplification reaction is used to amplify cDNA derived from mRNA expressed from rearranged BCR and/or TCR genomic DNA. In some embodiments, a multiplex amplification reaction is used to amplify at least a portion of a BCR and/or TCR CDR from cDNA derived from a biological sample. In some embodiments, a multiplex amplification reaction is used to amplify at least two CDRs of a BCR and/or TCR from cDNA derived from a biological sample. In some embodiments, a multiplex amplification reaction is used to amplify at least three CDRs of a BCR and/or TCR from cDNA derived from a biological sample. In some embodiments, the resulting amplicons are used to determine the nucleotide sequences of the BCR and/or TCR CDRs expressed in the sample. In some embodiments, determining the nucleotide sequences of such amplicons comprising at least 3 CDRs is used to identify and characterize novel BCR and/or TCR alleles.

In some embodiments, a multiplex amplification reaction is used to amplify BCR and/or TCR genomic DNA having undergone V(D)J rearrangement. In some embodiments, a multiplex amplification reaction is used to amplify nucleic acid molecule(s) comprising at least a portion of a BCR and/or TCR CDR from gDNA derived from a biological sample. In some embodiments, a multiplex amplification reaction is used to amplify nucleic acid molecule(s) comprising at least two CDRs of a BCR and/or TCR from gDNA derived from a biological sample. In some embodiments, a multiplex amplification reaction is used to amplify nucleic acid molecules comprising at least three CDRs of a BCR and/or TCR from gDNA derived from a biological sample. In some embodiments, the resulting amplicons are used to determine the nucleotide sequences of the rearranged BCR and/or TCR CDRs in the sample. In some embodiments, determining the nucleotide sequences of such amplicons comprising at least CDR3 is used to identify and characterize novel BCR and/or TCR alleles

In some embodiments of the multiplex amplification reactions, each primer set used target a same BCR or TCR region however the different primers in the set permit targeting the gene's different V(D)J gene rearrangements. For example, the primer set for amplification of the expressed IgH or the rearranged IgH gDNA are all designed to target the same region(s) from IgH mRNA or IgH gDNA, respectively, but the individual primers in the set lead to amplification of the various IgH VDJ gene combinations. In some embodiments, at least one primer or primer set is directed to a relatively conserved region (eg, a portion of the C gene) of an immune receptor gene and the other primer set includes a variety of primers directed to a more variable region of the same gene (eg, a portion of the V gene). In other embodiments, at least one primer set includes a variety of primers directed to at least a portion of J gene segments of an immune receptor gene and the other primer set includes a variety of primers directed to at least a portion of V gene segments of the same gene.

In some embodiments, a multiplex amplification reaction is used to amplify cDNA derived from mRNA expressed from rearranged BCR genomic DNA, including rearranged IgH, IgK, and IgL genomic DNA. In some embodiments, at least a portion of a BCR CDR, for example CDR3, is amplified from cDNA in a multiplex amplification reaction. In some embodiments, at least two CDR portions of BCR are amplified from cDNA in a multiplex amplification reaction. In certain embodiments, a multiplex amplification reaction is used to amplify at least the CDR1, CDR2, and CDR3 regions of a BCR cDNA. In some embodiments, the resulting amplicons are used to determine the expressed BCR CDR nucleotide sequence. In some embodiments, the resulting amplicons are used to determine the expressed BCR CDR nucleotide sequence and Ig isotype of the sequence. In some embodiments, the resulting amplicons are used to determine the expressed IgH CDR nucleotide sequence and the Ig isotype and Ig sub-isotype.

In some embodiments, a multiplex amplification reaction is used to amplify rearranged BCR genomic DNA, including rearranged IgH, IgK, and IgL genomic DNA. In some embodiments, at least a portion of a BCR CDR, for example CDR3, is amplified from gDNA in a multiplex amplification reaction. In some embodiments, at least two CDR portions of BCR are amplified from gDNA in a multiplex amplification reaction. In certain embodiments, a multiplex amplification reaction is used to amplify at least the CDR1, CDR2, and CDR3 regions of a rearranged BCR gDNA. In some embodiments, the resulting amplicons are used to determine the rearranged BCR CDR nucleotide sequence. In some embodiments, the resulting amplicons are used to determine the rearranged BCR CDR nucleotide sequence and Ig isotype of the sequence.

In some embodiments, multiplex amplification reactions are performed with primer sets designed to generate amplicons which include the expressed CDR1, CDR2, and/or CDR3 regions of the target immune receptor mRNA. In some embodiments, multiplex amplification reactions are performed using (i) one set of primers in which each primer is directed to at least a portion of the framework region FR1 of a V gene and (ii) at least one primer directed to a portion of at least one C gene of the target immune receptor. In other embodiments, multiplex amplification reactions are performed using (i) one set of primers in which each primer is directed to at least a portion of the framework region FR2 of a V gene and (ii) at least one primer directed to a portion of at least one C gene of the target immune receptor. In other embodiments, multiplex amplification reactions are performed using (i) one set of primers in which each primer is directed to at least a portion of the framework region FR3 of a V gene and (ii) at least one primer directed to a portion of at least one C gene of the target immune receptor. In some embodiments, multiplex amplification reactions are performed with primer sets designed to generate amplicons which include one or more expressed IgH isotypes of the target mRNA and such reactions are performed using (i) one of the FR1, FR2, or FR3 primer sets noted above and (ii) a set of primers in which each primer is directed to a portion of at least one C gene of IgA, IgD, IgE, IgG, and/or IgM. In some embodiments, the C gene-directed primer(s) is directed C gene coding sequences within about 200 nucleotides of the 5′ end of the C gene(s). In some embodiments, the C gene-directed primer(s) is directed C gene coding sequences within about 150 nucleotides of the 5′ end of the C gene(s). In some embodiments, the C gene-directed primer(s) is directed C gene coding sequences within about 100 nucleotides of the 5′ end of the C gene(s). In some embodiments, the C gene-directed primer(s) is directed C gene coding sequences within about 50 nucleotides, within about 50 to about 150, within about 75 to about 175, or within about 100 to about 200 nucleotides of the 5′ end of the C gene(s). In some embodiments, the C gene-directed primer(s) is directed to C gene coding sequencing which not only distinguishes the isotype but also permits determination of a sub-isotype. For example, in some embodiments, the C gene-directed primer(s) generates sufficient portions of the constant region in the amplicon so that a sub-isotype can be determined based on the determined sequence data. In some embodiments, the C gene-directed primer(s) include primers which are directed to IgG and/or IgA C gene coding sequences which permit identification of IgG1, IgG2, IgG3, IgG4, IgA1, and IgA2 sub-isotypes.

In some embodiments, the multiplex amplification reaction uses (i) a set of primers each of which anneals to at least a portion of the V gene FR1 region and (ii) at least one primer which anneals to a portion of the constant (C) gene to amplify BCR cDNA such that the resultant amplicons include the CDR1, CDR2, and CDR3 coding portions of the BCR mRNA. In certain embodiments, an FR1-directed primer set is combined with a set of at least two C gene-directed primers to generate amplicons which include at least the CDR1, CDR2, and CDR 3 coding portions of a BCR mRNA. In some embodiments, an IgH FR1-directed primer set is combined with a set of at least two C gene primers directed to coding portions of two different IgH isotypes to generate amplicons which include at least the CDR1, CDR2, and CDR 3 coding portions of IgH mRNA. In some embodiments, an IgH FR1-directed primer set is combined with at least three, at least four, or at least five primers directed to coding portions of different IgH isotypes. For example, exemplary primers specific for IgH V gene FR1 regions are shown in Table 3 and exemplary primers specific for IgH C genes are shown in Tables 6-10.

In some embodiments, the multiplex amplification reaction uses (i) a set of primers each of which anneals to at least a portion of the V gene FR2 region and (ii) at least one primer which anneals to a portion of the C gene to amplify BCR cDNA such that the resultant amplicons include the CDR2 and CDR3 coding portions of the BCR mRNA. In certain embodiments, such a FR2-directed primer set is combined with at least two C gene-directed primers to generate amplicons which include the CDR2 and CDR3 coding portions of a BCR mRNA. In some embodiments, an IgH FR2-directed primer set is combined with a set of at least two C gene primers directed to coding portions of two different IgH isotypes to generate amplicons with the CDR2 and CDR 3 coding portion portions of IgH mRNA. In some embodiments, an IgH FR2-directed primer set is combined with at least three, at least four, or at least five C gene primers directed to coding portions of different IgH isotypes. Exemplary FR2-directed primers include the BIOMED-2 primers developed and standardized by a consortium of European academic laboratories and research hospitals (van Dongen et al. (2003) Leukemia 17:2257-2327) and shown in Table 4. Exemplary primers specific for IgH C genes are shown in Tables 6-10.

In some embodiments, the multiplex amplification reaction uses (i) a set of primers each of which anneals to at least a portion of the V gene FR3 region and (ii) at least one primer which anneals to a portion of the C gene to amplify BCR cDNA such that the resultant amplicons include primarily the CDR3 coding portion of the BCR mRNA. In certain embodiments, such a FR3-directed primer set is combined with at least two C gene-directed primers to generate amplicons with the CDR 3 coding portion of a BCR mRNA. In some embodiments, an IgH FR3-directed primer set is combined with a set of at least two C gene primers directed to coding portions of two different IgH isotypes to generate amplicons with the CDR 3 coding portion of IgH mRNA. In some embodiments, an IgH FR3-directed primer set is combined with at least three, at least four, or at least five C gene primers directed to coding portions of different IgH isotypes. For example, exemplary primers specific for IgH V gene FR3 regions are shown in Table 2 and exemplary primers specific for IgH C genes are shown in Tables 6-10.

In some embodiments, multiplex amplification reactions are performed with primer sets designed to generate amplicons which include the CDR1, CDR2, and/or CDR3 regions of the target immune receptor mRNA or rearranged gDNA. In some embodiments, multiplex amplification reactions are performed using (i) one set of primers in which each primer is directed to at least a portion of the framework region FR1 of a V gene and (ii) one set of primers in which each primer is directed to at least a portion of the J gene of the target immune receptor. In other embodiments, multiplex amplification reactions are performed using (i) one set of primers in which each primer is directed to at least a portion of the framework region FR2 of a V gene and (ii) one set of primers in which each primer is directed to at least a portion of the J gene of the target immune receptor. In other embodiments, multiplex amplification reactions are performed using (i) one set of primers in which each primer is directed to at least a portion of the framework region FR3 of a V gene and (ii) one set of primers in which each primer is directed to at least a portion of the J gene of the target immune receptor.

In some embodiments, the multiplex amplification reaction uses (i) a set of primers each of which anneals to at least a portion of the V gene FR1 region and (ii) a set of primers which anneal to a portion of the J gene to amplify BCR nucleic acid such that the resultant amplicons include the CDR1, CDR2, and CDR3 coding portions of the BCR mRNA or rearranged gDNA. For example, exemplary primers specific for IgH V gene FR1 regions are shown in Table 3 and exemplary primers specific for IgH J genes are shown in Table 5.

In some embodiments, the multiplex amplification reaction uses (i) a set of primers each of which anneals to at least a portion of the V gene FR2 region and (ii) a set of primers which anneal to a portion of the J gene to amplify BCR nucleic acid such that the resultant amplicons include the CDR2 and CDR3 coding portions of the BCR mRNA or rearranged gDNA. For example, exemplary primers specific for IgH V gene FR2 regions are shown in Table 4 and exemplary primers specific for IgH J genes are shown in Table 5.

In some embodiments, the multiplex amplification reaction uses (i) a set of primers each of which anneals to at least a portion of the V gene FR3 region and (ii) a set of primers which anneal to a portion of the J gene to amplify BCR nucleic acid such that the resultant amplicons include primarily the CDR3 coding portion of the BCR mRNA or rearranged gDNA. For example, exemplary primers specific for the IgH V gene FR3 regions are shown in Table 2 and exemplary primers specific for IgH J genes are shown in Table 5.

In some embodiments, provided are compositions for multiplex amplification of at least a portion of an expressed BCR variable region. In some embodiments, the composition comprises a plurality of sets of primer pair reagents directed to a portion of a V gene framework region and a portion of a constant (C) gene of rearranged target immune receptor genes selected from the group consisting of immunoglobulin heavy chain (IgH), immunoglobulin light chain lambda (IgL), and immunoglobulin light chain kappa (IgK). In some embodiments, the composition comprises a plurality of sets of primer pair reagents directed to a portion of a V gene framework region and a portion of a J gene of rearranged target immune receptor genes selected from the group consisting of IgH, IgL, and IgK.

In some embodiments, the composition comprises (i) a plurality of sets of primer pair reagents directed to a portion of an IgH V gene framework region and a portion of an IgH C gene of rearranged IgH genes and (ii) a plurality of sets of primer pair reagents directed to a portion of a TCR beta V gene framework region and a portion of a TCR beta C gene of rearranged TCR beta genes. In some embodiments, the composition comprises (i) a plurality of sets of primer pair reagents directed to a portion of an IgH V gene framework region and a portion of an IgH J gene of rearranged IgH genes and (ii) a plurality of sets of primer pair reagents directed to a portion of a TCR beta V gene framework region and a portion of a TCR beta J gene of rearranged TCR beta genes.

Amplification by PCR is performed with at least two primers. For the methods provided herein, a set of primers is used that is sufficient to amplify all or a defined portion of the variable sequences at the locus of interest, which locus may include any or all of the aforementioned TCR and Immunoglobulin loci. In some embodiments, various parameters or criteria outlined herein may be used to select the set of target-specific primers for the multiplex amplification.

In some embodiments, primer sets used in the multiplex reactions are designed to amplify at least 50% of the known expressed or gDNA rearrangements at the locus of interest. In certain embodiments, primer sets used in the multiplex reactions are designed to amplify at least 75%, at least 85%, at least 90%, at least 95%, at least 97%, at least 98% or more of the known expressed or gDNA rearrangements at the locus of interest. For another example, use of 27 forward primers of Table 3, each directed to a portion of the FR1 region from different IgH V genes, in combination with at least one reverse primer of Tables 6-10, each directed to a portion of different IgH C genes, will amplify all of the currently known expressed IgH rearrangements for a given isotype. For another example, use of 68 forward primers of Table 2, each directed to a portion of the FR3 region from different IgH V genes, in combination with at least one reverse primer of Tables 6-10, each directed to a portion of different IgH C genes, will amplify all of the currently known expressed IgH rearrangements for a given isotype. For another example, use of 68 forward primers of Table 2, each directed to a portion of the FR3 region from different IgH V genes, in combination with 4 reverse primers of Table 5, each directed to a portion of different IgH J genes, will amplify all of the currently known expressed or gDNA IgH rearrangements. For another example, use of 27 forward primers of Table 3, each directed to a portion of the FR1 region from different IgH V genes, in combination with 4 reverse primers of Table 5, each directed to a portion of different IgH J genes, will amplify all of the currently known expressed or gDNA IgH rearrangements.

For example, such a multiplex amplification reaction includes at least 15, 20, 25, 30, 35, 40, 45, 50, 55, 60, 65, 70, 75, 80, 85, or 90, preferably 22, 23, 24, 25, 26, 27, 28, 29, 30, 34, 38, 42, 46, 50, 54, 58, or 62 reverse primers in which each reverse primer is directed to a sequence corresponding to at least a portion of one or more BCR V gene FR1 regions. In such embodiments, the plurality of reverse primers directed to the BCR V gene FR1 regions is combined with at least 1 forward primer directed to a sequence corresponding to at least a portion of a constant gene of the same BCR gene. In some embodiments, the plurality of reverse primers directed to the BCR V gene FR1 regions is combined with at least 2, at least 3, at least 4, at least 5, at least 6, at least 7, at least 8, at least 9, at least 10, at least 11, at least 12, at least 15, or about 2 to about 7, about 5 to about 20, about 5 to about 15, or about 7 to about 12 forward primers each directed to a sequence corresponding to at least a portion of at least one of the constant genes of the same BCR gene. In some embodiments of the multiplex amplification reactions, the BCR V gene FR1 directed primers may be the forward primers and the BCR C gene-directed primer(s) may be the reverse primer(s). Accordingly, in some embodiments, a multiplex amplification reaction includes at least 15, 20, 25, 30, 35, 40, 45, 50, 55, 60, 65, 70, 75, 80, 85, or 90, preferably 22, 23, 24, 25, 26, 27, 28, 29, 30, 34, 38, 42, 46, 50, 54, 58, or 62 forward primers in which each forward primer is directed to a sequence corresponding to at least a portion of one or more BCR V gene FR1 regions. In such embodiments, the plurality of forward primers directed to the BCR V gene FR1 regions is combined with at least 1 reverse primer directed to a sequence corresponding to at least a portion of a C gene of the same BCR gene. In some embodiments, the plurality of forward primers directed to the BCR V gene FR1 regions is combined with at least 2, at least 3, at least 4, at least 5, at least 6, at least 7, at least 8, at least 9, at least 10, at least 11, at least 12, at least 15, or about 2 to about 7, about 5 to about 20, about 5 to about 15, or about 7 to about 12 reverse primers each directed to a sequence corresponding to at least a portion of at least one of the C genes of the same BCR gene. In some embodiments, such FR1 and C gene amplification primer sets may be directed to IgH gene sequences. In some preferred embodiments, about 22 to about 35 reverse primers directed to different IgH V gene FR1 regions are combined with about 2 to about 8 forward primers directed to a portion of the IgH C genes. In other preferred embodiments, about 22 to about 35 reverse primers directed to different IgH V gene FR1 regions are combined with about 5 to about 15 forward primers directed to a portion of the IgH C genes. In other preferred embodiments, about 48 to about 60 reverse primers directed to different IgH V gene FR1 regions are combined with about 5 to about 15 forward primers directed to a portion of the IgH C genes. In some preferred embodiments, about 22 to about 35 forward primers directed to different IgH V gene FR1 regions are combined with about 2 to about 8 reverse primers directed to a portion of the IgH C genes. In other preferred embodiments, about 22 to about 35 forward primers directed to different IgH V gene FR1 regions are combined with about 5 to about 15 reverse primers directed to a portion of the IgH C genes. In yet other preferred embodiments, about 48 to about 60 forward primers directed to different IgH V gene FR1 regions are combined with about 5 to about 15 reverse primers directed to a portion of the IgH C genes. In some preferred embodiments, the forward primers directed to IgH V gene FR1 regions are selected from those listed in Table 3 and the reverse primers directed to the IgH C genes are selected from those listed in Tables 6-10. In other embodiments, the FR1 and C gene amplification primer sets may be directed to Ig light chain lambda, Ig light chain kappa, TCR alpha, TCR gamma, TCR delta, or TCR beta gene sequences.

In some embodiments, a multiplex amplification reaction includes at least 5, 10, 15, 20, 25, 30, 40, 50, 60, 70, 80, or 90 reverse primers in which each reverse primer is directed to a sequence corresponding to at least a portion of one or more BCR V gene FR2 regions. In such embodiments, the plurality of reverse primers directed to the BCR V gene FR2 regions is combined with at least 1 forward primer directed to a sequence corresponding to at least a portion of a C gene of the same BCR gene. In some embodiments, the plurality of reverse primers directed to the BCR V gene FR2 regions is combined with at least 2, at least 3, at least 4, at least 5, at least 6, at least 7, at least 8, at least 9, at least 10, at least 11, at least 12, at least 15, or about 2 to about 7, about 5 to about 20, about 5 to about 15, or about 7 to about 12 forward primers each directed to a sequence corresponding to at least a portion of at least one of the C genes of the same BCR gene. In some embodiments of the multiplex amplification reactions, the BCR V gene FR2 directed primers may be the forward primers and the BCR C gene-directed primer(s) may be the reverse primer(s). Accordingly, in some embodiments, a multiplex amplification reaction includes at least 5, 10, 15, 20, 25, 30, 40, 50, 60, 70, 80, or 90 forward primers in which each forward primer is directed to a sequence corresponding to at least a portion of one or more BCR V gene FR2 regions. In such embodiments, the plurality of forward primers directed to the BCR V gene FR2 regions is combined with at least 1 reverse primer directed to a sequence corresponding to at least a portion of a C gene of the same BCR gene. In some embodiments, the plurality of forward primers directed to the BCR V gene FR2 regions is combined with at least 2, at least 3, at least 4, at least 5, at least 6, at least 7, at least 8, at least 9, at least 10, at least 11, at least 12, at least 15, or about 2 to about 7, about 5 to about 20, about 5 to about 15, or about 7 to about 12 reverse primers each directed to a sequence corresponding to at least a portion of at least one of the C genes of the same BCR gene. In some embodiments, such FR2 and C gene amplification primer sets may be directed to IgH gene sequences. In some embodiments, about 5 to about 15 reverse primers directed to different IgH V gene FR2 regions are combined with about 2 to about 8 forward primers directed to a portion of the IgH C gene. In some embodiments, about 5 to about 15 reverse primers directed to different IgH V gene FR2 regions are combined with about 5 to about 15 forward primers directed to a portion of the IgH C gene. In some embodiments, about 5 to about 15 forward primers directed to different IgH V gene FR2 regions are combined with about 2 to about 8 reverse primers directed to a portion of the IgH C gene. In some embodiments, about 5 to about 15 forward primers directed to different IgH V gene FR2 regions are combined with about 5 to about 15 reverse primers directed to a portion of the IgH C gene. In some preferred embodiments, the forward primers directed to IgH V gene FR2 regions are selected from those listed in Table 4 and the reverse primers directed to the IgH C gene are selected from those listed in Tables 6-10. In other embodiments, the FR2 and C gene amplification primer sets may be directed to Ig light chain lambda, Ig light chain kappa, TCR alpha, TCR gamma, TCR delta, or TCR beta gene sequences.

In some embodiments, a multiplex amplification reaction includes at least 20, 25, 30, 40, 45, preferably 50, 55, 60, 65, 70, 75, 80, 85, or 90 reverse primers in which each reverse primer is directed to a sequence corresponding to at least a portion of one or more BCR V gene FR3 regions. In such embodiments, the plurality of reverse primers directed to the BCR V gene FR3 regions is combined with at least 1 forward primer directed to a sequence corresponding to at least a portion of a C gene of the same BCR gene. In some embodiments, the plurality of reverse primers directed to the BCR V gene FR3 regions is combined with at least 2, at least 3, at least 4, at least 5, at least 6, at least 7, at least 8, at least 9, at least 10, at least 11, at least 12, at least 15, or about 2 to about 7, about 5 to about 20, about 5 to about 15, or about 7 to about 12 forward primers each directed to a sequence corresponding to at least a portion of at least one of the C genes of the same BCR gene. In some embodiments of the multiplex amplification reactions, the BCR V gene FR3 directed primers may be the forward primers and the BCR C gene-directed primer(s) may be the reverse primer(s). Accordingly, in some embodiments, a multiplex amplification reaction includes at least 20, 25, 30, 40, 45, preferably 50, 55, 60, 65, 70, 75, 80, 85, or 90 reverse primers in which each forward primer is directed to a sequence corresponding to at least a portion of one or more BCR V gene FR3 regions. In such embodiments, the plurality of forward primers directed to the BCR V gene FR3 regions is combined with at least 1 reverse primer directed to a sequence corresponding to at least a portion of a C gene of the same BCR gene. In some embodiments, the plurality of forward primers directed to the BCR V gene FR3 regions is combined with at least 2, at least 3, at least 4, at least 5, at least 6, at least 7, at least 8, at least 9, at least 10, at least 11, at least 12, at least 15, or about 2 to about 7, about 5 to about 20, about 5 to about 15, or about 7 to about 12 reverse primers each directed to a sequence corresponding to at least a portion of at least one of the C genes of the same BCR gene. In some embodiments, such FR3 and C gene amplification primer sets may be directed to IgH gene sequences. In some preferred embodiments, about 62 to about 75 reverse primers directed to different IgH V gene FR3 regions are combined with about 2 to about 8 forward primers directed to a portion of IgH C genes. In other preferred embodiments, about 62 to about 75 reverse primers directed to different IgH V gene FR3 regions are combined with about 5 to about 15 forward primers directed to a portion of IgH C genes. In some preferred embodiments, about 62 to about 75 forward primers directed to different IgH V gene FR3 regions are combined with about 2 to about 8 reverse primers directed to a portion of IgH C genes. In other preferred embodiments, about 62 to about 75 forward primers directed to different IgH V gene FR3 regions are combined with about 5 to about 15 reverse primers directed to a portion of IgH C genes. In some preferred embodiments, the forward primers directed to IgH V gene FR3 regions are selected from those listed in Table 2 and the reverse primers directed to the IgH C gene are selected from those listed in Tables 6-10. In other embodiments, the FR3 and C gene amplification primer sets may be directed to Ig light chain lambda, Ig light chain kappa, TCR alpha, TCR gamma, TCR delta, and TCR beta gene sequences.

In some embodiments, such a multiplex amplification reaction includes at least 15, 20, 25, 30, 35, 40, 45, 50, 55, 60, 65, 70, 75, 80, 85, or 90, preferably 22, 23, 24, 25, 26, 27, 28, 29, 30, 34, 38, 42, 46, 50, 54, 58, or 62 reverse primers in which each reverse primer is directed to a sequence corresponding to at least a portion of one or more BCR V gene FR1 regions. In such embodiments, the plurality of reverse primers directed to the BCR V gene FR1 regions is combined with at least 2, 3, 4, 5, 6, 8, or about 3-6 forward primers directed to a sequence corresponding to at least a portion of a J gene of the same BCR gene. In some embodiments of the multiplex amplification reactions, the BCR V gene FR1-directed primers may be the forward primers and the BCR J gene-directed primers may be the reverse primers. Accordingly, in some embodiments, a multiplex amplification reaction includes at least 15, 20, 25, 30, 35, 40, 45, 50, 55, 60, 65, 70, 75, 80, 85, or 90, preferably 22, 23, 24, 25, 26, 27, 28, 29, 30, 34, 38, 42, 46, 50, 54, 58, or 62 forward primers in which each forward primer is directed to a sequence corresponding to at least a portion of one or more BCR V gene FR1 regions. In such embodiments, the plurality of forward primers directed to the BCR V gene FR1 regions is combined with at least 2, 3, 4, 5, 6, 8, or about 3-6 reverse primers directed to a sequence corresponding to at least a portion of a J gene of the same BCR gene. In some embodiments, such FR1 and J gene amplification primer sets may be directed to IgH gene sequences. In some preferred embodiments, about 22 to about 35 reverse primers directed to different IgH V gene FR1 regions are combined with about 3 to about 6 forward primers directed to different IgH J genes. In some preferred embodiments, about 22 to about 35 forward primers directed to different IgH V gene FR1 regions are combined with about 3 to about 6 reverse primers directed to different IgH J genes. In some preferred embodiments, the forward primers directed to IgH V gene FR1 regions are selected from those listed in Table 3 and the reverse primers directed to the IgH J gene are selected from those listed in Table 5. In other embodiments, the FR1 and J gene amplification primer sets may be directed to Ig light chain lambda, Ig light chain kappa, TCR alpha, TCR gamma, TCR delta, or TCR beta gene sequences.

In some embodiments, a multiplex amplification reaction includes at least 5, 10, 15, 20, 25, 30, 40, 50, 60, 70, 80, or 90 reverse primers in which each reverse primer is directed to a sequence corresponding to at least a portion of one or more BCR V gene FR2 regions. In such embodiments, the plurality of reverse primers directed to the BCR V gene FR2 regions is combined with at least 2, 3, 4, 5, 6, 8, or about 3-6 forward primers directed to a sequence corresponding to at least a portion of a J gene of the same BCR gene. In some embodiments of the multiplex amplification reactions, the BCR V gene FR2-directed primers may be the forward primers and the BCR J gene-directed primers may be the reverse primers. Accordingly, in some embodiments, a multiplex amplification reaction includes at least 5, 10, 15, 20, 25, 30, 40, 50, 60, 70, 80, or 90 forward primers in which each forward primer is directed to a sequence corresponding to at least a portion of one or more BCR V gene FR2 regions. In such embodiments, the plurality of forward primers directed to the BCR V gene FR2 regions is combined with at least 2, 3, 4, 5, 6, 8, or about 3-6 reverse primers directed to a sequence corresponding to at least a portion of a J gene of the same BCR gene. In some embodiments, such FR2 and J gene amplification primer sets may be directed to IgH gene sequences. In some preferred embodiments, about 5 to about 15 reverse primers directed to different IgH V gene FR2 regions are combined with about 3 to about 6 forward primers directed to different IgH J genes. In some preferred embodiments, about 5 to about 15 forward primers directed to different IgH V gene FR2 regions are combined with about 3 to about 6 reverse primers directed to different IgH J genes. In some preferred embodiments, the forward primers directed to IgH V gene FR2 regions are selected from those listed in Table 4 and the reverse primers directed to the IgH J gene are selected from those listed in Table 5. In other embodiments, the FR2 and J gene amplification primer sets may be directed to Ig light chain lambda, Ig light chain kappa, TCR alpha, TCR gamma, TCR delta, or TCR beta gene sequences.

In some embodiments, a multiplex amplification reaction includes at least 20, 25, 30, 40, 45, preferably 50, 55, 60, 65, 70, 75, 80, 85, or 90 reverse primers in which each reverse primer is directed to a sequence corresponding to at least a portion of one or more BCR V gene FR3 regions. In such embodiments, the plurality of reverse primers directed to the BCR V gene FR3 regions is combined with at least 2, 3, 4, 5, 6, 8, or about 3-6 forward primers directed to a sequence corresponding to at least a portion of a J gene of the same BCR gene. In some embodiments of the multiplex amplification reactions, the BCR V gene FR3-directed primers may be the forward primers and the BCR J gene-directed primers may be the reverse primers. Accordingly, in some embodiments, a multiplex amplification reaction includes at least 20, 25, 30, 40, 45, preferably 50, 55, 60, 65, 70, 75, 80, 85, or 90 forward primers in which each forward primer is directed to a sequence corresponding to at least a portion of one or more BCR V gene FR3 regions. In such embodiments, the plurality of forward primers directed to the BCR V gene FR3 regions is combined with at least 2, 3, 4, 5, 6, 8, or about 3-6 reverse primers directed to a sequence corresponding to at least a portion of a J gene of the same BCR gene. In some embodiments, such FR3 and J gene amplification primer sets may be directed to IgH gene sequences. In some preferred embodiments, about 62 to about 75 reverse primers directed to different IgH V gene FR3 regions are combined with about 3 to about 6 forward primers directed to different IgH J genes. In some preferred embodiments, about 62 to about 75 forward primers directed to different IgH V gene FR3 regions are combined with about 3 to about 6 reverse primers directed to different IgH J genes. In some preferred embodiments, the forward primers directed to IgH V gene FR3 regions are selected from those listed in Table 2 and the reverse primers directed to the IgH J gene are selected from those listed in Table 5. In other embodiments, the FR3 and J gene amplification primer sets may be directed to Ig light chain lambda, Ig light chain kappa, TCR alpha, TCR gamma, TCR delta, and TCR beta gene sequences.

In some embodiments, the concentration of the forward primer is about equal to that of the reverse primer in a multiplex amplification reaction. In other embodiments, the concentration of the forward primer is about twice that of the reverse primer in a multiplex amplification reaction. In other embodiments, the concentration of the forward primer is about half that of the reverse primer in a multiplex amplification reaction. In some embodiments, the concentration of each of the primers targeting the V gene FR region is about 5 nM to about 2000 nM. In some embodiments, the concentration of each of the primers targeting the V gene FR region is about 50 nM to about 800 nM. In some embodiments, the concentration of each of the primers targeting the V gene FR region is about 50 nM to about 400 nM or about 100 nM to about 500 nM. In some embodiments, the concentration of each of the primers targeting the V gene FR region is about 200 nM, about 400 nM, about 600 nM, or about 800 nM. In some embodiments, the concentration of each of the primers targeting the V gene FR region is about 5 nM, about 10 nM, about 50 nM, about 100 nM, about 150 nM. In some embodiments, the concentration of each of the primers targeting the V gene FR region is about 1000 nM, about 1250 nM, about 1500 nM, about 1750 nM, or about 2000 nM. In some embodiments, the concentration of each of the primers targeting the V gene FR region is about 50 nM to about 800 nM. In some embodiments, the concentration of each of the primers targeting the J gene is about 5 nM to about 2000 nM. In some embodiments, the concentration of each of the primers targeting the J gene is about 50 nM to about 800 nM. In some embodiments, the concentration of each of the primers targeting the J gene is about 50 nM to about 400 nM or about 100 nM to about 500 nM. In some embodiments, the concentration of each of the primers targeting the J gene is about 200 nM, about 400 nM, about 600 nM, or about 800 nM. In some embodiments, the concentration of each of the primers targeting the J gene is about 5 nM, about 10 nM, about 50 nM, about 100 nM, about 150 nM. In some embodiments, the concentration of each of the primers targeting the J gene is about 1000 nM, about 1250 nM, about 1500 nM, about 1750 nM, or about 2000 nM. In some embodiments, the concentration of each of the primers targeting the J gene is about 50 nM to about 800 nM. In some embodiments, the concentration of each of the primers targeting the C gene is about 5 nM to about 2000 nM. In some embodiments, the concentration of each of the primers targeting the C gene is about 50 nM to about 800 nM. In some embodiments, the concentration of each of the primers targeting the C gene is about 50 nM to about 400 nM or about 100 nM to about 500 nM. In some embodiments, the concentration of each of the primers targeting the C gene is about 200 nM, about 400 nM, about 600 nM, or about 800 nM. In some embodiments, the concentration of each of the primers targeting the C gene is about 5 nM, about 10 nM, about 50 nM, about 100 nM, about 150 nM. In some embodiments, the concentration of each of the primers targeting the C gene is about 1000 nM, about 1250 nM, about 1500 nM, about 1750 nM, or about 2000 nM. In some embodiments, the concentration of each of the primers targeting the C gene is about 50 nM to about 800 nM. In some embodiments, the concentration of each forward and reverse primer in a multiplex reaction is about 50 nM, about 100 nM, about 200 nM, or about 400 nM. In some embodiments, the concentration of each forward and reverse primer in a multiplex reaction is about 5 nM to about 2000 nM. In some embodiments, the concentration of each forward and reverse primer in a multiplex reaction is about 50 nM to about 800 nM. In some embodiments, the concentration of each forward and reverse primer in a multiplex reaction is about 50 nM to about 400 nM or about 100 nM to about 500 nM. In some embodiments, the concentration of each forward and reverse primer in a multiplex reaction is about 600 nM, about 800 nM, about 1000 nM, about 1250 nM, about 1500 nM, about 1750 nM, or about 2000 nM. In some embodiments, the concentration of each forward and reverse primer in a multiplex reaction is about 5 nM, about 10 nM, about 150 nM or 50 nM to about 800 nM.

In some embodiments, the V gene FR and C gene target-directed primers combine as amplification primer pairs to amplify target immune receptor cDNA sequences and generate target amplicons. Generally, the length of a target amplicon will depend upon which V gene primer set (eg, FR1, FR2, or FR3 directed primers) is paired with the C gene primer(s). Accordingly, in some embodiments, target amplicons can range from about 100 nucleotides (or bases or base pairs) in length to about 600 nucleotides (or bases or base pairs) in length. In some embodiments, target amplicons can range from about 80 nucleotides to about 600 nucleotides in length. In some embodiments, target amplicons are from about 200 to about 600 or about 300 to about 600 nucleotides in length. In some embodiments, target amplicons are about 80 to about 140, about 90 to about 130, or about 100 to about 120 nucleotides in length. In some embodiments, target amplicons are about 250 to about 275, about 250 to about 350, about 300 to about 350, about 310 to about 330, about 325 to about 375, about 300 to about 400, about 350 to about 400, about 350 to about 425, about 350 to about 450, about 380 to about 410, about 375 to about 425, about 400 to about 500, about 425 to about 500, about 450 to about 550, about 500 to about 600, about 400 to about 500, or about 400 to about 600 nucleotides in length. In some embodiments, target amplicons are about 80, about 100, about 120, about 140, about 200, about 250, about 275, about 300, about 320, about 350, about 375, about 400, about 425, about 450, about 500, about 550, or about 600 nucleotides in length. In some embodiments, IgH amplicons are about 100, about 80 to about 140, about 90 to about 130, or about 100 to about 120 nucleotides in length. In some embodiments, IgH amplicons are about 320, about 300 to about 350 or about 310 to about 330 nucleotides in length. In some embodiments, IgH amplicons are about 400, about 375 to about 425 or about 390 to about 410 nucleotides in length.

In some embodiments, the V gene FR and J gene target-directed primers combine as amplification primer pairs to amplify target immune receptor cDNA or rearranged gDNA sequences and generate target amplicons. Generally, the length of a target amplicon will depend upon which V gene primer set (eg, FR1, FR2, or FR3 directed primers) is paired with the J gene primers. Accordingly, in some embodiments, target amplicons can range from about 50 nucleotides to about 350 nucleotides in length. In some embodiments, target amplicons are about 50 to about 200, about 70 to about 170, about 200 to about 350, about 250 to about 320, about 270 to about 300, about 225 to about 300, about 250 to about 275, about 200 to about 235, about 200 to about 250, or about 175 to about 275 nucleotides in length. In some embodiments, IgH amplicons are about 80, about 60 to about 100, or about 70 to about 90 nucleotides in length. In some embodiments, IgH amplicons, such as those generated using V gene FR3- and J gene-directed primer pairs, are about 50 to about 200 nucleotides in length, preferably about 60 to about 160, about 65 to about 120, about 90 to about 120, about 70 to about 90 nucleotides, or about 80 nucleotides in length. In some embodiments, generating amplicons of such short lengths allows the provided methods and compositions to effectively detect and analyze the immune repertoire from highly degraded gDNA template material, such as that derived from an FFPE sample or cell-free DNA (cfDNA).

In some embodiments, amplification primers may include a barcode sequence, for example to distinguish or separate a plurality of amplified target sequences in a sample. In some embodiments, amplification primers may include two or more barcode sequences, for example to distinguish or separate a plurality of amplified target sequences in a sample. In some embodiments, amplification primers may include a tagging sequence that can assist in subsequent cataloguing, identification or sequencing of the generated amplicon. In some embodiments, the barcode sequence(s) or the tagging sequence(s) is incorporated into the amplified nucleotide sequence through inclusion in the amplification primer or by ligation of an adapter. Primers may further comprise nucleotides useful in subsequent sequencing, e.g. pyrosequencing. Such sequences are readily designed by commercially available software programs or companies.

In some embodiments, multiplex amplification is performed with target-directed amplification primers which do not include a tagging sequence. In other embodiments, multiplex amplification is performed with amplification primers each of which include a target-directed sequence and a tagging sequence such as, for example, the forward primer or primer set includes tagging sequence 1 and the reverse primer or primer set includes tagging sequence 2. In still other embodiments, multiplex amplification is performed with amplification primers where one primer or primer set includes target directed sequence and a tagging sequence and the other primer or primer set includes a target-directed sequence but does not include a tagging sequence, such as, for example, the forward primer or primer set includes a tagging sequence and the reverse primer or primer set does not include a tagging sequence.

Accordingly, in some embodiments, a plurality of target cDNA or gDNA template molecules are amplified in a single multiplex amplification reaction mixture with BCR and/or TCR directed amplification primers in which the forward and/or reverse primers include a tagging sequence and the resultant amplicons include the target BCR and/or TCR sequence and a tagging sequence on one or both ends. In some embodiments, the forward and/or reverse amplification primer or primer sets may also include a barcode and the one or more barcode is then included in the resultant amplicon.

In some embodiments, a plurality of target cDNA or gDNA template molecules are amplified in a single multiplex amplification reaction mixture with BCR and/or TCR directed amplification primers and the resultant amplicons contain only BCR and/or TCR sequences. In some embodiments, a tagging sequence is added to the ends of such amplicons through, for example, adapter ligation. In some embodiments, a barcode sequence is added to one or both ends of such amplicons through, for example, adapter ligation.

Nucleotide sequences suitable for use as barcodes and for barcoding libraries are known in the art. Adapters and amplification primers and primer sets including a barcode sequence are commercially available. Oligonucleotide adapters containing a barcode sequence are also commercially available including, for example, IonXpress™, IonCode™ and Ion Select barcode adapters (Thermo Fisher Scientific). Similarly, additional and other universal adapter/primer sequences described and known in the art (e.g., Illumina universal adapter/primer sequences, PacBio universal adapter/primer sequences, etc.) can be used in conjunction with the methods and compositions provided herein and the resultant amplicons sequenced using the associated analysis platform.

In some embodiments, two or more barcodes are added to amplicons when sequencing multiplexed samples. In some embodiments, at least two barcodes are added to amplicons prior to sequencing multiplexed samples to reduce the frequency of artefactual results (e.g., immune receptor gene rearrangements or clone identification) derived from barcode cross-contamination or barcode bleed-through between samples. In some embodiments, at least two bar codes are used to label samples when tracking low frequency clones of the immune repertoire. In some embodiments, at least two barcodes are added to amplicons when the assay is used to detect clones of frequency less than 1:1,000. In some embodiments, at least two barcodes are added to amplicons when the assay is used to detect clones of frequency less than 1:10,000. In other embodiments, at least two barcodes are added to amplicons when the assay is used to detect clones of frequency less than 1:20,000, less than 1:40,000, less than 1:100,000, less than 1:200,000, less than 1:400,000, less than 1:500,00, or less than 1:1,000,000. Methods for characterizing the immune repertoire which benefit from a high sequencing depth per clone and/or detection of clones at such low frequencies include, but are not limited to, monitoring a patient with a hyperproliferative disease undergoing treatment and testing for minimal residual disease following treatment.

In some embodiments, target-specific primers (e.g., the V gene FR1-, FR2- and FR3-directed primers, the J gene directed primers, and the C gene directed primers) used in the methods of the invention are selected or designed to satisfy any one or more of the following criteria: (1) includes two or more modified nucleotides within the primer sequence, at least one of which is included near or at the termini of the primer and at least one of which is included at, or about the center nucleotide position of the primer sequence; (2) length of about 15 to about 40 bases in length; (3) Tm of from above 60° C. to about 70° C.; (4) has low cross-reactivity with non-target sequences present in the sample of interest; (5) at least the first four nucleotides (going from 3′ to 5′ direction) are non-complementary to any sequence within any other primer present in the same reaction; and (6) non-complementarity to any consecutive stretch of at least 5 nucleotides within any other produced target amplicon. In some embodiments, the target-specific primers used in the methods provided are selected or designed to satisfy any 2, 3, 4, 5, or 6 of the above criteria.

In some embodiments, the target-specific primers used in the methods of the invention include one or more modified nucleotides having a cleavable group. In some embodiments, the target-specific primers used in the methods of the invention include two or more modified nucleotides having cleavable groups. In some embodiments, the target-specific primers comprise at least one modified nucleotide having a cleavable group selected from methylguanine, 8-oxo-guanine, xanthine, hypoxanthine, 5,6-dihydrouracil, uracil, 5-methylcytosine, thymine-dimer, 7-methylguanosine, 8-oxo-deoxyguanosine, xanthosine, inosine, dihydrouridine, bromodeoxyuridine, uridine or 5-methylcytidine.

In some embodiments, target amplicons using the amplification methods (and associated compositions, systems, and kits) disclosed herein, are used in the preparation of an immune receptor repertoire library. In some embodiments, the immune receptor repertoire library includes introducing adapter sequences to the termini of the target amplicon sequences. In certain embodiments, a method for preparing an immune receptor repertoire library includes generating target immune receptor amplicon molecules according to any of the multiplex amplification methods described herein, treating the amplicon molecule by digesting a modified nucleotide within the amplicon molecules' primer sequences, and ligating at least one adapter to at least one of the treated amplicon molecules, thereby producing a library of adapter-ligated target immune receptor amplicon molecules comprising the target immune receptor repertoire. In some embodiments, the steps of preparing the library are carried out in a single reaction vessel involving only addition steps. In certain embodiments, the method further includes clonally amplifying a portion of the at least one adapter-ligated target amplicon molecule.

In some embodiments, target amplicons using the methods (and associated compositions, systems, and kits) disclosed herein, are coupled to a downstream process, such as but not limited to, library preparation and nucleic acid sequencing. For example, target amplicons can be amplified using bridge amplification, emulsion PCR or isothermal amplification to generate a plurality of clonal templates suitable for nucleic acid sequencing. In some embodiments, the amplicon library is sequenced using any suitable DNA sequencing platform such as any next generation sequencing platform, including semi-conductor sequencing technology such as the Ion Torrent sequencing platform. In some embodiments, an amplicon library is sequenced using an Ion GeneStudio S5 540™ System or an Ion GeneStudio S5 520™ System or an Ion GeneStudio S5 530™ System or an Ion PGM 318™ System.

In some embodiments, sequencing of immune receptor amplicons generated using the methods (and associated compositions and kits) disclosed herein, produces contiguous sequence reads from about 200 to about 600 nucleotides in length. In some embodiments, contiguous read lengths are from about 300 to about 400 nucleotides. In some embodiments, contiguous read lengths are from about 350 to about 450 nucleotides. In some embodiments, read lengths average about 300 nucleotides, about 350 nucleotides, or about 400 nucleotides. In some embodiments, contiguous read lengths are from about 250 to about 350 nucleotides, about 275 to about 340, or about 295 to about 325 nucleotides in length. In some embodiments, read lengths average about 270, about 280, about 290, about 300, or about 325 nucleotides in length. In other embodiments, contiguous read lengths are from about 180 to about 300 nucleotides, about 200 to about 290 nucleotides, about 225 to about 280 nucleotides, or about 230 to about 250 nucleotides in length. In some embodiments, read lengths average about 200, about 220, about 230, about 240, or about 250 nucleotides in length. In other embodiments, contiguous read lengths are from about 70 to about 200 nucleotides, about 80 to about 150 nucleotides, about 90 to about 140 nucleotides, or about 100 to about 120 nucleotides in length. In some embodiments, contiguous read lengths are from about 50 to about 170 nucleotides, about 60 to about 160 nucleotides, about 60 to about 120 nucleotides, about 70 to about 100 nucleotides, about 70 to about 90 nucleotides, or about 80 nucleotides in length. In some embodiments, read lengths average about 70, about 80, about 90, about 100, about 110, or about 120 nucleotides. In some embodiments, the sequence read length include the amplicon sequence and a barcode sequence. In some embodiments, the sequence read length does not include a barcode sequence.

In some embodiments, the amplification primers and primer pairs are target-specific sequences that can amplify specific regions of a nucleic acid molecule. In some embodiments, the target-specific primers can amplify expressed RNA or cDNA. In some embodiments, the target-specific primers can amplify mammalian RNA, such as human RNA or cDNA prepared therefrom, or murine RNA or cDNA prepared therefrom. In some embodiments, the target-specific primers can amplify DNA, such as gDNA. In some embodiments, the target-specific primers can amplify mammalian DNA, such as human DNA or murine DNA.

In methods and compositions provided herein, for example those for determining, characterizing, and/or tracking the immune repertoire in a biological sample, the amount of input RNA or gDNA required for amplification of target sequences will depend in part on the fraction of immune receptor bearing cells (e.g., T cells or B cells) in the sample. For example, a higher fraction of B cells in the sample, such as samples enriched for B cells, permits use of a lower amount of input RNA or gDNA for amplification. In some embodiments, the amount of input RNA for amplification of one or more target sequences can be about 0.05 ng to about 10 micrograms. In some embodiments, the amount of input RNA used for multiplex amplification of one or more target sequences can be from about 5 ng to about 2 micrograms. In some embodiments, the amount of RNA used for multiplex amplification of one or more target sequences can be from about 5 ng to about 1 microgram or about 10 ng to about 1 microgram. In some embodiments, the amount of RNA used for multiplex amplification of one or more immune repertoire target sequences is about 1.5 micrograms, about 2 micrograms, about 2.5 micrograms, about 3 micrograms, about 3.5 micrograms, about 4.0 micrograms, about 5 micrograms, about 6 micrograms, about 7 micrograms, or about 10 micrograms. In some embodiments, the amount of RNA used for multiplex amplification of one or more immune repertoire target sequences is about 10 ng, about 25 ng, about 50 ng, about 100 ng, about 200 ng, about 250 ng, about 500 ng, about 750 ng, or about 1000 ng. In some embodiments, the amount of RNA used for multiplex amplification of one or more immune repertoire target sequences is from about 25 ng to about 500 ng RNA or from about 50 ng to about 200 ng RNA. In some embodiments, the amount of RNA used for multiplex amplification of one or more immune repertoire target sequences is from about 0.05 ng to about 10 ng RNA, from about 0.1 ng to about 5 ng RNA, from about 0.2 ng to about 2 ng RNA, or from about 0.5 ng to about 1 ng RNA. In some embodiments, the amount of RNA used for multiplex amplification of one or more immune repertoire target sequences is about 0.05 ng, about 0.1 ng, about 0.2 ng, about 0.5 ng, about 1.0 ng, about 2.0 ng, or about 5.0 ng.

As described herein, RNA from a biological sample is converted to cDNA, typically using reverse transcriptase in a reverse transcription reaction, prior to the multiplex amplification. In some embodiments, a reverse transcription reaction is performed with the input RNA and a portion of the cDNA from the reverse transcription reaction is used in the multiplex amplification reaction. In some embodiments, substantially all of the cDNA prepared from the input RNA is added to the multiplex amplification reaction. In other embodiments, a portion, such as about 80%, about 75%, about 66%, about 50%, about 33%, or about 25% of the cDNA prepared from the input RNA is added to the multiplex amplification reaction. In other embodiments, about 15%, about 10%, about 8%, about 6%, or about 5% of the cDNA prepared from the input RNA is added to the multiplex amplification reaction.

In some embodiments, the amount of cDNA from a sample added to the multiplex amplification reaction can be about 0.001 ng to about 5 micrograms. In some embodiments, the amount of cDNA used for multiplex amplification of one or more immune repertoire target sequences can be from about 0.01 ng to about 2 micrograms. In some embodiments, the amount of cDNA used for multiplex amplification of one or more target sequences can be from about 0.1 ng to about 1 microgram or about 1 ng to about 0.5 microgram. In some embodiments, the amount of cDNA used for multiplex amplification of one or more immune repertoire target sequences is about 0.5 ng, about 1 ng, about 5 ng, about 10 ng, about 25 ng, about 50 ng, about 100 ng, about 200 ng, about 250 ng, about 500 ng, about 750 ng, or about 1000 ng. In some embodiments, the amount of cDNA used for multiplex amplification of one or more immune repertoire target sequences is from about 0.01 ng to about 10 ng cDNA, from about 0.05 ng to about 5 ng cDNA, from about 0.1 ng to about 2 ng cDNA, or from about 0.01 ng to about 1 ng cDNA. In some embodiments, the amount of cDNA used for multiplex amplification of one or more immune repertoire target sequences is about 0.005 ng, about 0.01 ng, about 0.05 ng, about 0.1 ng, about 0.2 ng, about 0.5 ng, about 1.0 ng, about 2.0 ng, or about 5.0 ng.

In some embodiments, mRNA is obtained from a biological sample and converted to cDNA for amplification purposes using conventional methods. Methods and reagents for extracting or isolating nucleic acid from biological samples are well known and commercially available. In some embodiments, RNA extraction from biological samples is performed by any method described herein or otherwise known to those of skill in the art, e.g., methods involving proteinase K tissue digestion and alcohol-based nucleic acid precipitation, treatment with DNAse to digest contaminating DNA, and RNA purification using silica-gel-membrane technology, or any combination thereof. Exemplary methods for RNA extraction from biological samples using commercially available kits including RecoverAll™ Multi-Sample RNA/DNA Workflow (Invitrogen), RecoverAll™ Total Nucleic Acid Isolation Kit (Invitrogen), NucleoSpin® RNA blood (Macherey-Nagel), PAXgene® Blood RNA system, TRI Reagent™ (Invitrogen), PureLink™ RNA Micro Scale kit (Invitrogen), MagMAX™ FFPE DNA/RNA Ultra Kit (Applied Biosystems) ZR RNA MicroPrep™ kit (Zymo Research), RNeasy Micro kit (Qiagen), and ReliaPrep™ RNA Tissue miniPrep system (Promega).

In some embodiments, the amount of input gDNA for amplification of one or more target sequences can be about 0.1 ng to about 10 micrograms. In some embodiments, the amount of gDNA required for amplification of one or more target sequences can be from about 0.5 ng to about 5 micrograms. In some embodiments, the amount of gDNA required for amplification of one or more target sequences can be from about 1 ng to about 1 microgram or about 10 ng to about 1 microgram. In some embodiments, the amount of gDNA required for amplification of one or more immune repertoire target sequences is from about 10 ng to about 500 ng, about 25 ng to about 400 ng, or from about 50 ng to about 200 ng. In some embodiments, the amount of gDNA required for amplification of one or more target sequences is about 0.5 ng, about 1 ng, about 5 ng, about 10 ng, about 20 ng, about 50 ng, about 100 ng, or about 200 ng. In some embodiments, the amount of gDNA required for amplification of one or more immune repertoire target sequences is about 1 microgram, about 2 micrograms, about 3 micrograms, about 4.0 micrograms, or about 5 micrograms.

In some embodiments, gDNA is obtained from a biological sample using conventional methods. Methods and reagents for extracting or isolating nucleic acid from biological samples are well known and commercially available. In some embodiments, DNA extraction from biological samples is performed by any method described herein or otherwise known to those of skill in the art, e.g., methods involving proteinase K tissue digestion and alcohol-based nucleic acid precipitation, treatment with RNAse to digest contaminating RNA, and DNA purification using silica-gel-membrane technology, or any combination thereof. Exemplary methods for DNA extraction from biological samples using commercially available kits including Ion AmpliSeq™ Direct FFPE DNA Kit, MagMAX™ FFPE DNA/RNA Ultra Kit, TRI Reagent™ (Invitrogen), PureLink™ Genomic DNA Mini kit (Invitrogen), RecoverAll™ Total Nucleic Acid Isolation Kit (Invitrogen), MagMAX™ DNA Multi-Sample Kit (Invitrogen) and DNA extraction kits from BioChain Institute Inc. (e.g., FFPE Tissue DNA Extraction Kit, Genomic DNA Extraction Kit, Blood and Serum DNA Isolation Kit).

A sample or biological sample, as used herein, refers to a composition from an individual that contains or may contain cells related to the immune system. Exemplary biological samples, include without limitation, tissue (for example, lymph node, organ tissue, bone marrow), whole blood, synovial fluid, cerebral spinal fluid, tumor biopsy, and other clinical specimens containing cells. The sample may include normal and/or diseased cells and be a fine needle aspirate, fine needle biopsy, core sample, or other sample. In some embodiments, the biological sample may comprise hematopoietic cells, peripheral blood mononuclear cells (PBMCs), T cells, B cells, tumor infiltrating lymphocytes (“TILs”) or other lymphocytes. In some embodiments, the sample may be fresh (e.g., not preserved), frozen, or formalin-fixed paraffin-embedded tissue (FFPE). Some samples comprise cancer cells, such as carcinomas, melanomas, sarcomas, lymphomas, myelomas, leukemias, and the like, and the cancer cells may be circulating tumor cells. In some embodiments, the biological sample comprises cfDNA, such as found, for example, in blood or plasma.

The biological sample can be a mix of tissue or cell types, a preparation of cells enriched for at least one particular category or type of cell, or an isolated population of cells of a particular type or phenotype. Samples can be separated by centrifugation, elutriation, density gradient separation, apheresis, affinity selection, panning, FACS, centrifugation with Hypaque, etc. prior to analysis. Methods for sorting, enriching for, and isolating particular cell types are well-known and can be readily carried out by one of ordinary skill. In some embodiments, the sample may a preparation enriched for B cells.

In some embodiments, the provided methods and systems include processes for analysis of immune repertoire receptor cDNA or gDNA sequence data and for identification and/or removing PCR or sequencing-derived error(s) from the determined immune receptor sequence.

In some embodiments, the error correction strategy includes the following steps:
1) Align the sequenced rearrangement to a reference database of variable, diversity and joining/constant genes to produce a query sequence/reference sequence pair. Many alignment procedures may be used for this purpose including, for example, IgBLAST, a freely-available tool from the NCBI, and custom computer scripts.
2) Realign the reference and query sequences to each other, taking into account the flow order used for sequencing. The flow order provides information that allows one to identify and correct some types of erroneous alignments.
3) Identify the borders of the CDR3 region by their characteristic sequence motifs.
4) Over the aligned portion of the rearrangement corresponding to the variable gene and joining/constant genes, excluding the CDR3 region, identify indels in the query with respect to the reference and alter the mismatching query base position so that it is consistent with the reference.
5) For the CDR3 region, if the CDR3 length is not a multiple of three (indicative of an indel error):
   (a) Search the CDR3 for the homopolymer stretch having the highest probability of containing a sequence error, based on PHRED score (denoted e).
   (b) Obtain the probability of error over the entire CDR3 region based on PHRED score (denoted t)
   (c) If e/t is greater than a defined threshold, edit the homopolymer by either increasing or decreasing the length of the homopolymer by one base such that the CDR3 nucleotide length is a multiple of three.
   (d) As an alternative to steps a-c, search the CDR3 for the longest homopolymer, and if the length of the homopolymer is above a defined threshold, edit the homopolymer by either increasing or decreasing the length of the homopolymer by one base such that the CDR3 nucleotide length is a multiple of three.

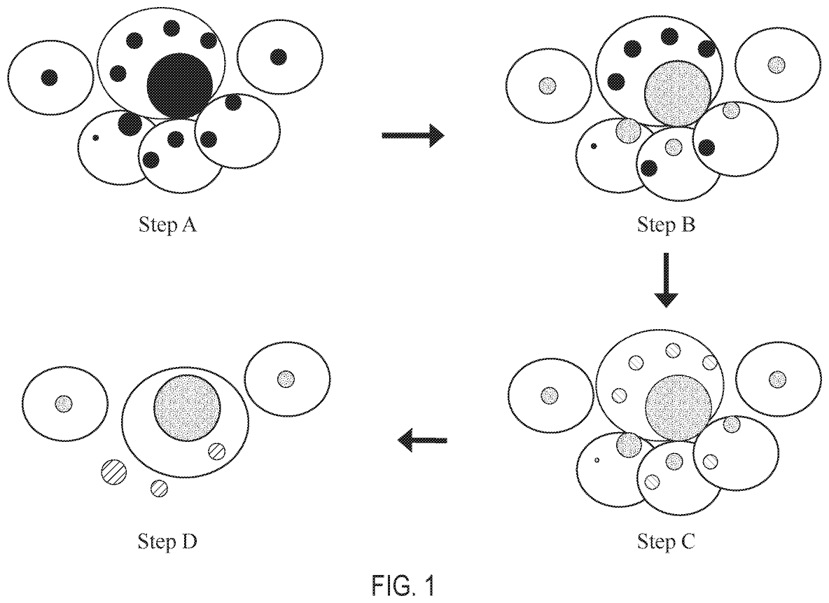

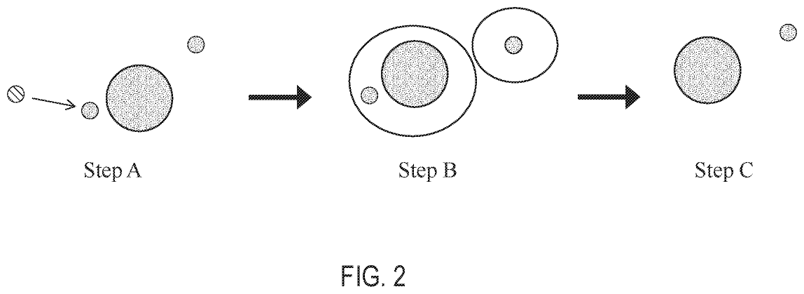

In some embodiments, methods are provided to identify B cell and/or T cell clones in repertoire data that are robust to PCR and sequencing error. Accordingly, the following describes steps that may be employed in such methods to identify B cell and/or T cell clones in a manner that is robust to PCR and sequencing error. Table 1 a diagram of an exemplary workflow for use in identifying and removing PCR or sequencing-derived errors from immune receptor sequencing data. Exemplary portions and embodiments of this workflow are also represented in FIGS. 1-2.

TABLE 1
SEQUENCE CORRECTION WORKFLOW

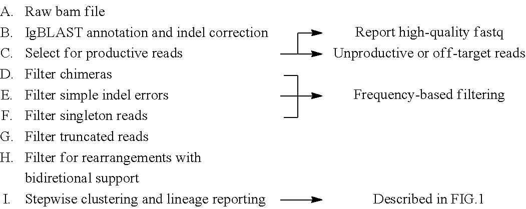

For a set of TCR or BCR sequences derived from mRNA or gDNA, where 1) each sequence has been annotated as a productive rearrangement, either natively or after error correction, such as previously described, and 2) each sequence has an identified V gene and CDR3 nucleotide region, in some embodiments, methods include the following:
1) Identify and exclude chimeric sequences. For each unique CDR3 nucleotide sequence present in the dataset, tally the number of reads having that CDR3 nucleotide sequence and any of the possible V genes. Any V gene-CDR3 combination making up less than 10% of total reads for that CDR3 nucleotide sequence is flagged as a chimera and eliminated from downstream analyses. As an example, for the sequences below having the same CDR3 nucleotide sequence, e.g., the sequences having TRBV3 and TRBV6 paired with CDR3nt sequence AATTGGT will be flagged as chimeric.

| V gene | CDR3nt | Read counts |
|---|---|---|
| TRBV2 | AATTGGT | 1000 |
| TRBV3 | AATTGGT | 10 |
| TRBV6 | AATTGGT | 3 |

2) Identify and exclude sequences containing simple indel errors. For each read in the dataset, obtain the homopolymer-collapsed representation of the CDR3 sequence of that read. For each set of reads having the same V gene and collapsed-CDR3 combination, tally the number of occurrences of each non-collapsed CDR3 nucleotide sequence. Any non-collapsed CDR3 sequence making up <10% of total reads for that read set is flagged as having a simple homopolymer error. As an example, three different V gene-CDR3 nucleotide sequences are presented that are identical after homopolymer collapsing of the CDR3 nucleotide sequence. The two less frequent V gene-CDR3 combinations make up <10% of total reads for the read set and will be flagged as containing a simple indel error. For example:

| | | Homopolymer | |
|---|---|---|---|
| V gene | CDR3nt | collapsed CDR3nt | Read counts |
| TRBV2 | AATTGGT | ATGT | 1000 |
| TRBV2 | AAATGGT | ATGT | 10 |
| TRBV2 | AAAATTTGGT | ATGT | 3 |
| | (SEQ ID NO: | | |
| | 602) | | |

3) Identify and exclude singleton reads. For each read in the dataset, tally the number of times that the exact read sequence is found in the dataset. Reads that appear only once in the dataset will be flagged as singleton reads.
4) Identify and exclude truncated reads. For each read in the dataset, determine whether the read possesses an annotated V gene FR1, CDR1, FR2, CDR2, and FR3 region, as indicated by the IgBLAST alignment of the read to the IgBLAST reference V gene set. Reads that do not possess the above regions are flagged as truncated if the region(s) is expected based on the particular V gene primer used for amplification.
5) Identify and exclude rearrangements lacking bidirectional support. For each read in the dataset, obtain the V gene and CDR3 sequence of the read as well as the strand orientation of the read (plus or minus strand). For each V gene-CDR3 combination in the dataset, tally the number of plus and minus strand reads having that V gene-CDR3nt combination. V gene-CDR3nt combinations that are only present in reads of one orientation will be deemed to be a spurious. All reads having a spurious V gene-CDR3nt combination will be flagged as lacking bidirectional support.
6) For genes that have not been flagged, perform stepwise clustering based on CDR3 nucleotide similarity. Separate the sequences into groups based on the V gene identity of the read, excluding allele information (v-gene groups). For each group:
   a. Arrange reads in each group into clusters using cd-hit-est and the following parameters:
   cd-hit-est-i vgene_groups.fa-o clustered_vgene_groups.cdhit-T 24-d 0-M 100000-B 0-r 0-g 1-S 0-U 2-uL 0.05-n 10-1 7. (The freely available software program cd-hit-est clusters a nucleotide dataset into clusters that meet a user-defined similarity threshold. (For code and instructions on cd-hit-est, see https://github.com/weizhongli/cdhit/wiki/3.-User %27s-Guide#CDHITEST).
   Where vgene_groups.fa is a fasta format file of the CDR3 nucleotide regions of sequences having the same V gene and clustered_vgene_groups.cdhit is the output, containing the subdivided sequences.
   b. Assign each sequence in a cluster the same clone ID, used to denote that members of the subgroup are believed to represent the same T cell clone or B cell clone.
   c. Chose a representative sequence for each cluster, such that the representative sequence is the sequence that appears the greatest number of times, or, in cases of a tie, is randomly chosen.
   d. Merge all other reads in the cluster into the representative sequence such that the number of reads for the representative sequence is increased according to the number of reads for the merged sequences.
   e. Compare the representative sequences within a v-gene group to each other on the basis of hamming distance. If a representative sequence is within a hamming distance of 1 to a representative sequence that is >50 times more abundant, merge that sequence into the more common representative sequence. If a representative sequence is within a hamming distance of 2 to a representative sequence that is >10000 times more abundant, merge that sequence into the more common representative sequence.
   f. Identify complex sequence errors. Homopolymer-collapse the representative sequences within each V gene group, then compare to each other using Levenshtein distances. If a representative sequence is within a Levenshtein distance of 1 to a representative sequence that is >50 times more abundant, merge that sequence into the more common representative sequence.
   g. Identify CDR3 misannotation errors. Homopolymer-collapse the representative sequences within each V gene group, then perform a pairwise comparison of each homopolymer-collapsed sequence. For each pair of sequences, determine whether one sequence is a subset of the other sequence. If so, merge the less abundant sequence into the more abundant sequence if the more abundance sequence is >500 fold more abundant.
7) Report cluster representatives to user.

In some embodiments, step 6 of the above workflow separates the rearrangement sequences into groups based on the V-gene identity (excluding allele information), and the CDR3 nucleotide length. In other embodiments, the J-gene identity and/or isotype identity is also used as part of the grouping criteria. Accordingly, in some embodiments, step 6 of the above workflow includes the following steps:
a. Arrange reads in each group into clusters using cd-hit-est and the following parameters:
   cd-hit-est-i vgene_groups.fa-o clustered_vgene_groups.cdhit-T 24-1 9-d 0-M 100000-B0-r0-g 1-S 15-U 2-uL.05-n 9.
   Where vgene_groups.fa is a fasta format file of the sequenced portion of the VDJ rearrangement.
   In some embodiments, the full sequence of the VDJ is considered for clustering as somatic hypermutation may occur throughout the VDJ region.
b. Assign each sequence in a cluster the same clone ID, used to denote that members of the subgroup are believed to represent the same T cell clone or B cell clone.
c. Chose a representative sequence for each cluster, such that the representative sequence is the sequence that appears the greatest number of times, or, in cases of a tie, is randomly chosen.
d. Merge all other reads in the cluster into the representative sequence such that the number of reads for the representative sequence is increased according to the number of reads for the merged sequences.
e. Compare the representative sequences within a v-gene group to each other on the basis of hamming distance. If a representative sequence is within a hamming distance of 1 to a representative sequence that is >50 times more abundant, merge that sequence into the more common representative sequence. If a representative sequence is within a hamming distance of 2 to a representative sequence that is >10000 times more abundant, merge that sequence into the more common representative sequence. In some embodiments, fold thresholds of >50/3 and >10000/3, among others are used to merge sequences of hamming distances 1 or 2, respectively. Reducing the fold thresholds can be useful when comparing sequences of the entire VDJ region rather than sequences of only the CDR3 region as the longer sequence has a greater chance of accumulating amplification and/or sequencing errors.
f. Identify complex sequence errors. Homopolymer-collapse the representative sequences within each V gene group, then compare to each other using Levenshtein distances. If a representative sequence is within a Levenshtein distance of 1 to a representative sequence that is >50 times more abundant, merge that sequence into the more common representative sequence.
g. Identify CDR3 misannotation errors. Homopolymer-collapse the representative sequences within each V gene group, then perform a pairwise comparison of each homopolymer-collapsed sequence. For each pair of sequences, determine whether one sequence is a subset of the other sequence. If so, merge the less abundant sequence into the more abundant sequence if the more abundance sequence is >500 fold more abundant.

In some embodiments, the provided workflows are not limited to the frequency ratio thresholds listed in the various steps, and other frequency ratio thresholds may be substituted for the representative frequency ratio thresholds included above. The frequency ratio refers to a ratio of the abundance value of the more common representative sequence to the abundance value of the less common representative sequence. The frequency ratio threshold gives the threshold at which the less common representative sequence is merged into the more common representative sequence. For example, in some embodiments, comparing the representative sequences within a v-gene group to each other on the basis of hamming distance may use a frequency ratio threshold other than those listed in step (e) above. For example and without limitation, frequency ratio thresholds of 1000, 5000, 20,000, etc may be used if a representative sequence is within a hamming distance of 2 to a representative sequence. For example and without limitation, frequency ratio thresholds of 20, 100, 200, etc may be used if a representative sequence is within a hamming distance of 1 to a representative sequence. The frequency ratio thresholds provided are representative of the general process of labeling the more abundant sequence of a similar pair as a correct sequence.

Similarly, when comparing the frequencies of two sequences at other steps in the workflows, eg, step (1), step (2), step (6f) and step (6g), frequency ratio thresholds other than those listed in the step above may be used.

As used herein, the term “homopolymer-collapsed sequence” is intended to represent a sequence where repeated bases are collapsed to a single base representative. As an example, for the non-collapsed sequence AAAATTTTTATCCCCCCCCGGG (SEQ ID NO: 603), the homopolymer-collapsed sequence is ATATCG.

As used herein, the terms “clone,” “clonotype,” “lineage,” or “rearrangement” are intended to describe a unique V gene nucleotide combination for an immune receptor, such as a TCR or BCR. For example, a unique V gene-CDR3 nucleotide combination.

As used herein, the term “productive reads” refers to a TCR or BCR sequence reads that have no stop codon and have in-frame variable gene and joining gene segments. Productive reads are biologically plausible in coding for a polypeptide.

As used herein, “chimeras” or chimeric sequences” refer to artefactual sequences that arise from template switching during target amplification, such as PCR. Chimeras typically present as a CDR3 sequence grafted onto an unrelated V gene, resulting in a CDR3 sequence that is associated with multiple V genes within a dataset. The chimeric sequence is usually far less abundant than the true sequence in the dataset.

As used herein, the term “indel” refers to an insertion and/or deletion of one or more nucleotide bases in a nucleic acid sequence. In coding regions of a nucleic acid sequence, unless the length of an indel is a multiple of 3, it will produce a frameshift when the sequence is translated. As used herein, “simple indel errors” are errors that do not alter the homopolymer-collapsed representation of the sequence. As used herein, “complex indel errors” are indel sequencing errors that alter the homopolymer-collapsed representation of the sequence and include, without limitation, errors that eliminate a homopolymer, insert a homopolymer into the sequence, or create a dyslexic-type error.

As used herein, “singleton reads” refer to sequence reads whose indel-corrected sequence appears only once in a dataset. Typically, singleton reads are enriched for reads containing a PCR or sequencing error.

As used herein, “truncated reads” refer to immune receptor sequence reads that are missing annotated V gene regions. For example, truncated reads include, without limitation, sequence reads that are missing annotated TCR or BCR V gene FR1, CDR1, FR2, CDR2, or FR3 regions. Such reads typically are missing a portion of the V gene sequence due to quality trimming. Truncated reads can give rise to artifacts if the truncation leads one to misidentify the V gene.

In the context of identified V gene-CDR3 sequences (clonotypes), “bidirectional support” indicates that a particular V gene-CDR3 sequence is found in at least one read that maps to the plus strand (proceeding from the V gene to constant gene) and at least one reads that maps to the minus strand (proceeding form the constant gene to the V gene). Systematic sequencing errors often lead to identification of V gene-CDR3 sequences having unidirectional support.

For a set of sequences that have been grouped according to a predetermined sequence similarity threshold to account for variation due to PCR or sequencing error, the “cluster representative” is the sequence that is chosen as most likely to be error free. This is typically the most abundant sequence.

As used herein, “IgBLAST annotation error” refers to rare events where the border of the CDR3 is identified to be in an incorrect adjacent position. These events typically add three bases to the 5′ or 3′ end of a CDR3 nucleotide sequence.

For two sequences of equal length, the “Hamming distance” is the number of positions at which the corresponding bases or amino acids are different. For any two sequences, the “Levenshtein distance” or the “edit distance” is the number of single base or amino acid edits required to make one nucleotide or amino acid sequence into another nucleotide or amino acid sequence.

In some embodiments in which J gene-directed primers are used in amplification of the immune receptor sequences, for example multiplex amplification with primers directed to V gene FR3 regions and primers directed to J genes, raw sequence reads derived from the assay undergo a J gene sequence inference process before any downstream analysis. In this process, the beginning and end of raw read sequences are interrogated for the presence of characteristic sequences of 10-30 nucleotides corresponding to the portion of the J gene sequences expected to exist after amplification with the J primer and any subsequent manipulation or processing (for example, digestion) of the amplicon termini prior to sequencing. The characteristic nucleotide sequences permit one to infer the sequence of the J primer, as well as the remaining portion of the J gene that was targeted since the sequence of each J gene is known. To complete the J gene sequence inference process, the inferred J gene sequence is added to the raw read to create an extended read that then spans the entire J gene. The extended read then contains the entire J gene sequence, the entire sequence of the CDR3 region, and at least a portion of the V gene sequence, which will be reported after downstream analysis. The portion of V gene sequence in the extended read will depend on the V gene-directed primers used for the multiplex amplification, for example, FR3-, FR2-, or FR1-directed primers.

Use of V gene FR3 and J gene primers to amplify expressed immune receptor sequences or rearranged immune receptor gDNA sequences yields a minimum length amplicon (for example, about 60-100 or about 80 nucleotides in length) while still producing data that allows for reporting of the entire CDR3 region. With the expectation of short amplicon length, reads of amplicons <100 nucleotides in length are not eliminated as low-quality and/or off target products during the sequence analysis workflow. However, the explicit search for the expected J gene sequences in the raw reads allows one to eliminate amplicons deriving from off-target amplifications by the J gene primers. In addition, this short amplicon length improves the performance of the assay on highly degraded template material, such as that derived from an FFPE or cfDNA sample.

In some embodiments, provided methods comprise sequencing an immune receptor library and subjecting the obtained sequence data to error identification and correction processes to generate rescued productive reads, and identifying productive and rescued productive sequence reads. In some embodiments, provided methods comprise sequencing an immune receptor library and subjecting the obtained sequence dataset to error identification and correction processes, identifying productive and rescued productive sequence reads, and grouping the sequence reads by clonotype to identify immune receptor clonotypes in the library.

In some embodiments, provided methods comprise sequencing a rearranged immune receptor DNA library and subjecting the obtained sequence data to error identification and correction processes for the V gene portions to generate rescued productive reads, and identifying productive, rescued productive, and unproductive sequence reads. In some embodiments, provided methods comprise sequencing a rearranged immune receptor DNA library and subjecting the obtained sequence dataset to error identification and correction processes for the V gene portions, identifying productive, rescued productive, and unproductive sequence reads, and grouping the sequence reads by clonotype to identify immune receptor clonotypes in the library. In some embodiments, both productive and unproductive sequence reads of rearranged immune receptor DNA are separately reported.

In some embodiments, the provided error identification and correction workflow is used for identifying and resolving PCR or sequencing-derived errors that lead to a sequence read being identified as from an unproductive rearrangement. In some embodiments, the provided error identification and correction workflow is applied to immune receptor sequence data generated from a sequencing platform in which indel or other frameshift-causing errors occur while generating the sequence data.

In some embodiments, the provided error identification and correction workflow is applied to sequence data generated by an Ion Torrent sequencing platform. In some embodiments, the provided error identification and correction workflow is applied to sequence data generated by Roche 454 Life Sciences sequencing platforms, PacBio sequencing platforms, and Oxford Nanopore sequencing platforms.

In some embodiments, the BCR repertoire analysis workflow includes an additional last step to identify clonal lineages in the sample. A clonal lineage represents a set of B cell clones (e.g., identified as having unique VDJ sequences) that derive from a common VDJ rearrangement but differ owing to somatic hypermutation and/or class switch recombination. It is generally assumed that members of a clonal lineage may be more likely to target the same antigen than members of different clonal lineages.

In some embodiments, the process of clonal lineage identification includes using a set of BCR clones (e.g., IgH clones) identified (for example as described herein) to perform the following:
1. Separate the clone sequences into groups where group members share the same variable gene (excluding allele information), the same CDR3 nucleotide length, and the same joining gene (excluding allele information). In some embodiments the above J-gene criterion may be omitted.
2. Arrange the clone sequences in each group into clusters based on the CDR3 nucleotide similarity of the clone sequences. Thresholds for CDR3 nucleotide similarity are about 0.70 to about 0.99. In some embodiments, the threshold for CDR3 nucleotide similarity is between about 0.80 to about 0.99. In some embodiments, the threshold for CDR3 nucleotide similarity is between about 0.80 to about 0.90. In certain embodiments, the threshold for CDR3 nucleotide similarity is about 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99.
   a. In some embodiments, the clustering is performed using cd-hit-est as described:
      cd-hit-est-i vgene_groups.fa-o clustered_vgene_groups.cdhit-T 24-1 9-d 0-M 100000-B 0-r 0-g 1-S 0-c 0.85-n 5, where vgene_groups.fa consists of the set of CDR3 nucleotide sequences of each clone within a group. Clones within the same cluster are considered members of the same clonal lineage.
   b. In some instances, somatic hypermutation may be extensive enough that the described clustering criteria may not group all clonal lineage members. For such cases, in some embodiments, an additional step is performed to merge clusters identified in (a). The additional step consists of searching for instances of shared somatic hypermutation-derived mutations in the variable gene between clonal lineages, then merging clonal lineages if the fraction and/or number of shared mutations is above a certain threshold. Variable gene mutations are identified by comparison of the variable gene sequence to the best matching variable gene sequence in the IMGT database, as described. In some embodiments, the threshold for number of shared mutations is 2 or more. In some embodiments, the threshold for number of shared mutations is 3 or more. In other embodiments, the threshold for number of shared mutations is 4, 5, 6, 7, 8, 9, 10 or more. In some embodiments, the fraction of shared mutations is about 0.15 to about 0.95. In some embodiments, the fraction of shared mutations is about 0.75 or about 0.85. In other embodiments, the fraction of shared mutations is about 0.15, 0.2, 0.25, 0.3, 0.35, 0.4, 0.5, 0.6, 0.7, 0.8, 0.9, or 0.95.

In some instances, a variable gene allele may be identified that is not represented in the IMGT database. In such instances, alignment to the IMGT database will indicate a mismatch that is not derived from somatic hypermutation. To avoid noise caused by such unannotated genetic variants, in some embodiments, an initial step is performed before (b) where one identifies all putative novel variable gene alleles in a sample, noting each position that differs from reference. In some embodiments, such positions are then excluded from consideration in the analysis described in (b). Methods for the identification of novel alleles from immune repertoire sequencing data have been described, for example, by Gadala-Maria et al. (2015) Proc. Natl. Acad. Sci. USA 112: E862-E870 and PCT Application Publication No. WO 2018/136562.

At the end of this clonal lineage identification process, each clone has been assigned to a clonal lineage. BCR repertoire features such as diversity, evenness, and convergence may be calculated with the clonal lineage as the unit of analysis. In some embodiments, clonal lineages features, such as the number of clones belonging to a lineage, the isotypes of those clones, the maximum and minimum frequency of the clones in a lineage, the maximum and minimum variable gene somatic hypermutation in a lineage, and others, are calculated and reported to the user.

In the absence of somatic hypermutation, BCR convergence may be calculated as the frequency of clones that are identical, or functionally identical, in amino acid sequence but different in nucleotide sequence. These represent clones that independently underwent VDJ recombination and generally assumed to have proliferated in response to a common antigen. However, somatic hypermutation can create distinct VDJ sequences that do not represent B cells that independently underwent VDJ recombination. To account for this a definition of convergence is used that takes into account the clonal lineage identification. For this purpose, “BCR convergence” is defined as the frequency of B cell clones that are members of different clonal lineages, as determined above, but are similar or identical in amino acid sequence. In some embodiments, two IGH rearrangements are considered convergent if they are assigned to separate clonal lineages but have the same variable gene (excluding allele information) and the same or similar CDR3 amino acid sequence. In other embodiments where sequencing covers all three CDR domains of the IGH chain, two IGH rearrangements may be considered convergent if they are assigned to separate clonal lineages but have the same variable gene (excluding allele information) and the same or similar CDR1, 2 and 3 amino acid sequence. In some embodiments, similar CDR amino acid sequences are within a Hamming or Levenshtein edit distance of 1. In other embodiments, similar CDR amino acid sequences are within a Hamming or Levenshtein edit distance of 2.

Accordingly, in some embodiments, functionally equivalent B cells are identified by searching for BCR clones having the same variable gene and CDR amino acid sequences that are within a Hamming or Levenshtein edit distance of 1 or 2. In some embodiments the program cd-hit may be used to identify clones having similar but functionally equivalent amino acid sequences. (For code and information on the program cd-hit, see https://github.com/weizhongli/cdhit/wiki/3.-User %27s-Guide) In some embodiments cd-hit is run using the following command:
cd-hit-i vgene_groups.fa-o clustered_vgene_groups.cdhit-T 24-1 5-d 0-M 100000-B 0-g 1-S 1-U 1-n 5, where vgene_groups.fa consists of the set of CDR3 amino acid sequences of clones having the same variable gene. Clones within the same cluster are considered to be functionally equivalent.

In some embodiments, the value for the parameter-S may be 0, 1, 2, or 3. In some embodiments, the value for the parameter-U may be 0, 1, 2, or 3.

In some embodiments, vgene_groups.fa consists of the set of CDR 1, 2 and 3 amino acid sequences of clones having the same variable gene. In some embodiments, vgene_groups.fa consists of the set of clones having both the same variable gene and the same CDR3 length.

In some embodiments, provided sequence analysis workflows include a downsampling analysis. For immune repertoire sequencing and subsequent analysis, use of downsampling analysis can help, for example, to eliminate variability owing to differences in sequencing depth across an assay. For example, an exemplary downsampling analysis for use with RNA or cDNA sequencing and analysis workflows applies the following procedure to the data: a) starting with the total set of productive+rescued productive reads, sequence reads are randomly removed down to one of several fixed read depths and b) this subset of reads is used to perform all downstream calculations (for example, clonotyping and calculation of secondary repertoire features including without limitation evenness, convergence, diversity, number and identity of clones detected, and clonal lineages).

In some embodiments, downsampling analysis identifies the point at which a particular sample is sequenced to saturation, for example, a point at which additional reads do not identify additional clones or lineages or add additional diversity to the detected repertoire. In some embodiments, downsampling allows the refining of sequencing depth or multiplexing among or between assays with similar sample types.

In some embodiments, the set of variable gene alleles detected by the assay methods and compositions provided may be used for de novo identification of haplotype groups within human populations. In particular embodiments, provided assay methods and compositions which include use of a plurality of V gene-specific primers and at least one C gene specific primer to amplify IgH CDR 1, 2, and 3 nucleotide sequences may be used to identify the IgH haplotype of a subject's BCR repertoire. For example, in some embodiments, methods and compositions provided which use at least set of primers comprising a plurality of V gene FR1 primers selected from Table 3 and at least one C gene primer selected from Tables 6-10 may be used to identify the IgH haplotype of a subject's BCR repertoire. Methods for identification of TCR haplotype groups are described in PCT Application No. PCT/US2019/023731, filed Mar. 22, 2019, the entirety of which is incorporated herein by reference, and may similarly be used in conjunction with the methods and compositions provided herein to identify IgH haplotype groups. In some embodiments, the set of variable gene alleles detected by amplifying and sequencing IgH CDR 1, 2, and 3 nucleotide sequences may be used to assign a sample to one of several pre-existing haplotype groups as part of a larger procedure for predicting the risk of autoimmune disease or adverse events following an immunotherapy. Methods for assigning a sample to a haplotype group in a procedure for predicting risk of autoimmune disease or adverse events following an immunotherapy are also described in PCT Application No. PCT/US2019/023731, filed Mar. 22, 2019 and incorporated herein by reference, and may similarly be used in conjunction with the methods and compositions provided herein to assign a sample to a IgH haplotype group, for example, for predicting such risks. In some embodiments, the IgH CDR 1, 2, 3 sequence data obtained using the provided assay methods and compositions may be used to infer phased IgH locus haplotypes (for example, Kidd et al. (2012) J. Immunol. 188(3): 1333-1340).

In some embodiments, provided methods comprise preparation and formation of a plurality of immune receptor-specific amplicons. In some embodiments, the method comprises hybridizing a plurality of V gene-specific primers and at least one C gene-specific primer to a cDNA molecule, extending a first primer (e.g., a V gene-specific primer) of the primer pair, denaturing the extended first primer from the cDNA molecule, hybridizing to the extended first primer product, a second primer (e.g., a C gene-specific primer) of the primer pair and extending the second primer, digesting the target-specific primer pairs to generate a plurality of target amplicons. In other embodiments, the method comprises hybridizing a plurality of V gene gene-specific primers and a plurality of J gene-specific primers to a cDNA molecule, extending a first primer (e.g., a V gene-specific primer) of the primer pair, denaturing the extended first primer from the cDNA molecule, hybridizing to the extended first primer product, a second primer (e.g., a J gene-specific primer) of the primer pair and extending the second primer, digesting the target-specific primer pairs to generate a plurality of target amplicons. In some embodiments, adapters are ligated to the ends of the target amplicons prior to performing a nick translation reaction to generate a plurality of target amplicons suitable for nucleic acid sequencing. In some embodiments, at least one of the ligated adapters includes at least one barcode sequence. In some embodiments, each adapter ligated to the ends of the target amplicons includes a barcode sequence. In some embodiments, the one or more target amplicons can be amplified using bridge amplification, emulsion PCR or isothermal amplification to generate a plurality of clonal templates suitable for nucleic acid sequencing.

In some embodiments, provided methods comprise preparation and formation of a plurality of immune receptor-specific amplicons. In some embodiments, the method comprises hybridizing a plurality of V gene gene-specific primers and a plurality of J gene-specific primers to a gDNA molecule, extending a first primer (eg, a V gene-specific primer) of the primer pair, denaturing the extended first primer from the gDNA molecule, hybridizing to the extended first primer product, a second primer (e.g., a J gene-specific primer) of the primer pair and extending the second primer, digesting the target-specific primer pairs to generate a plurality of target amplicons. In some embodiments, adapters are ligated to the ends of the target amplicons prior to performing a nick translation reaction to generate a plurality of target amplicons suitable for nucleic acid sequencing. In some embodiments, at least one of the ligated adapters includes at least one barcode sequence. In some embodiments, each adapter ligated to the ends of the target amplicons includes a barcode sequence. In some embodiments, the one or more target amplicons can be amplified using bridge amplification or emulsion PCR to generate a plurality of clonal templates suitable for nucleic acid sequencing.

In some embodiments, the disclosure provides methods for sequencing target amplicons and processing the sequence data to identify productive immune receptor rearrangements expressed in the biological sample from which the cDNA was derived. In other embodiments, the disclosure provides methods for sequencing target amplicons and processing the sequence data to identify productive immune receptor gene rearrangements gDNA from a biological sample. In embodiments in which J gene-directed primers are used to amplify the expressed immune receptor sequences or rearranged immune receptor gDNA sequences, processing the sequence data includes inferring the nucleotide sequence of the J gene primer used for amplification as well as the remaining portion of the J gene that was targeted, as described herein. In some embodiments, processing the sequence data includes performing provided error identification and correction steps to generate rescued productive sequences. In some embodiments, use of the provided error identification and correction workflow can result in a combination of productive reads and rescued productive reads being at least 50% of the sequencing reads for an immune receptor cDNA or gDNA sample. In some embodiments, use of the provided error identification and correction workflow can result in a combination of productive reads and rescued productive reads being at least 60%, at least 70%, at least 80%, at least 90%, or at least 95% of the sequencing reads for an immune receptor cDNA or gDNA sample. In some embodiments, use of the provided error identification and correction workflow can result in a combination of productive reads and rescued productive reads being about 50-60%, about 60-70%, about 70-80%, about 80-90%, about 50-80%, or about 60-90% of the sequencing reads for an immune receptor cDNA or gDNA sample. In some embodiments, use of the provided error identification and correction workflow can result in a combination of productive reads and rescued productive reads averaging about 50%, about 55%, about 60%, about 65%, about 70%, about 75%, about 80%, about 85%, about 90% of the sequencing reads for an immune receptor cDNA or gDNA sample.

With particular samples, the provided error identification and correction workflow can result in a combination of productive reads and rescued productive reads being less than 50% of the sequencing reads for an immune receptor cDNA or gDNA sample when particular samples are used. Such samples include, for example, those in which the RNA or gDNA is highly degraded such as FFPE samples and cfDNA samples, and those in which the number of target immune cells is very low such as, for example, samples with very low B cell count or samples from subjects experiencing severe leukopenia. Accordingly, in some embodiments, use of the provided error identification and correction workflow can result in a combination of productive reads and rescued productive reads being about 30-50%, about 40-50%, about 30-40%, about 40-60%, at least 30%, or at least 40% of the sequencing reads for an immune receptor cDNA or gDNA sample.

In certain embodiments, methods of the invention comprise the use of target immune receptor primer sets wherein the primers are directed to sequences of the same target immune receptor gene, e.g, BCR (immunoglobulin) and TCR genes. In some embodiments the immune receptor is an antibody receptor selected from the group consisting of heavy chain alpha, heavy chain delta, heavy chain epsilon, heavy chain gamma, heavy chain mu, light chain kappa, and light chain lambda. In some embodiments a T cell receptor is a T cell receptor selected from the group consisting of TCR alpha, TCR beta, TCR gamma, and TCR delta. In some embodiments, methods of the invention comprise the use of target immune receptor primer sets wherein at least one of the primer sets is directed to sequences of a BCR and another primer set is directed to sequences of a TCR, and both the BCR and TCR target nucleic acids from a sample are amplified in a single multiplex amplification reaction.

In certain embodiments, provided is a method for amplification of expression nucleic acid sequences of a BCR repertoire in a sample, comprising performing a multiplex amplification reaction to amplify BCR nucleic acid template molecules having a constant portion and a variable portion using at least one set of: i) a plurality of V gene primers directed to a majority of different V genes of at least one BCR coding sequence comprising at least a portion of a framework region within the V gene, and ii) one or more C gene primers directed to at least a portion of the respective target constant gene of the BCR coding sequence, wherein each set of i) and ii) primers directed to the same target immune receptor sequences is selected from the group consisting of IgH, IgL, and IgK, and wherein performing amplification using each set results in amplicons representing the entire repertoire of the respective immune receptor in the sample; thereby generating immune receptor amplicons comprising the repertoire of the BCR. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region.

In certain embodiments, provided is a method for amplification of expression nucleic acid sequences of an immune receptor repertoire in a sample, comprising performing a multiplex amplification reaction to amplify BCR nucleic acid template molecules having a constant portion and a variable portion using at least one set of: i) a plurality of V gene primers directed to a majority of different V genes of at least one BCR coding sequence comprising at least a portion of framework region 1 (FR1) within the V gene, and ii) one or more C gene primers directed to at least a portion of the respective target C gene of the BCR coding sequence, wherein each set of i) and ii) primers directed to the same target immune receptor sequences is selected from the group consisting of IgH, IgL, and IgK and wherein performing amplification using each set results in amplicons representing the entire repertoire of the respective immune receptor in the sample; thereby generating immune receptor amplicons comprising the repertoire of the BCR. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In some embodiments the one or more plurality of V gene primers of i) anneal to at least a portion of the framework region 1 of the template molecules. In certain embodiments the one or more C gene primers of ii) comprises at least two primers that anneal to at least a portion of a C gene portion of the template molecules. In some embodiments the one or more C gene primers of ii) comprises at least two primers each of which anneal to at least a portion of the C gene of IgA, IgD, IgG, IgM or IgE template molecules. In some embodiments the one or more C gene primers of ii) comprises at least one primer separately directed to a portion of the C gene of each of IgA, IgD, IgG, IgM and IgE template molecules. In particular embodiments at least one set of the generated amplicons includes complementarity determining regions CDR1, CDR2, and CDR3 of a BCR expression sequence. In some embodiments the amplicons are about 300 to about 600 nucleotides in length or at least about 350 to about 500 nucleotides in length. In some embodiments the nucleic acid template used in methods is cDNA produced by reverse transcribing nucleic acid molecules extracted from a biological sample.

In certain embodiments, methods are provided for providing sequence of the BCR repertoire in a sample, comprising performing a multiplex amplification reaction to amplify BCR nucleic acid template molecules having a constant portion and a variable portion using at least one set of primers comprising i) a plurality of V gene primers directed to a majority of different V genes of at least one BCR coding sequence comprising at least a portion of framework region 1 (FR1) within the V gene, and ii) one or more C gene primers directed to at least a portion of the respective target C gene(s) of the BCR coding sequence, wherein each set of i) and ii) primers directed to the same target immune receptor sequences is selected from the group consisting of IgH, an IgL, and an IgK, thereby generating BCR amplicon molecules. Sequencing of resulting BCR amplicon molecules is then performed and the sequences of the BCR amplicon molecules determined thereby provides sequence of the BCR repertoire in the sample. In particular embodiments, determining the sequence of the BCR amplicon molecules includes obtaining initial sequence reads, aligning the initial sequence read to a reference sequence and identifying a productive reads, correcting one or more indel errors to generate rescued productive sequence reads; and determining the sequences of the resulting BCR molecules. In particular embodiments the combination of productive reads and rescued productive reads is at least 50%, at least 60% at least 70% or at least 75% of the sequencing reads for the BCRs. In additional embodiments the method further comprises sequence read clustering and BCR clonotype reporting. In some embodiments, the sequences of the identified immune repertoire are compared to a contemporaneous or current version of the IMGT database and the sequence of at least one allelic variant absent from that IMGT database is identified. In some embodiments the average sequence read length is between 300 and 600 nucleotides, or is between 350 and 550 nucleotides, or is between 330 and 425 nucleotides, or is about 350 to about 425 nucleotides, depending in part on inclusion of any barcode sequence in the read length. In certain embodiments at least one set of the sequenced amplicons includes complementarity determining regions CDR1, CDR2, and CDR3 of a BCR expression sequence.

In some embodiments, methods provided utilize target BCR primer sets comprising V gene primers wherein the one or more of a plurality of V gene primers are directed to sequences over an FR1 region about 70 nucleotides in length. In other particular embodiments the one or more of a plurality of V gene primers are directed to sequences over an FR1 region about 50 nucleotides in length. In certain embodiments a target BCR primer set comprises V gene primers comprising about 18 to about 45 different FR1-directed primers. In some embodiments a target BCR primer set comprises V gene primers comprising about 22 to about 35 different FR1-directed primers. In some embodiments a target BCR primer set comprises V gene primers comprising about 25 to about 35 different FR1-directed primers. In certain embodiments a target BCR primer set comprises V gene primers comprising about 40 to about 65 different FR1-directed primers. In some embodiments a target BCR primer set comprises V gene primers comprising about 48 to about 60 different FR1-directed primers. In some embodiments the target BCR primer set comprises one or more C gene primers. In particular embodiments a target immune receptor primer set comprises at least 5 to about 15 C gene primers wherein each is directed to at least a portion of the same 50 nucleotide region within each of the target C genes. In particular embodiments a target immune receptor primer set comprises at least 2 to about 8 C gene primers wherein each is directed to at least a portion of the same 50 nucleotide region within each of the target C genes. In some embodiments a target BCR primer set comprises two or more C gene primers directed to different Ig isotype molecules, e.g., IgA, IgD, IgG, IgM and IgE. In some embodiments a target BCR primer set comprises at least five C gene primers each primer directed to a C gene of a different Ig isotype molecule.

In particular embodiments, methods of the invention comprise use of at least one set of primers comprising V gene primers i) and C gene primers ii) selected from Table 3 and from Tables 6-10, respectively. In certain embodiments method of the invention comprise use of at least one set of primers i) and ii) comprising about 15 to about 35 primers selected from Table 3 and about 5 to about 20 primers selected from Tables 6-10, respectively. In some embodiments the provided methods comprise use of at least one set of primers comprising i) about 22 to about 35 primers selected from Table 3 and ii) one or more primers selected from each of Tables 6-10. In certain embodiments method of the invention comprise use of at least one set of primers i) and ii) comprising about 40 to about 65 primers selected from Table 3 and about 5 to about 20 primers selected from Tables 6-10, respectively. In some embodiments the provided methods comprise use of at least one set of primers comprising i) about 48 to about 60 primers selected from Table 3 and ii) one or more primers selected from each of Tables 6-10. In other certain embodiments methods of the invention comprise use of at least one set of primers comprising i) primers selected from SEQ ID NOs: 137-283 and ii) primers selected from SEQ ID NOs: 448-459, 472-479, 488-513, 540-551, and 564-582. In other embodiments provided methods comprise use of at least one set of primers comprising i) primers selected from SEQ ID NOs: 284-430 and ii) primers selected from SEQ ID NOs: 460-471, 480-487, 514-539, 552-563, and 583-601. In some embodiments methods of the invention comprise use of at least one set of primers comprising i) primers selected from SEQ ID NOs: 137-283 and ii) primers selected from SEQ ID NOs: 460-471, 480-487, 514-539, 552-563, and 583-601 or comprising i) primers selected from SEQ ID NOs: 284-430 and ii) primers selected from SEQ ID NOs: 448-459, 472-479, 488-513, 540-551, and 564-582.

In some embodiments methods of the invention comprise the use of at least one set of primers i) and ii) comprising at least 20 or at least 25 primers selected from SEQ ID NOs: 137-283 and at least one primer selected from SEQ ID NOs: 448-459, 472-479, 488-513, 540-551, and 564-582. In some embodiments provided methods comprise the use of at least one set of primers i) and ii) comprising about 15 to about 35 primers selected from SEQ ID NOs: 137-283 and about 5 to about 15 primers selected from SEQ ID NOs: 448-459, 472-479, 488-513, 540-551, and 564-582. In some embodiments provided methods comprise the use of at least one set of primers comprising i) about 22 to about 35 primers selected from SEQ ID NOs: 137-283 and ii) at least one primer selected from SEQ ID NOs: 448-459, at least one primer selected from SEQ ID NOs: 472-479, at least one primer selected from SEQ ID NOs: 488-513, at least one primer selected from SEQ ID NOs: 540-551 and at least one primer selected from SEQ ID NOs: 564-582. In other embodiments methods of the invention comprise the use of at least one set of primers i) and ii) comprising at least 20 or at least 25 primers selected from SEQ ID NOs: 284-430 and at least one primer selected from SEQ ID NOs: 460-471, 480-487, 514-539, 552-563, and 583-601. In some embodiments provided methods comprise the use of at least one set of primers i) and ii) comprising about 15 to about 35 primers selected from SEQ ID NOs: 284-430 and about 5 to about 15 primers selected from SEQ ID NOs: 460-471, 480-487, 514-539, 552-563, and 583-601. In some embodiments provided methods comprise the use of at least one set of primers comprising i) about 22 to about 35 primers selected from SEQ ID NOs: 284-430 and ii) at least one primer selected from SEQ ID NOs: 460-471, at least one primer selected from 480-487, at least one primer selected from 514-539, at least one primer selected from 552-563, and at least one primer selected from 583-601. In some embodiments methods of the invention comprise the use of at least one set of primers i) and ii) comprising at least 20 or at least 25 primers selected from SEQ ID NOs: 284-430 and at least one primer selected from SEQ ID NOs: 448-459, 472-479, 488-513, 540-551, and 564-582. In other embodiments methods of the invention comprise the use of at least one set of primers i) and ii) comprising at least 20 or at least 25 primers selected from SEQ ID NOs: 137-283 and at least one primer selected from SEQ ID NOs: 460-471, 480-487, 514-539, 552-563, and 583-601.

In some embodiments methods of the invention comprise the use of at least one set of primers i) and ii) comprising at least 40 or at least 50 primers selected from SEQ ID NOs: 137-283 and at least one primer selected from SEQ ID NOs: 448-459, 472-479, 488-513, 540-551, and 564-582. In some embodiments provided methods comprise the use of at least one set of primers i) and ii) comprising about 40 to about 65 primers selected from SEQ ID NOs: 137-283 and about 5 to about 15 primers selected from SEQ ID NOs: 448-459, 472-479, 488-513, 540-551, and 564-582. In some embodiments provided methods comprise the use of at least one set of primers comprising i) about 48 to about 60 primers selected from SEQ ID NOs: 137-283 and ii) at least one primer selected from SEQ ID NOs: 448-459, at least one primer selected from SEQ ID NOs: 472-479, at least one primer selected from SEQ ID NOs: 488-513, at least one primer selected from SEQ ID NOs: 540-551 and at least one primer selected from SEQ ID NOs: 564-582. In other embodiments methods of the invention comprise the use of at least one set of primers i) and ii) comprising at least 40 or at least 50 primers selected from SEQ ID NOs: 284-430 and at least one primer selected from SEQ ID NOs: 460-471, 480-487, 514-539, 552-563, and 583-601. In some embodiments provided methods comprise the use of at least one set of primers i) and ii) comprising about 40 to about 65 primers selected from SEQ ID NOs: 284-430 and about 5 to about 15 primers selected from SEQ ID NOs: 460-471, 480-487, 514-539, 552-563, and 583-601. In some embodiments provided methods comprise the use of at least one set of primers comprising i) about 48 to about 60 primers selected from SEQ ID NOs: 284-430 and ii) at least one primer selected from SEQ ID NOs: 460-471, at least one primer selected from 480-487, at least one primer selected from 514-539, at least one primer selected from 552-563, and at least one primer selected from 583-601. In some embodiments methods of the invention comprise the use of at least one set of primers i) and ii) comprising at least 40 or at least 50 primers selected from SEQ ID NOs: 284-430 and at least one primer selected from SEQ ID NOs: 448-459, 472-479, 488-513, 540-551, and 564-582. In other embodiments methods of the invention comprise the use of at least one set of primers i) and ii) comprising at least 40 or at least 50 primers selected from SEQ ID NOs: 137-283 and at least one primer selected from SEQ ID NOs: 460-471, 480-487, 514-539, 552-563, and 583-601.

In certain embodiments, provided is a method for amplification of expression nucleic acid sequences of a BCR repertoire in a sample, comprising performing a multiplex amplification reaction to amplify BCR nucleic acid template molecules having a constant portion and a variable portion using at least one set of: i) a plurality of V gene primers directed to a majority of different V genes of at least one BCR coding sequence comprising at least a portion of framework region 3 (FR3) within the V gene, and ii) one or more C gene primers directed to at least a portion of the respective target C gene of the BCR coding sequence, wherein each set of i) and ii) primers directed to the same target immune receptor sequences is selected from the group consisting of IgH, an IgL, and an IgK, and wherein performing amplification using each set results in amplicons representing the entire repertoire of the respective immune receptor in the sample; thereby generating immune receptor amplicons comprising the BCR repertoire. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In more particular embodiments the one or more plurality of V gene primers of i) are directed to sequences over about a 40 to about a 60 nucleotide portion of the framework region. In some embodiments the one or more plurality of V gene primers of i) anneal to at least a portion of the framework 3 region of the template molecules. In certain embodiments the one or more C gene primers of ii) comprises at least two primers that anneal to at least a portion of the C gene of the BCR template molecules. In some embodiments the one or more C gene primers of ii) comprises at least two primers each of which anneal to at least a portion of the C gene of IgA, IgD, IgG, IgM or IgE template molecules. In some embodiments the one or more C gene primers of ii) comprises at least one primer separately directed to a portion of the C gene of each of IgA, IgD, IgG, IgM and IgE template molecules. In particular embodiments at least one set of the generated amplicons includes complementarity determining region CDR3 of a BCR expression sequence. In some embodiments the amplicons are about 80 to about 200 nucleotides in length, about 80 to about 140 nucleotides in length, about 90 to about 130 nucleotides in length or at least about 100 to about 120 nucleotides in length. In some embodiments the nucleic acid template used in methods is cDNA produced by reverse transcribing nucleic acid molecules extracted from a biological sample.

In certain embodiments, methods are provided for providing sequence of the BCR repertoire in a sample, comprising performing a multiplex amplification reaction to amplify BCR nucleic acid template molecules having a constant portion and a variable portion using at least one set of primers comprising i) a plurality of V gene primers directed to a majority of different V genes of at least one BCR coding sequence comprising at least a portion of framework region 3 (FR3) within the V gene, and ii) one or more C gene primers directed to at least a portion of the respective target C gene(s) of the BCR coding sequence, wherein each set of i) and ii) primers directed to the same target immune receptor sequences is selected from the group consisting of IgH, IgL, and IgK, thereby generating BCR amplicon molecules. Sequencing of resulting BCR amplicon molecules is then performed and the sequences of the BCR amplicon molecules determined thereby provides sequence of the BCR in the sample. In particular embodiments, determining the sequence of the BCR amplicon molecules includes obtaining initial sequence reads, aligning the initial sequence read to a reference sequence and identifying a productive reads, correcting one or more indel errors to generate rescued productive sequence reads; and determining the sequences of the resulting BCR molecules. In particular embodiments the combination of productive reads and rescued productive reads is at least 50%, at least 60% at least 70% or at least 75% of the sequencing reads for the BCRs. In additional embodiments the method further comprises sequence read clustering and BCR clonotype reporting. In some embodiments, the sequences of the identified BCR repertoire are compared to a contemporaneous or current version of the IMGT database and the sequence of at least one allelic variant absent from that IMGT database is identified. In some embodiments the average sequence read length is between 80 and 185 nucleotides, is between 115 and 200 nucleotides, is between 90 and 130 nucleotides, or is between about 100 and about 120 nucleotides, depending in part on inclusion of any barcode sequence in the read length. In certain embodiments at least one set of the sequenced amplicons includes complementarity determining region CDR3 of a BCR expression sequence.

In certain embodiments, methods provided utilize target BCR primer sets comprising V gene primers wherein the one or more of a plurality of V gene primers are directed to sequences over an FR3 region about 70 nucleotides in length. In particular embodiments, methods provided utilize target BCR primer sets comprising V gene primers wherein the one or more of a plurality of V gene primers are directed to sequences over an FR3 region about 50 nucleotides in length. In other particular embodiments the one or more of a plurality of V gene primers are directed to sequences over an FR3 region about 40 to about 60 nucleotides in length. In certain embodiments a target BCR primer set comprises V gene primers comprising about 50 to about 85 different FR3-directed primers. In certain embodiments a target BCR primer set comprises V gene primers comprising about 55 to about 80 different FR3-directed primers. In some embodiments, a target immune receptor primer set comprises V gene primers comprising about 62 to about 75 different FR3-directed primers. In some embodiments, a target BCR primer set comprises V gene primers comprising about 65, 66, 67, 68, 69, or 70 different FR3-directed primers. In some embodiments the target BCR primer set comprises one or more C gene primers. In particular embodiments a target immune receptor primer set comprises at least 5 to about 15 C gene primers wherein each is directed to at least a portion of the same 50 nucleotide region within each of the target C genes. In particular embodiments a target BCR primer set comprises at least 2 to about 8 C gene primers wherein each is directed to at least a portion of the same 50 nucleotide region within each of the target C genes. In some embodiments the one or more C gene primers of ii) comprises at least two primers each of which anneal to at least a portion of the C gene of IgA, IgD, IgG, IgM or IgE template molecules. In some embodiments the one or more C gene primers of ii) comprises at least one primer separately directed to a portion of the C gene of each of IgA, IgD, IgG, IgM and IgE template molecules.

In particular embodiments, methods of the invention comprise the use of at least one set of primers comprising V gene primers i) and C gene primers ii) selected from Table 2 and from Tables 6-10, respectively. In certain embodiments methods of the invention comprise use of at least one set of primers i) and ii) comprising about 55 to about 80 primers selected from Table 2 and about 5 to about 20 primers selected from Tables 6-10, respectively. In some embodiments the provided methods comprise use of at least one set of primers comprising i) about 62 to about 75 primers selected from Table 2 and ii) one or more primers selected from each of Tables 6-10. In other certain embodiments methods of the invention comprise the use of at least one set of primers i) and ii) comprising primers selected from SEQ ID NOs: 1-68 and 448-459, 472-479, 488-513, 540-551, and 564-582 or selected from SEQ ID NOs: 69-136 and 460-471, 480-487, 514-539, 552-563, and 583-601. In some embodiments methods of the invention comprise the use of at least one set of primers i) and ii) comprising primers selected from SEQ ID NOs: 1-68 and 460-471, 480-487, 514-539, 552-563, and 583-601 or selected from SEQ ID NOs: 69-136 and 448-459, 472-479, 488-513, 540-551, and 564-582.

In some embodiments methods of the invention comprise the use of at least one set of primers i) and ii) comprising at least 60 primers selected from SEQ ID NOs: 1-68 and at least one primer selected from SEQ ID NOs: 448-459, 472-479, 488-513, 540-551, and 564-582. In some embodiments provided methods comprise the use of at least one set of primers i) and ii) comprising at least 60 primers selected from SEQ ID NOs: 1-68 and about 5 to about 15 primers selected from SEQ ID NOs: 448-459, 472-479, 488-513, 540-551, and 564-582. In some embodiments provided methods comprise the use of at least one set of primers i) and ii) comprising at least 60 primers selected from SEQ ID NOs: 1-68 and at least one primer selected from SEQ ID NOs: 448-459, at least one primer selected from SEQ ID NOs: 472-479, at least one primer selected from SEQ ID NOs: 488-513, at least one primer selected from SEQ ID NOs: 540-551 and at least one primer selected from SEQ ID NOs: 564-582. In other embodiments methods of the invention comprise the use of at least one set of primers i) and ii) comprising at least 60 primers selected from SEQ ID NOs: 69-136 and at least one primer selected from SEQ ID NOs: 460-471, 480-487, 514-539, 552-563, and 583-601. In some embodiments provided methods comprise the use of at least one set of primers i) and ii) comprising at least 60 primers selected from SEQ ID NOs: 69-136 and about 5 to about 15 primers selected from SEQ ID NOs: 460-471, 480-487, 514-539, 552-563, and 583-601. In some embodiments provided methods comprise the use of at least one set of primers i) and ii) comprising at least 60 primers selected from SEQ ID NOs: 69-136 and at least one primer selected from SEQ ID NOs: 460-471, at least one primer selected from SEQ ID NOs: 480-487, at least one primer selected from SEQ ID NOs: 514-539, at least one primer selected from SEQ ID NOs: 552-563 and at least one primer selected from SEQ ID NOs: 583-601. In some embodiments methods of the invention comprise the use of at least one set of primers i) and ii) comprising at least 60 primers selected from SEQ ID NOs: 1-68 and at least one primer selected from SEQ ID NOs: 460-471, 480-487, 514-539, 552-563, and 583-601. In other embodiments methods of the invention comprise the use of at least one set of primers i) and ii) comprising at least 60 primers selected from SEQ ID NOs: 69-136 and at least one primer selected from SEQ ID NOs: 448-459, 472-479, 488-513, 540-551, and 564-582.

In certain embodiments, provided is a method for amplification of expression nucleic acid sequences of a BCR repertoire in a sample, comprising performing a multiplex amplification reaction to amplify BCR nucleic acid template molecules having a constant portion and a V gene portion using at least one set of: i) a plurality of V gene primers directed to a majority of different V genes of at least one BCR coding sequence comprising at least a portion of framework region 2 (FR2) within the V gene, and ii) one or more C gene primers directed to at least a portion of the C gene of the respective BCR coding sequence, wherein each set of i) and ii) primers directed to the same target immune receptor sequences is selected from the group consisting of IgH, IgL, and IgK, and wherein performing amplification using each set results in amplicons representing the entire repertoire of the respective immune receptor in the sample; thereby generating amplicons comprising the BCR repertoire. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In some embodiments the one or more plurality of V gene primers of i) anneal to at least a portion of the FR2 region of the BCR template molecules. In certain embodiments the one or more C gene primers of ii) comprises at least two primers that anneal to at least a portion of the constant portion C gene of the BCR template molecules. In some embodiments the one or more C gene primers of ii) comprises at least two primers each of which anneal to at least a portion of the C gene of IgA, IgD, IgG, IgM or IgE template molecules. In some embodiments the one or more C gene primers of ii) comprises at least one primer separately directed to a portion of the C gene of each of IgA, IgD, IgG, IgM and IgE template molecules.

In particular embodiments at least one set of the generated amplicons includes complementarity determining regions CDR2 and CDR3 of a BCR expression sequence. In some embodiments the amplicons are about 180 to about 375 nucleotides in length, about 200 to about 350 nucleotides, about 225 to about 325 nucleotides, or about 250 to about 300 nucleotides in length. In some embodiments the nucleic acid template used in methods is cDNA produced by reverse transcribing nucleic acid molecules extracted from a biological sample.

In certain embodiments, methods are provided for providing sequence of the BCR repertoire in a sample, comprising performing a multiplex amplification reaction to amplify BCR nucleic acid template molecules having a constant portion and a variable portion using at least one set of primers comprising i) a plurality of V gene primers directed to a majority of different V genes of at least one BCR coding sequence comprising at least a portion of FR2 within the V gene, and ii) one or more C gene primers directed to at least a portion of the respective target C gene of the BCR coding sequence, wherein each set of i) and ii) primers directed to the same target immune receptor sequences is selected from the group consisting of IgH, IgL, and IgK, thereby generating BCR amplicon molecules. Sequencing of resulting BCR amplicon molecules is then performed and the sequences of the BCR amplicon molecules determined thereby provides sequence of the BCR repertoire in the sample. In particular embodiments, determining the sequence of the BCR amplicon molecules includes obtaining initial sequence reads, aligning the initial sequence read to a reference sequence and identifying productive reads, correcting one or more indel errors to generate rescued productive sequence reads; and determining the sequences of the resulting BCR molecules. In particular embodiments the combination of productive reads and rescued productive reads is at least 40%, at least 50%, at least 60% at least 70% or at least 75% of the sequencing reads for the BCRs. In additional embodiments the method further comprises sequence read clustering and BCR clonotype reporting. In some embodiments, the sequences of the identified immune repertoire are compared to a contemporaneous or current version of the IMGT database and the sequence of at least one allelic variant absent from that IMGT database is identified. In some embodiments the average sequence read length is between about 200 and about 375 nucleotides, between about 250 and about 350 nucleotides, or between about 275 and about 350 nucleotides, depending in part on inclusion of any barcode sequence in the read length. In certain embodiments at least one set of the sequenced amplicons includes complementarity determining regions CDR2 and CDR3 of a BCR expression sequence.

In particular embodiments, methods provided utilize target BCR primer sets comprising V gene primers wherein the one or more of a plurality of V gene primers are directed to sequences over an FR2 region about 70 nucleotides in length. In other particular embodiments the one or more of a plurality of V gene primers are directed to sequences over an FR2 region about 50 nucleotides in length. In certain embodiments a target BCR primer set comprises V gene primers comprising about 4 to about 20 different FR2-directed primers. In some embodiments a target BCR primer set comprises V gene primers comprising about 5 to about 15 different FR2-directed primers. In some embodiments a target BCR primer set comprises V gene primers comprising about 5, 6, 7, 8, 9, 10, 11, or 12 different FR2-directed primers. In some embodiments the target BCR primer set comprises one or more C gene primers. In particular embodiments a target immune receptor primer set comprises at least 5 to about 15 C gene primers wherein each is directed to at least a portion of the same 50 nucleotide region within each of the target C genes. In particular embodiments a target BCR primer set comprises at least 2 to about 8 C gene primers wherein each is directed to at least a portion of the same 50 nucleotide region within each of the target C genes. In some embodiments the one or more C gene primers of ii) comprises at least two primers each of which anneal to at least a portion of the C gene of IgA, IgD, IgG, IgM or IgE template molecules. In some embodiments the one or more C gene primers of ii) comprises at least one primer separately directed to a portion of the C gene of each of IgA, IgD, IgG, IgM and IgE template molecules.

In particular embodiments, methods of the invention comprise use of at least one set of primers comprising V gene primers i) and C gene primers ii) selected from Table 4 and from Tables 6-10, respectively. In certain other embodiments methods of the invention comprise use of at least one set of primers i) and ii) comprising primers selected from SEQ ID NOs: 431-437 and 448-459, 472-479, 488-513, 540-551, and 564-582. In other embodiments methods of the invention comprise use of at least one set of primers i) and ii) comprising primers selected from SEQ ID NOs: 431-437 and 460-471, 480-487, 514-539, 552-563, and 583-601. In some embodiments methods of the invention comprise the use of at least one set of primers i) and ii) comprising at least 5 primers selected from SEQ ID NOs: 431-437 and at least one primer selected from SEQ ID NOs: 448-459, 472-479, 488-513, 540-551, and 564-582. In some embodiments provided methods comprise the use of at least one set of primers i) and ii) comprising at least 5 primers selected from SEQ ID NOs: 431-437 and at least one primer selected from SEQ ID NOs: 448-459, at least one primer selected from SEQ ID NOs: 472-479, at least one primer selected from SEQ ID NOs: 488-513, at least one primer selected from SEQ ID NOs: 540-551 and at least one primer selected from SEQ ID NOs: 564-582. In other embodiments methods of the invention comprise the use of at least one set of primers i) and ii) comprising at least 5 primers selected from SEQ ID NOs: 431-437 and at least one primer selected from SEQ ID NOs: 460-471, 480-487, 514-539, 552-563, and 583-601. In some embodiments provided methods comprise the use of at least one set of primers i) and ii) comprising at least 5 primers selected from SEQ ID NOs: 431-437 and at least one primer selected from SEQ ID NOs: 460-471, at least one primer selected from SEQ ID NOs: 480-487, at least one primer selected from SEQ ID NOs: 514-539, at least one primer selected from SEQ ID NOs: 552-563 and at least one primer selected from SEQ ID NOs: 583-601.

In certain embodiments, provided is a method for amplification of expression nucleic acid sequences of a BCR repertoire in a sample, comprising performing a multiplex amplification reaction to amplify BCR nucleic acid template molecules having a J gene portion and a V gene portion using at least one set of: i) a plurality of V gene primers directed to a majority of different V genes of a BCR coding sequence comprising at least a portion of a framework region within the V gene, and ii) a plurality of J gene primers directed to a majority of different J genes of the respective target immune receptor coding sequence, wherein each set of i) and ii) primers directed to the same target immune receptor sequences is selected from the group consisting of IgH, IgL, and IgK, and wherein performing amplification using each set results in amplicons representing the entire repertoire of the respective immune receptor in the sample; thereby generating amplicons comprising the repertoire of the BCR. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In particular embodiments the one or more plurality of J gene primers of ii) are directed to sequences over about a 50 nucleotide portion of the J gene. In more particular embodiments the one or more plurality of J gene primers of ii) are directed to sequences over about a 30 nucleotide portion of the J gene. In certain embodiments, the one or more plurality of J gene primers of ii) are directed to sequences completely within the J gene.

In certain embodiments, provided is a method for amplification of expression nucleic acid sequences of a BCR repertoire in a sample, comprising performing a multiplex amplification reaction to amplify BCR nucleic acid template molecules having a J gene portion and a V gene portion using at least one set of: i) a plurality of V gene primers directed to a majority of different V genes of at least one BCR coding sequence comprising at least a portion of framework region 3 (FR3) within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK, and wherein performing amplification using each set results in amplicons representing the entire repertoire of the respective immune receptor in the sample; thereby generating BCR amplicons comprising the repertoire of the BCR. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In more particular embodiments the one or more plurality of V gene primers of i) are directed to sequences over about a 40 to about a 60 nucleotide portion of the framework region. In some embodiments the one or more plurality of V gene primers of i) anneal to at least a portion of the framework 3 region of the template molecules. In certain embodiments the plurality of J gene primers of ii) comprises at least two primers that anneal to at least a portion of the J gene portion of the template molecules. In some embodiments the plurality of J gene primers of ii) comprises at least 2 to about 8 primers that anneal to at least a portion of the J gene portion of the template molecules. In some embodiments the plurality of J gene primers of ii) comprises about 4 primers that anneal to at least a portion of the J gene portion of the template molecules. In some embodiments the plurality of J gene primers of ii) comprises about 3 to about 6 primers that anneal to at least a portion of the J gene portion of the template molecules. In particular embodiments at least one set of the generated amplicons includes complementarity determining region CDR3 of a BCR expression sequence. In some embodiments the amplicons are about 60 to about 160 nucleotides in length, about 70 to about 100 nucleotides in length, about 100 to about 120 nucleotides in length, at least about 70 to about 90 nucleotides in length, about 80 to about 90 nucleotides in length, or about 80 nucleotides in length. In some embodiments the nucleic acid template used in methods is cDNA produced by reverse transcribing nucleic acid molecules extracted from a biological sample.

In certain embodiments, methods are provided for providing sequence of the BCR repertoire in a sample, comprising performing a multiplex amplification reaction to amplify BCR nucleic acid template molecules having a J gene portion and a V gene portion using at least one set of primers comprising i) a plurality of V gene primers directed to a majority of different V genes of at least one BCR coding sequence comprising at least a portion of framework region 3 (FR3) within the V gene, and ii) a plurality of J gene primers directed to a majority of different J genes of the respective target immune receptor coding sequence, wherein each set of i) and ii) primers directed to the same target immune receptor sequences is selected from the group consisting of IgH, IgL, and IgK, thereby generating BCR amplicon molecules. Sequencing of resulting BCR amplicon molecules is then performed and the sequences of the immune receptor amplicon molecules determined thereby provides sequence of the BCR repertoire in the sample. In some embodiments, determining the sequence of the BCR amplicon molecules includes obtaining initial sequence reads, aligning the initial sequence read to a reference sequence, identifying productive reads, correcting one or more indel errors to generate rescued productive sequence reads, and determining the sequences of the resulting immune receptor molecules. In particular embodiments, determining the sequence of the BCR amplicon molecules includes obtaining initial sequence reads, adding the inferred J gene sequence to the sequence read to create an extended sequence read, aligning the extended sequence read to a reference sequence and identifying productive reads, correcting one or more indel errors to generate rescued productive sequence reads, and determining the sequences of the resulting BCR molecules. In particular embodiments the combination of productive reads and rescued productive reads is at least 50%, at least 60% at least 70% or at least 75% of the sequencing reads for the BCRs. In additional embodiments the method further comprises sequence read clustering and BCR clonotype reporting. In some embodiments, the sequences of the identified BCR repertoire are compared to a contemporaneous or current version of the IMGT database and the sequence of at least one allelic variant absent from that IMGT database is identified. In some embodiments the sequence read lengths are about 60 to about 185 nucleotides, depending in part on inclusion of any barcode sequence in the read length. In some embodiments the average sequence read length is between 90 and 120 nucleotides, is between 70 and 90 nucleotides, or is between about 75 and about 85 nucleotides, or is about 80 nucleotides. In certain embodiments at least one set of the sequenced amplicons includes complementarity determining region CDR3 of a BCR expression sequence.

In particular embodiments, methods provided utilize target BCR primer sets comprising V gene primers wherein the one or more of a plurality of V gene primers are directed to sequences over an FR3 region about 50 nucleotides in length. In other embodiments the one or more of a plurality of V gene primers are directed to sequences over an FR3 region about 70 nucleotides in length. In other particular embodiments the one or more of a plurality of V gene primers are directed to sequences over an FR3 region about 40 to about 60 nucleotides in length. In certain embodiments a target BCR primer set comprises V gene primers comprising about 50 to about 85 different FR3-directed primers. In certain embodiments a target BCR primer set comprises V gene primers comprising about 55 to about 80 different FR3-directed primers. In some embodiments, a target immune receptor primer set comprises V gene primers comprising about 62 to about 75 different FR3-directed primers. In some embodiments, a target BCR primer set comprises V gene primers comprising about 65, 66, 67, 68, 69, or 70 different FR3-directed primers. In some embodiments the target BCR primer set comprises a plurality of J gene primers. In some embodiments a target BCR primer set comprises at least two J gene primers wherein each is directed to at least a portion of a J gene within target polynucleotides. In some embodiments a target BCR primer set comprises 2 to about 8 J gene primers wherein each is directed to at least a portion of a J gene within target polynucleotides. In some embodiments a target BCR primer set comprises about 3 to about 6 different J gene primers wherein each is directed to at least a portion of a J gene within target polynucleotides. In some embodiments a target BCR primer set comprises about 2, 3, 4, 5, 6, 7 or 8 different J gene primers. In particular embodiments a target immune receptor primer set comprises about 4 J gene primers wherein each is directed to at least a portion of a J gene within target polynucleotides.

In particular embodiments, methods of the invention comprise the use of at least one set of primers comprising V gene primers i) and J gene primers ii) selected from Tables 2 and 5, respectively. In certain other embodiments methods of the invention comprise the use of at least one set of primers i) and ii) comprising primers selected from SEQ ID NOs: 1-68 and 438-442 or selected from SEQ ID NOs: 69-136 and 443-447. In certain other embodiments methods of the invention comprise the use of at least one set of primers i) and ii) comprising primers selected from SEQ ID NOs: 1-68 and 443-447 or selected from SEQ ID NOs: 69-136 and 438-442.

In some embodiments methods of the invention comprise the use of at least one set of primers i) and ii) comprising at least 60 primers selected from SEQ ID NOs: 1-68 and at least 2 primers, at least 3 primers, or at least 4 primers selected from SEQ ID NOs: 438-442. In some embodiments methods of the invention comprise the use of at least one set of primers i) and ii) comprising at least 60 primers selected from SEQ ID NOs: 69-136 and at least 2 primers, at least 3 primers, or at least 4 primers selected from SEQ ID NOs: 443-447. In some embodiments methods of the invention comprise the use of at least one set of primers i) and ii) comprising at least 60 primers selected from SEQ ID NOs: 69-136 and at least 2 primers, at least 3 primers, or at least 4 primers selected from SEQ ID NOs: 438-442. In some embodiments methods of the invention comprise the use of at least one set of primers i) and ii) comprising at least 60 primers selected from SEQ ID NOs: 1-68 and at least 2 primers, at least 3 primers, or at least 4 primers selected from SEQ ID NOs: 443-447.

In certain embodiments, provided is a method for amplification of expression nucleic acid sequences of a BCR repertoire in a sample, comprising performing a multiplex amplification reaction to amplify BCR nucleic acid template molecules having a J gene portion and a V gene portion using at least one set of: i) a plurality of V gene primers directed to a majority of different V genes of at least one BCR coding sequence comprising at least a portion of framework region 1 (FR1) within the V gene, and ii) a plurality of J gene primers directed to a majority of different J genes of the respective target immune receptor coding sequence, wherein each set of i) and ii) primers directed to the same target immune receptor sequences is selected from the group consisting of IgH, IgL, and IgK, and wherein performing amplification using each set results in amplicons representing the entire repertoire of the respective immune receptor in the sample; thereby generating BCR amplicons comprising the repertoire of the BCR. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In some embodiments the one or more plurality of V gene primers of i) anneal to at least a portion of the framework 1 region of the template molecules. In certain embodiments the plurality of J gene primers of ii) comprises at least two primers that anneal to at least a portion of the J gene portion of the template molecules. In some embodiments the plurality of J gene primers of ii) comprises at least 2 to about 8 primers that anneal to at least a portion of the J gene portion of the template molecules. In some embodiments the plurality of J gene primers of ii) comprises about 4 primers that anneal to at least a portion of the J gene portion of the template molecules. In some embodiments the plurality of J gene primers of ii) comprises about 3 to about 6 primers that anneal to at least a portion of the J gene portion of the template molecules. In particular embodiments at least one set of the generated amplicons includes complementarity determining regions CDR1, CDR2, and CDR3 of a BCR expression sequence. In some embodiments the amplicons are about 220 to about 350 nucleotides in length, about 225 to about 300 nucleotides, about 250 to about 325 nucleotides, about 250 to about 275 nucleotides, or about 270 to about 300 nucleotides in length. In some embodiments the nucleic acid template used in methods is cDNA produced by reverse transcribing nucleic acid molecules extracted from a biological sample.

In certain embodiments, methods are provided for providing sequence of the BCR repertoire in a sample, comprising performing a multiplex amplification reaction to amplify BCR nucleic acid template molecules having a J gene portion and a V gene portion using at least one set of primers comprising i) a plurality of V gene primers directed to a majority of different V genes of at least one BCR coding sequence comprising at least a portion of framework region 1 (FR1) within the V gene, and ii) a plurality of J gene primers directed to a majority of different J genes of the respective target immune receptor coding sequence, wherein each set of i) and ii) primers directed to the same target immune receptor sequences is selected from the group consisting of IgH, IgL, and IgK, thereby generating BCR amplicon molecules. Sequencing of resulting immune receptor amplicon molecules is then performed and the sequences of the BCR amplicon molecules determined thereby provides sequence of the BCR repertoire in the sample. In some embodiments, determining the sequence of the BCR amplicon molecules includes obtaining initial sequence reads, aligning the initial sequence read to a reference sequence, identifying productive reads, correcting one or more indel errors to generate rescued productive sequence reads, and determining the sequences of the resulting immune receptor molecules. In particular embodiments, determining the sequence of the BCR amplicon molecules includes obtaining initial sequence reads, adding the inferred J gene sequence to the sequence read to create an extended sequence read, aligning the extended sequence read to a reference sequence and identifying productive reads, correcting one or more indel errors to generate rescued productive sequence reads, and determining the sequences of the resulting BCR molecules. In particular embodiments the combination of productive reads and rescued productive reads is at least 50%, at least 60% at least 70% or at least 75% of the sequencing reads for the immune receptors. In additional embodiments the method further comprises sequence read clustering and BCR clonotype reporting. In some embodiments, the sequences of the identified immune repertoire are compared to a contemporaneous or current version of the IMGT database and the sequence of at least one allelic variant absent from that IMGT database is identified. In some embodiments the average sequence read length is between 200 and 350 nucleotides, between 225 and 325 nucleotides, between 250 and 300 nucleotides, between 270 and 300 nucleotides, or is between 295 and 325 nucleotides, depending in part on inclusion of any barcode sequence in the read length. In certain embodiments at least one set of the sequenced amplicons includes complementarity determining regions CDR1, CDR2, and CDR3 of a BCR expression sequence.

In particular embodiments, methods provided utilize target BCR primer sets comprising V gene primers wherein the one or more of a plurality of V gene primers are directed to sequences over an FR1 region about 70 nucleotides in length. In other certain embodiments the one or more of a plurality of V gene primers are directed to sequences over an FR1 region about 80 nucleotides in length. In other particular embodiments the one or more of a plurality of V gene primers are directed to sequences over an FR1 region about 50 nucleotides in length. In certain embodiments a target BCR primer set comprises V gene primers comprising about 18 to about 45 different FR1-directed primers. In some embodiments a target BCR primer set comprises V gene primers comprising about 22 to about 35 different FR1-directed primers. In some embodiments a target BCR primer set comprises V gene primers comprising about 25 to about 35 different FR1-directed primers. In certain embodiments a target BCR primer set comprises V gene primers comprising about 40 to about 65 different FR1-directed primers. In some embodiments a target BCR primer set comprises V gene primers comprising about 48 to about 60 different FR1-directed primers. In some embodiments the target BCR primer set comprises a plurality of J gene primers. In some embodiments a target BCR primer set comprises at least two J gene primers wherein each is directed to at least a portion of a J gene within target polynucleotides. In some embodiments a target BCR primer set comprises 2 to about 8 J gene primers wherein each is directed to at least a portion of a J gene within target polynucleotides. In some embodiments a target BCR primer set comprises about 3 to about 6 different J gene primers wherein each is directed to at least a portion of a J gene within target polynucleotides. In some embodiments a target BCR primer set comprises about 2, 3, 4, 5, 6, 7 or 8 different J gene primers. In particular embodiments a target immune receptor primer set comprises about 4 J gene primers wherein each is directed to at least a portion of a J gene within target polynucleotides.

In particular embodiments, methods of the invention comprise use of at least one set of primers comprising V gene primers i) and J gene primers ii) selected from Tables 3 and 5, respectively. In certain other embodiments methods of the invention comprise use of at least one set of primers i) and ii) comprising primers selected from SEQ ID NOs: 137-283 and 438-442 or selected from SEQ ID NOs: 284-430 and 443-447. In other embodiments methods of the invention comprise use of at least one set of primers i) and ii) comprising primers selected from SEQ ID NOs: 137-283 and 443-447 or selected from SEQ ID NOs: 284-430 and 438-442. In some embodiments methods of the invention comprise the use of at least one set of primers i) and ii) comprising at least 20 or at least 25 primers selected from SEQ ID NOs: 137-283 and at least 2 primers, at least 3 primers, or at least 4 primers selected from SEQ ID NOs: 438-442. In some embodiments provided methods comprise the use of at least one set of primers i) and ii) comprising about 15 to about 35 primers selected from SEQ ID NOs: 137-283 and at least 2 primers, at least 3 primers, or at least 4 primers selected from SEQ ID NOs: 438-442. In some embodiments provided methods comprise the use of at least one set of primers i) and ii) comprising about 22 to about 35 primers selected from SEQ ID NOs: 137-283 and at least 2 primers, at least 3 primers, or at least 4 primers selected from SEQ ID NOs: 438-442. In other embodiments methods of the invention comprise the use of at least one set of primers i) and ii) comprising at least 20 or at least 25 primers selected from SEQ ID NOs: 284-430 and at least 2 primers, at least 3 primers, or at least 4 primers selected from SEQ ID NOs: 443-447. In some embodiments provided methods comprise the use of at least one set of primers i) and ii) comprising about 15 to about 35 primers selected from SEQ ID NOs: 284-430 and at least 2 primers, at least 3 primers, or at least 4 primers selected from SEQ ID NOs: 443-447. In some embodiments provided methods comprise the use of at least one set of primers i) and ii) comprising about 22 to about 35 primers selected from SEQ ID NOs: 284-430 and at least 2 primers, at least 3 primers, or at least 4 primers selected from SEQ ID NOs: 443-447. In some embodiments methods of the invention comprise the use of at least one set of primers i) and ii) comprising at least 20 or at least 25 primers selected from SEQ ID NOs: 137-283 and at least 2 primers, at least 3 primers, or at least 4 primers selected from SEQ ID NOs: 443-447. In other embodiments methods of the invention comprise the use of at least one set of primers i) and ii) comprising at least 20 or at least 25 primers selected from SEQ ID NOs: 284-430 and at least 2 primers, at least 3 primers, or at least 4 primers selected from SEQ ID NOs: 438-442.

In some embodiments methods of the invention comprise the use of at least one set of primers i) and ii) comprising at least 40 or at least 50 primers selected from SEQ ID NOs: 137-283 and at least 2 primers, at least 3 primers, or at least 4 primers selected from SEQ ID NOs: 438-442. In some embodiments provided methods comprise the use of at least one set of primers i) and ii) comprising about 40 to about 65 primers selected from SEQ ID NOs: 137-283 and at least 2 primers, at least 3 primers, or at least 4 primers selected from SEQ ID NOs: 438-442. In some embodiments provided methods comprise the use of at least one set of primers i) and ii) comprising about 48 to about 60 primers selected from SEQ ID NOs: 137-283 and at least 2 primers, at least 3 primers, or at least 4 primers selected from SEQ ID NOs: 438-442. In other embodiments methods of the invention comprise the use of at least one set of primers i) and ii) comprising at least 40 or at least 50 primers selected from SEQ ID NOs: 284-430 and at least 2 primers, at least 3 primers, or at least 4 primers selected from SEQ ID NOs: 443-447. In some embodiments provided methods comprise the use of at least one set of primers i) and ii) comprising about 40 to about 65 primers selected from SEQ ID NOs: 284-430 and at least 2 primers, at least 3 primers, or at least 4 primers selected from SEQ ID NOs: 443-447. In some embodiments provided methods comprise the use of at least one set of primers i) and ii) comprising about 48 to about 60 primers selected from SEQ ID NOs: 284-430 and at least 2 primers, at least 3 primers, or at least 4 primers selected from SEQ ID NOs: 443-447. In some embodiments methods of the invention comprise the use of at least one set of primers i) and ii) comprising at least 40 or at least 50 primers selected from SEQ ID NOs: 137-283 and at least 2 primers, at least 3 primers, or at least 4 primers selected from SEQ ID NOs: 443-447. In other embodiments methods of the invention comprise the use of at least one set of primers i) and ii) comprising at least 40 or at least 50 primers selected from SEQ ID NOs: 284-430 and at least 2 primers, at least 3 primers, or at least 4 primers selected from SEQ ID NOs: 438-442.

In certain embodiments, provided is a method for amplification of expression nucleic acid sequences of a BCR repertoire in a sample, comprising performing a multiplex amplification reaction to amplify BCR nucleic acid template molecules having a J gene portion and a V gene portion using at least one set of: i) a plurality of V gene primers directed to a majority of different V genes of a BCR coding sequence comprising at least a portion of framework region 2 (FR2) within the V gene, and ii) a plurality of J gene primers directed to a majority of different J genes of the respective target immune receptor coding sequence, wherein each set of i) and ii) primers directed to the same target immune receptor sequences is selected from the group consisting of IgH, IgL, and IgK, and wherein performing amplification using each set results in amplicons representing the entire repertoire of the respective immune receptor in the sample; thereby generating immune receptor amplicons comprising the repertoire of the BCR. In particular embodiments the one or more plurality of V gene primers of i) are directed to sequences over about an 80 nucleotide portion of the framework region. In more particular embodiments the one or more plurality of V gene primers of i) are directed to sequences over about a 50 nucleotide portion of the framework region. In some embodiments the one or more plurality of V gene primers of i) anneal to at least a portion of the FR2 region of the template molecules. In certain embodiments the plurality of J gene primers of ii) comprise at least ten primers that anneal to at least a portion of the J gene of the template molecules. In some embodiments the plurality of J gene primers of ii) comprises about 14 primers that anneal to at least a portion of the J gene portion of the template molecules. In some embodiments the plurality of J gene primers of ii) at least two primers that anneal to at least a portion of the J gene portion of the template molecules. In some embodiments the plurality of J gene primers of ii) comprises at least 2 to about 8 primers that anneal to at least a portion of the J gene portion of the template molecules. In some embodiments the plurality of J gene primers of ii) comprises about 4 primers that anneal to at least a portion of the J gene portion of the template molecules. In some embodiments the plurality of J gene primers of ii) comprises about 3 to about 6 primers that anneal to at least a portion of the J gene portion of the template molecules. In particular embodiments at least one set of the generated amplicons includes complementarity determining regions CDR2 and CDR3 of a BCR gene sequence. In some embodiments the amplicons are about 160 to about 270 nucleotides in length, about 180 to about 250 nucleotides, or about 195 to about 225 nucleotides in length. In some embodiments the nucleic acid template used in methods is cDNA produced by reverse transcribing nucleic acid molecules extracted from a biological sample.

In certain embodiments, methods are provided for providing sequence of the BCR repertoire in a sample, comprising performing a multiplex amplification reaction to amplify BCR nucleic acid template molecules having a J gene portion and a V gene portion using at least one set of primers comprising i) a plurality of V gene primers directed to a majority of different V genes of at least one BCR coding sequence comprising at least a portion of FR2 within the V gene, and ii) a plurality of J gene primers directed to a majority of different J genes of the respective target immune receptor coding sequence, wherein each set of i) and ii) primers directed to the same target immune receptor sequences is selected from the group consisting of IgH, IgL, and IgK, thereby generating BCR amplicon molecules.

Sequencing of resulting immune receptor amplicon molecules is then performed and the sequences of the BCR amplicon molecules determined thereby provides sequence of the BCR repertoire in the sample. In some embodiments, determining the sequence of the BCR amplicon molecules includes obtaining initial sequence reads, aligning the initial sequence read to a reference sequence, identifying productive reads, correcting one or more indel errors to generate rescued productive sequence reads, and determining the sequences of the resulting immune receptor molecules. In particular embodiments, determining the sequence of the BCR amplicon molecules includes obtaining initial sequence reads, adding the inferred J gene sequence to the sequence read to create an extended sequence read, aligning the extended sequence read to a reference sequence and identifying productive reads, correcting one or more indel errors to generate rescued productive sequence reads, and determining the sequences of the resulting BCR molecules. In particular embodiments the combination of productive reads and rescued productive reads is at least 40%, at least 50%, at least 60% at least 70% or at least 75% of the sequencing reads for the BCRs. In additional embodiments the method further comprises sequence read clustering and BCR clonotype reporting. In some embodiments, the sequences of the identified immune repertoire are compared to a contemporaneous or current version of the IMGT database and the sequence of at least one allelic variant absent from that IMGT database is identified. In some embodiments the average sequence read length is between 160 and 300 nucleotides, between 180 and 280 nucleotides, between 200 and 260 nucleotides, or between 225 and 270 nucleotides, depending in part on inclusion of any barcode sequence in the read length. In certain embodiments at least one set of the sequenced amplicons includes complementarity determining regions CDR2 and CDR3 of a BCR expression sequence.

In particular embodiments, methods provided utilize target BCR primer sets comprising V gene primers wherein the one or more of a plurality of V gene primers are directed to sequences over an FR2 region about 70 nucleotides in length. In other particular embodiments the one or more of a plurality of V gene primers are directed to sequences over an FR2 region about 50 nucleotides in length. In certain embodiments a target BCR primer set comprises V gene primers comprising about 4 to about 20 different FR2-directed primers. In some embodiments a target BCR primer set comprises V gene primers comprising about 5 to about 15 different FR2-directed primers. In some embodiments a target BCR primer set comprises V gene primers comprising about 5, 6, 7, 8, 9, 10, 11, or 12 different FR2-directed primers. In some embodiments the target BCR primer set comprises a plurality of J gene primers. In some embodiments a target BCR primer set comprises at least two J gene primers wherein each is directed to at least a portion of a J gene within target polynucleotides. In some embodiments a target BCR primer set comprises 2 to about 8 J gene primers wherein each is directed to at least a portion of a J gene within target polynucleotides. In some embodiments a target BCR primer set comprises about 3 to about 6 different J gene primers wherein each is directed to at least a portion of a J gene within target polynucleotides. In some embodiments a target BCR primer set comprises about 2, 3, 4, 5, 6, 7 or 8 different J gene primers. In particular embodiments a target immune receptor primer set comprises about 4 J gene primers wherein each is directed to at least a portion of a J gene within target polynucleotides.

In particular embodiments, methods of the invention comprise use of at least one set of primers comprising V gene primers i) and J gene primers ii) selected from Tables 4 and 5, respectively. In certain other embodiments methods of the invention comprise use of at least one set of primers i) and ii) comprising primer selected from SEQ ID NOs: 431-437 and 438-442 or selected from SEQ ID NOs: 431-437 and 443-447. In some embodiments methods of the invention comprise the use of at least one set of primers i) and ii) comprising at least 5 primers selected from SEQ ID NOs: 431-437 and at least 2 primers, at least 3 primers, or at least 4 primers selected from SEQ ID NOs: 438-442. In other embodiments methods of the invention comprise the use of at least one set of primers i) and ii) comprising at least 5 primers selected from SEQ ID NOs: 431-437 and at least 2 primers, at least 3 primers, or at least 4 primers selected from SEQ ID NOs: 443-447.

In certain embodiments, methods of the invention comprise use of a biological sample selected from the group consisting of hematopoietic cells, lymphocytes, and tumor cells. In some embodiments the biological sample is selected from the group consisting of peripheral blood mononuclear cells (PBMCs), T cells, B cells, circulating tumor cells, and tumor infiltrating lymphocytes (herein “TILs” or “TIL”). In some embodiments, the biological sample comprises B cells undergoing ex vivo activation and/or expansion. In some embodiments, the biological sample comprises cfDNA, such as found, for example, in blood or plasma. In some embodiments, the biological sample is selected from the group consisting of tissue (for example, lymph node, organ tissue, bone marrow), whole blood, synovial fluid, cerebral spinal fluid, tumor biopsy, and other clinical specimens containing cells.

In some embodiments, methods, compositions, and systems are provided for determining the immune repertoire of a biological sample by assessing both expressed immune receptor RNA and rearranged immune receptor genomic DNA (gDNA) from a biological sample. In some embodiments, the sample RNA and gDNA may be assessed concurrently and following reverse transcription of the RNA to form cDNA, the cDNA and gDNA may be amplified in the same multiplex amplification reaction. In some embodiments, cDNA from the sample RNA and the sample gDNA may undergo multiplex amplification in separate reactions. In some embodiments, cDNA from the sample RNA and sample gDNA may undergo multiplex amplification with parallel primer pools. In some embodiments, the same BCR-directed primer pools are used to assess the BCR repertoire of gDNA and RNA from the sample. In some embodiments, different immune receptor-directed primer pools are used to assess the immune repertoire of gDNA and RNA from the sample. In some embodiments, multiplex amplification reactions are performed separately with cDNA from the sample RNA and with sample gDNA to amplify the same or different target immune receptor molecules from the sample and the resulting immune receptor amplicons are sequenced, thereby providing sequence of the expressed immune receptor RNA and rearranged immune receptor gDNA of a biological sample.

In some embodiments, different immune receptor-directed primer pools are used to assess the immune repertoire of gDNA and/or RNA from the sample. In some embodiments, multiplex amplification reactions are performed with a set of IgH primers provided herein and with a set of TCR beta-directed primers, for example as described in PCT Application No. PCT/US2018/014111, filed Jan. 17, 2018, and PCT Application No. PCT/US2018/049259, filed Aug. 31, 2018, the entirety of each of which is incorporated herein by reference, or commercially available as Oncomine™ TCR Beta-SR Assay DNA, Oncomine™ TCR Beta-SR Assay RNA, and Oncomine™ TCR Beta-LR Assay (Thermo Fisher Scientific). The ability to assess both the BCR (eg, IgH) and TCR (eg, TCR beta) repertoires from a sample using a single multiplex amplification reaction is useful in saving time and limited biological sample and is applicable in many of the methods described herein, including methods related to allergy and autoimmunity, vaccine development and use, and immune-oncology. For example, combining B cell repertoire analysis with T cell repertoire analysis may be used to improve detection of changes in the immune repertoire following administration of immunotherapy, such as checkpoint blockade or checkpoint inhibitor immunotherapy, potentially indicating a response to the immunotherapy. Also, combining B cell repertoire analysis with T cell repertoire analysis may be used to improve evaluation of vaccine efficacy. Exemplary immune repertoire changes in response to immunotherapy or in response to vaccine administration include, without limitation, a decrease in T and B cell evenness following treatment (for example without limitation, at day 7-14 post treatment) in comparison to the pretreatment evenness values, and an increase in the representation of IgG1 expressing B cells following treatment(s) in comparison to the pretreatment values.

In some embodiments, the methods and compositions provided are used to identify and/or characterize an immune repertoire of a subject. In some embodiments, methods and compositions provided are used to identify and characterize novel or non-canonical BCR alleles of a subject's immune repertoire. In some embodiments, the sequences of the identified immune repertoire are compared to a contemporaneous or current version of the IMGT database and the sequence of at least one allelic variant absent from that IMGT database is identified. In some embodiments, identified allelic variants absent from the IMGT database are subjected to evidence-based filtering using, for example, criteria such as clone number support, sequence read support and/or number of individuals having the allelic variant. Allelic variants identified and reported as absent from IMGT may be compared to other databases containing immune repertoire sequence information, such as NCBI NR database and Lym1K database, to cross-validate the reported novel or non-canonical BCR alleles. Characterizing the existence of undocumented or non-canonical IgH polymorphism, for example, may help with understanding factors that influence autoimmune disease, infectious disease, and response to immunotherapy. In some embodiments, the sequences of novel or non-canonical BCR alleles identified as described herein may be used to generate recombinant BCR nucleic acids or molecules. In other embodiments accordingly, provided are methods for making recombinant nucleic acids encoding identified novel IgH allelic variants. In some embodiments, provided are methods for making recombinant IgH allelic variant molecules and for making recombinant cells which express the same.

In some embodiments, methods and compositions provided are used to identify and characterize novel or non-canonical BCR alleles of a subject's immune repertoire. In some embodiments, a patient's immune repertoire may be identified or characterized before and/or after a therapeutic treatment, for example treatment for a cancer or immune disorder. In some embodiments, identification or characterization of an immune repertoire may be used to assess the effect or efficacy of a treatment, to modify therapeutic regimens, and/or to optimize the selection of therapeutic agents. In some embodiments, identification or characterization of the immune repertoire may be used to assess a patient's response to an immunotherapy, a cancer vaccine and/or other immune-based treatment or combination(s) thereof. In some embodiments, identification or characterization of the immune repertoire may indicate a patient's likelihood to respond to a therapeutic agent or may indicate a patient's likelihood to not be responsive to a therapeutic agent.

In some embodiments, a patient's BCR repertoire may be identified or characterized to monitor progression and/or treatment of hyperproliferative diseases, including detection of residual disease following patient treatment, monitor progression and/or treatment of autoimmune disease, transplantation monitoring, and to monitor conditions of antigenic stimulation, including following vaccination, exposure to bacterial, fungal, parasitic, or viral antigens, or infection by bacteria, fungi, parasites or virus. In some embodiments, identification or characterization of the BCR repertoire may be used to assess a patient's response to an anti-infective or anti-inflammatory therapy.

In some embodiments, methods and compositions are provided for identifying and/or characterizing immune repertoire clonal populations in a sample from a subject, comprising performing one or more multiplex amplification reactions with the sample or with cDNA prepared from the sample to amplify immune repertoire nucleic acid template molecules having a constant portion and a variable portion using at least one set of primers comprising i) a plurality of V gene primers directed to a majority of different V genes of at least one BCR coding sequence comprising at least a portion of framework region 1 (FR1) within the V gene, and ii) one or more C gene primers directed to at least a portion of a respective target C gene of the immune receptor coding sequence, wherein each set of i) and ii) primers directed to the same target immune receptor sequences is selected from the group consisting of IgH, IgL, and IgK, thereby generating BCR amplicon molecules. The method further comprises sequencing the resulting BCR amplicon molecules, determining the sequences of the BCR amplicon molecules, and identifying one or more immune repertoire clonal populations for the target BCR from the sample. In particular, embodiments determining the sequence of the immune receptor amplicon molecules includes obtaining initial sequence reads, aligning the initial sequence read to a reference sequence and identifying productive reads, correcting one or more indel errors to generate rescued productive sequence reads; and determining the sequences of the resulting immune receptor molecules. In other embodiments of such methods and compositions, the one or more multiplex amplification reaction is performed using at least one set of primers comprising i) a plurality of V gene primers directed to a majority of different V genes of at least one BCR coding sequence comprising at least a portion of framework region 3 (FR3) within the V gene, and ii) one or more C gene primers directed to at least a portion of a respective target C gene of the BCR coding sequence, wherein each set of i) and ii) primers directed to the same target immune receptor sequences is selected from the group consisting of IgH, IgL, and IgK. In other embodiments of such methods and compositions, the one or more multiplex amplification reaction is performed using at least one set of primers comprising i) a plurality of V gene primers directed to a majority of different V genes of at least one BCR coding sequence comprising at least a portion of framework region 2 (FR2) within the V gene, and ii) one or more C gene primers directed to at least a portion of a respective target C gene of the BCR coding sequence, wherein each set of i) and ii) primers directed to the same target immune receptor sequences is selected from the group consisting of IgH, IgL, and IgK.

In some embodiments, methods and compositions are provided for identifying and/or characterizing immune repertoire clonal populations in a sample from a subject, comprising performing one or more multiplex amplification reactions with the sample or with cDNA prepared from the sample to amplify immune repertoire nucleic acid template molecules having a J gene portion and a V gene portion using at least one set of primers comprising i) a plurality of V gene primers directed to a majority of different V genes of at least one BCR coding sequence comprising at least a portion of framework region 3 (FR3) within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK, thereby generating BCR amplicon molecules. The method further comprises sequencing the resulting BCR amplicon molecules, determining the sequences of the BCR amplicon molecules, and identifying one or more immune repertoire clonal populations for the target BCR from the sample. In particular, embodiments determining the sequence of the immune receptor amplicon molecules includes obtaining initial sequence reads, adding the inferred J gene sequence to the sequence read to create an extended sequence read, aligning the extended sequence read to a reference sequence and identifying productive reads, correcting one or more indel errors to generate rescued productive sequence reads, and determining the sequences of the resulting immune receptor molecules. In other embodiments of such methods and compositions, the multiplex amplification reaction is performed using at least one set of primers comprising i) a plurality of V gene primers directed to a majority of different V genes of at least one BCR coding sequence comprising at least a portion of framework region 1 (FR1) within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK. In other embodiments of such methods and compositions, the multiplex amplification reaction is performed using at least one set of primers comprising i) a plurality of V gene primers directed to a majority of different V genes of at least one BCR coding sequence comprising at least a portion of framework region 2 (FR2) within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK.

In some embodiments, accordingly, methods, compositions and workflows provided are for use, without limitation, in assessing clonality, diversity and richness of B cell populations. For example, clonal expansion may identify B cells that are responding to antigen challenge and longitudinal analysis may be used to evaluate efficacy of vaccination. In some embodiments, methods, compositions and workflows provided are for use in identifying clonal lineages with many members. For example, clonal lineages with many members may represent B cells that are responding to chronic antigen stimulation. In some embodiments, methods, compositions and workflows provided are for use in identifying antigen-specific B cells. For example, comparing the IgH repertoire across groups of individuals who have been exposed to the same antigen may reveal shared IgH amino acid motifs indicative of antigen specific IgH chains. In some embodiments, methods, compositions and workflows provided are for use in evaluating clonal overlap. For example, clonal overlap analysis may reveal B cell trafficking and developmental relationships between populations of B cells. In some embodiments, methods, compositions and workflows provided are for use in determining VDJ sequence of dominant clones, including in longitudinal analysis. In some embodiments, methods, compositions and workflows provided are for use in identifying malignant subclones via clonal lineage analysis. For example, for some B cell malignancies (e.g., follicular lymphoma), somatic hypermutation is ongoing, leading to the presence of malignant subclones having different but related IgH sequences that may be tracked with the provided methods, compositions and workflows.

In some embodiments, methods, compositions and workflows provided are for use in evaluating clonal evolution. For example, analysis of clonal lineages may reveal isotype switching and IgH residues important for antigen binding. In some embodiments, methods, compositions and workflows provided are for use in evaluating isotype abundance. For example, over or under representation of certain isotypes may indicate disease or immunodeficiency such as, without limitation, elevated IgG1 in response to viral infection, elevated IgE in allergy, and missing or underrepresented isotypes may indicate primary immunodeficiency. In some embodiments, methods, compositions and workflows provided are for use in quantifying somatic hypermutation. For example, the frequency of somatic hypermutation provides insight into the stage of B cell development at which malignant transformation occurred.

In some embodiments, methods and compositions provided are used to identify and/or characterize somatic hypermutations (SHM) within a BCR repertoire or clonal populations. In some embodiments, methods and compositions provided are used to identify and/or screen for rare BCR clones or subclones, for example those having somatically hypermutated VDJ rearrangements. In some embodiments, identification, quantification and/or characterization of rare BCR clones may provide biomarkers for a given condition or treatment response. Accordingly, in some embodiments, methods and compositions provided herein are used to identify, screen for and/or characterize BCR clones as biomarkers using samples obtained for example from retrospective or longitudinal subject studies.

In some embodiments, methods for identifying and/or characterizing BCR clonal lineages and SHM comprise performing one or more multiplex amplification reaction with a subject's sample to amplify BCR nucleic acid template molecules having a constant portion and a variable portion using at least one set of primers directed to a majority of different V genes of at least one BCR coding sequence comprising at least a portion of FR1, FR2 or FR3 within the V gene, and one or more C gene primers directed to at least a portion of a respective target C gene of the BCR coding sequence, sequencing the resultant BCR amplicons, and performing VDJ sequence analysis provided herein to identify and/or quantify SMH and clonal lineages for the target BCR from the sample. In other embodiments, methods for identifying and/or characterizing BCR clonal lineages and SHM comprise performing one or more multiplex amplification reaction with a subject's sample to amplify BCR nucleic acid template molecules having a J gene portion and a variable portion using at least one set of primers directed to a majority of different V genes of at least one BCR coding sequence comprising at least a portion of FR1, FR2 or FR3 within the V gene, and a plurality of J gene primers directed to a majority of different J genes of the respective target BCR coding sequence, sequencing the resultant BCR amplicons, and performing VDJ sequence analysis provided herein to identify SHM and clonal lineages for the target BCR from the sample.

In some embodiments, methods and compositions provided are used for identifying, quantifying, characterizing and/or monitoring isotype (or sub-isotype) class or isotype class switching within a BCR repertoire or B cell clonal lineage. In some embodiments, such methods comprise performing one or more multiplex amplification reaction with a subject's sample to amplify IgH nucleic acid template molecules having a constant portion and a variable portion using at least one set of primers directed to a majority of different IgH V gene coding sequences comprising at least a portion of FR1, FR2 or FR3 within the V gene, and one or more C gene primers directed to at least a portion of a C gene of the IgH coding sequence, sequencing the resultant amplicons, performing sequence analysis provided herein to identify the IgH isotype class(es) of the BCR repertoire or clonal lineages of the sample. In some embodiments, the primer set comprises one or more primers directed to at least a portion of a C gene of a single isotype, e.g., IgE. In other embodiments, the primer set comprises at least two primers each directed to at least a portion of a C gene of two different isotypes. In other embodiments, the primer set comprises at least one primer separately directed to at least a portion of a C gene of IgA, IgD, IgG, IgM and IgE isotype classes.

In certain embodiments, the methods and compositions provided are used to monitor changes in BCR repertoire clonal populations and clonal lineages, for example changes in clonal expansion, changes in clonal contraction, changes in relative ratios of clones or clonal populations within a BCR repertoire, changes in expansion or contraction of clonal lineages, changes in somatic hypermutation and/or isotype class switching within a repertoire. In some embodiments, the provided methods and compositions are used to monitor changes in BCR repertoire clonal populations or clonal lineages (e.g., clonal population or lineage expansion, clonal population or lineage contraction, clonal population or lineage changes in relative ratios, changes in somatic hypermutation and/or class switching) in response to tumor growth. In some embodiments, the provided methods and compositions are used to monitor changes in BCR repertoire clonal populations (e.g., clonal population or lineage expansion, clonal population or lineage contraction, clonal population or lineage changes in relative ratios, changes in somatic hypermutation and/or class switching) in response to tumor treatment. In some embodiments, the provided methods and compositions provided are used to monitor changes in BCR repertoire clonal populations or clonal lineages (e.g., clonal population or lineage expansion, clonal population or lineage contraction, clonal population or lineage changes in relative ratios, changes in somatic hypermutation and/or class switching) during a remission period. For many lymphoid malignancies, a clonal B cell receptor sequence can be used a biomarker for the malignant cells of the particular cancer (e.g., leukemia) and to monitor residual disease, tumor expansion, contraction, and/or treatment response. In certain embodiments a clonal B cell receptor may be identified and further characterized to confirm a new utility in therapeutic, biomarker and/or diagnostic use.

In some embodiments, methods and compositions are provided for monitoring changes in BCR clonal populations in a subject, comprising performing one or more multiplex amplification reaction with a subject's sample to amplify BCR nucleic acid template molecules having a constant portion and a variable portion using at least one set of primers directed to a majority of different V genes of at least one BCR coding sequence comprising at least a portion of FR1, FR2 or FR3 within the V gene, and ii) one or more C gene primers directed to at least a portion of a respective target C gene of the BCR coding sequence, sequencing the resultant BCR amplicons, identifying immune repertoire clonal populations for the target BCR from the sample, and comparing the identified BCR repertoire clonal populations to those identified in samples obtained from the subject at a different time. In some embodiments, methods and compositions are provided for monitoring changes in BCR clonal populations in a subject, comprising performing one or more multiplex amplification reaction with a subject's sample to amplify immune repertoire nucleic acid template molecules having a J gene portion and a V gene portion using at least one set of primers directed to a majority of different V genes of at least one BCR coding sequence comprising at least a portion of FR1, FR2 or FR3 within the V gene, and a plurality of J gene primers directed to a majority of different J genes of the respective target BCR coding sequence, sequencing the resultant BCR amplicons, identifying immune repertoire clonal populations for the target BCR from the sample, and comparing the identified immune repertoire clonal populations to those identified in samples obtained from the subject at a different time. In various embodiments, the one or more multiplex amplification reactions performed in such methods may be a single multiplex amplification reaction or may be two or more multiplex amplification reactions performed in parallel, for example parallel, highly multiplexed amplification reactions performed with different primer pools. Samples for use in monitoring changes in BCR repertoire clonal populations include, without limitation, samples obtained prior to a diagnosis, samples obtained at any stage of diagnosis, samples obtained during a remission, samples obtained at any time prior to a treatment (pre-treatment sample), samples obtained at any time following completion of treatment (post-treatment sample), and samples obtained during the course of treatment.

In certain embodiments, methods and compositions are provided for identifying and/or characterizing the BCR repertoire of a patient to monitor progression and/or treatment of the patient's hyperproliferative disease. In some embodiments, the methods and compositions provided are used for minimal residual disease (MRD) monitoring for a patient following treatment. In some embodiments, the methods and compositions provided allow for the deep sequencing of the patient BCR repertoire useful for MRD measurements and for identifying rare BCR clones. In some embodiments, monitoring MRD includes assessing somatic hypermutation of the BCR repertoire. In some embodiments, the methods and compositions are used to identify and/or track B cell lineage malignancies or T cell lineage malignancies. In some embodiments, the methods and compositions are used to detect and/or monitor MRD in patients diagnosed with leukemia or lymphoma, including without limitation, acute lymphoblastic leukemia, chronic myeloid leukemia, chronic lymphocytic leukemia, chronic myelogenous leukemia, cutaneous T cell lymphoma, B cell lymphoma, mantle cell lymphoma, and multiple myeloma. In some embodiments, the methods and compositions are used to detect and/or monitor MRD in patients diagnosed with solid tumors, including without limitation, breast cancer, lung cancer, colorectal, and neuroblastoma. In some embodiments, the methods and compositions are used to detect and/or monitor MRD in patients following cancer treatment including without limitation bone marrow transplant, lymphocyte infusion, adoptive T-cell therapy, other cell-based immunotherapy, and antibody-based immunotherapy.

In some embodiments, methods and compositions are provided for identifying and/or characterizing the BCR repertoire of a patient to monitor progression and/or treatment of the patient's hyperproliferative disease, comprising performing one or more multiplex amplification reactions with a sample from the patient or with cDNA prepared from the sample to amplify BCR nucleic acid template molecules having a constant portion and a variable portion using at least one set of primers comprising i) a plurality of V gene primers directed to a majority of different V genes of at least one BCR coding sequence comprising at least a portion of framework region 1 (FR1) within the V gene, and ii) one or more C gene primers directed to at least a portion of a respective target C gene of the BCR coding sequence, wherein each set of i) and ii) primers directed to the same target immune receptor sequences is selected from the group consisting of IgH, IgL, and IgK, thereby generating BCR amplicon molecules. The method further comprises sequencing the resulting BCR amplicon molecules, determining the sequences of the BCR amplicon molecules, and identifying immune repertoire for the target BCR from the sample. In particular, embodiments determining the sequence of the immune receptor amplicon molecules includes obtaining initial sequence reads, aligning the initial sequence read to a reference sequence and identifying productive reads, correcting one or more indel errors to generate rescued productive sequence reads; and determining the sequences of the resulting immune receptor molecules. In other embodiments of such methods and compositions, the multiplex amplification reaction is performed using at least one set of primers comprising i) a plurality of V gene primers directed to a majority of different V genes of at least one BCR coding sequence comprising at least a portion of FR3 within the V gene, and ii) one or more C gene primers directed to at least a portion of a respective target C gene of the BCR coding sequence, wherein each set of i) and ii) primers directed to the same target immune receptor sequences is selected from the group consisting of IgH, IgL, and IgK. In other embodiments of such methods and compositions, the multiplex amplification reaction is performed using at least one set of primers comprising i) a plurality of V gene primers directed to a majority of different V genes of at least one BCR coding sequence comprising at least a portion of FR2 within the V gene, and ii) one or more C gene primers directed to at least a portion of a respective target C gene of the BCR coding sequence, wherein each set of i) and ii) primers directed to the same target immune receptor sequences is selected from the group consisting of IgH, IgL, and IgK.

In some embodiments, methods and compositions are provided for identifying and/or characterizing the BCR repertoire of a patient to monitor progression and/or treatment of the patient's hyperproliferative disease, comprising performing one or more multiplex amplification reaction with a sample from the patient or with cDNA prepared from the sample to amplify immune repertoire nucleic acid template molecules having a J gene portion and a V gene portion using at least one set of primers comprising i) a plurality of V gene primers directed to a majority of different V genes of at least one BCR coding sequence comprising at least a portion of framework region 3 (FR3) within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK, thereby generating BCR amplicon molecules. The method further comprises sequencing the resulting BCR amplicon molecules, determining the sequences of the BCR amplicon molecules, and identifying immune repertoire for the target BCR from the sample. In particular, embodiments determining the sequence of the immune receptor amplicon molecules includes obtaining initial sequence reads, adding the inferred J gene sequence to the sequence read to create an extended sequence read, aligning the extended sequence read to a reference sequence and identifying productive reads, correcting one or more indel errors to generate rescued productive sequence reads; and determining the sequences of the resulting immune receptor molecules. In other embodiments of such methods and compositions, the multiplex amplification reaction is performed using at least one set of primers comprising i) a plurality of V gene primers directed to a majority of different V genes of at least one BCR coding sequence comprising at least a portion of FR1 within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK. In other embodiments of such methods and compositions, the multiplex amplification reaction is performed using at least one set of primers comprising i) a plurality of V gene primers directed to a majority of different V genes of at least one BCR coding sequence comprising at least a portion of FR2 within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK.

In some embodiments, methods and compositions are provided for MRD monitoring for a patient having a hyperproliferative disease, comprising performing one or more multiplex amplification reaction with a patient's sample to amplify BCR nucleic acid template molecules having a constant portion and a variable portion using at least one set of primers directed to a majority of different V genes of at least one BCR coding sequence comprising at least a portion of FR1, FR2 or FR3 within the V gene, and ii) one or more C gene primers directed to at least a portion of a respective target C gene of the BCR coding sequence, sequencing the resultant BCR amplicons, identifying immune repertoire sequences for the target BCR, and detecting the presence or absence of BCR sequence(s) in the sample associated with the hyperproliferative disease. In some embodiments, methods and compositions are provided for MRD monitoring for a patient having a hyperproliferative disease, comprising performing one or more multiplex amplification reaction with a patient's sample to amplify immune repertoire nucleic acid template molecules having a J gene portion and a V gene portion using at least one set of primers directed to a majority of different V genes of at least one BCR coding sequence comprising at least a portion of FR1, FR2 or FR3 within the V gene, and a plurality of J gene primers directed to a majority of different J genes of the respective target BCR coding sequence, sequencing the resultant BCR amplicons, identifying immune repertoire sequences for the target BCR, and detecting the presence or absence of immune receptor sequence(s) in the sample associated with the hyperproliferative disease. In various embodiments, the one or more multiplex amplification reactions performed in such methods may be a single multiplex amplification reaction or may be two or more multiplex amplification reactions performed in parallel, for example parallel, highly multiplexed amplification reactions performed with different primer pools. Samples for use in MRD monitoring include, without limitation, samples obtained during a remission, samples obtained at any time following completion of treatment (post-treatment sample), and samples obtained during the course of treatment.

In certain embodiments, methods and compositions are provided for identifying and/or characterizing the BCR repertoire of a subject in response to a treatment. In some embodiments, the methods and compositions are used to characterize and/or monitor populations or clones of tumor infiltrating lymphocytes (TILs) before, during, and/or following tumor treatment. In some embodiments, profiling immune receptor repertoires of TILs provides characterization and/or assessment of the tumor microenvironment. In some embodiments, the methods and compositions for determining immune repertoire are used to identify and/or track therapeutic T cell population(s) and B cell population(s). In some embodiments, the methods and compositions provided are used to identify and/or monitor the persistence of cell-based therapies following patient treatment, including but not limited to, presence (e.g., persistent presence) of engineered T cell populations including without limitation CAR-T cell populations, TCR engineered T cell populations, persistent CAR-T expression, presence (e.g., persistent presence) of administered TIL populations, TIL expression (e.g., persistent expression) following adoptive T-cell therapy, and/or immune reconstitution after allogeneic hematopoietic cell transplantation.

In some embodiments, the methods and compositions provided are used to characterize and/or monitor B cell clones or populations present in patient sample following administration of cell-based therapies to the patient, including but not limited to, e.g., cancer vaccine cells, CAR-T, TIL, and/or other engineered cell-based therapy. In some embodiments, the provided methods and compositions are used to characterize and/or monitor BCR repertoire in a patient sample following cell-based therapies in order to assess and/or monitor the patient's response to the administered cell-based therapy. Samples for use in such characterizing and/or monitoring following cell-based therapy include, without limitation, circulating blood cells, circulating tumor cells, TILs, tissue, cfDNA, and tumor sample(s) from a patient.

In some embodiments, methods and compositions are provided for monitoring cell-based therapy for a patient receiving such therapy, comprising performing one or more multiplex amplification reactions with a patient's sample to amplify BCR nucleic acid template molecules having a constant portion and a variable portion using at least one set of primers directed to a majority of different V genes of at least one BCR coding sequence comprising at least a portion of FR1, FR2 or FR3 within the V gene, and ii) one or more C gene primers directed to at least a portion of a respective target C gene of the BCR coding sequence, sequencing the resultant BCR amplicons, identifying immune repertoire sequences for the target BCR, and detecting the presence or absence of BCR sequence(s) in the sample associated with the cell-based therapy. In some embodiments, methods and compositions are provided for monitoring cell-based therapy for a patient receiving such therapy, comprising performing one or more multiplex amplification reactions with a patient's sample to amplify BCR repertoire nucleic acid template molecules having a J gene portion and a V gene portion using at least one set of primers directed to a majority of different V genes of at least one BCR coding sequence comprising at least a portion of FR1, FR2 or FR3 within the V gene, and a plurality of J gene primers directed to a majority of different J genes of the respective target BCR coding sequence, sequencing the resultant BCR amplicons, identifying immune repertoire sequences for the target BCR, and detecting the presence or absence of BCR sequence(s) in the sample associated with the cell-based therapy.

In some embodiments, methods and compositions are provided for monitoring a patient's response following administration of a cell-based therapy, comprising performing one or more multiplex amplification reactions with a patient's sample to amplify BCR repertoire nucleic acid template molecules having a constant portion and a variable portion using at least one set of primers directed to a majority of different V genes of at least one BCR coding sequence comprising at least a portion of FR1, FR2 or FR3 within the V gene, and ii) one or more C gene primers directed to at least a portion of a respective target C gene of the BCR coding sequence, sequencing the resultant BCR amplicons, identifying immune repertoire sequences for the target BCR, and comparing the identified BCR repertoire to the immune receptor sequence(s) identified in samples obtained from the patient at a different time. In some embodiments, methods and compositions are provided for monitoring a patient's response following administration of a cell-based therapy, comprising performing one or more multiplex amplification reactions with a patient's sample to amplify BCR repertoire nucleic acid template molecules having a J gene portion and a V gene portion using at least one set of primers directed to a majority of different V genes of at least one BCR coding sequence comprising at least a portion of FR1, FR2 or FR3 within the V gene, and a plurality of J gene primers directed to a majority of different J genes of the respective target BCR coding sequence, sequencing the resultant BCR amplicons, identifying immune repertoire sequences for the target BCR, and comparing the identified BCR repertoire to the immune receptor sequence(s) identified in samples obtained from the patient at a different time. Cell-based therapies suitable for such monitoring include, without limitation, CAR-T cells, TCR engineered T cells, TILs, and other enriched autologous cells. In various embodiments, the one or more multiplex amplification reactions performed in such methods may be a single multiplex amplification reaction or may be two or more multiplex amplification reactions performed in parallel, for example parallel, highly multiplexed amplification reactions performed with different primer pools. Samples for use in such monitoring include, without limitation, samples obtained prior to a diagnosis, samples obtained at any stage of diagnosis, samples obtained during a remission, samples obtained at any time prior to a treatment (pre-treatment sample), samples obtained at any time following completion of treatment (post-treatment sample), and samples obtained during the course of treatment.

In some embodiments, the methods and compositions for determining B cell receptor repertoires, or B cell and T cell receptor repertoires, are used to measure and/or assess immunocompetence before, during, and/or following a treatment, including without limitation, solid organ transplant or bone marrow transplant.

In certain embodiments, the methods and compositions provided are used to identify and/characterize a BCR repertoire of a subject in response to a therapeutic treatment including without limitation, an immunotherapy, an anti-allergy treatment, and an anti-infectious agent treatment. Accordingly, in some embodiments, methods and compositions provided are used to identify BCR repertoire or clonal lineage biomarkers or signatures of a treatment response, such as a favorable response to a therapeutic treatment (e.g., successful vaccination) or an deleterious response (e.g., an immune system-mediated adverse event). In some embodiments, methods and compositions are provided for identifying and/or characterizing the BCR repertoire of a subject in response to a treatment, comprising obtaining a sample from the subject following initiation of a treatment, performing one or more multiplex amplification reactions with the sample or with cDNA prepared from the sample to amplify BCR nucleic acid template molecules having a constant portion and a variable portion using at least one set of primers comprising i) a plurality of V gene primers directed to a majority of different V genes of at least one BCR coding sequence comprising at least a portion of framework region 1 (FR1) within the V gene, and ii) one or more C gene primers directed to at least a portion of a respective target C gene of the BCR coding sequence, wherein each set of i) and ii) primers directed to the same target immune receptor sequences is selected from the group consisting of IgH, IgL, and IgK, thereby generating BCR amplicon molecules. The method further comprises sequencing the resulting BCR amplicon molecules, determining the sequences of the BCR amplicon molecules, and identifying immune repertoire for the target BCR from the sample. In some embodiments, the method further comprises comparing the identified BCR repertoire from the sample obtained following treatment initiation to the BCR repertoire from a sample of the patient obtained prior to treatment. In particular, embodiments determining the sequence of the BCR amplicon molecules includes obtaining initial sequence reads, aligning the initial sequence read to a reference sequence and identifying productive reads, correcting one or more indel errors to generate rescued productive sequence reads; and determining the sequences of the resulting immune receptor molecules. In other embodiments of such methods and compositions, the multiplex amplification reaction is performed using at least one set of primers comprising i) a plurality of V gene primers directed to a majority of different V genes of at least one BCR coding sequence comprising at least a portion of FR3 within the V gene, and ii) one or more C gene primers directed to at least a portion of a respective target C gene of the BCR coding sequence, wherein each set of i) and ii) primers directed to the same target immune receptor sequences is selected from the group consisting of IgH, IgL, and IgK. In other embodiments of such methods and compositions, the multiplex amplification reaction is performed using at least one set of primers comprising i) a plurality of V gene primers directed to a majority of different V genes of at least one BCR coding sequence comprising at least a portion of FR2 within the V gene, and ii) one or more C gene primers directed to at least a portion of a respective target C gene of the BCR coding sequence, wherein each set of i) and ii) primers directed to the same target immune receptor sequences is selected from the group consisting of IgH, IgL, and IgK.

In some embodiments, methods and compositions are provided for identifying and/or characterizing the BCR repertoire of a subject in response to a treatment, comprising obtaining a sample from the subject following initiation of a treatment, performing one or more multiplex amplification reactions with the sample or with cDNA prepared from the sample to amplify BCR nucleic acid template molecules having a J gene portion and a V gene portion using at least one set of primers comprising i) a plurality of V gene primers directed to a majority of different V genes of at least one BCR coding sequence comprising at least a portion of framework region 3 (FR3) within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK, thereby generating BCR amplicon molecules. The method further comprises sequencing the resulting BCR amplicon molecules, determining the sequences of the BCR amplicon molecules, and identifying immune repertoire for the target BCR from the sample. In some embodiments, the method further comprises comparing the identified BCR repertoire from the sample obtained following treatment initiation to the BCR repertoire from a sample of the patient obtained prior to treatment. In particular, embodiments determining the sequence of the BCR amplicon molecules includes obtaining initial sequence reads, adding the inferred J gene sequence to the sequence read to create an extended sequence read, aligning the extended sequence read to a reference sequence and identifying productive reads, correcting one or more indel errors to generate rescued productive sequence reads; and determining the sequences of the resulting BCR molecules. In other embodiments of such methods and compositions, the multiplex amplification reaction is performed using at least one set of primers comprising i) a plurality of V gene primers directed to a majority of different V genes of at least one BCR coding sequence comprising at least a portion of FR1 within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK. In other embodiments of such methods and compositions, the multiplex amplification reaction is performed using at least one set of primers comprising i) a plurality of V gene primers directed to a majority of different V genes of at least one BCR coding sequence comprising at least a portion of FR2 within the V gene, and ii) a plurality of J gene primers directed to a majority of different J genes of the respective target BCR coding sequence, wherein each set of i) and ii) primers directed to the same target immune receptor sequences is selected from the group consisting of IgH, IgL, and IgK.

In some embodiments, methods and compositions are provided for monitoring changes in the BCR repertoire of a subject in response to a treatment, comprising performing one or more multiplex amplification reactions with a subject's or patient's sample to amplify BCR nucleic acid template molecules having a constant portion and a variable portion using at least one set of primers directed to a majority of different V genes of at least one BCR coding sequence comprising at least a portion of FR1, FR2 or FR3 within the V gene, and one or more C gene primers directed to at least a portion of a respective target C gene of the BCR coding sequence, sequencing the resultant BCR amplicons, identifying immune repertoire sequences for the target BCR from the sample, and comparing the identified BCR repertoire to those identified in samples obtained from the subject at a different time. In some embodiments, methods and compositions are provided for monitoring changes in the BCR repertoire of a subject in response to a treatment, comprising performing one or more multiplex amplification reactions with a subject's or patient's sample to amplify BCR nucleic acid template molecules having a J gene portion and a V gene portion using at least one set of primers directed to a majority of different V genes of at least one BCR coding sequence comprising at least a portion of FR1, FR2 or FR3 within the V gene, and a plurality of J gene primers directed to a majority of different J genes of the respective target BCR coding sequence, sequencing the resultant BCR amplicons, identifying immune repertoire sequences for the target BCR from the sample, and comparing the identified BCR repertoire to those identified in samples obtained from the subject at a different time. In various embodiments, the one or more multiplex amplification reactions performed in such methods may be a single multiplex amplification reaction or may be two or more multiplex amplification reactions performed in parallel, for example parallel, highly multiplexed amplification reactions performed with different primer pools. Samples for use in monitoring changes in BCR repertoire include, without limitation, samples obtained prior to a diagnosis, samples obtained at any stage of diagnosis, samples obtained during a remission, samples obtained at any time prior to a treatment (pre-treatment sample), samples obtained at any time following completion of treatment (post-treatment sample), and samples obtained during the course of treatment.

In certain embodiments, the methods and compositions provided are used to characterize and/or monitor BCR repertoires associated with immune system-mediated adverse event(s), including without limitation, those associated with inflammatory conditions, autoimmune reactions, and/or autoimmune diseases or disorders. In some embodiments, the methods and compositions provided are used to identify and/or monitor B cell, or B cell and T cell, immune repertoires associated with chronic autoimmune diseases or disorders including, without limitation, multiple sclerosis, Type I diabetes, narcolepsy, rheumatoid arthritis, ankylosing spondylitis, asthma, and SLE. In some embodiments, a systemic sample, such as a blood sample, is used to determine the immune repertoire(s) of an individual with an autoimmune condition. In some embodiments, a localized sample, such as a fluid sample from an affected joint or region of swelling, is used to determine the immune repertoire(s) of an individual with an autoimmune condition. In some embodiments, comparison of the immune repertoire found in a localized or affected area sample to the immune repertoire found in the systemic sample can identify clonal T or B cell populations to be targeted for removal.

In some embodiments, methods and compositions are provided for identifying and/or monitoring a BCR repertoire associated with progression and/or treatment of a patient's immune system-mediated adverse event(s), comprising performing one or more multiplex amplification reactions with a patient's sample to amplify BCR repertoire nucleic acid template molecules having a constant portion and a variable portion using at least one set of primers directed to a majority of different V genes of at least one BCR coding sequence comprising at least a portion of FR1, FR2 or FR3 within the V gene, and one or more C gene primers directed to at least a portion of a respective target C gene of the BCR coding sequence, sequencing the resultant BCR amplicons, identifying immune repertoire sequences for the target BCR from the sample, and comparing the identified BCR repertoire to the BCR repertoire(s) identified in samples obtained from the patient at a different time. In some embodiments, methods and compositions are provided for identifying and/or monitoring a BCR repertoire associated with progression and/or treatment of a patient's immune system-mediated adverse event(s), comprising performing one or more multiplex amplification reactions with a patient's sample to amplify BCR nucleic acid template molecules having a J gene portion and a V gene portion using at least one set of primers directed to a majority of different V genes of at least one BCR coding sequence comprising at least a portion of FR1, FR2 or FR3 within the V gene, and a plurality of J gene primers directed to a majority of different J genes of the respective target BCR coding sequence, sequencing the resultant BCR amplicons, identifying BCR sequences for the target immune receptor from the sample, and comparing the identified BCR repertoire to the BCR repertoire(s) identified in samples obtained from the patient at a different time. In various embodiments, the one or more multiplex amplification reactions performed in such methods may be a single multiplex amplification reaction or may be two or more multiplex amplification reactions performed in parallel, for example parallel, highly multiplexed amplification reactions performed with different primer pools. Samples for use in monitoring changes in immune repertoire associated with immune system-mediated adverse event(s) include, without limitation, samples obtained prior to a diagnosis, samples obtained at any stage of diagnosis, samples obtained during a remission, samples obtained at any time prior to a treatment (pre-treatment sample), samples obtained at any time following completion of treatment (post-treatment sample), and samples obtained during the course of treatment.

In some embodiments, the methods and compositions provided are used to characterize and/or monitor immune repertoires associated with passive immunity, including naturally acquired passive immunity and artificially acquired passive immunity therapies. For example, the methods and compositions provided may be used to identify and/or monitor protective antibodies that provide passive immunity to the recipient following transfer of antibody-mediated immunity to the recipient, including without limitation, antibody-mediated immunity conveyed from a mother to a fetus during pregnancy or to an infant through breast-feeding, or conveyed via administration of antibodies to a recipient. In another example, the methods and compositions provided may be used to identify and/or monitor B cell and/or T cell immune repertoires associated with passive transfer of cell-mediated immunity to a recipient, such as the administration of mature circulating lymphocytes to a recipient histocompatible with the donor. In some embodiments, the methods and compositions provided are used to monitor the duration of passive immunity in a recipient.

In some embodiments, the methods and compositions provided are used to characterize and/or monitor immune repertoires associated with active immunity or vaccination therapies. For example, following exposure to a vaccine or infectious agent, the methods and compositions provided may be used to identify and/or monitor protective antibodies or protective clonal B cell populations, or clonal B cell and T cell populations, that may provide active immunity to the exposed individual. In some embodiments, the methods and compositions provided are used to monitor the duration of B cell clones, or B cell and T cell clones, which contribute to immunity in an exposed individual. In some embodiments, the methods and compositions provided are used to identify and/or monitor B cell and/or T cell immune repertoires associated with exposure to bacterial, fungal, parasitic, or viral antigens. In some embodiments, the methods and compositions provided are used to identify and/or monitor B cell and/or T cell immune repertoires associated with bacterial, fungal, parasitic, or viral infection. Accordingly, in some embodiments, methods and composition provided are for use in vaccine development, including without limitation identifying and/or characterizing one or responses to a vaccine candidate, and assessing one or more responses to a vaccine for quality or regulatory purposes.

In some embodiments, methods and compositions are provided for monitoring changes in the BCR repertoire following exposure to a vaccine or infectious agent, comprising performing one or more multiplex amplification reactions with an exposed subject's sample to amplify BCR repertoire nucleic acid template molecules having a constant portion and a variable portion using at least one set of primers directed to a majority of different V genes of at least one BCR coding sequence comprising at least a portion of FR1, FR2 or FR3 within the V gene, and one or more C gene primers directed to at least a portion of a respective target C gene of the BCR coding sequence, sequencing the resultant BCR amplicons, identifying immune repertoire sequences for the target BCR from the sample, and comparing the identified BCR repertoire to the BCR repertoire(s) identified in samples obtained from the subject at a different time (e.g., prior to exposure or after the sample being tested was obtained). In some embodiments, methods and compositions are provided for monitoring changes in the BCR repertoire following exposure to a vaccine or infectious agent, comprising performing one or more multiplex amplification reactions with an exposed subject's sample to amplify BCR repertoire nucleic acid template molecules having a J gene portion and a V gene portion using at least one set of primers directed to a majority of different V genes of at least one BCR coding sequence comprising at least a portion of FR1, FR2 or FR3 within the V gene, and a plurality of J gene primers directed to a majority of different J genes of the respective target BCR coding sequence, sequencing the resultant BCR amplicons, identifying BCR sequences for the target immune receptor from the sample, and comparing the identified BCR repertoire to the BCR repertoire(s) identified in samples obtained from the patient at a different time. In certain embodiments, methods and compositions are provided for monitoring changes in the BCR repertoire following exposure to a vaccine or infectious agent, comprising performing one or more multiplex amplification reactions with cDNA prepared from the exposed subject's sample to amplify IgH nucleic acid template molecules having a constant portion and a variable portion using at least one set of primers comprising i) a plurality of V gene primers directed to a majority of different IgH V genes comprising at least a portion of FR1, FR2 or FR3 within the V gene, and one or more C gene primers directed to at least a portion of an IgH C gene, sequencing the resultant BCR amplicons, identifying expressed IgH repertoire sequences from the sample including the repertoire isotype information, and comparing the identified IgH repertoire to the IgH repertoire(s) identified in samples obtained from the subject at a different time (e.g., prior to exposure or after the sample being tested was obtained). In some embodiments, the primer set comprises one or more primers directed to at least a portion of a C gene of a single isotype, e.g., IgG. In other embodiments, the primer set comprises at least two primers each directed to at least a portion of a C gene of two different isotypes. In other embodiments, the primer set comprises at least one primer separately directed to at least a portion of a C gene of IgA, IgD, IgG, IgM and IgE isotype classes. Accordingly, methods and compositions may be used to monitor changes in B cell repertoire (including isotype class switching) and assess a subject's response to vaccine exposure.

In certain embodiments, methods and compositions are provided for identifying and/or characterizing the IgE repertoire of a subject following exposure to an allergen or an agent that induces an allergy reaction or response, comprising performing one or more multiplex amplification reactions with an exposed subject's sample to amplify IgH repertoire nucleic acid template molecules having a constant portion and a variable portion using at least one set of primers directed to a majority of different IgH V genes comprising at least a portion of FR1, FR2 or FR3 within the V gene, and one or more C gene primers directed to at least a portion of an IgE gene coding sequence, sequencing the resultant IgH amplicons, identifying expressed IgE immune repertoire sequences from the sample. In some embodiments, methods and compositions are provided for monitoring changes in the IgE repertoire of a subject following exposure to an allergen or an agent that induces an allergy reaction or response, comprising performing one or more multiplex amplification reactions with an exposed subject's sample to amplify IgH repertoire nucleic acid template molecules having a constant portion and a variable portion using at least one set of primers directed to a majority of different IgH V genes comprising at least a portion of FR1, FR2 or FR3 within the V gene, and one or more C gene primers directed to at least a portion of an IgE gene coding sequence, sequencing the resultant IgH amplicons, identifying expressed IgE immune repertoire sequences from the sample, and comparing the identified IgE repertoire to the IgE repertoire(s) identified in samples obtained from the subject at a different time (e.g., prior to exposure or after the sample being tested was obtained). In some embodiments, the at least one primer set of such methods and compositions comprises additional C gene primers directed to at least a portion of other IgH isotypes, such as IgG, IgM, IgA, and/or IgD directed primers. In other embodiments, the primer set comprises at least one primer separately directed to at least a portion of a C gene of IgA, IgD, IgG, IgM and IgE isotype classes. Accordingly, methods and compositions may be used to monitor changes in the IgE repertoire within the total BCR repertoire (including isotype class switching) and assess a subject's allergy reaction or response to allergen exposure. In some embodiments, such methods and compositions are used to determine and/or monitor isotype switching origins of IgE-expressing B cells within the repertoire.

In some embodiments, the methods and compositions provided are used to screen or characterize lymphocyte populations which are grown and/or activated in vitro for use as immunotherapeutic agents or in immunotherapeutic-based regimens. In some embodiments, the methods and compositions provided are used to screen or characterize TIL populations or other harvested B cell populations which are grown and/or activated in vitro. In some embodiments, determining the IgH sequence of a BCR facilitates identification and production of antigen-specific B cells. In some embodiments, the methods and compositions provided are used to screen or characterize engineered B cell populations which are grown and/or activated in vitro, for use, for example, in immunotherapy or antibody production. In some embodiments, the methods and compositions provided are used to assess cell populations by monitoring BCR repertoires during ex vivo workflows for manufacturing engineered cell preparations, for example, for quality control or regulatory testing purposes.

In some embodiments, the sequences of novel or non-canonical BCR alleles identified as described herein may be used to generate recombinant BCR nucleic acids or molecules. In some embodiments, the methods and compositions provided are used in the screening and/or production of recombinant antibody libraries. Compositions provided which are directed to identifying BCRs can be used to rapidly evaluate recombinant antibody library size and composition to identify antibodies of interest.

In some embodiments, profiling immune receptor repertoires as provided herein may be combined with profiling immune response gene expression to provide characterization of the tumor microenvironment. In some embodiments, combining or correlating a tumor sample's BCR repertoire profile with a targeted immune response gene expression profile provides a more thorough analysis of the tumor microenvironment and may suggest or provide guidance for immunotherapy treatments.

Suitable cells for analysis include, without limitation, various hematopoietic cells, lymphocytes, and tumor cells, such as peripheral blood mononuclear cells (PBMCs), T cells, B cells, circulating tumor cells, and tumor infiltrating lymphocytes (TILs). Lymphocytes expressing immunoglobulin include pre-B cells, B-cells, e.g. memory B cells, and plasma cells. Lymphocytes expressing T cell receptors include thymocytes, NK cells, pre-T cells and T cells, where many subsets of T cells are known in the art, e.g. Th1, Th2, Th17, CTL, T reg, etc. For example, in some embodiments, a sample comprising PBMCs may be used as a source for antibody immune repertoire analysis. The sample may contain, for example, lymphocytes, monocytes, and macrophages as well as antibodies and other biological constituents.

Analysis of the BCR repertoire is of interest for conditions involving cellular proliferation and antigenic exposure, including without limitation, the presence of cancer, exposure to cancer antigens, exposure to antigens from an infectious agent, exposure to vaccines, exposure to allergens, exposure to food stuffs, presence of a graft or transplant, and the presence of autoimmune activity or disease. Conditions associated with immunodeficiency are also of interest for analysis, including congenital and acquired immunodeficiency syndromes.

B cell lineage malignancies of interest include, without limitation, multiple myeloma; acute lymphocytic leukemia (ALL); relapsed/refractory B cell ALL, chronic lymphocytic leukemia (CLL); diffuse large B cell lymphoma; mucosa-associated lymphatic tissue lymphoma (MALT); small cell lymphocytic lymphoma; mantle cell lymphoma (MCL); Burkitt lymphoma; mediastinal large B cell lymphoma; Waldenström macroglobulinemia; nodal marginal zone B cell lymphoma (NMZL); splenic marginal zone lymphoma (SMZL); intravascular large B-cell lymphoma; primary effusion lymphoma; lymphomatoid granulomatosis, etc. Non-malignant B cell hyperproliferative conditions include monoclonal B cell lymphocytosis (MBL).

T cell lineage malignancies of interest include, without limitation, precursor T-cell lymphoblastic lymphoma; T-cell prolymphocytic leukemia; T-cell granular lymphocytic leukemia; aggressive NK cell leukemia; adult T-cell lymphoma/leukemia (HTLV 1-positive); extranodal NK/T-cell lymphoma; enteropathy-type T-cell lymphoma; hepatosplenic γδ T-cell lymphoma; subcutaneous panniculitis-like T-cell lymphoma; mycosis fungoides/Sezary syndrome; anaplastic large cell lymphoma, T/null cell; peripheral T-cell lymphoma; angioimmunoblastic T-cell lymphoma; chronic lymphocytic leukemia (CLL); acute lymphocytic leukemia (ALL); prolymphocytic leukemia; and hairy cell leukemia.

Other malignancies of interest include, without limitation, acute myeloid leukemia, head and neck cancers, brain cancer, breast cancer, ovarian cancer, cervical cancer, colorectal cancer, endometrial cancer, gallbladder cancer, gastric cancer, bladder cancer, prostate cancer, testicular cancer, liver cancer, lung cancer, kidney (renal cell) cancer, esophageal cancer, pancreatic cancer, thyroid cancer, bile duct cancer, pituitary tumor, wilms tumor, kaposi sarcoma, osteosarcoma, thymus cancer, skin cancer, heart cancer, oral and larynx cancer, neuroblastoma and non-hodgkin lymphoma.

Neurological inflammatory conditions are of interest, e.g. Alzheimer's Disease, Parkinson's Disease, Lou Gehrig's Disease, etc. and demyelinating diseases, such as multiple sclerosis, chronic inflammatory demyelinating polyneuropathy, etc. as well as inflammatory conditions such as rheumatoid arthritis. Systemic lupus erythematosus (SLE) is an autoimmune disease characterized by polyclonal B cell activation, which results in a variety of anti-protein and non-protein autoantibodies (see Kotzin et al. (1996) Cell 85:303-306). These autoantibodies form immune complexes that deposit in multiple organ systems, causing tissue damage. An autoimmune component may be ascribed to atherosclerosis, where candidate autoantigens include Hsp60, oxidized LDL, and 2-Glycoprotein I (2GPI).

A sample for use in the methods described herein may be one that is collected from a subject with a malignancy or hyperproliferative condition, including lymphomas, leukemias, and plasmacytomas. A lymphoma is a solid neoplasm of lymphocyte origin, and is most often found in the lymphoid tissue. Thus, for example, a biopsy from a lymph node, e.g. a tonsil, containing such a lymphoma would constitute a suitable biopsy. Samples may be obtained from a subject or patient at one or a plurality of time points in the progression of disease and/or treatment of the disease.

In some embodiments, the disclosure provides methods for performing target-specific multiplex PCR on a cDNA sample having a plurality of expressed immune receptor target sequences using primers having a cleavable group.

In certain embodiments, library and/or template preparation to be sequenced are prepared automatically from a population of nucleic acid samples using the compositions provided herein using an automated systems, e.g., the Ion ChefUM system.

As used herein, the term “subject” includes a person, a patient, an individual, someone being evaluated, etc.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive- or and not to an exclusive- or.

As used herein, “antigen” refers to any substance that, when introduced into a body, e.g., of a subject, can stimulate an immune response, such as the production of an antibody or T cell receptor that recognizes the antigen. Antigens include molecules such as nucleic acids, lipids, ribonucleoprotein complexes, protein complexes, proteins, polypeptides, peptides and naturally occurring or synthetic modifications of such molecules against which an immune response involving T and/or B lymphocytes can be generated. With regard to autoimmune disease, the antigens herein are often referred to as autoantigens. With regard to allergic disease the antigens herein are often referred to as allergens. Autoantigens are any molecule produced by the organism that can be the target of an immunologic response, including peptides, polypeptides, and proteins encoded within the genome of the organism and post-translationally-generated modifications of these peptides, polypeptides, and proteins. Such molecules also include carbohydrates, lipids and other molecules produced by the organism. Antigens also include vaccine antigens, which include, without limitation, pathogen antigens, cancer associated antigens, allergens, and the like.

As used herein, “amplify”, “amplifying” or “amplification reaction” and their derivatives, refer to any action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further including any of the amplification processes known to one of ordinary skill in the art. In some embodiments, the amplification reaction includes PCR.

As used herein, “amplification conditions” and its derivatives, refers to conditions suitable for amplifying one or more nucleic acid sequences. Such amplification can be linear or exponential. In some embodiments, the amplification conditions can include isothermal conditions or alternatively can include thermocycling conditions, or a combination of isothermal and thermocycling conditions. In some embodiments, the conditions suitable for amplifying one or more nucleic acid sequences includes PCR conditions. Typically, the amplification conditions refer to a reaction mixture that is sufficient to amplify nucleic acids such as one or more target sequences, or to amplify an amplified target sequence ligated to one or more adapters, e.g., an adapter-ligated amplified target sequence. Amplification conditions include a catalyst for amplification or for nucleic acid synthesis, for example a polymerase; a primer that possesses some degree of complementarity to the nucleic acid to be amplified; and nucleotides, such as deoxyribonucleotide triphosphates (dNTPs) to promote extension of the primer once hybridized to the nucleic acid. The amplification conditions can require hybridization or annealing of a primer to a nucleic acid, extension of the primer and a denaturing step in which the extended primer is separated from the nucleic acid sequence undergoing amplification. Typically, but not necessarily, amplification conditions can include thermocycling; in some embodiments, amplification conditions include a plurality of cycles where the steps of annealing, extending and separating are repeated. Typically, the amplification conditions include cations such as Mg²⁺ or Mn²⁺ (e.g., MgCl₂, etc) and can also include various modifiers of ionic strength.

As used herein, “target sequence” or “target sequence of interest” and its derivatives, refers to any single or double-stranded nucleic acid sequence that can be amplified or synthesized according to the disclosure, including any nucleic acid sequence suspected or expected to be present in a sample. In some embodiments, the target sequence is present in double-stranded form and includes at least a portion of the particular nucleotide sequence to be amplified or synthesized, or its complement, prior to the addition of target-specific primers or appended adapters. Target sequences can include the nucleic acids to which primers useful in the amplification or synthesis reaction can hybridize prior to extension by a polymerase. In some embodiments, the term refers to a nucleic acid sequence whose sequence identity, ordering or location of nucleotides is determined by one or more of the methods of the disclosure.

As defined herein, “sample” and its derivatives, is used in its broadest sense and includes any specimen, culture and the like that is suspected of including a target. In some embodiments, the sample comprises cDNA, RNA, PNA, LNA, chimeric, hybrid, or multiplex-forms of nucleic acids. The sample can include any biological, clinical, surgical, agricultural, atmospheric or aquatic-based specimen containing one or more nucleic acids. The term also includes any isolated nucleic acid sample such as expressed RNA, fresh-frozen or formalin-fixed paraffin-embedded nucleic acid specimen.

As used herein, “contacting” and its derivatives, when used in reference to two or more components, refers to any process whereby the approach, proximity, mixture or commingling of the referenced components is promoted or achieved without necessarily requiring physical contact of such components, and includes mixing of solutions containing any one or more of the referenced components with each other. The referenced components may be contacted in any particular order or combination and the particular order of recitation of components is not limiting. For example, “contacting A with B and C” encompasses embodiments where A is first contacted with B then C, as well as embodiments where C is contacted with A then B, as well as embodiments where a mixture of A and C is contacted with B, and the like. Furthermore, such contacting does not necessarily require that the end result of the contacting process be a mixture including all of the referenced components, as long as at some point during the contacting process all of the referenced components are simultaneously present or simultaneously included in the same mixture or solution. Where one or more of the referenced components to be contacted includes a plurality (e.g., “contacting a target sequence with a plurality of target-specific primers and a polymerase”), then each member of the plurality can be viewed as an individual component of the contacting process, such that the contacting can include contacting of any one or more members of the plurality with any other member of the plurality and/or with any other referenced component (e.g., some but not all of the plurality of target specific primers can be contacted with a target sequence, then a polymerase, and then with other members of the plurality of target-specific primers) in any order or combination.

As used herein, the term “primer” and its derivatives refer to any polynucleotide that can hybridize to a target sequence of interest. In some embodiments, the primer can also serve to prime nucleic acid synthesis. Typically, the primer functions as a substrate onto which nucleotides can be polymerized by a polymerase; in some embodiments, however, the primer can become incorporated into the synthesized nucleic acid strand and provide a site to which another primer can hybridize to prime synthesis of a new strand that is complementary to the synthesized nucleic acid molecule. The primer may be comprised of any combination of nucleotides or analogs thereof, which may be optionally linked to form a linear polymer of any suitable length. In some embodiments, the primer is a single-stranded oligonucleotide or polynucleotide. (For purposes of this disclosure, the terms ‘polynucleotide” and “oligonucleotide” are used interchangeably herein and do not necessarily indicate any difference in length between the two). In some embodiments, the primer is single-stranded but it can also be double-stranded. The primer optionally occurs naturally, as in a purified restriction digest, or can be produced synthetically. In some embodiments, the primer acts as a point of initiation for amplification or synthesis when exposed to amplification or synthesis conditions; such amplification or synthesis can occur in a template-dependent fashion and optionally results in formation of a primer extension product that is complementary to at least a portion of the target sequence. Exemplary amplification or synthesis conditions can include contacting the primer with a polynucleotide template (e.g., a template including a target sequence), nucleotides and an inducing agent such as a polymerase at a suitable temperature and pH to induce polymerization of nucleotides onto an end of the target-specific primer. If double-stranded, the primer can optionally be treated to separate its strands before being used to prepare primer extension products. In some embodiments, the primer is an oligodeoxyribonucleotide or an oligoribonucleotide. In some embodiments, the primer can include one or more nucleotide analogs. The exact length and/or composition, including sequence, of the target-specific primer can influence many properties, including melting temperature (Tm), GC content, formation of secondary structures, repeat nucleotide motifs, length of predicted primer extension products, extent of coverage across a nucleic acid molecule of interest, number of primers present in a single amplification or synthesis reaction, presence of nucleotide analogs or modified nucleotides within the primers, and the like. In some embodiments, a primer can be paired with a compatible primer within an amplification or synthesis reaction to form a primer pair consisting or a forward primer and a reverse primer. In some embodiments, the forward primer of the primer pair includes a sequence that is substantially complementary to at least a portion of a strand of a nucleic acid molecule, and the reverse primer of the primer of the primer pair includes a sequence that is substantially identical to at least of portion of the strand. In some embodiments, the forward primer and the reverse primer are capable of hybridizing to opposite strands of a nucleic acid duplex. Optionally, the forward primer primes synthesis of a first nucleic acid strand, and the reverse primer primes synthesis of a second nucleic acid strand, wherein the first and second strands are substantially complementary to each other, or can hybridize to form a double-stranded nucleic acid molecule. In some embodiments, one end of an amplification or synthesis product is defined by the forward primer and the other end of the amplification or synthesis product is defined by the reverse primer. In some embodiments, where the amplification or synthesis of lengthy primer extension products is required, such as amplifying an exon, coding region, or gene, several primer pairs can be created than span the desired length to enable sufficient amplification of the region. In some embodiments, a primer can include one or more cleavable groups. In some embodiments, primer lengths are in the range of about 10 to about 60 nucleotides, about 12 to about 50 nucleotides and about 15 to about 40 nucleotides in length. Typically, a primer is capable of hybridizing to a corresponding target sequence and undergoing primer extension when exposed to amplification conditions in the presence of dNTPs and a polymerase. In some embodiments, the primer includes one or more cleavable groups at one or more locations within the primer.

As used herein, “target-specific primer” and its derivatives, refers to a single stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or identical, to at least a portion of a nucleic acid molecule that includes a target sequence. In such instances, the target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement. In some embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the target sequence itself; in other embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the nucleic acid molecule other than the target sequence. In some embodiments, the target-specific primer is substantially non-complementary to other target sequences present in the sample; optionally, the target-specific primer is substantially non-complementary to other nucleic acid molecules present in the sample. In some embodiments, nucleic acid molecules present in the sample that do not include or correspond to a target sequence (or to a complement of the target sequence) are referred to as “non-specific” sequences or “non-specific nucleic acids”. In some embodiments, the target-specific primer is designed to include a nucleotide sequence that is substantially complementary to at least a portion of its corresponding target sequence. In some embodiments, a target-specific primer is at least 95% complementary, or at least 99% complementary, or identical, across its entire length to at least a portion of a nucleic acid molecule that includes its corresponding target sequence. In some embodiments, a target-specific primer is at least 90%, at least 95% complementary, at least 98% complementary or at least 99% complementary, or identical, across its entire length to at least a portion of its corresponding target sequence. In some embodiments, a forward target-specific primer and a reverse target-specific primer define a target-specific primer pair that are used to amplify the target sequence via template-dependent primer extension. Typically, each primer of a target-specific primer pair includes at least one sequence that is substantially complementary to at least a portion of a nucleic acid molecule including a corresponding target sequence but that is less than 50% complementary to at least one other target sequence in the sample. In some embodiments, amplification is performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence. In some embodiments, the target-specific primer is substantially non-complementary at its 3′ end or its 5′ end to any other target-specific primer present in an amplification reaction. In some embodiments, the target-specific primer can include minimal cross hybridization to other target-specific primers in the amplification reaction. In some embodiments, target-specific primers include minimal cross-hybridization to non-specific sequences in the amplification reaction mixture. In some embodiments, the target-specific primers include minimal self-complementarity. In some embodiments, the target-specific primers can include one or more cleavable groups located at the 3′ end. In some embodiments, the target-specific primers can include one or more cleavable groups located near or about a central nucleotide of the target-specific primer. In some embodiments, one of more targets-specific primers includes only non-cleavable nucleotides at the 5′ end of the target-specific primer. In some embodiments, a target specific primer includes minimal nucleotide sequence overlap at the 3′end or the 5′ end of the primer as compared to one or more different target-specific primers, optionally in the same amplification reaction. In some embodiments 1, 2, 3, 4, 5, 6, 7, 8, 9, 10 or more, target-specific primers in a single reaction mixture include one or more of the above embodiments. In some embodiments, substantially all of the plurality of target-specific primers in a single reaction mixture includes one or more of the above embodiments.

As used herein, “polymerase” and its derivatives, refers to any enzyme that can catalyze the polymerization of nucleotides (including analogs thereof) into a nucleic acid strand. Typically but not necessarily, such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase is a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also refers to fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase is optionally reactivated, for example through the use of heat, chemicals or re-addition of new amounts of polymerase into a reaction mixture. In some embodiments, the polymerase can include a hot-start polymerase or an aptamer based polymerase that optionally is reactivated.

As used herein, the term “nucleotide” and its variants comprises any compound, including without limitation any naturally occurring nucleotide or analog thereof, which can bind selectively to, or is polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. In some embodiments, the nucleotide can optionally include a chain of phosphorus atoms comprising three, four, five, six, seven, eight, nine, ten or more phosphorus atoms. In some embodiments, the phosphorus chain is attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain is linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain has side groups having O, BH₃, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in U.S. Pat. No. 7,405,281. In some embodiments, the nucleotide comprises a label and referred to herein as a “labeled nucleotide”; the label of the labeled nucleotide is referred to herein as a “nucleotide label.” In some embodiments, the label is in the form of a fluorescent dye attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. alpha-thio-nucleotide 5′-triphosphates. For a review of nucleic acid chemistry, see: Shabarova, Z. and Bogdanov, A. Advanced Organic Chemistry of Nucleic Acids, VCH, New York, 1994.

The term “extension” and its variants, as used herein, when used in reference to a given primer, comprises any in vivo or in vitro enzymatic activity characteristic of a given polymerase that relates to polymerization of one or more nucleotides onto an end of an existing nucleic acid molecule. Typically but not necessarily such primer extension occurs in a template-dependent fashion; during template-dependent extension, the order and selection of bases is driven by established base pairing rules, which can include Watson-Crick type base pairing rules or alternatively (and especially in the case of extension reactions involving nucleotide analogs) by some other type of base pairing paradigm. In one non-limiting example, extension occurs via polymerization of nucleotides on the 3′OH end of the nucleic acid molecule by the polymerase.

The term “portion” and its variants, as used herein, when used in reference to a given nucleic acid molecule, for example a primer or a template nucleic acid molecule, comprises any number of contiguous nucleotides within the length of the nucleic acid molecule, including the partial or entire length of the nucleic acid molecule.

The terms “identity” and “identical” and their variants, as used herein, when used in reference to two or more nucleic acid sequences, refer to similarity in sequence of the two or more sequences (e.g., nucleotide or polypeptide sequences). In the context of two or more homologous sequences, the percent identity or homology of the sequences or subsequences thereof indicates the percentage of all monomeric units (e.g., nucleotides or amino acids) that are the same (i.e., about 70% identity, preferably 75%, 80%, 85%, 90%, 95%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The terms “complementary” and “complement” and their variants, as used herein, refer to any two or more nucleic acid sequences (e.g., portions or entireties of template nucleic acid molecules, target sequences and/or primers) that can undergo cumulative base pairing at two or more individual corresponding positions in antiparallel orientation, as in a hybridized duplex. Such base pairing can proceed according to any set of established rules, for example according to Watson-Crick base pairing rules or according to some other base pairing paradigm. Optionally there can be “complete” or “total” complementarity between a first and second nucleic acid sequence where each nucleotide in the first nucleic acid sequence can undergo a stabilizing base pairing interaction with a nucleotide in the corresponding antiparallel position on the second nucleic acid sequence. “Partial” complementarity describes nucleic acid sequences in which at least 20%, but less than 100%, of the residues of one nucleic acid sequence are complementary to residues in the other nucleic acid sequence. In some embodiments, at least 50%, but less than 100%, of the residues of one nucleic acid sequence are complementary to residues in the other nucleic acid sequence. In some embodiments, at least 70%, 80%, 90%, 95% or 98%, but less than 100%, of the residues of one nucleic acid sequence are complementary to residues in the other nucleic acid sequence. Sequences are said to be “substantially complementary” when at least 85% of the residues of one nucleic acid sequence are complementary to residues in the other nucleic acid sequence. In some embodiments, two complementary or substantially complementary sequences are capable of hybridizing to each other under standard or stringent hybridization conditions. “Non-complementary” describes nucleic acid sequences in which less than 20% of the residues of one nucleic acid sequence are complementary to residues in the other nucleic acid sequence. Sequences are said to be “substantially non-complementary” when less than 15% of the residues of one nucleic acid sequence are complementary to residues in the other nucleic acid sequence. In some embodiments, two non-complementary or substantially non-complementary sequences cannot hybridize to each other under standard or stringent hybridization conditions. A “mismatch” is present at any position in the sequences where two opposed nucleotides are not complementary. Complementary nucleotides include nucleotides that are efficiently incorporated by DNA polymerases opposite each other during DNA replication under physiological conditions. In a typical embodiment, complementary nucleotides can form base pairs with each other, such as the A-T/U and G-C base pairs formed through specific Watson-Crick type hydrogen bonding, or base pairs formed through some other type of base pairing paradigm, between the nucleobases of nucleotides and/or polynucleotides in positions antiparallel to each other. The complementarity of other artificial base pairs can be based on other types of hydrogen bonding and/or hydrophobicity of bases and/or shape complementarity between bases.

As used herein, “amplified target sequences” and its derivatives, refers to a nucleic acid sequence produced by the amplification of/amplifying the target sequences using target-specific primers and the methods provided herein. The amplified target sequences may be either of the same sense (the positive strand produced in the second round and subsequent even-numbered rounds of amplification) or antisense (i.e., the negative strand produced during the first and subsequent odd-numbered rounds of amplification) with respect to the target sequences. In some embodiments, the amplified target sequences is less than 50% complementary to any portion of another amplified target sequence in the reaction. In other embodiments, the amplified target sequences is greater than 50%, greater than 60%, greater than 70%, greater than 80%, or greater than 90% complementary to any portion of another amplified target sequence in the reaction.

As used herein, the terms “ligating”, “ligation” and their derivatives refer to the act or process for covalently linking two or more molecules together, for example, covalently linking two or more nucleic acid molecules to each other. In some embodiments, ligation includes joining nicks between adjacent nucleotides of nucleic acids. In some embodiments, ligation includes forming a covalent bond between an end of a first and an end of a second nucleic acid molecule. In some embodiments, for example embodiments wherein the nucleic acid molecules to be ligated include conventional nucleotide residues, the ligation can include forming a covalent bond between a 5′ phosphate group of one nucleic acid and a 3′ hydroxyl group of a second nucleic acid thereby forming a ligated nucleic acid molecule. In some embodiments, any means for joining nicks or bonding a 5′phosphate to a 3′ hydroxyl between adjacent nucleotides can be employed. In an exemplary embodiment, an enzyme such as a ligase is used. For the purposes of this disclosure, an amplified target sequence can be ligated to an adapter to generate an adapter-ligated amplified target sequence.

As used herein, “ligase” and its derivatives, refers to any agent capable of catalyzing the ligation of two substrate molecules. In some embodiments, the ligase includes an enzyme capable of catalyzing the joining of nicks between adjacent nucleotides of a nucleic acid. In some embodiments, the ligase includes an enzyme capable of catalyzing the formation of a covalent bond between a 5′ phosphate of one nucleic acid molecule to a 3′ hydroxyl of another nucleic acid molecule thereby forming a ligated nucleic acid molecule. In some embodiments, the ligase is an isothermal ligase. In some embodiments, the ligase is a thermostable ligase. Suitable ligases may include, but not limited to, T4 DNA ligase, T4 RNA ligase, and E. coli DNA ligase.

As used herein, “ligation conditions” and its derivatives, refers to conditions suitable for ligating two molecules to each other. In some embodiments, the ligation conditions are suitable for sealing nicks or gaps between nucleic acids. As defined herein, a “nick” or “gap” refers to a nucleic acid molecule that lacks a directly bound 5′ phosphate of a mononucleotide pentose ring to a 3′ hydroxyl of a neighboring mononucleotide pentose ring within internal nucleotides of a nucleic acid sequence. As used herein, the term nick or gap is consistent with the use of the term in the art. Typically, a nick or gap is ligated in the presence of an enzyme, such as ligase at an appropriate temperature and pH. In some embodiments, T4 DNA ligase can join a nick between nucleic acids at a temperature of about 70-72° C.

As used herein, “blunt-end ligation” and its derivatives, refers to ligation of two blunt-end double-stranded nucleic acid molecules to each other. A “blunt end” refers to an end of a double-stranded nucleic acid molecule wherein substantially all of the nucleotides in the end of one strand of the nucleic acid molecule are base paired with opposing nucleotides in the other strand of the same nucleic acid molecule. A nucleic acid molecule is not blunt ended if it has an end that includes a single-stranded portion greater than two nucleotides in length, referred to herein as an “overhang”. In some embodiments, the end of nucleic acid molecule does not include any single stranded portion, such that every nucleotide in one strand of the end is based paired with opposing nucleotides in the other strand of the same nucleic acid molecule. In some embodiments, the ends of the two blunt ended nucleic acid molecules that become ligated to each other do not include any overlapping, shared or complementary sequence. Typically, blunted-end ligation excludes the use of additional oligonucleotide adapters to assist in the ligation of the double-stranded amplified target sequence to the double-stranded adapter, such as patch oligonucleotides as described in US Pat. Publication No. 2010/0129874. In some embodiments, blunt-ended ligation includes a nick translation reaction to seal a nick created during the ligation process.

As used herein, the terms “adapter” or “adapter and its complements” and their derivatives, refers to any linear oligonucleotide which is ligated to a nucleic acid molecule of the disclosure. Optionally, the adapter includes a nucleic acid sequence that is not substantially complementary to the 3′ end or the 5′ end of at least one target sequences within the sample. In some embodiments, the adapter is substantially non-complementary to the 3′ end or the 5′ end of any target sequence present in the sample. In some embodiments, the adapter includes any single stranded or double-stranded linear oligonucleotide that is not substantially complementary to an amplified target sequence. In some embodiments, the adapter is substantially non-complementary to at least one, some or all of the nucleic acid molecules of the sample. In some embodiments, suitable adapter lengths are in the range of about 10-100 nucleotides, about 12-60 nucleotides and about 15-50 nucleotides in length. An adapter can include any combination of nucleotides and/or nucleic acids. In some embodiments, the adapter can include one or more cleavable groups at one or more locations. In another embodiment, the adapter can include a sequence that is substantially identical, or substantially complementary, to at least a portion of a primer, for example a universal primer. The structure and properties of universal amplification primers are well known to those skilled in the art and can be implemented for utilization in conjunction with provided methods and compositions to adapt to specific analysis platforms (e.g., as described herein universal P1 and A primers have been described in the art and utilized for sequencing on Ion Torrent sequencing platforms). Similarly, additional and other universal adaptor/primer sequences described and known in the art (e.g., Illumina universal adaptor/primer sequences, PacBio universal adaptor/primer sequences, etc.) can be used in conjunction with the methods and compositions provided herein. In some embodiments, the adapter can include a barcode or tag to assist with downstream cataloguing, identification or sequencing. In some embodiments, a single-stranded adapter can act as a substrate for amplification when ligated to an amplified target sequence, particularly in the presence of a polymerase and dNTPs under suitable temperature and pH.

In some embodiments, an adapter is ligated to a polynucleotide through a blunt-end ligation. In other embodiments, an adapter is ligated to a polynucleotide via nucleotide overhangs on the ends of the adapter and the polynucleotide. For overhang ligation, an adapter may have a nucleotide overhang added to the 3′ and/or 5′ ends of the respective strands if the polynucleotides to which the adapters are to be ligated (eg, amplicons) have a complementary overhang added to the 3′ and/or 5′ ends of the respective strands. For example, adenine nucleotides can be added to the 3′ terminus of an end-repaired PCR product. Adapters having with an overhang formed by thymine nucleotides can then dock with the A-overhang of the amplicon and be ligated to the amplicon by a DNA ligase, such as T4 DNA ligase.

As used herein, “reamplifying” or “reamplification” and their derivatives refer to any process whereby at least a portion of an amplified nucleic acid molecule is further amplified via any suitable amplification process (referred to in some embodiments as a “secondary” amplification or “reamplification”, thereby producing a reamplified nucleic acid molecule. The secondary amplification need not be identical to the original amplification process whereby the amplified nucleic acid molecule was produced; nor need the reamplified nucleic acid molecule be completely identical or completely complementary to the amplified nucleic acid molecule; all that is required is that the reamplified nucleic acid molecule include at least a portion of the amplified nucleic acid molecule or its complement. For example, the reamplification can involve the use of different amplification conditions and/or different primers, including different target-specific primers than the primary amplification.

As defined herein, a “cleavable group” refers to any moiety that once incorporated into a nucleic acid can be cleaved under appropriate conditions. For example, a cleavable group can be incorporated into a target-specific primer, an amplified sequence, an adapter or a nucleic acid molecule of the sample. In an exemplary embodiment, a target-specific primer can include a cleavable group that becomes incorporated into the amplified product and is subsequently cleaved after amplification, thereby removing a portion, or all, of the target-specific primer from the amplified product. The cleavable group can be cleaved or otherwise removed from a target-specific primer, an amplified sequence, an adapter or a nucleic acid molecule of the sample by any acceptable means. For example, a cleavable group can be removed from a target-specific primer, an amplified sequence, an adapter or a nucleic acid molecule of the sample by enzymatic, thermal, photo-oxidative or chemical treatment. In one embodiment, a cleavable group can include a nucleobase that is not naturally occurring. For example, an oligodeoxyribonucleotide can include one or more RNA nucleobases, such as uracil that can be removed by a uracil glycosylase. In some embodiments, a cleavable group can include one or more modified nucleobases (such as 7-methylguanine, 8-oxo-guanine, xanthine, hypoxanthine, 5,6-dihydrouracil or 5-methylcytosine) or one or more modified nucleosides (i.e., 7-methylguanosine, 8-oxo-deoxyguanosine, xanthosine, inosine, dihydrouridine or 5-methylcytidine). The modified nucleobases or nucleotides can be removed from the nucleic acid by enzymatic, chemical or thermal means. In one embodiment, a cleavable group can include a moiety that can be removed from a primer after amplification (or synthesis) upon exposure to ultraviolet light (i.e., bromodeoxyuridine). In another embodiment, a cleavable group can include methylated cytosine. Typically, methylated cytosine can be cleaved from a primer for example, after induction of amplification (or synthesis), upon sodium bisulfite treatment. In some embodiments, a cleavable moiety can include a restriction site. For example, a primer or target sequence can include a nucleic acid sequence that is specific to one or more restriction enzymes, and following amplification (or synthesis), the primer or target sequence can be treated with the one or more restriction enzymes such that the cleavable group is removed. Typically, one or more cleavable groups can be included at one or more locations with a target-specific primer, an amplified sequence, an adapter or a nucleic acid molecule of the sample.

As used herein, “cleavage step” and its derivatives, refers to any process by which a cleavable group is cleaved or otherwise removed from a target-specific primer, an amplified sequence, an adapter or a nucleic acid molecule of the sample. In some embodiments, the cleavage step involves a chemical, thermal, photo-oxidative or digestive process.

As used herein, the term “hybridization” is consistent with its use in the art, and refers to the process whereby two nucleic acid molecules undergo base pairing interactions. Two nucleic acid molecule molecules are said to be hybridized when any portion of one nucleic acid molecule is base paired with any portion of the other nucleic acid molecule; it is not necessarily required that the two nucleic acid molecules be hybridized across their entire respective lengths and in some embodiments, at least one of the nucleic acid molecules can include portions that are not hybridized to the other nucleic acid molecule. The phrase “hybridizing under stringent conditions” and its variants refers to conditions under which hybridization of a target-specific primer to a target sequence occurs in the presence of high hybridization temperature and low ionic strength. In one exemplary embodiment, stringent hybridization conditions include an aqueous environment containing about 30 mM magnesium sulfate, about 300 mM Tris-sulfate at pH 8.9, and about 90 mM ammonium sulfate at about 60-68° C., or equivalents thereof. As used herein, the phrase “standard hybridization conditions” and its variants refers to conditions under which hybridization of a primer to an oligonucleotide (i.e., a target sequence), occurs in the presence of low hybridization temperature and high ionic strength. In one exemplary embodiment, standard hybridization conditions include an aqueous environment containing about 100 mM magnesium sulfate, about 500 mM Tris-sulfate at pH 8.9, and about 200 mM ammonium sulfate at about 50-55° C., or equivalents thereof.

As used herein, “GC content” and its derivatives, refers to the cytosine and guanine content of a nucleic acid molecule. The GC content of a target-specific primer (or adapter) of the disclosure is 85% or lower. More typically, the GC content of a target-specific primer or adapter of the disclosure is between 15-85%.

As used herein, the term “end” and its variants, when used in reference to a nucleic acid molecule, for example a target sequence or amplified target sequence, can include the terminal 30 nucleotides, the terminal 20 and even more typically the terminal 15 nucleotides of the nucleic acid molecule. A linear nucleic acid molecule comprised of linked series of contiguous nucleotides typically includes at least two ends. In some embodiments, one end of the nucleic acid molecule can include a 3′ hydroxyl group or its equivalent, and is referred to as the “3′ end” and its derivatives. Optionally, the 3′ end includes a 3′ hydroxyl group that is not linked to a 5′ phosphate group of a mononucleotide pentose ring. Typically, the 3′ end includes one or more 5′ linked nucleotides located adjacent to the nucleotide including the unlinked 3′ hydroxyl group, typically the 30 nucleotides located adjacent to the 3′ hydroxyl, typically the terminal 20 and even more typically the terminal 15 nucleotides. One or more linked nucleotides can be represented as a percentage of the nucleotides present in the oligonucleotide or can be provided as a number of linked nucleotides adjacent to the unlinked 3′ hydroxyl. For example, the 3′ end can include less than 50% of the nucleotide length of the oligonucleotide. In some embodiments, the 3′ end does not include any unlinked 3′ hydroxyl group but can include any moiety capable of serving as a site for attachment of nucleotides via primer extension and/or nucleotide polymerization. In some embodiments, the term “3′ end” for example when referring to a target-specific primer, can include the terminal 10 nucleotides, the terminal 5 nucleotides, the terminal 4, 3, 2 or fewer nucleotides at the 3′end. In some embodiments, the term “3′ end” when referring to a target-specific primer can include nucleotides located at nucleotide positions 10 or fewer from the 3′ terminus.

As used herein, “5′ end”, and its derivatives, refers to an end of a nucleic acid molecule, for example a target sequence or amplified target sequence, which includes a free 5′ phosphate group or its equivalent. In some embodiments, the 5′ end includes a 5′ phosphate group that is not linked to a 3′ hydroxyl of a neighboring mononucleotide pentose ring. Typically, the 5′ end includes to one or more linked nucleotides located adjacent to the 5′ phosphate, typically the 30 nucleotides located adjacent to the nucleotide including the 5′ phosphate group, typically the terminal 20 and even more typically the terminal 15 nucleotides. One or more linked nucleotides can be represented as a percentage of the nucleotides present in the oligonucleotide or can be provided as a number of linked nucleotides adjacent to the 5′ phosphate. For example, the 5′ end can be less than 50% of the nucleotide length of an oligonucleotide. In another exemplary embodiment, the 5′ end can include about 15 nucleotides adjacent to the nucleotide including the terminal 5′ phosphate. In some embodiments, the 5′ end does not include any unlinked 5′ phosphate group but can include any moiety capable of serving as a site of attachment to a 3′ hydroxyl group, or to the 3′end of another nucleic acid molecule. In some embodiments, the term “5′ end” for example when referring to a target-specific primer, can include the terminal 10 nucleotides, the terminal 5 nucleotides, the terminal 4, 3, 2 or fewer nucleotides at the 5′end. In some embodiments, the term “5′ end” when referring to a target-specific primer can include nucleotides located at positions 10 or fewer from the 5′ terminus. In some embodiments, the 5′ end of a target-specific primer can include only non-cleavable nucleotides, for example nucleotides that do not contain one or more cleavable groups as disclosed herein, or a cleavable nucleotide as would be readily determined by one of ordinary skill in the art.

As used herein, “DNA barcode” and its derivatives, refers to a unique short (e.g., 6-14 nucleotide) nucleic acid sequence within an adapter that can act as a ‘key’ to distinguish or separate a plurality of amplified target sequences in a sample. For the purposes of this disclosure, a DNA barcode can be incorporated into the nucleotide sequence of an adapter.

As used herein, the phrases “two rounds of target-specific hybridization” or “two rounds of target-specific selection” and their derivatives refers to any process whereby the same target sequence is subjected to two consecutive rounds of hybridization-based target-specific selection, wherein a target sequence is hybridized to a target-specific sequence. Each round of hybridization based target-specific selection can include multiple target-specific hybridizations to at least some portion of a target-specific sequence. In one exemplary embodiment, a round of target-specific selection includes a first target-specific hybridization involving a first region of the target sequence and a second target-specific hybridization involving a second region of the target sequence. The first and second regions can be the same or different. In some embodiments, each round of hybridization-based target-specific selection can include use of two target specific oligonucleotides (e.g., a forward target-specific primer and a reverse target-specific primer), such that each round of selection includes two target-specific hybridizations.

As used herein, “comparable maximal minimum melting temperatures” and its derivatives, refers to the melting temperature (Tₘ) of each nucleic acid fragment for a single adapter or target-specific primer after cleavage of the cleavable groups. The hybridization temperature of each nucleic acid fragment generated by a single adapter or target-specific primer is compared to determine the maximal minimum temperature required preventing hybridization of any nucleic acid fragment from the target-specific primer or adapter to the target sequence. Once the maximal hybridization temperature is known, it is possible to manipulate the adapter or target-specific primer, for example by moving the location of the cleavable group along the length of the primer, to achieve a comparable maximal minimum melting temperature with respect to each nucleic acid fragment.

As used herein, “addition only” and its derivatives, refers to a series of steps in which reagents and components are added to a first or single reaction mixture. Typically, the series of steps excludes the removal of the reaction mixture from a first vessel to a second vessel in order to complete the series of steps. An addition only process excludes the manipulation of the reaction mixture outside the vessel containing the reaction mixture. Typically, an addition-only process is amenable to automation and high-throughput.

As used herein, “synthesizing” and its derivatives, refers to a reaction involving nucleotide polymerization by a polymerase, optionally in a template-dependent fashion. Polymerases synthesize an oligonucleotide via transfer of a nucleoside monophosphate from a nucleoside triphosphate (NTP), deoxynucleoside triphosphate (dNTP) or dideoxynucleoside triphosphate (ddNTP) to the 3′ hydroxyl of an extending oligonucleotide chain. For the purposes of this disclosure, synthesizing includes to the serial extension of a hybridized adapter or a target-specific primer via transfer of a nucleoside monophosphate from a deoxynucleoside triphosphate.

As used herein, “polymerizing conditions” and its derivatives, refers to conditions suitable for nucleotide polymerization. In typical embodiments, such nucleotide polymerization is catalyzed by a polymerase. In some embodiments, polymerizing conditions include conditions for primer extension, optionally in a template-dependent manner, resulting in the generation of a synthesized nucleic acid sequence. In some embodiments, the polymerizing conditions include PCR. Typically, the polymerizing conditions include use of a reaction mixture that is sufficient to synthesize nucleic acids and includes a polymerase and nucleotides. The polymerizing conditions can include conditions for annealing of a target-specific primer to a target sequence and extension of the primer in a template dependent manner in the presence of a polymerase. In some embodiments, polymerizing conditions are practiced using thermocycling. Additionally, polymerizing conditions can include a plurality of cycles where the steps of annealing, extending, and separating the two nucleic strands are repeated. Typically, the polymerizing conditions include a cation such as MgCl₂. Polymerization of one or more nucleotides to form a nucleic acid strand includes that the nucleotides be linked to each other via phosphodiester bonds, however, alternative linkages may be possible in the context of particular nucleotide analogs.

As used herein, the term “nucleic acid” refers to natural nucleic acids, artificial nucleic acids, analogs thereof, or combinations thereof, including polynucleotides and oligonucleotides. As used herein, the terms “polynucleotide” and “oligonucleotide” are used interchangeably and mean single-stranded and double-stranded polymers of nucleotides including, but not limited to, 2′-deoxyribonucleotides (nucleic acid) and ribonucleotides (RNA) linked by internucleotide phosphodiester bond linkages, e.g. 3′-5′ and 2′-5′, inverted linkages, e.g. 3′-3′ and 5′-5′, branched structures, or analog nucleic acids. Polynucleotides have associated counter ions, such as H⁺, NH⁴⁺, trialkylammonium, Mg²⁺, Na⁺ and the like. An oligonucleotide can be composed entirely of deoxyribonucleotides, entirely of ribonucleotides, or chimeric mixtures thereof. Oligonucleotides can be comprised of nucleobase and sugar analogs. Polynucleotides typically range in size from a few monomeric units, e.g. 5-40, when they are more commonly frequently referred to in the art as oligonucleotides, to several thousands of monomeric nucleotide units, when they are more commonly referred to in the art as polynucleotides; for purposes of this disclosure, however, both oligonucleotides and polynucleotides may be of any suitable length. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U’ denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

As defined herein, the term “nick translation” and its variants comprise the translocation of one or more nicks or gaps within a nucleic acid strand to a new position along the nucleic acid strand. In some embodiments, a nick is formed when a double stranded adapter is ligated to a double stranded amplified target sequence. In one example, the primer can include at its 5′ end, a phosphate group that can ligate to the double stranded amplified target sequence, leaving a nick between the adapter and the amplified target sequence in the complementary strand. In some embodiments, nick translation results in the movement of the nick to the 3′ end of the nucleic acid strand. In some embodiments, moving the nick can include performing a nick translation reaction on the adapter-ligated amplified target sequence. In some embodiments, the nick translation reaction is a coupled 5′ to 3′ DNA polymerization/degradation reaction, or coupled to a 5′ to 3′ DNA polymerization/strand displacement reaction. In some embodiments, moving the nick can include performing a DNA strand extension reaction at the nick site. In some embodiments, moving the nick can include performing a single strand exonuclease reaction on the nick to form a single stranded portion of the adapter-ligated amplified target sequence and performing a DNA strand extension reaction on the single stranded portion of the adapter-ligated amplified target sequence to a new position. In some embodiments, a nick is formed in the nucleic acid strand opposite the site of ligation.

As used herein, the term “polymerase chain reaction” (“PCR”) refers to the method of K. B. Mullis U.S. Pat. Nos. 4,683,195 and 4,683,202, hereby incorporated by reference, which describe a method for increasing the concentration of a segment of a polynucleotide of interest in a mixture of expressed RNA or cDNA without cloning or purification. This process for amplifying the polynucleotide of interest consists of introducing a large excess of two oligonucleotide primers to the DNA mixture containing the desired polynucleotide of interest, followed by a precise sequence of thermal cycling in the presence of a DNA polymerase. The two primers are complementary to their respective strands of the double stranded polynucleotide of interest. To effect amplification, the mixture is denatured and the primers then annealed to their complementary sequences within the polynucleotide of interest molecule. Following annealing, the primers are extended with a polymerase to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “PCR”. Because the desired amplified segments of the polynucleotide of interest become the predominant nucleic acid sequences (in terms of concentration) in the mixture, they are said to be “PCR amplified”. As defined herein, target nucleic acid molecules within a sample including a plurality of target nucleic acid molecules are amplified via PCR. In a modification to the method discussed above, the target nucleic acid molecules are PCR amplified using a plurality of different primer pairs, in some cases, one or more primer pairs per target nucleic acid molecule of interest, thereby forming a multiplex PCR reaction. In some embodiments provided herein, multiplex PCR amplifications are performed using a plurality of different primer pairs, in typical cases, one primer pair per target nucleic acid molecule. Using multiplex PCR, it is possible to simultaneously amplify multiple nucleic acid molecules of interest from a sample to form amplified target sequences. It is also possible to detect the amplified target sequences by several different methodologies (e.g., quantitation with a bioanalyzer or qPCR, hybridization with a labeled probe; incorporation of biotinylated primers followed by avidin-enzyme conjugate detection; incorporation of ³²P-labeled deoxynucleotide triphosphates, such as dCTP or dATP, into the amplified target sequence). Any oligonucleotide sequence can be amplified with the appropriate set of primers, thereby allowing for the amplification of target nucleic acid molecules from RNA, cDNA, formalin-fixed paraffin-embedded DNA, fine-needle biopsies and various other sources. In particular, the amplified target sequences created by the multiplex PCR process as disclosed herein, are themselves efficient substrates for subsequent PCR amplification or various downstream assays or manipulations.

As defined herein “multiplex amplification” refers to selective and non-random amplification of two or more target sequences within a sample using at least one target-specific primer. In some embodiments, multiplex amplification is performed such that some or all of the target sequences are amplified within a single reaction vessel. The “plexy” or “plex” of a given multiplex amplification refers to the number of different target-specific sequences that are amplified during that single multiplex amplification. In some embodiments, the plexy is about 12-plex, 24-plex, 48-plex, 74-plex, 96-plex, 120-plex, 144-plex, 168-plex, 192-plex, 216-plex, 240-plex, 264-plex, 288-plex, 312-plex, 336-plex, 360-plex, 384-plex, or 398-plex. In some embodiments, highly multiplexed amplification reactions include reactions with a plexy of greater than 12-plex.

In some embodiments, the amplified target sequences are formed via PCR. Extension of target-specific primers can be accomplished using one or more DNA polymerases. In one embodiment, the polymerase is any Family A DNA polymerase (also known as pol I family) or any Family B DNA polymerase. In some embodiments, the DNA polymerase is a recombinant form capable of extending target-specific primers with superior accuracy and yield as compared to a non-recombinant DNA polymerase. For example, the polymerase can include a high-fidelity polymerase or thermostable polymerase. In some embodiments, conditions for extension of target-specific primers can include ‘Hot Start’ conditions, for example Hot Start polymerases, such as Amplitaq Gold® DNA polymerase (Applied Biosciences), Platinum® Taq DNA Polymerase High Fidelity (Invitrogen) or KOD Hot Start DNA polymerase (EMD Biosciences). A ‘Hot Start’ polymerase includes a thermostable polymerase and one or more antibodies that inhibit DNA polymerase and 3′-5′ exonuclease activities at ambient temperature. In some instances, ‘Hot Start’ conditions can include an aptamer.

In some embodiments, the polymerase is an enzyme such as Taq polymerase (from Thermus aquaticus), Tfi polymerase (from Thermus filiformis), Bst polymerase (from Bacillus stearothermophilus), Pfu polymerase (from Pyrococcus furiosus), Tth polymerase (from Thermus thermophilus), Pow polymerase (from Pyrococcus woesei), Tli polymerase (from Thermococcus litoralis), Ultima polymerase (from Thermotoga maritima), KOD polymerase (from Thermococcus kodakaraensis), Pol I and II polymerases (from Pyrococcus abyssi) and Pab (from Pyrococcus abyssi). In some embodiments, the DNA polymerase can include at least one polymerase such as Amplitaq Gold @DNA polymerase (Applied Biosciences), Stoffel fragment of Amplitaq® DNA Polymerase (Roche), KOD polymerase (EMD Biosciences), KOD Hot Start polymerase (EMD Biosciences), Deep Vent™ DNA polymerase (New England Biolabs), Phusion polymerase (New England Biolabs), Klentaq1 polymerase (DNA Polymerase Technology, Inc), Klentaq Long Accuracy polymerase (DNA Polymerase Technology, Inc), Omni KlenTaq™ DNA polymerase (DNA Polymerase Technology, Inc), Omni KlenTaq™ LA DNA polymerase (DNA Polymerase Technology, Ihc), Platinum® Taq DNA Polymerase (Invitrogen), Hemo Klentaq™ (New England Biolabs), Platinum® Taq DNA Polymerase High Fidelity (Invitrogen), Platinum® Pfx (Invitrogen), Accuprime™ Pfx (Invitrogen), or Accuprime™ Taq DNA Polymerase High Fidelity (Invitrogen).

In some embodiments, the DNA polymerase is a thermostable DNA polymerase. In some embodiments, the mixture of dNTPs is applied concurrently, or sequentially, in a random or defined order. In some embodiments, the amount of DNA polymerase present in the multiplex reaction is significantly higher than the amount of DNA polymerase used in a corresponding single plex PCR reaction. As defined herein, the term “significantly higher” refers to an at least 3-fold greater concentration of DNA polymerase present in the multiplex PCR reaction as compared to a corresponding single plex PCR reaction.

In some embodiments, the amplification reaction does not include a circularization of amplification product, for example as disclosed by rolling circle amplification.

The practice of the present subject matter may employ, unless otherwise indicated, conventional techniques and descriptions of organic chemistry, molecular biology (including recombinant techniques), cell biology, and biochemistry, which are within the skill of the art. Such conventional techniques include, but are not limited to, preparation of synthetic polynucleotides, polymerization techniques, chemical and physical analysis of polymer particles, preparation of nucleic acid libraries, nucleic acid sequencing and analysis, and the like. Specific illustrations of suitable techniques can be used by reference to the examples provided herein. Other equivalent conventional procedures can also be used. Such conventional techniques and descriptions can be found in standard laboratory manuals such as Genome Analysis: A Laboratory Manual Series (Vols. I-IV), PCR Primer: A Laboratory Manual, and Molecular Cloning: A Laboratory Manual (all from Cold Spring Harbor Laboratory Press), Hermanson, Bioconjugate Techniques, Second Edition (Academic Press, 2008); Merkus, Particle Size Measurements (Springer, 2009); Rubinstein and Colby, Polymer Physics (Oxford University Press, 2003); and the like.

According to various exemplary embodiments, one or more features of any one or more of the above-discussed teachings and/or exemplary embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various exemplary embodiments, one or more features of any one or more of the above-discussed teachings and/or exemplary embodiments may be performed or implemented using appropriately configured and/or programmed non-transitory machine-readable medium or article that may store an instruction or a set of instructions that, if executed by a machine, may cause the machine to perform a method and/or operations in accordance with the exemplary embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, Flash memory, hard drive, tape, CDROM, etc.). Moreover, memory can incorporate electronic, magnetic, optical, and/or other types of storage media. Memory can have a distributed architecture where various components are situated remote from one another, but are still accessed by the processor. The instructions may include any suitable type of code, such as source code, compiled code, interpreted code, executable code, static code, dynamic code, encrypted code, etc., implemented using any suitable high-level, low-level, object-oriented, visual, compiled and/or interpreted programming language.

According to various exemplary embodiments, one or more features of any one or more of the above-discussed teachings and/or exemplary embodiments may be performed or implemented at least partly using a distributed, clustered, remote, or cloud computing resource.

According to various exemplary embodiments, one or more features of any one or more of the above-discussed teachings and/or exemplary embodiments may be performed or implemented using a source program, executable program (object code), script, or any other entity comprising a set of instructions to be performed. When a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, Pascal, Basic, Fortran, Cobol, Perl, Java, and Ada.

According to various exemplary embodiments, one or more of the above-discussed exemplary embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such exemplary embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

Various additional exemplary embodiments may be derived by repeating, adding, or substituting any generically or specifically described features and/or components and/or substances and/or steps and/or operating conditions set forth in one or more of the above-described exemplary embodiments. Further, it should be understood that an order of steps or order for performing certain actions is immaterial so long as the objective of the steps or action remains achievable, unless specifically stated otherwise. Furthermore, two or more steps or actions can be conducted simultaneously so long as the objective of the steps or action remains achievable, unless specifically stated otherwise. Moreover, any one or more feature, component, aspect, step, or other characteristic mentioned in one of the above-discussed exemplary embodiments may be considered to be a potential optional feature, component, aspect, step, or other characteristic of any other of the above-discussed exemplary embodiments so long as the objective of such any other of the above-discussed exemplary embodiments remains achievable, unless specifically stated otherwise.

In certain embodiments, compositions of the invention comprise target BCR primer sets wherein the primers are directed to sequences of the same target BCR gene. In some embodiments the immune receptor is an antibody receptor selected from the group consisting of heavy chain alpha, heavy chain delta, heavy chain epsilon, heavy chain gamma, heavy chain mu, light chain kappa, and light chain lambda. In some embodiments, a target BCR primer set can be combined with a primer set directed to a TCR selected from the group consisting of TCR alpha, TCR beta, TCR gamma, and TCR delta.

In some embodiments, compositions of the invention comprise target BCR primer sets selected to have various parameters or criteria outlined herein. In some embodiments, compositions of the invention comprise a plurality of target-specific primers (e.g., V gene FR1-, FR2- and FR3-directed primers, the J gene directed primers, and the C gene directed primers) of about 15 nucleotides to about 40 nucleotides in length and having at least two or more following criteria: a cleavable group located at a 3′ end of substantially all of the plurality of primers, a cleavable group located near or about a central nucleotide of substantially all of the plurality of primers, substantially all of the plurality of primers at a 5′ end including only non-cleavable nucleotides, minimal cross-hybridization to substantially all of the primers in the plurality of primers, minimal cross-hybridization to non-specific sequences present in a sample, minimal self-complementarity, and minimal nucleotide sequence overlap at a 3′ end or a 5′ end of substantially all of the primers in the plurality of primers. In some embodiments, the composition can include primers with any 3, 4, 5, 6 or 7 of the above criteria.

In some embodiments, composition comprise a plurality of target-specific primers of about 15 nucleotides to about 40 nucleotides in length having two or more of the following criteria: a cleavable group located near or about a central nucleotide of substantially all of the plurality of primers, substantially all of the plurality of primers at a 5′ end including only non-cleavable nucleotides, substantially all of the plurality of primers having less than 20% of the nucleotides across the primer's entire length containing a cleavable group, at least one primer having a complementary nucleic acid sequence across its entire length to a target sequence present in a sample, minimal cross-hybridization to substantially all of the primers in the plurality of primers, minimal cross-hybridization to non-specific sequences present in a sample, and minimal nucleotide sequence overlap at a 3′ end or a 5′ end of substantially all of the primers in the plurality of primers. In some embodiments, the composition can include primers with any 3, 4, 5, 6 or 7 of the above criteria.

In some embodiments, target-specific primers (e.g., the V gene FR1-, FR2- and FR3-directed primers, the J gene directed primers, and the C gene directed primers) used in the compositions of the invention are selected or designed to satisfy any one or more of the following criteria: (1) includes two or more modified nucleotides within the primer sequence, at least one of which is included near or at the termini of the primer and at least one of which is included at, or about the center nucleotide position of the primer sequence; (2) length of about 15 to about 40 bases in length; (3) Tₘ of from above 60° C. to about 70° C.; (4) low cross-reactivity with non-target sequences present in the sample; (5) at least the first four nucleotides (going from 3′ to 5′ direction) are non-complementary to any sequence within any other primer present in the composition; and (6) non-complementary to any consecutive stretch of at least 5 nucleotides within any other sequence targeted for amplification with the primers. In some embodiments, the target-specific primers used in the compositions are selected or designed to satisfy any 2, 3, 4, 5, or 6 of the above criteria. In some embodiments, the two or more modified nucleotides have cleavable groups. In some embodiments, each of the plurality of target-specific primers comprises two or more modified nucleotides selected from a cleavable group of methylguanine, 8-oxo-guanine, xanthine, hypoxanthine, 5,6-dihydrouracil, uracil, 5-methylcytosine, thymine-dimer, 7-methylguanosine, 8-oxo-deoxyguanosine, xanthosine, inosine, dihydrouridine, bromodeoxyuridine, uridine or 5-methylcytidine.

In some embodiments compositions are provided for analysis of an immune repertoire in a sample, comprising at least one set of i) a plurality of V gene primers directed to a majority of different V genes of at least one BCR coding sequence comprising at least a portion of framework region 1 (FR1) within the V gene; and ii) one or more C gene primers directed to at least a portion of a respective target C gene of the BCR coding sequence, wherein each set of i) and ii) primers directed to the same target immune receptor sequences is selected from the group consisting of IgH, IgL, and IgK and wherein each set of i) and ii) primers directed to the same target BCR is configured to amplify the target BCR repertoire. In certain embodiments a single set of primers comprising i) and ii) is encompassed within a composition. In more particular embodiments such set comprises primers directed to IgH. In still other embodiments at least two sets of primers are encompassed in a composition wherein the sets are directed to IgH and TCR beta.

In particular embodiments, compositions provided include target BCR primer sets comprising one or more of a plurality of V gene primers directed to a sequence over an FR1 region about 70 nucleotides in length. In other particular embodiments, the one or more of a plurality of V gene primers are directed to sequences over an FR1 region about 50 nucleotides in length. In certain embodiments a target BCR primer set comprises V gene primers of about 18 to about 45 different FR1-directed primers. In some embodiments a target BCR primer set comprises V gene primers of about 22 to about 35 different FR1-directed primers. In some embodiments a target BCR primer set comprises V gene primers of about 25 to about 35 different FR1-directed primers. In certain embodiments a target BCR primer set comprises V gene primers of about 40 to about 65 different FR1-directed primers. In some embodiments a target BCR primer set comprises V gene primers of about 48 to about 60 different FR1-directed primers. In some embodiments the target BCR primer set comprises one or more C gene primers. In particular embodiments a target BCR primer set comprises at least 5 to about 15 C gene primers wherein each is directed to at least a portion of the same 50 nucleotide region within each of the target C genes. In particular embodiments a target BCR primer set comprises at least 2 to about 8 C gene primers wherein each is directed to at least a portion of the same 50 nucleotide region within each of the target C genes. In some embodiments a target BCR primer set comprises two or more C gene primers directed to different Ig isotype molecules, e.g., IgA, IgD, IgG, IgM and IgE. In some embodiments a target BCR primer set comprises at least 5 C gene primers each primer directed to a C gene of a different Ig isotype molecule.

In particular embodiments, compositions of the invention comprise at least one set of primers comprising V gene primers i) and C gene primers ii) selected from Table 3 and from Tables 6-10, respectively. In certain embodiments compositions of the invention comprise at least one set of primers i) and ii) comprising about 15 to about 35 primers selected from Table 3 and about 5 to about 20 primers selected from Tables 6-10, respectively. In some embodiments provided compositions comprise at least one set of primers comprising i) about 22 to about 35 primers selected from Table 3 and ii) one or more primers selected from each of Tables 6-10. In certain embodiments compositions of the invention comprise at least one set of primers i) and ii) comprising about 40 to about 65 primers selected from Table 3 and about 5 to about 20 primers selected from each of Tables 6-10, respectively. In some embodiments provided compositions comprise at least one set of primers comprising i) about 48 to about 65 primers selected from Table 3 and ii) one or more primers selected from each of Tables 6-10. In other certain embodiments compositions of the invention comprise at least one set of primers comprising i) primers selected from SEQ ID NOs: 137-283 and ii) primers selected from SEQ ID NOs: 448-459, 472-479, 488-513, 540-551, and 564-582. In other embodiments provided compositions comprise at least one set of primers comprising i) primers selected from SEQ ID NOs: 284-430 and ii) primers selected from SEQ ID NOs: 460-471, 480-487, 514-539, 552-563, and 583-601. In some embodiments compositions of the invention comprise at least one set of primers comprising i) primers selected from SEQ ID NOs: 137-283 and ii) primers selected from SEQ ID NOs: 460-471, 480-487, 514-539, 552-563, and 583-601 or comprising i) primers selected from SEQ ID NOs: 284-430 and ii) primers selected from SEQ ID NOs: 448-459, 472-479, 488-513, 540-551, and 564-582.

In some embodiments compositions of the invention comprise at least one set of primers i) and ii) comprising at least 20 or at least 25 primers selected from SEQ ID NOs: 137-283 and at least one primer selected from SEQ ID NOs: 448-459, 472-479, 488-513, 540-551, and 564-582. In some embodiments provided compositions comprise at least one set of primers i) and ii) comprising about 15 to about 35 primers selected from SEQ ID NOs: 137-283 and about 5 to about 15 primers selected from SEQ ID NOs: 448-459, 472-479, 488-513, 540-551, and 564-582. In some embodiments provided compositions comprise at least one set of primers comprising i) about 22 to about 35 primers selected from SEQ ID NOs: 137-283 and ii) at least one primer selected from SEQ ID NOs: 448-459, at least one primer selected from SEQ ID NOs: 472-479, at least one primer selected from SEQ ID NOs: 488-513, at least one primer selected from SEQ ID Nos: 540-551, and at least one primer selected from SEQ ID NOs:564-582. In other embodiments compositions of the invention comprise at least one set of primers i) and ii) comprising at least 20 or at least 25 primers selected from SEQ ID NOs: 284-430 and at least one primer selected from SEQ ID NOs: 460-471, 480-487, 514-539, 552-563, and 583-601. In some embodiments provided compositions comprise at least one set of primers i) and ii) comprising about 15 to about 35 primers selected from SEQ ID NOs: 284-430 and about 5 to about 15 primers selected from SEQ ID NOs: 460-471, 480-487, 514-539, 552-563, and 583-601. In some embodiments provided compositions comprise at least one set of primers comprising i) about 22 to about 35 primers selected from SEQ ID NOs: 284-430 and ii) at least one primer selected from SEQ ID NOs: 460-471, at least one primer selected from SEQ ID NOs: 480-487, at least one primer selected from SEQ ID NOs: 514-539, at least one primer selected from SEQ ID Nos: 552-563, and at least one primer selected from SEQ ID NOs:583-601. In some embodiments compositions of the invention comprise at least one set of primers i) and ii) comprising at least 20 or at least 25 primers selected from SEQ ID NOs: 137-283 and at least one primer selected from SEQ ID NOs: 460-471, 480-487, 514-539, 552-563, and 583-601. In other embodiments compositions of the invention comprise at least one set of primers i) and ii) comprising at least 20 or at least 25 primers selected from SEQ ID NOs: 284-430 and at least one primer selected from SEQ ID NOs: 448-459, 472-479, 488-513, 540-551, and 564-582.

In some embodiments compositions of the invention comprise at least one set of primers i) and ii) comprising at least 40 or at least 50 primers selected from SEQ ID NOs: 137-283 and at least one primer selected from SEQ ID NOs: 448-459, 472-479, 488-513, 540-551, and 564-582. In some embodiments provided compositions comprise at least one set of primers i) and ii) comprising about 40 to about 65 primers selected from SEQ ID NOs: 137-283 and about 5 to about 15 primers selected from SEQ ID NOs: 448-459, 472-479, 488-513, 540-551, and 564-582. In some embodiments provided compositions comprise at least one set of primers comprising i) about 48 to about 60 primers selected from SEQ ID NOs: 137-283 and ii) at least one primer selected from SEQ ID NOs: 448-459, at least one primer selected from SEQ ID NOs: 472-479, at least one primer selected from SEQ ID NOs: 488-513, at least one primer selected from SEQ ID Nos: 540-551, and at least one primer selected from SEQ ID NOs:564-582. In other embodiments compositions of the invention comprise at least one set of primers i) and ii) comprising at least 40 or at least 50 primers selected from SEQ ID NOs: 284-430 and at least one primer selected from SEQ ID NOs: 460-471, 480-487, 514-539, 552-563, and 583-601. In some embodiments provided compositions comprise at least one set of primers i) and ii) comprising about 40 to about 65 primers selected from SEQ ID NOs: 284-430 and about 5 to about 15 primers selected from SEQ ID NOs: 460-471, 480-487, 514-539, 552-563, and 583-601. In some embodiments provided compositions comprise at least one set of primers comprising i) about 48 to about 60 primers selected from SEQ ID NOs: 284-430 and ii) at least one primer selected from SEQ ID NOs: 460-471, at least one primer selected from SEQ ID NOs: 480-487, at least one primer selected from SEQ ID NOs: 514-539, at least one primer selected from SEQ ID Nos: 552-563, and at least one primer selected from SEQ ID NOs:583-601. In some embodiments compositions of the invention comprise at least one set of primers i) and ii) comprising at least 40 or at least 50 primers selected from SEQ ID NOs: 137-283 and at least one primer selected from SEQ ID NOs: 460-471, 480-487, 514-539, 552-563, and 583-601. In other embodiments compositions of the invention comprise at least one set of primers i) and ii) comprising at least 40 or at least 50 primers selected from SEQ ID NOs: 284-430 and at least one primer selected from SEQ ID NOs: 448-459, 472-479, 488-513, 540-551, and 564-582.

In some embodiments compositions are provided for analysis of a BCR repertoire in a sample, comprising at least one set of i) a plurality of V gene primers directed to a majority of different V genes of at least one BCR coding sequence comprising at least a portion of framework region 3 (FR3) within the V gene; and ii) one or more C gene primers directed to at least a portion of the respective target C gene of the BCR coding sequence, wherein each set of i) and ii) primers directed to the same target immune receptor sequences is selected from the group consisting of IgH, IgL, and IgK and wherein each set of i) and ii) primers directed to the same target immune receptor is configured to amplify the target BCR repertoire. In certain embodiments a single set of primers comprising i) and ii) is encompassed within a composition. In more particular embodiments such set comprises primers directed to IgH. In still other embodiments at least two sets of primers are encompassed in a composition wherein the sets are directed to IgH and TCR beta.

In certain embodiments, compositions provided include target BCR primer sets comprising V gene primers wherein the one or more of a plurality of V gene primers are directed to sequences over an FR3 region about 70 nucleotides in length. In particular embodiments, compositions provided include target BCR primer sets comprising V gene primers wherein the one or more of a plurality of V gene primers are directed to sequences over an FR3 region about 50 nucleotides in length. In other particular embodiments the one or more of a plurality of V gene primers are directed to sequences over an FR3 region about 40 to about 60 nucleotides in length. In certain embodiments a target BCR primer set comprises V gene primers of about 50 to about 85 different FR3-directed primers. In certain embodiments a target BCR primer set comprises V gene primers of about 55 to about 80 different FR3-directed primers. In some embodiments a target BCR primer set comprises V gene primers of about 62 to about 75 different FR3-directed primers. In some embodiments, a target BCR primer set comprises V gene primers of about 65, 66, 67, 68, 69, or 70 different FR3-directed primers. In some embodiments the target BCR primer set comprises one or more C gene primers. In particular embodiments a target BCR primer set comprises at least 5 to about 15 C gene primers wherein each is directed to at least a portion of the same 50 nucleotide region within each of the target C genes. In particular embodiments a target immune receptor primer set comprises at least 2 to about 8 C gene primers wherein each is directed to at least a portion of the same 50 nucleotide region within each of the target C genes. In some embodiments a target BCR primer set comprises two or more C gene primers directed to different Ig isotype molecules, e.g., IgA, IgD, IgG, IgM and IgE. In some embodiments a target BCR primer set comprises at least 5 C gene primers each primer directed to a C gene of a different Ig isotype molecule.

In particular embodiments, compositions of the invention comprise at least one set of primers comprising V gene primers i) and C gene primers ii) selected from Table 2 and from Tables 6-10, respectively. In certain embodiments compositions of the invention comprise at least one set of primers i) and ii) comprising about 55 to about 80 primers selected from Table 2 and about 5 to about 20 primers selected from Tables 6-10, respectively. In some embodiments provided compositions comprise at least one set of primers comprising i) about 62 to about 75 primers selected from Table 2 and ii) one or more primers selected from each of Tables 6-10. In certain other embodiments compositions of the invention comprise at least one set of primers i) and ii) comprising primers selected from SEQ ID NOs: 1-68 and 448-459, 472-479, 488-513, 540-551, and 564-582 or selected from SEQ ID NOs: 69-136 and 460-471, 480-487, 514-539, 552-563, and 583-601. In some embodiments compositions of the invention comprise at least one set of primers i) and ii) comprising primers selected from SEQ ID NOs: 1-68 and 460-471, 480-487, 514-539, 552-563, and 583-601 or selected from SEQ ID NOs: 69-136 and 448-459, 472-479, 488-513, 540-551, and 564-582.

In some embodiments compositions of the invention comprise at least one set of primers i) and ii) comprising at least 60 primers selected from SEQ ID NOs: 1-68 and at least one primer selected from SEQ ID NOs: 448-459, 472-479, 488-513, 540-551, and 564-582. In some embodiments provided compositions comprise at least one set of primers i) and ii) comprising at least 60 primers selected from SEQ ID NOs: 1-68 and about 5 to about 15 primers selected from SEQ ID NOs: 448-459, 472-479, 488-513, 540-551, and 564-582. In some embodiments provided compositions comprise at least one set of primers comprising i) at least 60 primers selected from SEQ ID NOs: 1-68 and ii) at least one primer selected from SEQ ID NOs: 448-459, at least one primer selected from SEQ ID NOs: 472-479, at least one primer selected from SEQ ID NOs: 488-513, at least one primer selected from SEQ ID Nos: 540-551, and at least one primer selected from SEQ ID NOs:564-582. In other embodiments compositions of the invention comprise at least one set of primers i) and ii) comprising at least 60 primers selected from SEQ ID NOs: 69-136 and at least one primer selected from SEQ ID NOs: 460-471, 480-487, 514-539, 552-563, and 583-601. In some embodiments provided compositions comprise at least one set of primers i) and ii) comprising at least 60 primers selected from SEQ ID NOs: 69-136 and about 5 to about 15 primers selected from SEQ ID NOs: 460-471, 480-487, 514-539, 552-563, and 583-601. In some embodiments provided compositions comprise at least one set of primers comprising i) at least 60 primers selected from SEQ ID NOs: 69-136 and ii) at least one primer selected from SEQ ID NOs: 460-471, at least one primer selected from SEQ ID NOs: 480-487, at least one primer selected from SEQ ID NOs: 514-539, at least one primer selected from SEQ ID Nos: 552-563, and at least one primer selected from SEQ ID NOs:583-601. In some embodiments compositions of the invention comprise at least one set of primers i) and ii) comprising at least 60 primers selected from SEQ ID NOs: 1-68 and at least one primer selected from SEQ ID NOs: 460-471, 480-487, 514-539, 552-563, and 583-601. In other embodiments compositions of the invention comprise at least one set of primers i) and ii) comprising at least 60 primers selected from SEQ ID NOs: 69-136 and at least one primer selected from SEQ ID NOs: 448-459, 472-479, 488-513, 540-551, and 564-582.

In some embodiments compositions are provided for analysis of a BCR repertoire in a sample, comprising at least one set of i) a plurality of V gene primers directed to a majority of different V genes of at least one BCR coding sequence comprising at least a portion of FR2 within the V gene; and ii) one or more C gene primers directed to at least a portion of the respective target C gene of the BCR coding sequence, wherein each set of i) and ii) primers directed to the same target immune receptor sequences is selected from the group consisting of IgH, IgL, and IgK and wherein each set of i) and ii) primers directed to the same target immune receptor is configured to amplify the target BCR repertoire. In certain embodiments a single set of primers comprising i) and ii) is encompassed within a composition. In more particular embodiments such set comprises primers directed to IgH. In still other embodiments at least two sets of primers are encompassed in a composition wherein the sets are directed to IgH and TCR beta.

In particular embodiments, compositions provided include target BCR primer sets comprising V gene primers wherein the one or more of a plurality of V gene primers are directed to sequences over an FR2 region about 70 nucleotides in length. In other particular embodiments the one or more of a plurality of V gene primers are directed to sequences over an FR2 region about 50 nucleotides in length. In certain embodiments a target BCR primer set comprises V gene primers of about 5 to about 15 different FR2-directed primers. In some embodiments a target BCR primer set comprises V gene primers of about 5, 6, 7, 8, 9, 10, 11, or 12 different FR2-directed primers. In some embodiments the target BCR primer set comprises one or more C gene primers. In particular embodiments a target BCR primer set comprises at least 5 to about 15 C gene primers wherein each is directed to at least a portion of the same 50 nucleotide region within each of the target C genes. In particular embodiments a target BCR primer set comprises at least 2 to about 8 C gene primers wherein each is directed to at least a portion of the same 50 nucleotide region within each of the target C genes. In some embodiments a target BCR primer set comprises two or more C gene primers directed to different Ig isotype molecules, e.g., IgA, IgD, IgG, IgM and IgE. In some embodiments a target BCR primer set comprises at least 5 C gene primers each primer directed to a C gene of a different Ig isotype molecule.

In particular embodiments, compositions of the invention comprise at least one set of primers comprising V gene primers i) and C gene primers ii) selected from Table 4 and from Tables 6-10, respectively. In certain other embodiments compositions of the invention comprise at least one set of primers i) and ii) comprising primers selected from SEQ ID NOs: 431-437 and 448-459, 472-479, 488-513, 540-551, and 564-582. In other embodiments compositions of the invention comprise at least one set of primers i) and ii) comprising primers selected from SEQ ID NOs: 431-437 and 460-471, 480-487, 514-539, 552-563, and 583-601. In other embodiments compositions of the invention comprise at least one set of primers i) and ii) comprising at least 5 primers selected from SEQ ID NOs: 431-437 and at least one primer selected from SEQ ID NOs: 448, 472, 488, 540, 541, 564 and 565. In other embodiments compositions of the invention comprise at least one set of primers i) and ii) comprising at least 5 primers selected from SEQ ID NOs: 431-437 and at least one primer selected from SEQ ID NOs: 460, 480, 514, 552, 553, 583, and 584.

In some embodiments compositions are provided for analysis of a BCR repertoire in a sample, comprising at least one set of i) a plurality of V gene primers directed to a majority of different V genes of at least one BCR coding sequence comprising at least a portion of framework region 3 (FR3) within the V gene; and ii) a plurality of J gene primers directed to a majority of different J genes of the respective target BCR coding sequence, wherein each set of i) and ii) primers directed to the same target immune receptor sequences is selected from the group consisting of IgH, IgL, and IgK and wherein each set of i) and ii) primers directed to the same target immune receptor is configured to amplify the target BCR repertoire. In certain embodiments a single set of primers comprising i) and ii) is encompassed within a composition. In more particular embodiments such set comprises primers directed to IgH. In still other embodiments at least two sets of primers are encompassed in a composition wherein the sets are directed to IgH and TCR beta.

In particular embodiments, compositions provided include target BCR primer sets comprising V gene primers wherein the one or more of a plurality of V gene primers are directed to sequences over an FR3 region about 50 nucleotides in length. In other embodiments the one or more of a plurality of V gene primers are directed to sequences over an FR3 region about 70 nucleotides in length. In other particular embodiments the one or more of a plurality of V gene primers are directed to sequences over an FR3 region about 40 to about 60 nucleotides in length. In some embodiments a target BCR primer set comprises V gene primers comprising about 50 to about 85 different FR3-directed primers. In certain embodiments a target BCR primer set comprises V gene primers comprising about 55 to about 80 different FR3-directed primers. In some embodiments a target BCR primer set comprises V gene primers comprising about 62 to about 75 different FR3-directed primers. In some embodiments, a target BCR primer set comprises V gene primers comprising about 65, 66, 67, 68, 69, or 70 different FR3-directed primers. In some embodiments the target BCR primer set comprises a plurality of J gene primers. In some embodiments a target BCR primer set comprises at least 2 J gene primers wherein each is directed to at least a portion of a J gene within target polynucleotides. In certain embodiments a target BCR primer set comprises 2 to about 8 J gene primers wherein each is directed to at least a portion of a J gene within target polynucleotides. In some embodiments a target BCR primer set comprises about 3 to about 6 different J gene primers wherein each is directed to at least a portion of a J gene within target polynucleotides. In some embodiments a target BCR primer set comprises about 2, 3, 4, 5, 6, 7 or 8 different J gene primers wherein each is directed to at least a portion of a J gene within target polynucleotides. In some embodiments a target BCR primer set comprises about 4 J gene primers wherein each is directed to at least a portion of the J gene portion within target polynucleotides.

In particular embodiments, compositions of the invention comprise at least one set of primers comprising V gene primers i) and J gene primers ii) selected from Tables 2 and 5, respectively. In certain embodiments compositions of the invention comprise at least one set of primers i) and ii) comprising primers selected from SEQ ID NOs: 1-68 and 438-442 or selected from SEQ ID NOs: 69-136 and 443-447. In other certain embodiments compositions of the invention comprise at least one set of primers i) and ii) comprising primers selected from SEQ ID NOs: 1-68 and 443-447 or selected from SEQ ID NOs: 69-136 and 438-442.

In some embodiments compositions of the invention comprise at least one set of primers i) and ii) comprising at least 60 primers selected from SEQ ID NOs: 1-68 and at least 2 primers, at least 3 primers, or at least 4 primers selected from SEQ ID NOs: 438-442. In some embodiments compositions of the invention comprise at least one set of primers i) and ii) comprising at least 60 primers selected from SEQ ID NOs: 69-136 and at least 2 primers, at least 3 primers, or at least 4 primers selected from SEQ ID NOs: 443-447. In some embodiments compositions of the invention comprise at least one set of primers i) and ii) comprising at least 60 primers selected from SEQ ID NOs: 1-68 and at least 2 primers, at least 3 primers, or at least 4 primers selected from SEQ ID NOs: 443-447. In some embodiments compositions of the invention comprise at least one set of primers i) and ii) comprising at least 60 primers selected from SEQ ID NOs: 69-136 and at least 2 primers, at least 3 primers, or at least 4 primers selected from SEQ ID NOs: 438-442.

In some embodiments compositions are provided for analysis of a BCR repertoire in a sample, comprising at least one set of i) a plurality of V gene primers directed to a majority of different V genes of at least one BCR coding sequence comprising at least a portion of framework region 1 (FR1) within the V gene; and ii) a plurality of J gene primers directed to a majority of different J genes of the respective target BCR coding sequence, wherein each set of i) and ii) primers directed to the same target immune receptor sequences is selected from the group consisting of IgH, IgL, and IgK and wherein each set of i) and ii) primers directed to the same target immune receptor is configured to amplify the target BCR repertoire. In certain embodiments a single set of primers comprising i) and ii) is encompassed within a composition. In more particular embodiments such set comprises primers directed to IgH. In still other embodiments at least two sets of primers are encompassed in a composition wherein the sets are directed to IgH and TCR beta.

In particular embodiments, compositions provided include target BCR primer sets comprising one or more of a plurality of V gene primers directed to a sequence over an FR1 region about 70 nucleotides in length. In other embodiments the one or more of a plurality of V gene primers are directed to sequences over an FR1 region about 80 nucleotides in length. In other particular embodiments, the one or more of a plurality of V gene primers are directed to sequences over an FR1 region about 50 nucleotides in length. In certain embodiments a target BCR primer set comprises V gene primers comprising about 18 to about 45 different FR1-directed primers. In some embodiments a target BCR primer set comprises V gene primers of about 22 to about 35 different FR1-directed primers. In some embodiments a target BCR primer set comprises V gene primers of about 25 to about 35 different FR1-directed primers. In certain embodiments a target BCR primer set comprises V gene primers of about 40 to about 65 different FR1-directed primers. In some embodiments a target BCR primer set comprises V gene primers of about 48 to about 60 different FR1-directed primers. In some embodiments the target BCR primer set comprises a plurality of J gene primers. In some embodiments a target BCR primer set comprises at least 2 J gene primers wherein each is directed to at least a portion of a J gene within target polynucleotides. In certain embodiments a target BCR primer set comprises 2 to about 8 J gene primers wherein each is directed to at least a portion of a J gene within target polynucleotides. In some embodiments a target BCR primer set comprises about 3 to about 6 different J gene primers wherein each is directed to at least a portion of a J gene within target polynucleotides. In some embodiments a target BCR primer set comprises about 2, 3, 4, 5, 6, 7 or 8 different J gene primers wherein each is directed to at least a portion of a J gene within target polynucleotides. In some embodiments a target BCR primer set comprises about 4 J gene primers wherein each is directed to at least a portion of the J gene portion within target polynucleotides.

In particular embodiments, compositions of the invention comprise at least one set of primers comprising V gene primers i) and J gene primers ii) selected from Tables 3 and 5, respectively. In certain other embodiments compositions of the invention comprise at least one set of primers i) and ii) comprising primers selected from SEQ ID NOs: 137-283 and 438-442 or selected from SEQ ID NOs: 284-430 and 443-447. In other embodiments compositions of the invention comprise at least one set of primers i) and ii) comprising primers selected from SEQ ID NOs: 137-283 and 443-447 or selected from SEQ ID NOs: 284-430 and 438-442.

In some embodiments compositions of the invention comprise at least one set of primers i) and ii) comprising at least 20 or at least 25 primers selected from SEQ ID NOs: 137-283 and at least 2 primers, at least 3 primers, or at least 4 primers selected from SEQ ID NOs: 438-442. In some embodiments provided compositions comprise at least one set of primers i) and ii) comprising about 15 to about 35 primers selected from SEQ ID NOs: 137-283 and at least 2 primers, at least 3 primers, or at least 4 primers selected from SEQ ID NOs: 438-442. In some embodiments provided compositions comprise at least one set of primers i) and ii) comprising about 22 to about 35 primers selected from SEQ ID NOs: 137-283 and at least 2 primers, at least 3 primers, or at least 4 primers selected from SEQ ID NOs: 438-442. In some embodiments compositions of the invention comprise at least one set of primers i) and ii) comprising at least 20 or at least 25 primers selected from SEQ ID NOs: 284-430 and at least 2 primers, at least 3 primers, or at least 4 primers selected from SEQ ID NOs: 443-447. In some embodiments provided compositions comprise at least one set of primers i) and ii) comprising about 15 to about 35 primers selected from SEQ ID NOs: 284-430 and at least 2 primers, at least 3 primers, or at least 4 primers selected from SEQ ID NOs: 443-447. In some embodiments provided compositions comprise at least one set of primers i) and ii) comprising about 22 to about 35 primers selected from SEQ ID NOs: 284-430 and at least 2 primers, at least 3 primers, or at least 4 primers selected from SEQ ID NOs: 443-447. In some embodiments compositions of the invention comprise at least one set of primers i) and ii) comprising at least 20 or at least 25 primers selected from SEQ ID NOs: 137-283 and at least 2 primers, at least 3 primers, or at least 4 primers selected from SEQ ID NOs: 443-447. In some embodiments compositions of the invention comprise at least one set of primers i) and ii) comprising at least 20 or at least 25 primers selected from SEQ ID NOs: 284-430 and at least 2 primers, at least 3 primers, or at least 4 primers selected from SEQ ID NOs: 438-442.

In some embodiments compositions of the invention comprise at least one set of primers i) and ii) comprising at least 40 or at least 50 primers selected from SEQ ID NOs: 137-283 and at least 2 primers, at least 3 primers, or at least 4 primers selected from SEQ ID NOs: 438-442. In some embodiments provided compositions comprise at least one set of primers i) and ii) comprising about 40 to about 65 primers selected from SEQ ID NOs: 137-283 and at least 2 primers, at least 3 primers, or at least 4 primers selected from SEQ ID NOs: 438-442. In some embodiments provided compositions comprise at least one set of primers i) and ii) comprising about 48 to about 60 primers selected from SEQ ID NOs: 137-283 and at least 2 primers, at least 3 primers, or at least 4 primers selected from SEQ ID NOs: 438-442. In some embodiments compositions of the invention comprise at least one set of primers i) and ii) comprising at least 40 or at least 50 primers selected from SEQ ID NOs: 284-430 and at least 2 primers, at least 3 primers, or at least 4 primers selected from SEQ ID NOs: 443-447. In some embodiments provided compositions comprise at least one set of primers i) and ii) comprising about 40 to about 65 primers selected from SEQ ID NOs: 284-430 and at least 2 primers, at least 3 primers, or at least 4 primers selected from SEQ ID NOs: 443-447. In some embodiments provided compositions comprise at least one set of primers i) and ii) comprising about 48 to about 60 primers selected from SEQ ID NOs: 284-430 and at least 2 primers, at least 3 primers, or at least 4 primers selected from SEQ ID NOs: 443-447. In some embodiments compositions of the invention comprise at least one set of primers i) and ii) comprising at least 40 or at least 50 primers selected from SEQ ID NOs: 137-283 and at least 2 primers, at least 3 primers, or at least 4 primers selected from SEQ ID NOs: 443-447. In some embodiments compositions of the invention comprise at least one set of primers i) and ii) comprising at least 40 or at least 50 primers selected from SEQ ID NOs: 284-430 and at least 2 primers, at least 3 primers, or at least 4 primers selected from SEQ ID NOs: 438-442.

In some embodiments compositions are provided for analysis of a BCR repertoire in a sample, comprising at least one set of i) a plurality of V gene primers directed to a majority of different V genes of at least one BCR coding sequence comprising at least a portion of FR2 within the V gene; and ii) a plurality of J gene primers directed to a majority of different J genes of the respective target BCR coding sequence, wherein each set of i) and ii) primers directed to the same target immune receptor sequences is selected from the group consisting of IgH, IgL, and IgK and wherein each set of i) and ii) primers directed to the same target immune receptor is configured to amplify the target BCR repertoire. In certain embodiments a single set of primers comprising i) and ii) is encompassed within a composition. In more particular embodiments such set comprises primers directed to IgH. In still other embodiments at least two sets of primers are encompassed in a composition wherein the sets are directed to IgH and TCR beta.

In particular embodiments, compositions provided include target BCR primer sets comprising V gene primers wherein the one or more of a plurality of V gene primers are directed to sequences over an FR2 region about 70 nucleotides in length. In other particular embodiments the one or more of a plurality of V gene primers are directed to sequences over an FR2 region about 50 nucleotides in length. In certain embodiments a target BCR primer set comprises V gene primers comprising about 5 to about 15 different FR2-directed primers. In some embodiments a target BCR primer set comprises V gene primers of about 5, 6, 7, 8, 9, 10, 11, or 12 different FR2-directed primers. In some embodiments the target BCR primer set comprises a plurality of J gene primers. In some embodiments a target BCR primer set comprises at least 2 J gene primers wherein each is directed to at least a portion of a J gene within target polynucleotides. In certain embodiments a target BCR primer set comprises 2 to about 8 J gene primers wherein each is directed to at least a portion of a J gene within target polynucleotides. In some embodiments a target BCR primer set comprises about 3 to about 6 different J gene primers wherein each is directed to at least a portion of a J gene within target polynucleotides. In some embodiments a target BCR primer set comprises about 2, 3, 4, 5, 6, 7 or 8 different J gene primers wherein each is directed to at least a portion of a J gene within target polynucleotides. In some embodiments a target BCR primer set comprises about 4 J gene primers wherein each is directed to at least a portion of the J gene portion within target polynucleotides.

In particular embodiments, compositions of the invention comprise at least one set of primers comprising V gene primers i) and J gene primers ii) selected from Tables 4 and 5, respectively. In some embodiments compositions of the invention comprise at least one set of primers i) and ii) comprising primers selected from SEQ ID NOs: 431-437 and 438-442 or selected from SEQ ID NOs: 431-437 and 443-447. In some embodiments compositions of the invention comprise at least one set of primers i) and ii) comprising at least 5 primers selected from SEQ ID NOs: 431-437 and at least 2 primers, at least 3 primers, or at least 4 primers selected from SEQ ID NOs: 438-442. In other embodiments compositions of the invention comprise at least one set of primers i) and ii) comprising at least 5 primers selected from SEQ ID NOs: 431-437 and at least 2 primers, at least 3 primers, or at least 4 primers selected from SEQ ID NOs: 443-447.

In some embodiments, multiple different primers including at least one modified nucleotide can be used in a single amplification reaction. For example, multiplexed primers including modified nucleotides can be added to the amplification reaction mixture, where each primer (or set of primers) selectively hybridizes to, and promotes amplification of different rearranged target nucleic acid molecules within the nucleic acid population. In some embodiments, the target specific primers can include at least one uracil nucleotide.

In some embodiments, multiplex amplification may be performed using PCR and cycles of denaturation, primer annealing, and polymerase extension steps at set temperatures for set times. In some embodiments, about 12 cycles to about 30 cycles are used to generate the amplicon library in the multiplex amplification reaction. In some embodiments, 13 cycles, 14 cycles, 15 cycles, 16 cycles, 17 cycles, 18 cycles, 19 cycles, preferably 20 cycles, 23 cycles, or 25 cycles are used to generate the amplicon library in the multiplex amplification reaction. In some embodiments, 17-25 cycles are used to generate the amplicon library in the multiplex amplification reaction.

In some embodiments, the amplification reactions are conducted in parallel within a single reaction phase (for example, within the same amplification reaction mixture within a single well or tube). In some instances, an amplification reaction can generate a mixture of products including both the intended amplicon product as well as unintended, unwanted, nonspecific amplification artifacts such as primer-dimers. Post amplification, the reactions are then treated with any suitable agent that will selectively cleave or otherwise selectively destroy the nucleotide linkages of the modified nucleotides within the excess unincorporated primers and the amplification artifacts without cleaving or destroying the specification amplification products. For example, the primers can include uracil-containing nucleobases that can be selectively cleaved using UNG/UDG (optionally with heat and/or alkali). In some embodiments, the primers can include uracil-containing nucleotides that can be selectively cleaved using UNG and Fpg. In some embodiments, the cleavage treatment includes exposure to oxidizing conditions for selective cleavage of dithiols, treatment with RNAse H for selective cleavage of modified nucleotides including RNA-specific moieties (e.g., ribose sugars, etc.), and the like. This cleavage treatment can effectively fragment the original amplification primers and non-specific amplification products into small nucleic acid fragments that include relatively few nucleotides each. Such fragments are typically incapable of promoting further amplification at elevated temperatures. Such fragments can also be removed relatively easily from the reaction pool through the various post-amplification cleanup procedures known in the art (e.g., spin columns, NaEtOH precipitation, etc).

In some embodiments, amplification products following cleavage or other selective destruction of the nucleotide linkages of the modified nucleotides are optionally treated to generate amplification products that possess a phosphate at the 5′ termini. In some embodiments, the phosphorylation treatment includes enzymatic manipulation to produce 5′ phosphorylated amplification products. In one embodiment, enzymes such as polymerases can be used to generate 5′ phosphorylated amplification products. For example, T4 polymerase can be used to prepare 5′ phosphorylated amplicon products. Klenow can be used in conjunction with one or more other enzymes to produce amplification products with a 5′ phosphate. In some embodiments, other enzymes known in the art can be used to prepare amplification products with a 5′ phosphate group. For example, incubation of uracil nucleotide containing amplification products with the enzyme UDG, Fpg and T4 polymerase can be used to generate amplification products with a phosphate at the 5′ termini. It will be apparent to one of skill in the art that other techniques, other than those specifically described herein, can be applied to generate phosphorylated amplicons. It is understood that such variations and modifications that are applied to practice the methods, systems, kits, compositions and apparatuses disclosed herein, without resorting to undue experimentation are considered within the scope of the disclosure.

In some embodiments, primers that are incorporated in the intended (specific) amplification products, these primers are similarly cleaved or destroyed, resulting in the formation of “sticky ends” (e.g., 5′ or 3′ overhangs) within the specific amplification products. Such “sticky ends” can be addressed in several ways. For example, if the specific amplification products are to be cloned, the overhang regions can be designed to complement overhangs introduced into the cloning vector, thereby enabling sticky ended ligations that are more rapid and efficient than blunt ended ligations. Alternatively, the overhangs may need to be repaired (as with several next-generation sequencing methods). Such repair can be accomplished either through secondary amplification reactions using only forward and reverse amplification primers (e.g., correspond to A and P1 primers) comprised of only natural bases. In this manner, subsequent rounds of amplification rebuild the double-stranded templates, with nascent copies of the amplicon possessing the complete sequence of the original strands prior to primer destruction. Alternatively, the sticky ends can be removed using some forms of fill-in and ligation processing, wherein the forward and reverse primers are annealed to the templates. A polymerase can then be employed to extend the primers, and then a ligase, optionally a thermostable ligase, can be utilized to connect the resulting nucleic acid strands. This could obviously be also accomplished through various other reaction pathways, such as cyclical extend-ligation, etc. In some embodiments, the ligation step can be performed using one or more DNA ligases.

In some embodiments, the amplicon library prepared using target-specific primer pairs can be used in downstream enrichment applications such as emulsion PCR, bridge PCR or isothermal amplification. In some embodiments, the amplicon library can be used in an enrichment application and a sequencing application. For example, an amplicon library can be sequenced using any suitable DNA sequencing platform, including any suitable next generation DNA sequencing platform. In some embodiments, an amplicon library can be sequenced using an Ion PGM Sequencer or an Ion GeneStudio S5 Sequencer (Thermo Fisher Scientific). In some embodiments, a PGM Sequencer or an S5 Sequencer can be coupled to server that applies parameters or software to determine the sequence of the amplified target nucleic acid molecules. In some embodiments, the amplicon library can be prepared, enriched and sequenced in less than 24 hours. In some embodiments, the amplicon library can be prepared, enriched and sequenced in approximately 9 hours.

In some embodiments, methods for generating an amplicon library can include: amplifying cDNA of immune receptor genes using V gene-specific and C gene-specific primers to generate amplicons; purifying the amplicons from the input DNA and primers; phosphorylating the amplicons; ligating adapters to the phosphorylated amplicons; purifying the ligated amplicons; nick-translating the amplified amplicons; and purifying the nick-translated amplicons to generate the amplicon library. In some embodiments, methods for generating an amplicon library can include: amplifying cDNA of immune receptor genes using V gene-specific and J gene-specific primers to generate amplicons; purifying the amplicons from the input DNA and primers; phosphorylating the amplicons; ligating adapters to the phosphorylated amplicons; purifying the ligated amplicons; nick-translating the amplified amplicons; and purifying the nick-translated amplicons to generate the amplicon library. In some embodiments, additional amplicon library manipulations can be conducted following the step of amplification of rearranged immune receptor gene targets to generate the amplicons. In some embodiments, any combination of additional reactions can be conducted in any order, and can include: purifying; phosphorylating; ligating adapters; nick-translating; amplification and/or sequencing. In some embodiments, any of these reactions can be omitted or can be repeated. It will be readily apparent to one of skill in the art that the method can repeat or omit any one or more of the above steps. It will also be apparent to one of skill in the art that the order and combination of steps may be modified to generate the required amplicon library, and is not therefore limited to the exemplary methods provided.

A phosphorylated amplicon can be joined to an adapter to conduct a nick translation reaction, subsequent downstream amplification (e.g., template preparation), or for attachment to particles (e.g., beads), or both. For example, an adapter that is joined to a phosphorylated amplicon can anneal to an oligonucleotide capture primer which is attached to a particle, and a primer extension reaction can be conducted to generate a complimentary copy of the amplicon attached to the particle or surface, thereby attaching an amplicon to a surface or particle. Adapters can have one or more amplification primer hybridization sites, sequencing primer hybridization sites, barcode sequences, and combinations thereof. In some embodiments, amplicons prepared by the methods disclosed herein can be joined to one or more Ion Torrent™ compatible adapters to construct an amplicon library. Amplicons generated by such methods can be joined to one or more adapters for library construction to be compatible with a next generation sequencing platform. For example, the amplicons produced by the teachings of the present disclosure can be attached to adapters provided in the Ion AmpliSeq™ Library Kit 2.0 or Ion AmpliSeq™ Library Kit Plus (Thermo Fisher Scientific).

In some embodiments, amplification of immune receptor cDNA or rearranged gDNA can be conducted using a 5× Ion AmpliSeq™ HiFi Master Mix. In some embodiments, the 5× Ion AmpliSeq™ HiFi Master Mix can include glycerol, dNTPs, and a DNA polymerase such as Platinum™ Taq DNA polymerase High Fidelity. In some embodiments, the 5× Ion AmpliSeq™ HiFi Master Mix can further include at least one of the following: a preservative, magnesium chloride, magnesium sulfate, tris-sulfate and/or ammonium sulfate.

In some embodiments, the immune receptor rearranged gDNA multiplex amplification reaction further includes at least one PCR additive to improve on-target amplification, amplification yield, and/or the percentage of productive sequencing reads. In some embodiments, the at least one PCR additive includes at least one of potassium chloride or additional dNTPs (e.g., dATP, dCTP, dGTP, dTTP). In some embodiments, the dNTPs as a PCR additive is an equimolar mixture of dNTPs. In some embodiments, the dNTP mix as a PCR additive is an equimolar mixture of dATP, dCTP, dGTP, and dTTP. In some embodiments, about 0.2 mM to about 5.0 mM dNTPs is added to the multiplex amplification reaction. In some embodiments, amplification of rearranged immune receptor gDNA can be conducted using a 5× Ion AmpliSeq™ HiFi Master Mix and an additional about 0.2 mM to about 5.0 mM dNTPs in the reaction mixture. In some embodiments, amplification of rearranged immune receptor gDNA can be conducted using a 5× Ion AmpliSeq™ HiFi Master Mix and an additional about 0.5 mM to about 4 mM, about 0.5 mM to about 3 mM, about 0.5 mM to about 2.5 mM, about 0.5 mM to about 1.0 mM, about 0.75 mM to about 1.25 mM, about 1.0 mM to about 1.5 mM, about 1.0 to about 2.0 mM, about 2.0 mM to about 3.0 mM, about 1.25 to about 1.75 mM, about 1.3 to about 1.8 mM, about 1.4 mM to about 1.7 mM, or about 1.5 to about 2.0 mM dNTPs in the reaction mixture. In some embodiments, amplification of rearranged immune receptor gDNA can be conducted using a 5× Ion AmpliSeq™ HiFi Master Mix and an additional about 0.2 mM, about 0.4 mM, about 0.6 mM, about 0.8 mM, about 1.0 mM, about 1.2 mM, about 1.4 mM, about 1.6 mM, about 1.8 mM, about 2.0 mM, about 2.2 mM, about 2.4 mM, about 2.6 mM, about 2.8 mM, about 3.0 mM, about 3.5 mM, or about 4.0 mM dNTPs in the reaction mixture. In some embodiments, about 10 mM to about 200 mM potassium chloride is added to the multiplex amplification reaction. In some embodiments, amplification of rearranged immune receptor gDNA can be conducted using a 5× Ion AmpliSeq™ HiFi Master Mix and an additional about 10 mM to about 200 mM potassium chloride in the reaction mixture. In some embodiments, amplification of rearranged immune receptor gDNA can be conducted using a 5× Ion AmpliSeq™ HiFi Master Mix and an additional about 10 mM to about 60 mM, about 20 mM to about 70 mM, about 30 mM to about 80 mM, about 40 mM to about 90 mM, about 50 mM to about 100 mM, about 60 mM to about 120 mM, about 80 mM to about 140 mM, about 50 mM to about 150 mM, about 150 mM to about 200 mM or about 100 mM to about 200 mM potassium chloride in the reaction mixture. In some embodiments, amplification of rearranged immune receptor gDNA can be conducted using a 5× Ion AmpliSeq™ HiFi Master Mix and an additional about 10 mM, about 20 mM, about 30 mM, about 40 mM, about 50 mM, about 60 mM, about 70 mM, about 80 mM, about 90 mM, about 100 mM, about 120 mM, about 140 mM, about 150 mM, about 160 mM, about 180 mM, or about 200 mM potassium chloride in the reaction mixture.

In some embodiments, phosphorylation of the amplicons can be conducted using a FuPa reagent. In some embodiments, the FuPa reagent can include a DNA polymerase, a DNA ligase, at least one uracil cleaving or modifying enzyme, and/or a storage buffer. In some embodiments, the FuPa reagent can further include at least one of the following: a preservative and/or a detergent.

In some embodiments, phosphorylation of the amplicons can be conducted using a FuPa reagent. In some embodiments, the FuPa reagent can include a DNA polymerase, at least one uracil cleaving or modifying enzyme, an antibody and/or a storage buffer. In some embodiments, the FuPa reagent can further include at least one of the following: a preservative and/or a detergent. In some embodiments, the antibody is provided to inhibit the DNA polymerase and 3′-5′ exonuclease activities at ambient temperature.

In some embodiments, the amplicon library produced by the teachings of the present disclosure are sufficient in yield to be used in a variety of downstream applications including the Ion Chef™ instrument and the Ion S5™ Sequencing Systems (Thermo Fisher Scientific).

It will be apparent to one of ordinary skill in the art that numerous other techniques, platforms or methods for clonal amplification such as wildfire PCR and bridge amplification can be used in conjunction with the amplified target sequences of the present disclosure. It is also envisaged that one of ordinary skill in art upon further refinement or optimization of the conditions provided herein can proceed directly to nucleic acid sequencing (for example using Ion PGM™ System or Ion S5™ System or Ion Proton™ System sequencers, Thermo Fisher Scientific) without performing a clonal amplification step.

In some embodiments, at least one of the amplified targets sequences to be clonally amplified can be attached to a support or particle. The support can be comprised of any suitable material and have any suitable shape, including, for example, planar, spheroid or particulate. In some embodiments, the support is a scaffolded polymer particle as described in U.S. Published App. No. 20100304982, hereby incorporated by reference in its entirety.

In some embodiments, a kit is provided for amplifying multiple immune receptor expression sequences from a population of nucleic acid molecules in a single reaction. In some embodiments, the kit includes a plurality of target-specific primer pairs containing one or more cleavable groups, one or more DNA polymerases, a mixture of dNTPs and at least one cleaving reagent. In one embodiment, the cleavable group is 8-oxo-deoxyguanosine, deoxyuridine or bromodeoxyuridine. In some embodiments, the at least one cleaving reagent includes RNaseH, uracil DNA glycosylase, Fpg or alkali. In one embodiment, the cleaving reagent is uracil DNA glycosylase. In some embodiments, the kit is provided to perform multiplex PCR in a single reaction chamber or vessel. In some embodiments, the kit includes at least one DNA polymerase, which is a thermostable DNA polymerase. In some embodiments, the concentration of the one or more DNA polymerases is present in a 3-fold excess as compared to a single PCR reaction. In some embodiments, the final concentration of each target-specific primer pair is present at about 5 nM to about 2000 nM. In some embodiments, the final concentration of each target-specific primer pair is present at about 25 nM to about 50 nM or about 100 nM to about 800 nM. In some embodiments, the final concentration of each target-specific primer pair is present at about 50 nM to about 400 nM or about 50 nM to about 200 nM. In some embodiments, the final concentration of each target-specific primer pair is present at about 200 nM or about 400 nM. In some embodiments, the kit provides amplification of immune repertoire expression sequences from TCR beta, TCR alpha, TCR gamma, TCR delta, immunoglobulin heavy chain gamma, immunoglobulin heavy chain mu, immunoglobulin heavy chain alpha, immunoglobulin heavy chain delta, immunoglobulin heavy chain epsilon, immunoglobulin light chain lambda, or immunoglobulin light chain kappa from a population of nucleic acid molecules in a single reaction chamber. In particular embodiments, a provided kit is a test kit. In some embodiments, the kit further comprises one or more adapters, barcodes, and/or antibodies.

TABLE 2
IgH V gene FR3

| Sequence | SEQ ID NO |
|---|---|
| CATGCAGCTGAGCAGCC | 1 |
| CATGGAGCTGAGCAGGC | 2 |
| GGAGCTGAGCAGCCTGA | 3 |
| AGAGCTGAGCAGCCTGA | 4 |
| GGAGCTGAGGAGCCTGA | 5 |
| CACAGACCTGAGCAGCCT | 6 |
| TGGAGCTAAGCAGCCTGA | 7 |
| CATGGAGCTGAGGAGCCTA | 8 |
| ATGGAGCTAAGCAGTCTGAGATC | 9 |
| TCCTTACCATGACCAACATGGAC | 10 |
| GTGGTCCTTACAATGACCAACATG | 11 |
| GGTTCTAACAGTGATCAACATGGAC | 12 |
| ACACGGCGTATCTGCAAATGAA | 13 |
| CACGCTGTATGTCCAAATGAGC | 14 |
| AACACGCTCTACCTGCAAATGAA | 15 |
| GAACTCACCGTATCTGCAAACGAA | 16 |
| GAACACGCTGTATGTTCAAATGAG | 17 |
| GAACACGCTGTTTCTGCAAATGAA | 18 |
| AAGCATCGCCTATCTGCAAATGAA | 19 |
| GAACACCCTGTATCTGCAAACGAA | 20 |
| AGAACACGCTGTATCTGCAAATGAG | 21 |
| GGAACTCCCTGTATCTGCAAAAGAA | 22 |
| GCAAGTCCCTGTATCTGCAAAAGAA | 23 |
| GAACACGCTGCATCTTCAAATGAAC | 24 |
| GAACTCACTCCGTTTGCAAATGAAC | 25 |
| GAACACGCTGTATCTGCAAATGAAC | 26 |
| AAGAACACGCTGTATCTTCAAATGGG | 27 |
| AAGAACTCCCTCTATCTGCAAGTGAA | 28 |
| AAGAACAGGCTGTATCTGCAAATGAA | 29 |
| CAAAAACTCCCTGTATCTGCAAATGAA | 30 |
| TAAGAACTCACCGTATCTCCAAACGAA | 31 |
| CCAGAATTCACTGTCTCTGCAAATGAA | 32 |
| CAGGAACTTCCTGTATCAGCAAATGAA | 33 |
| CAAGAACACGCTGTATCTTCAAATGAG | 34 |
| CAAGAACTCCCTGTATCTGCAAATGAA | 35 |
| CAAGAACTCACTCTGTTTGCAAATGAA | 36 |
| CAAGAACTCACTGTATCTGCAAATGAAC | 37 |
| CAAAAAACACGCTGTATCTGCAAATGAT | 38 |
| CAAAGAACACGATGTATCTGCAAATGAG | 39 |
| CCAAGAACTCACTGTATTTGCTAATGAA | 40 |
| CCAAGAACTCACTGTATTTGCAAATGAA | 41 |
| CCAATAACTCACCGTATCTGCAAATGAA | 42 |
| CAAAAAACACGCTGTATCTGCAAATGAA | 43 |
| CAAGAACACGCTGTATCTTCAAATGAAC | 44 |
| CAAAAGCATCACCTATCTGCAAATGAAC | 45 |
| CAAGAACACACTTCATCTGCAAATGAAC | 46 |
| CAAAAGCATCACCTATCTGCAAATGAAG | 47 |
| TCAAAGAACTCACTGTATCTGCAAATGAA | 48 |
| GCTAAGAACTCTCTGTATCTGCAAATGAA | 49 |
| CCAAGAACTCCTTGTATCTTCAAATGAAC | 50 |
| CCAAGAAGTCCTTGTATCTTCAAATGAAC | 51 |
| GCCAAGAAGTCCTTGTATCTTCATATGAAC | 52 |
| CAGTTCCCCCTGAAGCTGA | 53 |
| CAGTTCTCCCTGAAGCTGGG | 54 |
| CAGTTCTCCCTGAAGCCGAG | 55 |
| CCAGTTCTCCCTGAAGCTGAG | 56 |
| AACCAGTTCTCCCTGAACCTGA | 57 |
| AACCACTTCTCCCTGAAGCTGA | 58 |
| GAACCAATTCTCCCTGAAGCTGA | 59 |
| AGAACCAGTTCTACCTGAAGCTGA | 60 |
| AGAAGCAGTTCTACCTGAAGCTGA | 61 |
| GCAGTGGAGCAGCCTGA | 62 |
| CAGTTCTCCCTGCAGCTGA | 63 |
| ATCTGCAGATCAGCACGCTAA | 64 |
| TATCTGCAGATCAGCAGCCTAA | 65 |
| TGTCTTCAGATCAGCAGCCTAA | 66 |
| TACCTGCAGATCAGCAGCCTAA | 67 |
| TATCTGCAGATCTGCAGCCTAA | 68 |
| CAUGCAGCUGAGCAGCC | 69 |
| CAUGGAGCUGAGCAGGC | 70 |
| GGAGCUGAGCAGCCUGA | 71 |
| AGAGCUGAGCAGCCUGA | 72 |
| GGAGCUGAGGAGCCUGA | 73 |
| CACAGACCTGAGCAGCCT | 74 |
| TGGAGCUAAGCAGCCUGA | 75 |
| CATGGAGCUGAGGAGCCUA | 76 |
| ATGGAGCUAAGCAGTCTGAGAUC | 77 |
| TCCTTACCAUGACCAACAUGGAC | 78 |
| GTGGTCCTUACAATGACCAACAUG | 79 |
| GGTTCTAACAGUGATCAACAUGGAC | 80 |
| ACACGGCGUATCTGCAAAUGAA | 81 |
| CACGCTGUATGTCCAAAUGAGC | 82 |
| AACACGCTCUACCTGCAAAUGAA | 83 |
| GAACUCACCGTATCUGCAAACGAA | 84 |
| GAACACGCUGTATGTTCAAAUGAG | 85 |
| GAACACGCUGTTTCTGCAAAUGAA | 86 |
| AAGCATCGCCUATCTGCAAAUGAA | 87 |
| GAACACCCUGTATCUGCAAACGAA | 88 |
| AGAACACGCUGTATCTGCAAAUGAG | 89 |
| GGAACUCCCTGTATCUGCAAAAGAA | 90 |
| GCAAGUCCCTGTATCUGCAAAAGAA | 91 |
| GAACACGCUGCATCTTCAAAUGAAC | 92 |
| GAACTCACUCCGTTTGCAAAUGAAC | 93 |
| GAACACGCUGTATCTGCAAAUGAAC | 94 |
| AAGAACACGCUGTATCTTCAAAUGGG | 95 |
| AAGAACTCCCUCTATCTGCAAGUGAA | 96 |
| AAGAACAGGCUGTATCTGCAAAUGAA | 97 |
| CAAAAACTCCCUGTATCTGCAAAUGAA | 98 |
| TAAGAACUCACCGTATCUCCAAACGAA | 99 |
| CCAGAATTCACUGTCTCTGCAAAUGAA | 100 |
| CAGGAACTTCCUGTATCAGCAAAUGAA | 101 |
| CAAGAACACGCUGTATCTTCAAAUGAG | 102 |
| CAAGAACTCCCUGTATCTGCAAAUGAA | 103 |
| CAAGAACTCACUCTGTTTGCAAAUGAA | 104 |
| CAAGAACTCACUGTATCTGCAAAUGAAC | 105 |
| CAAAAAACACGCUGTATCTGCAAAUGAT | 106 |
| CAAAGAACACGAUGTATCTGCAAAUGAG | 107 |
| CCAAGAACTCACUGTATTTGCTAAUGAA | 108 |
| CCAAGAACTCACUGTATTTGCAAAUGAA | 109 |
| CCAATAACTCACCGUATCTGCAAAUGAA | 110 |
| CAAAAAACACGCUGTATCTGCAAAUGAA | 111 |
| CAAGAACACGCUGTATCTTCAAAUGAAC | 112 |
| CAAAAGCATCACCUATCTGCAAAUGAAC | 113 |
| CAAGAACACACUTCATCTGCAAAUGAAC | 114 |
| CAAAAGCATCACCUATCTGCAAAUGAAG | 115 |
| TCAAAGAACTCACUGTATCTGCAAAUGAA | 116 |
| GCTAAGAACTCUCTGTATCTGCAAAUGAA | 117 |
| CCAAGAACTCCUTGTATCTTCAAAUGAAC | 118 |
| CCAAGAAGTCCUTGTATCTTCAAAUGAAC | 119 |
| GCCAAGAAGTCCUTGTATCTTCATAUGAAC | 120 |
| CAGTTCCCCCUGAAGCUGA | 121 |
| CAGTTCUCCCTGAAGCUGGG | 122 |
| CAGUTCTCCCUGAAGCCGAG | 123 |
| CCAGTTCUCCCTGAAGCUGAG | 124 |
| AACCAGTTCUCCCTGAACCUGA | 125 |
| AACCACTTCUCCCTGAAGCUGA | 126 |
| GAACCAATTCUCCCTGAAGCUGA | 127 |
| AGAACCAGTTCUACCTGAAGCUGA | 128 |
| AGAAGCAGTTCUACCTGAAGCUGA | 129 |
| GCAGUGGAGCAGCCUGA | 130 |
| CAGTTCUCCCTGCAGCUGA | 131 |
| ATCTGCAGAUCAGCACGCUAA | 132 |
| TATCTGCAGAUCAGCAGCCUAA | 133 |
| TGTCTTCAGAUCAGCAGCCUAA | 134 |
| TACCTGCAGAUCAGCAGCCUAA | 135 |
| TATCTGCAGAUCTGCAGCCUAA | 136 |

TABLE 3
IgH V gene FR1

| Sequence | SEQ ID NO |
|---|---|
| AGTGAAGGTTTCCTGCAAGGCAT | 137 |
| AGTGAAGGTCTCCTGCAAGGTTT | 138 |
| AGTGAAGGTTTCCTGCAAGGCTT | 139 |
| AGTGAAGGTCTCCTGCAAGGCTT | 140 |
| AGTGAAAATCTCCTGCAAGGTTT | 141 |
| GGTGAAGGTCTCCTGCAAGGCTT | 142 |
| ACGCTGACCTGCACCTTCT | 143 |
| ACGCTGACCCGCACCTTCT | 144 |
| ACACTGACCTGCGCCTTCT | 145 |
| ACGCTGACCTGCACCGTCT | 146 |
| ACACTGACCTGCACCTTCT | 147 |
| CTGAAACTCTCCTGTGCAGCCT | 148 |
| CTGAGACTCTCCTTTGCAGCCT | 149 |
| CTGAGACTCTCCTGTTCAGCCT | 150 |
| CTTAGACTCTCCTGTGCAGCCT | 151 |
| CTGAGACTCTCCTGTGCAGCCT | 152 |
| CTGAGACTCTCCTGTGCAGCGT | 153 |
| CTGAGACTCTCCTGTACAGCTT | 154 |
| GTCCCTCACCTGCGCTGTCT | 155 |
| GTCCCTCACCTGTACTGTCT | 156 |
| GTCCCTCACCTGCACTGTCT | 157 |
| GTCCCTCACCTGCGCTATCT | 158 |
| GTCCCTCACCTGCACTGTCA | 159 |
| GTCTCTGAAGATCTCCTGTAAGGGTT | 160 |
| GTCTCTGAGGATCTCCTGTAAGGGTT | 161 |
| CCTCTCACTCACCTGTGCCATCT | 162 |
| AGTGAAGGTTTCCTGCAAGGCTT | 163 |
| GGGCTGAGGTGAAGAAGCTTG | 164 |
| GAGCTGAGGTGAAGAAGCCTG | 165 |
| GGTCTGAGTTGAAGAAGCCTG | 166 |
| GGGCTGAGGTGAAGAAGACTG | 167 |
| GGCCTGAGGTGAAGAAGCCTG | 168 |
| GGGCTGAGGTGAAGAAGCCTG | 169 |
| GTCTGGTCCTACGCTGGTAAAA | 170 |
| GTCTGGTCCTGCGCTGGTGAAA | 171 |
| GTCTGGTCCTGTGCTGGTGAAA | 172 |
| GTCTGGTCCTACGCTGGTGAAA | 173 |
| GGTCCCTTAGACTCTCCTGTG | 174 |
| GGTCCCTGAGACTCTCCTGTA | 175 |
| CGTCCCTGAGACTCTCCTGTA | 176 |
| GGTCCCTGAAACTCTCCTGTG | 177 |
| GGTCCCTGAGACTCTCCTTTG | 178 |
| GGGCCCTGAGACTCTCCTGTG | 179 |
| GGTCCCTGAGACTCTCCTGTT | 180 |
| GGTCCCTGAGACTCTCCTGTG | 181 |
| GGGCGCAGGACTGTTGAAG | 182 |
| AGGTCCAGGACTGGTGAAG | 183 |
| CGGCTCAGGACTGGTGAAG | 184 |
| GGGCCCAGGACTGTTGAAG | 185 |
| GGGCCCAGGACTGGTGAAG | 186 |
| AGGTCCGGGACTGGTGAAG | 187 |
| GCAGTCTGGAGCAGAGGTGAAA | 188 |
| GCAGTCCGGAGCAGAGGTGAAA | 189 |
| GGCTGAGGTGAAGAAGCCTGG | 190 |
| AGCTGAGGTGAAGAAGCCTGG | 191 |
| GCCTGAGGTGAAGAAGCCTGG | 192 |
| GGCTGAGGTGAAGAAGCTTGG | 193 |
| GGCTGAGGTGAAGAAGACTGG | 194 |
| GTCTGAGTTGAAGAAGCCTGG | 195 |
| GTCCTACGCTGGTGAAACCC | 196 |
| GTCCTGTGCTGGTGAAACCC | 197 |
| GTCCTGCGCTGGTGAAACCC | 198 |
| GTCCTACGCTGGTAAAACCC | 199 |
| GTCCAGGACTGGTGAAGCCC | 200 |
| GCCCAGGACTGTTGAAGCCT | 201 |
| GTCCGGGACTGGTGAAGCCC | 202 |
| GCTCAGGACTGGTGAAGCCT | 203 |
| GCCCAGGACTGGTGAAGCCT | 204 |
| GCGCAGGACTGTTGAAGCCT | 205 |
| GCAGTCCGGAGCAGAGGTGAAAA | 206 |
| GCAGTCTGGAGCAGAGGTGAAAA | 207 |
| GGTCTGAGTTGAAGAAGCCTGG | 208 |
| GGCCTGAGGTGAAGAAGCCTGG | 209 |
| GGGCTGAGGTGAAGAAGACTGG | 210 |
| GGGCTGAGGTGAAGAAGCTTGG | 211 |
| GAGCTGAGGTGAAGAAGCCTGG | 212 |
| GGGCTGAGGTGAAGAAGCCTGG | 213 |
| CCTGCGCTGGTGAAACCCAC | 214 |
| CCTGTGCTGGTGAAACCCAC | 215 |
| CCTACGCTGGTGAAACCCAC | 216 |
| CCTACGCTGGTAAAACCCAC | 217 |
| GGCTGAGGTGAAGAAGCCTGGG | 218 |
| GCCTGAGGTGAAGAAGCCTGGG | 219 |
| GGCTGAGGTGAAGAAGCTTGGG | 220 |
| AGCTGAGGTGAAGAAGCCTGGG | 221 |
| GTCTGAGTTGAAGAAGCCTGGG | 222 |
| GGCTGAGGTGAAGAAGACTGGG | 223 |
| TGCGCTGGTGAAACCCACAC | 224 |
| TACGCTGGTAAAACCCACAC | 225 |
| TGTGCTGGTGAAACCCACAG | 226 |
| TACGCTGGTGAAACCCACAC | 227 |
| TCCTCGGTGAAGGTCTCCTGCA | 228 |
| GCCTCAGTGAAGGTCTCCTGCA | 229 |
| TCCTCAGTGAAGGTCTCCTGCA | 230 |
| ACCTCAGTGAAGGTCTCCTGCA | 231 |
| GCCTCAGTGAAGGTTTCCTGCA | 232 |
| TCCTCAGTGAAGGTTTCCTGCA | 233 |
| GCTACAGTGAAAATCTCCTGCA | 234 |
| CAGACCCTCACACTGACCTG | 235 |
| GAGACCCTCACGCTGACCTG | 236 |
| CAGACCCTCACGCTGACCTG | 237 |
| CAGACCCTCACGCTGACCCG | 238 |
| CTGTCCCTCACCTGTACTGT | 239 |
| CCGTCCCTCACCTGCACTGT | 240 |
| CTCTCACTCACCTGTGCCAT | 241 |
| CTGTCCCTCACCTGCACTGT | 242 |
| CTGTCCCTCACCTGCGCTAT | 243 |
| CTGTCCCTCACCTGCGCTGT | 244 |
| GGGAGTCTCTGAGGATCTCCTGTA | 245 |
| GGGAGTCTCTGAAGATCTCCTGTA | 246 |
| TCGGTGAAGGTCTCCTGCAAGG | 247 |
| TCAGTGAAGGTTTCCTGCAAGG | 248 |
| ACAGTGAAAATCTCCTGCAAGG | 249 |
| TCAGTGAAGGTCTCCTGCAAGG | 250 |
| TCACGCTGACCTGCACCGT | 251 |
| TCACGCTGACCCGCACCTT | 252 |
| TCACACTGACCTGCGCCTT | 253 |
| TCACACTGACCTGCACCTT | 254 |
| TCACGCTGACCTGCACCTT | 255 |
| CCCTGAGACTCTCCTGTACAGC | 256 |
| CCCTGAGACTCTCCTGTTCAGC | 257 |
| CCCTGAGACTCTCCTTTGCAGC | 258 |
| CCCTGAAACTCTCCTGTGCAGC | 259 |
| CCCTGAGACTCTCCTGTGCAGC | 260 |
| CCCTTAGACTCTCCTGTGCAGC | 261 |
| GAGTCTCTGAAGATCTCCTGTAAGGG | 262 |
| GAGTCTCTGAGGATCTCCTGTAAGGG | 263 |
| GTGAAGGTCTCCTGCAAGGCTTCT | 264 |
| GTGAAAATCTCCTGCAAGGTTTCT | 265 |
| GTGAAGGTTTCCTGCAAGGCATCT | 266 |
| GTGAAGGTTTCCTGCAAGGCTTCT | 267 |
| GTGAAGGTTTCCTGCAAGGCTTCC | 268 |
| GTGAAGGTCTCCTGCAAGGTTTCC | 269 |
| ACGCTGACCCGCACCTTCTC | 270 |
| ACGCTGACCTGCACCTTCTC | 271 |
| ACGCTGACCTGCACCGTCTC | 272 |
| ACACTGACCTGCGCCTTCTC | 273 |
| ACACTGACCTGCACCTTCTC | 274 |
| GTCCCTCACCTGCACTGTCTC | 275 |
| GTCCCTCACCTGTACTGTCTC | 276 |
| GTCCCTCACCTGCGCTATCTC | 277 |
| GTCCCTCACCTGCACTGTCAC | 278 |
| CTCACTCACCTGTGCCATCTC | 279 |
| GTCCCTCACCTGCGCTGTCTA | 280 |
| GTCCCTCACCTGCGCTGTCTC | 281 |
| GTCTCTGAGGATCTCCTGTAAGGGTTC | 282 |
| GTCTCTGAAGATCTCCTGTAAGGGTTC | 283 |
| AGTGAAGGUTTCCUGCAAGGCAT | 284 |
| AGTGAAGGTCUCCTGCAAGGTUT | 285 |
| AGTGAAGGTTUCCTGCAAGGCUT | 286 |
| AGTGAAGGTCUCCTGCAAGGCUT | 287 |
| AGTGAAAATCUCCTGCAAGGTUT | 288 |
| GGTGAAGGTCUCCTGCAAGGCUT | 289 |
| ACGCTGACCUGCACCTUCT | 290 |
| ACGCUGACCCGCACCTUCT | 291 |
| ACACTGACCUGCGCCTUCT | 292 |
| ACGCTGACCUGCACCGUCT | 293 |
| ACACTGACCUGCACCTUCT | 294 |
| CTGAAACUCTCCTGUGCAGCCT | 295 |
| CTGAGACUCTCCTTUGCAGCCT | 296 |
| CTGAGACUCTCCTGTUCAGCCT | 297 |
| CTTAGACUCTCCTGUGCAGCCT | 298 |
| CTGAGACUCTCCTGUGCAGCCT | 299 |
| CTGAGACUCTCCTGUGCAGCGT | 300 |
| CTGAGACTCUCCTGTACAGCUT | 301 |
| GTCCCTCACCUGCGCTGUCT | 302 |
| GTCCCUCACCTGTACTGUCT | 303 |
| GTCCCTCACCUGCACTGUCT | 304 |
| GTCCCTCACCUGCGCTAUCT | 305 |
| GTCCCTCACCUGCACTGUCA | 306 |
| GTCTCTGAAGAUCTCCTGTAAGGGUT | 307 |
| GTCTCTGAGGAUCTCCTGTAAGGGUT | 308 |
| CCTCTCACUCACCTGTGCCAUCT | 309 |
| AGTGAAGGTTUCCTGCAAGGCUT | 310 |
| GGGCTGAGGUGAAGAAGCTUG | 311 |
| GAGCTGAGGUGAAGAAGCCUG | 312 |
| GGTCTGAGTUGAAGAAGCCUG | 313 |
| GGGCTGAGGUGAAGAAGACUG | 314 |
| GGCCTGAGGUGAAGAAGCCUG | 315 |
| GGGCTGAGGUGAAGAAGCCUG | 316 |
| GTCTGGTCCUACGCTGGUAAAA | 317 |
| GTCTGGTCCUGCGCTGGUGAAA | 318 |
| GTCTGGTCCUGTGCTGGUGAAA | 319 |
| GTCTGGTCCUACGCTGGUGAAA | 320 |
| GGTCCCTUAGACTCTCCTGUG | 321 |
| GGTCCCUGAGACTCTCCTGUA | 322 |
| CGTCCCUGAGACTCTCCTGUA | 323 |
| GGTCCCUGAAACTCTCCTGUG | 324 |
| GGTCCCUGAGACTCTCCTTUG | 325 |
| GGGCCCUGAGACTCTCCTGUG | 326 |
| GGTCCCUGAGACTCTCCTGUT | 327 |
| GGTCCCUGAGACTCTCCTGUG | 328 |
| GGGCGCAGGACUGTUGAAG | 329 |
| AGGUCCAGGACTGGUGAAG | 330 |
| CGGCUCAGGACTGGUGAAG | 331 |
| GGGCCCAGGACUGTUGAAG | 332 |
| GGGCCCAGGACUGGUGAAG | 333 |
| AGGUCCGGGACTGGUGAAG | 334 |
| GCAGTCUGGAGCAGAGGUGAAA | 335 |
| GCAGUCCGGAGCAGAGGUGAAA | 336 |
| GGCTGAGGUGAAGAAGCCUGG | 337 |
| AGCTGAGGUGAAGAAGCCUGG | 338 |
| GCCTGAGGUGAAGAAGCCUGG | 339 |
| GGCTGAGGUGAAGAAGCTUGG | 340 |
| GGCTGAGGUGAAGAAGACUGG | 341 |
| GTCTGAGTUGAAGAAGCCUGG | 342 |
| GTCCUACGCTGGUGAAACCC | 343 |
| GTCCTGUGCTGGUGAAACCC | 344 |
| GTCCUGCGCTGGUGAAACCC | 345 |
| GTCCUACGCTGGUAAAACCC | 346 |
| GTCCAGGACUGGUGAAGCCC | 347 |
| GCCCAGGACUGTUGAAGCCT | 348 |
| GTCCGGGACUGGUGAAGCCC | 349 |
| GCUCAGGACTGGUGAAGCCT | 350 |
| GCCCAGGACUGGUGAAGCCT | 351 |
| GCGCAGGACUGTUGAAGCCT | 352 |
| GCAGUCCGGAGCAGAGGUGAAAA | 353 |
| GCAGTCUGGAGCAGAGGUGAAAA | 354 |
| GGTCTGAGTUGAAGAAGCCUGG | 355 |
| GGCCTGAGGUGAAGAAGCCUGG | 356 |
| GGGCTGAGGUGAAGAAGACUGG | 357 |
| GGGCTGAGGUGAAGAAGCTUGG | 358 |
| GAGCTGAGGUGAAGAAGCCUGG | 359 |
| GGGCTGAGGUGAAGAAGCCUGG | 360 |
| CCUGCGCTGGUGAAACCCAC | 361 |
| CCUGTGCTGGUGAAACCCAC | 362 |
| CCUACGCTGGUGAAACCCAC | 363 |
| CCUACGCTGGUAAAACCCAC | 364 |
| GGCTGAGGUGAAGAAGCCUGGG | 365 |
| GCCTGAGGUGAAGAAGCCUGGG | 366 |
| GGCTGAGGUGAAGAAGCTUGGG | 367 |
| AGCTGAGGUGAAGAAGCCUGGG | 368 |
| GTCTGAGTUGAAGAAGCCUGGG | 369 |
| GGCTGAGGUGAAGAAGACUGGG | 370 |
| TGCGCUGGUGAAACCCACAC | 371 |
| TACGCUGGUAAAACCCACAC | 372 |
| TGUGCTGGUGAAACCCACAG | 373 |
| TACGCUGGUGAAACCCACAC | 374 |
| TCCTCGGUGAAGGTCTCCUGCA | 375 |
| GCCTCAGUGAAGGTCTCCUGCA | 376 |
| TCCTCAGUGAAGGTCTCCUGCA | 377 |
| ACCTCAGUGAAGGTCTCCUGCA | 378 |
| GCCTCAGUGAAGGTTTCCUGCA | 379 |
| TCCTCAGUGAAGGTTTCCUGCA | 380 |
| GCTACAGUGAAAATCTCCUGCA | 381 |
| CAGACCCUCACACTGACCUG | 382 |
| GAGACCCUCACGCTGACCUG | 383 |
| CAGACCCUCACGCTGACCUG | 384 |
| CAGACCCUCACGCUGACCCG | 385 |
| CTGTCCCUCACCTGTACUGT | 386 |
| CCGTCCCUCACCTGCACUGT | 387 |
| CTCTCACUCACCTGUGCCAT | 388 |
| CTGTCCCUCACCTGCACUGT | 389 |
| CTGTCCCUCACCTGCGCUAT | 390 |
| CTGTCCCUCACCTGCGCUGT | 391 |
| GGGAGTCTCUGAGGATCTCCTGUA | 392 |
| GGGAGTCTCUGAAGATCTCCTGUA | 393 |
| TCGGUGAAGGTCTCCUGCAAGG | 394 |
| TCAGUGAAGGTTTCCUGCAAGG | 395 |
| ACAGTGAAAAUCTCCUGCAAGG | 396 |
| TCAGTGAAGGUCTCCUGCAAGG | 397 |
| TCACGCUGACCUGCACCGT | 398 |
| TCACGCUGACCCGCACCUT | 399 |
| TCACACTGACCUGCGCCUT | 400 |
| TCACACUGACCTGCACCUT | 401 |
| TCACGCUGACCTGCACCUT | 402 |
| CCCTGAGACUCTCCTGUACAGC | 403 |
| CCCTGAGACUCTCCTGTUCAGC | 404 |
| CCCTGAGACUCTCCTTUGCAGC | 405 |
| CCCTGAAACUCTCCTGUGCAGC | 406 |
| CCCTGAGACUCTCCTGUGCAGC | 407 |
| CCCTTAGACUCTCCTGUGCAGC | 408 |
| GAGTCTCUGAAGATCTCCTGUAAGGG | 409 |
| GAGTCTCUGAGGATCTCCTGUAAGGG | 410 |
| GTGAAGGTCUCCTGCAAGGCTUCT | 411 |
| GTGAAAATCUCCTGCAAGGTTUCT | 412 |
| GTGAAGGTTUCCTGCAAGGCAUCT | 413 |
| GTGAAGGTTUCCTGCAAGGCTUCT | 414 |
| GTGAAGGTTUCCTGCAAGGCTUCC | 415 |
| GTGAAGGTCUCCTGCAAGGTTUCC | 416 |
| ACGCUGACCCGCACCTTCUC | 417 |
| ACGCTGACCUGCACCTTCUC | 418 |
| ACGCTGACCUGCACCGTCUC | 419 |
| ACACTGACCUGCGCCTTCUC | 420 |
| ACACTGACCUGCACCTTCUC | 421 |
| GTCCCTCACCUGCACTGTCUC | 422 |
| GTCCCTCACCUGTACTGTCUC | 423 |
| GTCCCTCACCUGCGCTATCUC | 424 |
| GTCCCTCACCUGCACTGUCAC | 425 |
| CTCACTCACCUGTGCCATCUC | 426 |
| GTCCCTCACCUGCGCTGTCUA | 427 |
| GTCCCTCACCUGCGCTGTCUC | 428 |
| GTCTCTGAGGAUCTCCTGTAAGGGTUC | 429 |
| GTCTCTGAAGATCUCCTGTAAGGGTUC | 430 |

TABLE 4
IgH V gene FR2

| Sequence | SEQ ID NO |
|---|---|
| CTGGGTGCGACAGGCCCCTGGACAA | 431 |
| TGGATCCGTCAGCCCCCAGGGAAGG | 432 |
| GGTCCGCCAGGCTCCAGGGAA | 433 |
| TGGATCCGCCAGCCCCCAGGGAAGG | 434 |
| GGGTGCGCCAGATGCCCGGGAAAGG | 435 |
| TGGATCAGGCAGTCCCCATCGAGAG | 436 |
| TTGGGTGCGACAGGCCCCTGGACAA | 437 |

TABLE 5
IgH J gene

| Sequence | SEQ ID NO |
|---|---|
| GAGGAGACGGTGACCGTG | 438 |
| GAGACAGTGACCAGGGTGC | 439 |
| GAGACGGTGACCATTGTCC | 440 |
| TGAGGAGACGGTGACCAGG | 441 |
| CCAGTGGCAGAGGAGTCCATTC | 442 |
| GAGGAGACGGUGACCGUG | 443 |
| GAGACAGUGACCAGGGUGC | 444 |
| GAGACGGUGACCATTGUCC | 445 |
| TGAGGAGACGGUGACCAGG | 446 |
| CCAGUGGCAGAGGAGTCCATUC | 447 |

TABLE 6
IgH C gene A isotype

| Sequence | SEQ ID NO |
|---|---|
| AGGTCACACTGAGTGGCTCC | 448 |
| ACGACCACGTTCCCATCTTG | 449 |
| ATGACCACGTTCCCATCTGG | 450 |
| GCGATGACCACGTTCCCATCTG | 451 |
| GCGACGACCACGTTCCCATCTT | 452 |
| AGGCGATGACCACGTTCCCATC | 453 |
| ATGCGACGACCACGTTCCCATC | 454 |
| ATGCGACGACCACGTTCCCAT | 455 |
| AGGCGATGACCACGTTCCCAT | 456 |
| GCTTTCGCTCCAGGTCACACT | 457 |
| CGCTTTCGCTCCAGGTCACAC | 458 |
| CGCTTTCGCTCCAGGTCACA | 459 |
| AGGTCACACUGAGTGGCUCC | 460 |
| ACGACCACGUTCCCATCTUG | 461 |
| ATGACCACGUTCCCATCUGG | 462 |
| GCGATGACCACGUTCCCATCUG | 463 |
| GCGACGACCACGUTCCCATCUT | 464 |
| AGGCGAUGACCACGTTCCCAUC | 465 |
| ATGCGACGACCACGUTCCCAUC | 466 |
| AUGCGACGACCACGTUCCCAT | 467 |
| AGGCGAUGACCACGTUCCCAT | 468 |
| GCTTTCGCUCCAGGUCACACT | 469 |
| CGCTTTCGCUCCAGGUCACAC | 470 |
| CGCTTTCGCUCCAGGUCACA | 471 |

TABLE 7
IgH C gene D isotype

| Sequence | SEQ ID NO |
|---|---|
| GACAGTCACGGACGTTGGG | 472 |
| ACCACAGGGCTGTTATCCTTTGG | 473 |
| ACCACAGGGCTGTTATCCTTTG | 474 |
| CAGGACCACAGGGCTGTTATCCTT | 475 |
| CAGGACCACAGGGCTGTTATCCT | 476 |
| CCCATGTACCAGGTGACAGTCAC | 477 |
| CCCATGTACCAGGTGACAGTCA | 478 |
| CCCATGTACCAGGTGACAGTC | 479 |
| GACAGUCACGGACGTUGGG | 480 |
| ACCACAGGGCUGTTATCCTTUGG | 481 |
| ACCACAGGGCUGTTATCCTTUG | 482 |
| CAGGACCACAGGGCUGTTATCCUT | 483 |
| CAGGACCACAGGGCUGTTAUCCT | 484 |
| CCCATGUACCAGGTGACAGUCAC | 485 |
| CCCATGUACCAGGTGACAGUCA | 486 |
| CCCATGUACCAGGTGACAGUC | 487 |

TABLE 8
IgH C gene G isotype

| Sequence | SEQ ID NO |
|---|---|
| CGACACCGTCACCGGTTC | 488 |
| GGCCGCTGTGCTCTCGGAGGTG | 489 |
| GGCCGCTGTGCCCCCAGAGGTG | 490 |
| GGCGGCTGTGCTCTCGGAGGTG | 491 |
| GGCCGCTGTGCCCTCAGAGACG | 492 |
| GGCTGCTGTGCCCCCAGAGGTG | 493 |
| GGCCGCTGTGCCCTCAGAGAC | 494 |
| GGCTGCTGTGCCCCCAGAGGT | 495 |
| GGCGGCTGTGCTCTCGGAGGT | 496 |
| GGCCGCTGTGCTCTCGGAGGT | 497 |
| GGCCGCTGTGCCCCCAGAGGT | 498 |
| GGGCTGCTGTGCCCCCAGAG | 499 |
| GGGCGGCTGTGCTCTCGGAG | 500 |
| GGGCCGCTGTGCCCCCAGAG | 501 |
| GGGCCGCTGTGCCCTCAGAG | 502 |
| GGGCCGCTGTGCTCTCGGAG | 503 |
| CAGGGCGGCTGTGCTCTCGGA | 504 |
| CAGGGCCGCTGTGCCCTCAGA | 505 |
| CAGGGCTGCTGTGCCCCCAGA | 506 |
| CAGGGCCGCTGTGCCCCCAGA | 507 |
| CAGGGCCGCTGTGCTCTCGGA | 508 |
| CTGAGTTCCACGACACCGTC | 509 |
| CCCTGAGTTCCACGACACCGT | 510 |
| GCCTGAGTTCCACGACACCGT | 511 |
| CCCTGAGTTCCACGACACCG | 512 |
| GCCTGAGTTCCACGACACCG | 513 |
| CGACACCGUCACCGGTUC | 514 |
| GGCCGCTGUGCTCTCGGAGGUG | 515 |
| GGCCGCTGUGCCCCCAGAGGUG | 516 |
| GGCGGCTGUGCTCTCGGAGGUG | 517 |
| GGCCGCUGTGCCCUCAGAGACG | 518 |
| GGCTGCTGUGCCCCCAGAGGUG | 519 |
| GGCCGCUGTGCCCUCAGAGAC | 520 |
| GGCUGCTGUGCCCCCAGAGGT | 521 |
| GGCGGCUGTGCTCUCGGAGGT | 522 |
| GGCCGCUGTGCTCUCGGAGGT | 523 |
| GGCCGCUGUGCCCCCAGAGGT | 524 |
| GGGCUGCTGUGCCCCCAGAG | 525 |
| GGGCGGCUGTGCTCUCGGAG | 526 |
| GGGCCGCUGUGCCCCCAGAG | 527 |
| GGGCCGCUGTGCCCUCAGAG | 528 |
| GGGCCGCUGTGCTCUCGGAG | 529 |
| CAGGGCGGCUGTGCTCUCGGA | 530 |
| CAGGGCCGCUGTGCCCUCAGA | 531 |
| CAGGGCUGCTGUGCCCCCAGA | 532 |
| CAGGGCCGCUGUGCCCCCAGA | 533 |
| CAGGGCCGCUGTGCTCUCGGA | 534 |
| CTGAGTUCCACGACACCGUC | 535 |
| CCCUGAGTUCCACGACACCGT | 536 |
| GCCUGAGTUCCACGACACCGT | 537 |
| CCCUGAGTUCCACGACACCG | 538 |
| GCCUGAGTUCCACGACACCG | 539 |

TABLE 9
IgH C gene M isotype

| Sequence | SEQ ID NO |
|---|---|
| GGAGAAAGTGATGGAGTCGGGA | 540 |
| GGACAAAGTGATGGAGTCGGGA | 541 |
| GTCCTGTGCGAGGCAGCCAAC | 542 |
| GTCCTGTGCGAGGCAGCCAA | 543 |
| AGGAAGTCCTGTGCGAGGCAGCCA | 544 |
| AGGAAGTCCTGTGCGAGGCAGCC | 545 |
| GTATTTCCAGGAGAAAGTGAT | 546 |
| GTATTTCCAGGACAAAGTGAT | 547 |
| TGTATTTCCAGGACAAAGTGA | 548 |
| TGTATTTCCAGGAGAAAGTGA | 549 |
| TGTATTTCCAGGACAAAGTG | 550 |
| TGTATTTCCAGGAGAAAGTG | 551 |
| GGAGAAAGUGATGGAGUCGGGA | 552 |
| GGACAAAGUGATGGAGUCGGGA | 553 |
| GUCCTGUGCGAGGCAGCCAAC | 554 |
| GUCCTGUGCGAGGCAGCCAA | 555 |
| AGGAAGUCCTGUGCGAGGCAGCCA | 556 |
| AGGAAGUCCTGUGCGAGGCAGCC | 557 |
| GTATTUCCAGGAGAAAGUGAT | 558 |
| GTATTUCCAGGACAAAGUGAT | 559 |
| TGTATTUCCAGGACAAAGUGA | 560 |
| TGTATTUCCAGGAGAAAGUGA | 561 |
| TGTATTUCCAGGACAAAGUG | 562 |
| TGTATTUCCAGGAGAAAGUG | 563 |

TABLE 10
IgH C gene E isotype

| Sequence | SEQ ID NO |
|---|---|
| GGAAGTAGCCTGTGGCCAG | 564 |
| GGAAGTAGCCCGTGGCCAG | 565 |
| GAGGTGGCATCAGAGGCAAT | 566 |
| GAGGTGGCATTGGAGGGAAT | 567 |
| GAGGTGGCATTGGAGGGAA | 568 |
| GAGGTGGCATCAGAGGCAA | 569 |
| TGGAGGTGGCATCAGAGGC | 570 |
| CGGAGGTGGCATTGGAGGG | 571 |
| TGGAGGTGGCATCAGAGG | 572 |
| CGGAGGTGGCATTGGAGG | 573 |
| GCCTGTGTCCCAGGTCACCATCACCGG | 574 |
| GCCTGTGTCCCAGGTCACAGTCACTGA | 575 |
| GCCTGTGTCGCAGGTCACCATCACCGG | 576 |
| GCCTGTGTCCCAGGTCACCATCACCG | 577 |
| GCCTGTGTCGCAGGTCACCATCACCG | 578 |
| GCCTGTGTCCCAGGTCACAGTCACTG | 579 |
| AGCCTGTGTCCCAGGTCACCATCACC | 580 |
| AGCCTGTGTCGCAGGTCACCATCACC | 581 |
| AGCCTGTGTCCCAGGTCACAGTCACT | 582 |
| GGAAGUAGCCTGUGGCCAG | 583 |
| GGAAGUAGCCCGUGGCCAG | 584 |
| GAGGUGGCAUCAGAGGCAAT | 585 |
| GAGGUGGCATUGGAGGGAAT | 586 |
| GAGGUGGCATUGGAGGGAA | 587 |
| GAGGUGGCAUCAGAGGCAA | 588 |
| TGGAGGUGGCAUCAGAGGC | 589 |
| CGGAGGUGGCATUGGAGGG | 590 |
| TGGAGGUGGCAUCAGAGG | 591 |
| CGGAGGUGGCATUGGAGG | 592 |
| GCCTGTGUCCCAGGTCACCAUCACCGG | 593 |
| GCCTGTGTCCCAGGUCACAGTCACUGA | 594 |
| GCCTGTGUCGCAGGTCACCAUCACCGG | 595 |
| GCCTGTGUCCCAGGTCACCAUCACCG | 596 |
| GCCTGTGUCGCAGGTCACCAUCACCG | 597 |
| GCCTGTGTCCCAGGUCACAGTCACUG | 598 |
| AGCCTGTGUCCCAGGTCACCAUCACC | 599 |
| AGCCTGTGUCGCAGGTCACCAUCACC | 600 |
| AGCCTGTGUCCCAGGTCACAGUCACT | 601 |

The following description of various exemplary embodiments is exemplary and explanatory only and is not to be construed as limiting or restrictive in any way. Other embodiments, features, objects, and advantages of the present teachings will be apparent from the description and accompanying drawings, and from the claims.

Although the present description described in detail certain exemplary embodiments, other embodiments are also possible and within the scope of the present invention. Variations and modifications will be apparent to those skilled in the art from consideration of the specification and figures and practice of the teachings described in the specification and figures, and the claims.

### EXAMPLES

Provided immune repertoire compositions include, without limitation, reagents designed for library preparation and sequencing of expressed IgH sequences and rearranged genomic IgH sequences. Generally, RNAs extracted from samples (e.g., blood samples, sorted cell samples, normal tissue sample, tumor samples, (e.g., fresh, frozen, FFPE, of various types)) were reverse transcribed; gDNA was extracted from samples; libraries were generated, templates prepared, e.g., using Ion ChefUM or Ion OneTouch™ 2 System, then prepared templates were sequenced using next generation sequencing technology, e.g., an Ion S5™ System, an Ion PGM™ System and sequence analysis was performed using Ion Reporter™ software. Kits suitable for extracting and/or isolating RNA and genomic DNA from biological samples are commercially available from, for example, Thermo Fisher Scientific and BioChain Institute Inc.

### Example 1

Total RNA from peripheral blood leukocytes was reverse transcribed to cDNA with SuperScript™ IV VILO™ Master Mix (Thermo Fisher Scientific) according to manufacturer instructions. The cDNA (25 ng or 50 ng) was used in multiplex PCR to amplify IgH and/or TCR beta CDR3 domain sequences. In one multiplex PCR, sets of forward and reverse primers selected from Tables 2 and 5 were used as primer pairs in amplifying sequences from the V gene FR3 region to the J gene of IgH cDNA. In another multiplex PCR, these same sets of IgH forward and reverse FR3-J primers were combined with the TCR beta FR3-J primer panel from the Oncomine™ TCR Beta-SR Assay (RNA) in amplifying the FR3-J regions of IgH and TCR beta in the same reaction. In another multiplex PCR, the Oncomine™ TCR Beta-SR Assay,RNA (Thermo Fisher Scientific was used in amplifying sequences the FR3-J region of TCR beta cDNA. In the exemplary IgH V gene FR3-J amplification reactions, the multiplex primer set included 68 different IGHV forward primers SEQ ID NOs: 69-136 and 4 different IGHJ reverse primers SEQ ID NOs: 443-446.

Typically, the multiplex amplification reactions were performed as follows. To a single well of a 96-well PCR plate was added 10 microliters prepared cDNA (25 ng or 50 ng), 4 microliters of 2 μM forward and reverse primer pool, 4 microliters of 5× Ion AmpliSeq™ HiFi Mix (an amplification reaction mixture that can include glycerol, dNTPs, and Platinum® Taq High Fidelity DNA Polymerase (Invitrogen, Catalog No. 11304)), and 2 microliters DNase/RNase free water to bring the final reaction volume to 20 microliters. The IgH+TCR beta (1 pool) reaction was prepared in the same manner. The Oncomine™ TCR Beta-SR Assay, RNA was performed according to manufacturer instructions.

The PCR plate was sealed, reaction mixtures mixed, and loaded into a thermal cycler (e.g., Veriti™ 96-well thermal cycler (Applied Biosystems)) and run on the following temperature profile to generate the amplicon library. An initial holding stage was performed at 95° C. for 2 minutes, followed by about 20 cycles of a denaturing stage at 95° C. for 15 seconds, an annealing stage at 60° C. for 45 seconds, and an extending stage for 72° C. for 45 seconds. After cycling, a final extension 72° C. for 10 minutes was performed and the amplicon library was held at 10° C. until proceeding. Typically, about 20 cycles are used to generate the amplicon library. For some applications, up to 30 cycles can be used.

The amplicon sample was briefly centrifuged to collect contents before proceeding. To the amplicon library (˜20 microliters), 2 microliters of FuPa reagent was added. The reaction mixture was sealed, mixed thoroughly to ensure uniformity and incubated at 50° C. for 10 minutes, 55° C. for 10 minutes, 60° C. for 20 minutes, then held at 10° C. for up to 1 hour. The sample was briefly centrifuged to collect contents before proceeding.

After incubation, the reaction mixture proceeded directly to a ligation step. Here, the reaction mixture now containing the phosphorylated amplicon library was combined with 2 microliters of Ion Select Barcode Adapters, 5 μM each (Thermo Fisher Scientific), 4 microliters of AmpliSeq Plus Switch Solution (sold as a component of the Ion AmpliSeq™ Library Kit Plus, Thermo Fisher Scientific) and 2 microliters of DNA ligase, added last (sold as a component of the Ion AmpliSeq™ Library Kit Plus, Thermo Fisher Scientific), then incubated at the following: 22° C. for 30 minutes, 68° C. for 5 minutes, 72° C. for 5 minutes, then held at 10° C. for up to 1 hour. The sample was briefly centrifuged to collect contents before proceeding.

After the incubation step, 45 microliters (1.5× sample volume) of room temperature AMPure® XP beads (Beckman Coulter) was added to ligated DNA and the mixture was pipetted thoroughly to mix the bead suspension with the DNA. The mixture was incubated at room temperature for 5 minutes, placed on a magnetic rack such as a DynaMag™-96 side magnet (Invitrogen, Part No. 12331D) for two minutes. After the solution had cleared, the supernatant was discarded. Without removing the plate from the magnetic rack, 150 microliters of freshly prepared 70% ethanol was introduced into the sample and incubated while gently rotating the tube on the magnetic rack. After the solution cleared, the supernatant was discarded without disturbing the pellet. A second ethanol wash was performed, the supernatant discarded, and any remaining ethanol was removed by pulse-spinning the tube and carefully removing residual ethanol while not disturbing the pellet. The pellet was air-dried for about 5 minutes at room temperature. The ligated DNA was eluted from the beads in 50 microliters of low TE buffer.

The eluted libraries were quantitated by qPCR using the Ion Library TaqMan® Quantitation Kit (Ion Torrent, Cat. No. 4468802), according to manufacturer instructions. After quantification, the libraries were diluted to a concentration of about 25 μM.

The libraries were normalized to 20 μM and aliquots of the final libraries were used in template preparation and chip loading using the Ion Chef™ instrument according to the manufacturer instructions. Sequencing was performed using Ion 540™ chips on the Ion S5™ System according to manufacturer instructions, and gene sequence analysis was performed with the Ion Torrent Suite™ software. Since the sequences were generated from use of J gene primers, they were subjected to a J gene sequence inference process involving adding the inferred J gene sequence to the sequence read to create an extended sequence read, aligning the extended sequence read to a reference sequence, and identifying productive reads, as described herein. In addition, all of the generated sequence data was further subjected to the error identification and removal programs provided herein.

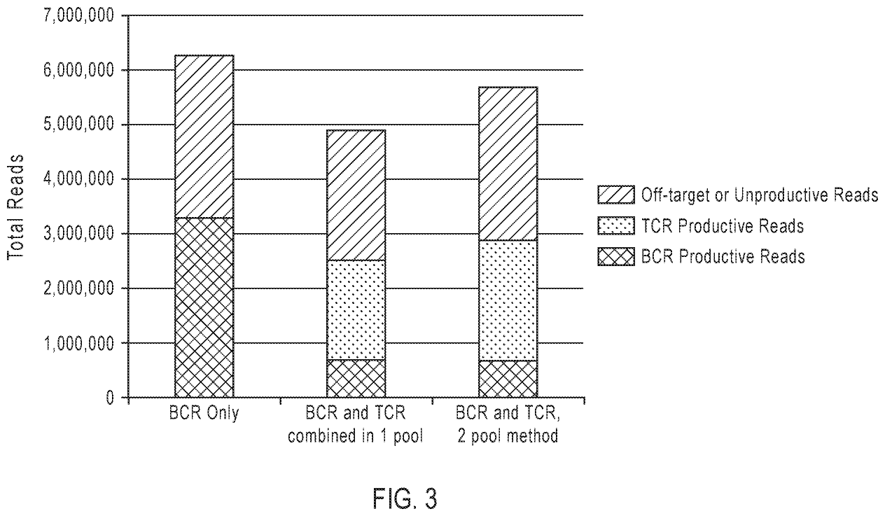

Sequencing of 12 libraries resulted in an average of 6M reads per sample. As shown in FIG. 3, up to 66% of the total reads were productive when the IgH assay was performed alone. When the IgH (BCR) and TCR beta (TCR) assays were combined, about 12% of the reads were productive IgH reads and about 58% of the reads were TCR beta productive reads.

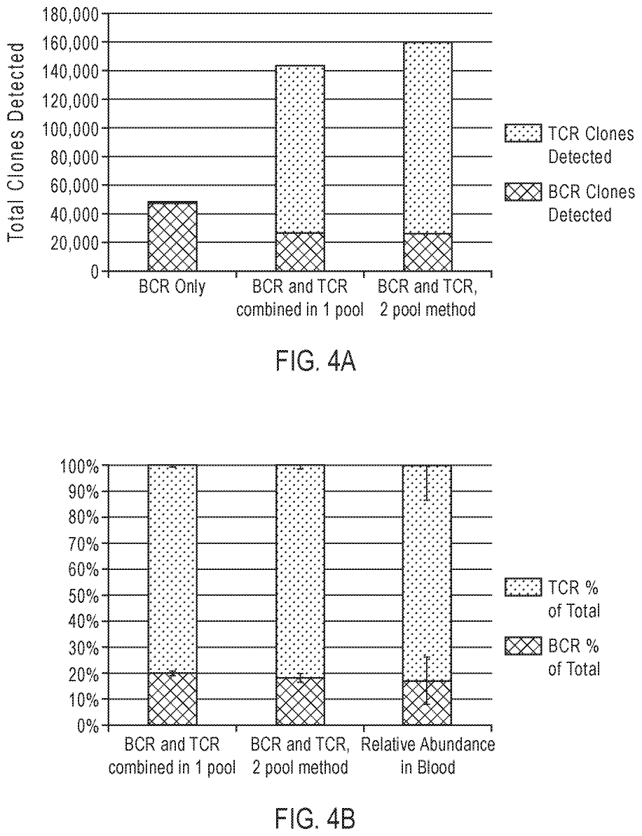

The total number of B cell and T cell clones detected was proportional to the relative abundance of lymphocytes in blood. As shown in FIG. 4A, about 45,000 BCR clones were identified when the IgH assay was run alone and about 30,000 BCR clones were identified when the IgH and TCR beta primers were combined. This difference in the number of BCR clones detected between the two assays is to be expected because the libraries were normalized prior to sequencing, and the read depth for BCR is lowered. The similarities in number of clones when the BCR and TCR assays are combined in one pool versus amplified in two separate pools shows that there is no primer interference in the combined amplification reaction. The population of IgH (BCR) and TCR beta (TCR) clones is proportional to the relative abundance of B and T cells in peripheral blood (FIG. 4B). The relative proportion of T-lymphocytes and B-lymphocytes can range from 61-85% and 7-23% respectively (Palmer et al. (2006) BMC Genomics 7:115).

### Example 2

Leukocyte genomic DNA was isolated from a biological sample and used in multiplex PCR to assess the IgH immune repertoire in the sample. In a multiplex PCR, sets of forward primers from the IgH V gene FR3 region and reverse primers from the IgH J gene were used as primer pairs in amplifying sequences from the V gene FR3 region to the J gene of rearranged IgH gDNA. Exemplary primer sets included sets of forward and reverse primers selected from Tables 2 and 5, such as a multiplex primer set including IGHV forward primers SEQ ID NOs: 69-136 and IGHJ reverse primers SEQ ID NOs: 443-446.

To a single well of a 96-well PCR plate was added 250 ng leukocyte gDNA, 4 microliters of 1 μM Primer Mix (FR3 forward primers and J reverse primers, 1 μM each), 4 microliters of 5× Ion AmpliSeq™ HiFi Mix (Invitrogen, Catalog No. 11304), 2 microliters of dNTP Mix (dGTP, dCTP, dATP, and dTTP; 7.5 mM each) and DNase/RNase free water to bring the final reaction volume to 20 microliters.

The PCR plate was sealed, reaction mixtures mixed, and the plate loaded into a thermal cycler (e.g., Veriti™ 96-well thermal cycler (Applied Biosystems)) and run on the following temperature profile to generate the amplicon library. An initial holding stage was performed at 95° C. for 2 minutes, followed by about 25 cycles of a denaturing stage at 95° C. for 30 seconds, an annealing stage at 60° C. for 45 seconds, and an extending stage for 72° C. for 45 seconds. After cycling, a final extension 72° C. for 10 minutes was performed and the amplicon library was held at 10° C. until proceeding. For some applications, cycles used can range from about 17 to about 30 cycles. Amplicon and library preparation, chip loading, sequencing and sequence data processing was performed as described in Example 1. The leukocyte gDNA assay yielded about 1.3M sequence reads, of which about 50% were productive. The mean sequence read length was 103 nucleotides and about 1000 IgH clones were identified.

### Example 3

The IgH V gene FR3 primers of Table 2 and the IgH J gene primers of Table 5 were designed to amplify all of the currently known expressed or gDNA human IgH rearrangements. In multiplex PCR, a pool of forward and reverse primers selected from Tables 2 and 5 were used as primer pairs in amplifying sequences from the V gene FR3 region to the J gene of IgH cDNA. In the exemplary IgH V gene FR3-J amplification reactions, the multiplex primer set pool included forward primers SEQ ID NOs: 69-136 and reverse primers SEQ ID NOs: 443-446. Assays were performed on cDNA from a variety of human cell and tissue samples.

Total RNA from human adult normal peripheral blood leukocytes (from BioChain Institute, Inc.) was reverse transcribed to cDNA with SuperScript™ IV VILO™ Master Mix (Thermo Fisher Scientific) according to manufacturer instructions. To a single well of a 96-well PCR plate was added 10 microliters prepared cDNA (50 ng), 4 microliters of 1 μM forward and reverse primer pool, 4 microliters of 5× Ion AmpliSeq™ HiFi Mix (Thermo Fisher Scientific), and 2 microliters DNase/RNase free water to bring the final reaction volume to 20 microliters. The PCR plate was sealed, reaction mixtures mixed, and loaded into a thermal cycler (e.g., Veriti™ 96-well thermal cycler (Applied Biosystems)) and run on the following temperature profile to generate the amplicon library. An initial holding stage was performed at 95° C. for 2 minutes, followed by about 20 cycles of a denaturing stage at 95° C. for 15 seconds, an annealing stage at 60° C. for 45 seconds, and an extending stage for 72° C. for 45 seconds. After cycling, a final extension 72° C. for 10 minutes was performed and the amplicon library was held at 10° C. until proceeding. Typically, about 20 cycles are used to generate the amplicon library. For some applications (eg., more or less cDNA starting material, FFPE sourced RNA, etc), cycle number may be reduced (e.g., −3) or increased (e.g., +3, +6, up to 30 cycles).

The amplicon sample was briefly centrifuged to collect contents before proceeding. To the amplicon library (˜20 microliters), 2 microliters of FuPa reagent was added. The reaction mixture was sealed, mixed thoroughly to ensure uniformity and incubated at 50° C. for 10 minutes, 55° C. for 10 minutes, 60° C. for 20 minutes, then held at 10° C. for up to 1 hour. The sample was briefly centrifuged to collect contents before proceeding to a ligation step. The reaction mixture now containing the phosphorylated amplicon library was combined with 2 microliters of Ion Torrent™ Dual Barcode Adapters (Thermo Fisher Scientific), 4 microliters of AmpliSeq Plus Switch Solution (sold as a component of the Ion AmpliSeq™ Library Kit Plus, Thermo Fisher Scientific) and 2 microliters of DNA ligase, added last (sold as a component of the Ion AmpliSeq™ Library Kit Plus, Thermo Fisher Scientific), then incubated at the following: 22° C. for 30 minutes, 68° C. for 5 minutes, 72° C. for 5 minutes, then held at 10° C. for up to 1 hour. The sample was briefly centrifuged to collect contents before proceeding to a library purification step.

After the ligation step incubation, 45 microliters (1.5× sample volume) of room temperature AMPure® XP beads (Beckman Coulter) was added to ligated DNA and the mixture was pipetted thoroughly to mix the bead suspension with the DNA. The mixture was incubated at room temperature for 5 minutes, placed on a magnetic rack such as a DynaMag™-96 side magnet (Invitrogen, Part No. 12331D) for two minutes. After the solution had cleared, the supernatant was discarded. Without removing the plate from the magnetic rack, 150 microliters of freshly prepared 70% ethanol was introduced into the sample, and incubated while gently rotating the tube on the magnetic rack. After the solution cleared, the supernatant was discarded without disturbing the pellet. A second ethanol wash was performed, the supernatant discarded, and any remaining ethanol was removed by pulse-spinning the tube and carefully removing residual ethanol while not disturbing the pellet. The pellet was air-dried for about 5 minutes at room temperature. The ligated DNA was eluted from the beads in 50 microliters of low TE buffer.

The eluted libraries were quantitated by qPCR using the Ion Library TaqMan® Quantitation Kit (Ion Torrent, Cat. No. 4468802), according to manufacturer instructions. After quantification, the libraries were diluted to a concentration of about 25 μM.

The libraries were normalized to 25 μM and aliquots of the final libraries were used in template preparation and chip loading using the Ion Chef™ instrument according to the manufacturer instructions. Sequencing was performed using Ion 540™ chips on the Ion S5™ System according to manufacturer instructions, and gene sequence analysis was performed with the Ion Torrent Suite™ software. Since the sequences were generated from use of J gene primers, they were subjected to a J gene sequence inference process involving adding the inferred J gene sequence to the sequence read to create an extended sequence read, aligning the extended sequence read to a reference sequence, and identifying productive reads, as described herein. In addition, all of the generated sequence data was further subjected to the error identification and removal programs provided herein.

Exemplary results from the IgH FR3-J assay using 4 separate tubes of peripheral blood leukocyte RNA as starting sample are shown in Table 11. Each of these assays yielded 2-3 M raw sequence reads, of which about 75% were productive. Evenness, also referred to as Clone Normalized Shannon Entropy, describes how even clone representation is in the sample; the closer to 1.0, the more evenly sized the clonal populations are.

TABLE 11

| | Total | | % | | | |
|---|---|---|---|---|---|---|
| | Productive | Mean Read | Productive | Clones | Clone Shannon | |
| Library | Reads | Length (nt) | Reads | Identified | Diversity | Evenness |
| PBL-1 | 1,626,423 | 89 | 75.71% | 105,259 | 15.2988632 | 0.916987 |
| PBL-2 | 1,001,081 | 90 | 75.43% | 86,922 | 14.950752 | 0.911218 |
| PBL-3 | 1,355,021 | 90 | 72.73% | 97,824 | 15.133181 | 0.912853 |
| PBL-4 | 855,367 | 89 | 75.88% | 78,174 | 14.759806 | 0.908050 |

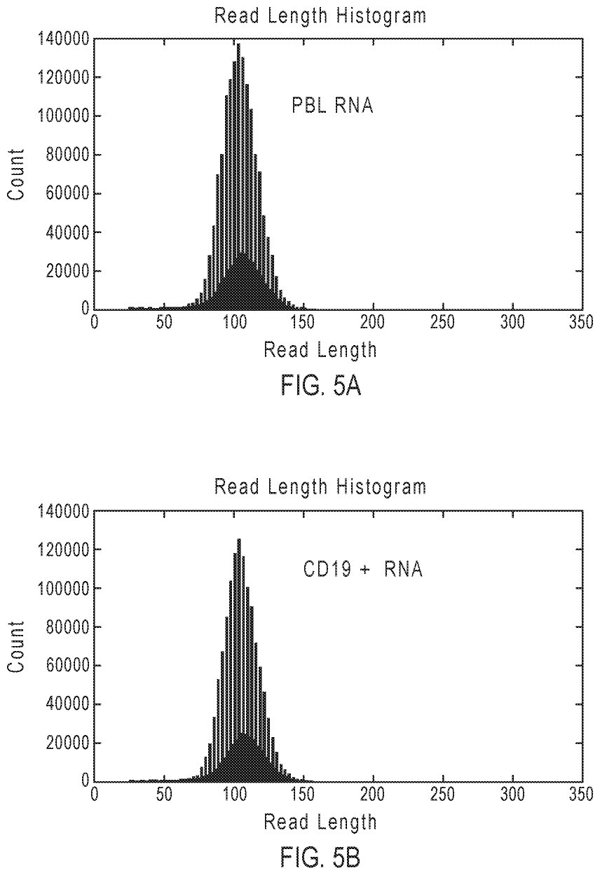

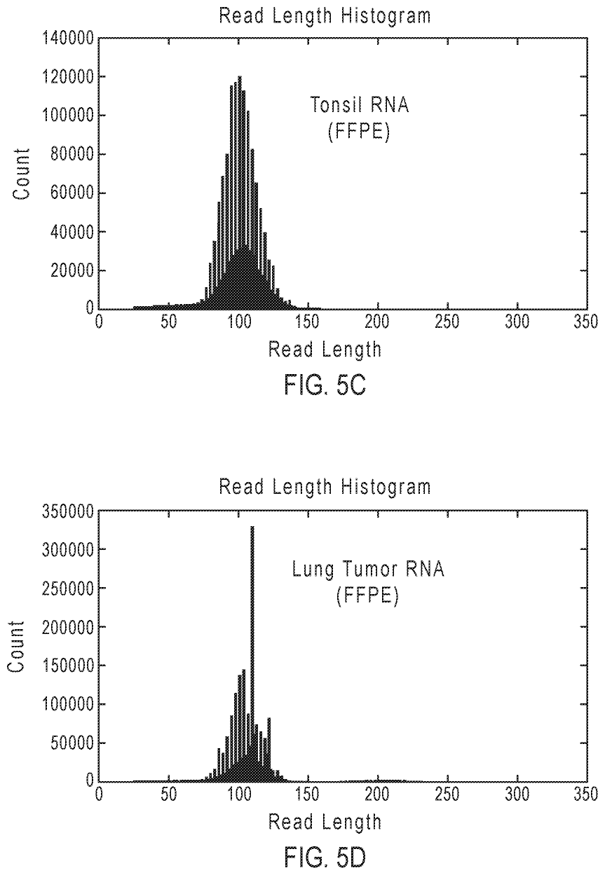

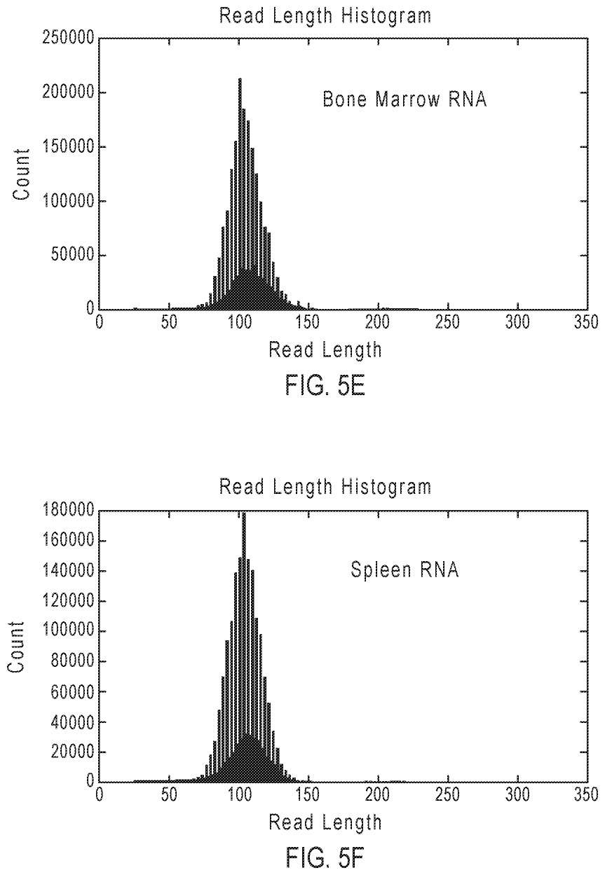

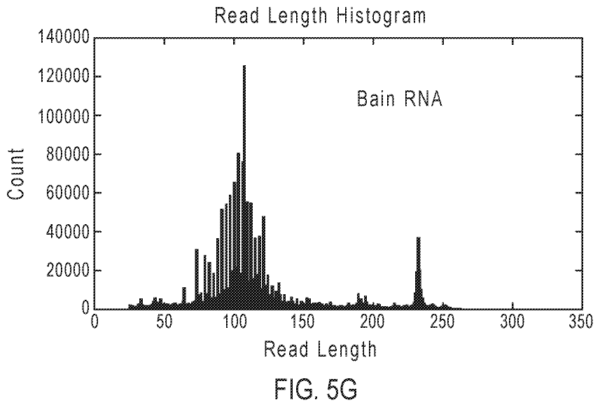

Total RNA was obtained from human cell and tissue samples (BioChain Institute, Inc.) which vary in the amount of B cells typically present in such a sample. The RNA samples included those extracted from isolated CD19+ cells, normal spleen tissue, peripheral blood leukocytes (PBLs), bone marrow, normal brain tissue, lung tumor tissue (FFPE), tonsil (FFPE), and Jurkat cells (a T cell line). For each total RNA sample, cDNA was prepared and subjected to multiplex amplification using the primer set including SEQ ID NOs: 69-136 and 443-446, as described above. Libraries were prepared from the resulting amplicons and the libraries sequenced as described above. Assay results for the various samples are shown in Table 12 and FIGS. 5A-5G depict read length histograms obtained for the PBL, CD19+ cell, tonsil FFPE, lung tumor FFPE, bone marrow, spleen, and brain RNA samples. Other than that from the Jurkat cell and normal brain tissue samples, each of these assays resulted in about 70-80% of the sequence reads being productive IgH reads. The Jurkat cell RNA sample served as a negative control in that it is a T cell line which does not express IgH.

TABLE 12

| | | Mean | % | | Clone | |
|---|---|---|---|---|---|---|
| | | Read | Produc- | Clones | Shannon | |
| | Total | Length | tive | Iden- | Diver- | Even- |
| Sample | Reads | (nt) | Reads | tified | sity | ness |
| PBL | 2,042,495 | 103 | 77.5 | 14136 | 13.1433 | 0.9533 |
| Tonsil | 1,912,924 | 99 | 71.0 | 5392 | 11.8700 | 0.9575 |
| (FFPE) | | | | | | |
| CD19 + | 2,173,396 | 103 | 77.4 | 47757 | 15.0880 | 0.9707 |
| RNA | | | | | | |
| Bone | 2,074,903 | 104 | 77.5 | 43524 | 13.2147 | 0.8576 |
| Marrow | | | | | | |
| Lung | 2,236,253 | 107 | 73.4 | 9750 | 8.2858 | 0.6253 |
| Tumor | | | | | | |
| (FFPE) | | | | | | |
| Normal | 3,674,828 | 103 | 76.7 | 29613 | 13.9319 | 0.9379 |
| Spleen | | | | | | |
| Normal | 1,870,257 | 118 | 41.6 | 2479 | 10.4404 | 0.9259 |
| Brain | | | | | | |
| Jurkat | 1,747,982 | 146 | 0.0002 | 1 | 0 | 0 |
| RNA | | | | | | |

### Example 4

The IgH V gene FR1 primers of Table 3 and the IgH C gene primers of Tables 6-10 were designed to amplify all of the currently known expressed human IgH rearrangements. A variety of primer sets for amplifying sequences from the V gene FR1 region to the C gene of IgH cDNA were generated using forward primers selected from Table 3 and reverse primers selected from Tables 6-10. Some of the primer sets included at least one primer directed to the C gene for each of IgH isotypes IgA, IgD, IgG, IgM and IgE. Exemplary FR1-C primer set panels are described in Table 13 with each primer in the set at a 1 micromolar concentration.

TABLE 13

| FR1-C Primer Set | SEQ ID NOs |
|---|---|
| 1 | 311-336, 461, 462, 481, 515-519, 554, 585, 586 |
| 2 | 321-328, 337-354, 463, 464, 482, 520-524, 555, 587, |
| | 588 |
| 3 | 321-328, 335, 336, 347-352, 355-364, 465, 466, 483, |
| | 525-529, 556, 589, 590 |
| 4 | 321-328, 347-354, 365-374, 467, 468, 484, 530-534, |
| | 557, 591, 592 |
| 5 | 321-328, 375-393, 469, 485, 535, 558, 559, 593-595 |
| 6 | 386-391, 394-410, 470, 486, 536, 537, 560, 561, |
| | 596-598 |
| 7 | 295-301, 411-430, 471, 487, 538, 539, 562, 563, |
| | 599-601 |
| 8 | 321-328, 375-393, 469, 485, 535, 558, 559, 593, 595 |
| 9 | 295-301, 321-328, 375-393, 411-430, 469, 485, 535, |
| | 558, 559, 593, 595 |

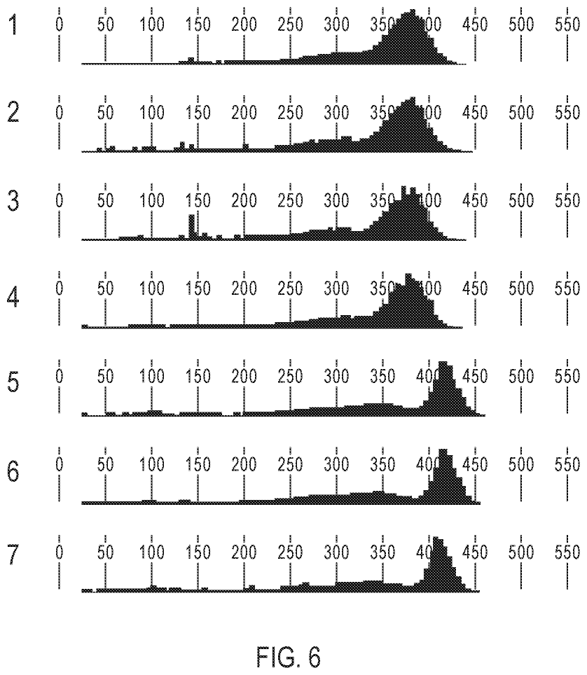

cDNA was prepared from PBL total RNA as described in Example 3. Multiplex amplification reactions were performed using 50 ng of PBL cDNA and a primer set of Table 13, libraries were prepared from the resulting amplicons and the libraries sequenced as described in Example 3. FIG. 6 depicts resulting sequence read lengths obtained for exemplary primer sets 1-7. Exemplary assay results from these primer sets are shown in Table 14.

TABLE 14

| | | Mean | % | | Clone | |
|---|---|---|---|---|---|---|
| Library | | Read | Produc- | Clones | Shannon | |
| (primer | Total | Length | tive | Iden- | Diver- | Even- |
| set) | Reads | (nt) | Reads | tified | sity | ness |
| 1 | 504,425 | 338 | 51.97 | 3635 | 9.9723 | 0.8431 |
| 2 | 554,200 | 317 | 44.15 | 3360 | 9.9317 | 0.8478 |
| 3 | 710,813 | 324 | 48.69 | 6138 | 10.5166 | 0.8357 |
| 4 | 575,606 | 303 | 38.96 | 3024 | 9.55 | 0.8258 |
| 5 | 520,054 | 341 | 45.46 | 2629 | 9.4513 | 0.8320 |
| 6 | 573,796 | 358 | 49.21 | 2934 | 9.3869 | 0.8149 |
| 7 | 534,119 | 321 | 39.24 | 980 | 7.9047 | 0.7955 |

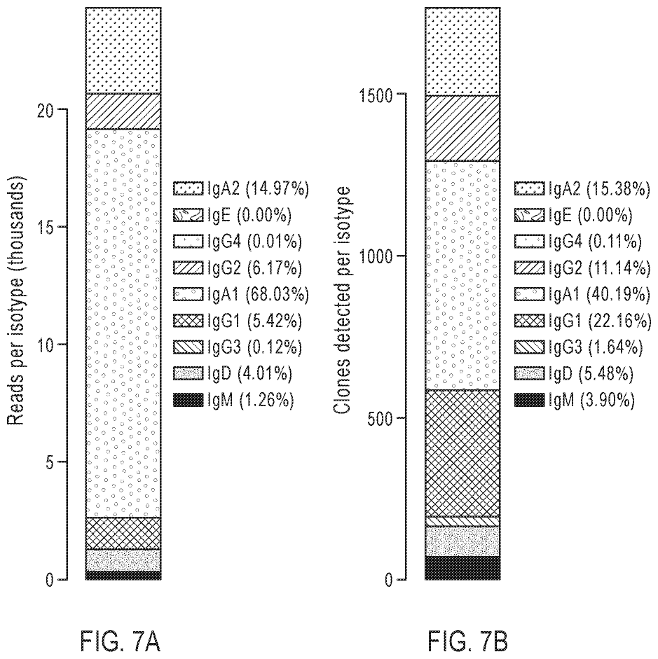

B cell repertoire profiling using one or more C gene primers selected from Table 6-10 in the provided multiplex amplification and sequencing analysis workflows result in isotype characterization of the detected IgH clone sequences. With use of a primer set including at least one primer directed to the C gene for each of isotypes IgA, IgD, IgG, IgM and IgE, all isotypes of the B cell receptor repertoire and clonal lineages in the sample are detected and characterized. FIGS. 7A-7B show exemplary isotype usage results from a PBL sample as detected using the primer set 8 of Table 13 in an IgH V gene FR1-C multiplex amplification reaction (with 25-50 ng cDNA input) and sequencing analysis as described above. The bar plots of FIG. 7 show the summary of the total isotype representation within the PBL sample, reported by number of reads per isotype (FIG. 7A) and clones and lineages per isotype (FIG. 7B). The isotype representation obtained (IgA and IgG more highly represented than IgM, IgD, and IgE) is expected given the higher expression of IgA and IgG isotypes in the PBL RNA.

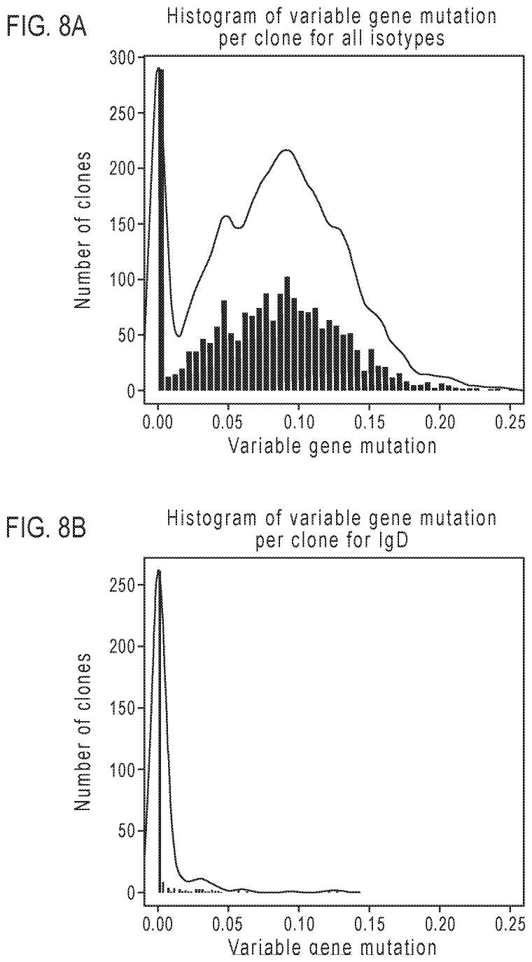

Bioinformatics workflows provided herein identify somatic hypermutations of clonal lineages and generate somatic hypermutation profiles of a sample. For example, FIGS. 8A-8B show an exemplary SMH profile from a PBL RNA sample assayed using the primer set 8 of Table 13 in an IgH V gene FR1-C multiplex amplification reaction (with 25-50 ng cDNA input) and sequencing analysis as described above. FIG. 8A shows a histogram of IgH V-gene mutation rate for all isotypes of the sample and reports an expected population of clones with no SHM and a distribution of clones with SHM up to about 15%. The SMH profile shown in FIG. 8B represents IgH V-gene mutation for isotype IgD, a naive B cell isotype where a low rate of SHM is expected.

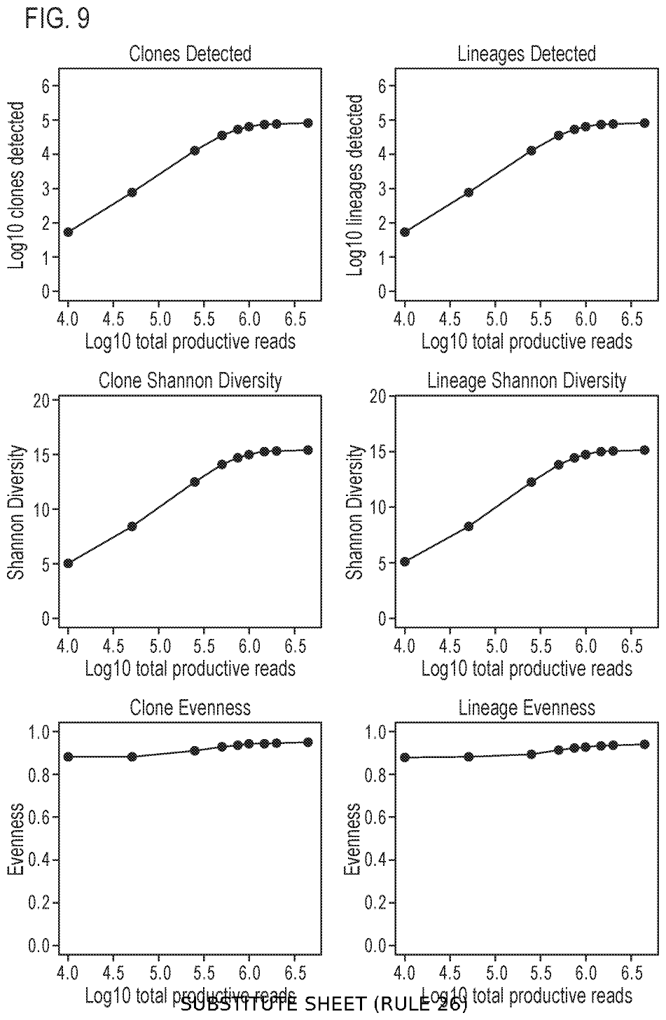

Sequence analysis workflows provided herein may include a downsampling analysis which can help, for example, to eliminate variability owing to differences in sequencing depth across an assay. For such downsampling analysis, sequence reads are randomly removed down to fixed read depths prior to clonal analysis. For such an analysis, a PBL RNA sample was assayed in an IgH V gene FR1-C multiplex amplification reaction using 25-50 ng cDNA input and the primer set 9 of Table 13 and the sequencing workflow as described above. The generated sequence data was subjected to the error identification and removal steps provided herein and the set of productive reads (i.e., productive and rescued productive reads) for the sample was identified. Downsampling to each of the selected fixed depths was performed using the entire set of productive reads as the starting point and then randomly selecting a fixed number of reads. Downsampling was performed to the following fixed depths: 10K, 50K, 250K, 500K, 750K, 1M, 1.5M, and 2M reads. Since each downsampling analysis starts with the entire set of productive reads, the reads for a lower downsampling depth are not necessarily a subset of those in a higher downsampling depth. Clonal analysis was performed with each of the downsampled data sets as well as with the total productive read data set. Results of exemplary clonal analysis following the application of downsampling to the IgH sequence reads is shown in FIG. 9. The graphs of FIG. 9 depict the number of clones and lineages detected, clone and lineage Shannon diversity, and clone and lineage evenness for each of the downsampled data sets and for the total productive reads for the sample (rightmost point on each graph). Downsampling analysis permits the user to identify the point at which a particular sample is sequenced to saturation, meaning a point at which additional reads do not identify additional clones or lineages or add additional diversity to the detected repertoire. For example, downsampling allows the user to refine sequencing depth or multiplexing between assays or in future assays with similar sample types.

### Example 5

A library of 20 control plasmids (Table 15) was generated to represent a set of various IgH sequences and used to assess performance of the assays and workflows provided herein. Each control plasmid contained the VDJ region to C gene CH1 domain (about the first 300 bp of the constant gene) of an IgH cDNA from one of 11 chronic lymphocytic leukemia (CLL) or one of 9 members of a broadly neutralizing HIV-1 antibody (bnAb) lineage (Liao et al. (2013) Nature 496:469-476). The library of control plasmids included representatives of all IgH isotypes and CLL rearrangements to maximize the V gene diversity, including germline and mutated rearrangements. Each plasmid has a total IgH insert length of about 650 bp. Some plasmids in the control plasmid library were designed with nucleotide mutations within the FR1 region that hinder primer binding (plasmid 18) or with isotype errors (plasmids 17 and 20). The control library also included a plasmid (plasmid 19) generated with an out-of-frame, non-functional receptor sequence that should be filtered out as a non-productive read in the sequence analysis workflow.

TABLE 15

| Plasmid | | Insert |
|---|---|---|
| ID | IgH VDJ-C insert | source |
| 1 | KC575857.1_IGHV4-59*01_93.4_IGHG1*01 | bnAb |
| 2 | JX432218.1_IGHV3-9*01_96.3_IGHA1 | CLL |
| 3 | AF021964.1_IGHV3-30*18_91.6_IGHG1*01 | CLL |
| 4 | KC575856.1_IGHV4-59*01_92.8_IGHG3*01 | bnAb |
| 5 | JX432219.1_IGHV3-21*01_964.2_IGHA2*01 | CLL |
| 6 | AF021966.1_IGHV3-33*01_91.2_IGHG2*01 | CLL |
| 7 | KC575854.1_IGHV4-59*01_98.3_IGHD*01 | bnAb |
| 8 | KC575852.1_IGHV4-59*01_83.3_IGHA2*01 | bnAb |
| 9 | KC575849.1_IGHV4-59*01_91.4_IGHG2*01 | bnAb |
| 10 | JX432222.1_IGHV1-46*03_100_IGHG3*01 | CLL |
| 11 | KC575846.1_IGHV4-59*01_86.2_IGHE*01 | bnAb |
| 12 | AF021958.1_IGHV4-39*01_100_IGHM*01 | CLL |
| 13 | KC575862.1_IGHV4-59*01_96.9_IGHM*01 | bnAb |
| 14 | AF021967.1_IGHV1-18*01_100_IGHD*01 | CLL |
| 15 | KC575850.1_IGHV4-59*01_91.4_IGHG4*01 | bnAb |
| 16 | KC575846.1_IGHV4-59*01_86.2_IGHE*01 | bnAb |
| 17 | Z95484.1_IGHV3-48*03_89.5_IGHG4*01 | CLL |
| | (isotype error) | |
| 18 | AF021956.1_IGHV1-3*01_89.2_IGHE*01 | CLL |
| | (FR1 mutations) | |
| 19 | JX432220.1_IGHV3-23*01_89.5_IGHA1 | CLL |
| | (non-functional) | |
| 20 | JX432165.1_IGHV4-34*01_98.6_IGHM*01 | CLL |
| | (isotype error) | |

To assess the limit of detection for the assay and workflow, control libraries were prepared using pooled plasmids at single known input concentrations in a background of 100 ng leukocyte cDNA. Plasmid input concentrations ranged from 10 pg to 0.00001 pg (equivalent to 5M copies to about 5 copies). The control plasmids were linearized (individually or in bulk) or left intact prior to use in the assays. The pooled libraries were amplified in multiplex reactions using the primer set 8 or primer set 9 of Table 13 with each primer at 200 nM, and amplification and sequencing was performed as described in Example 4. The generated sequence data was subjected to the error identification and removal programs provided herein.

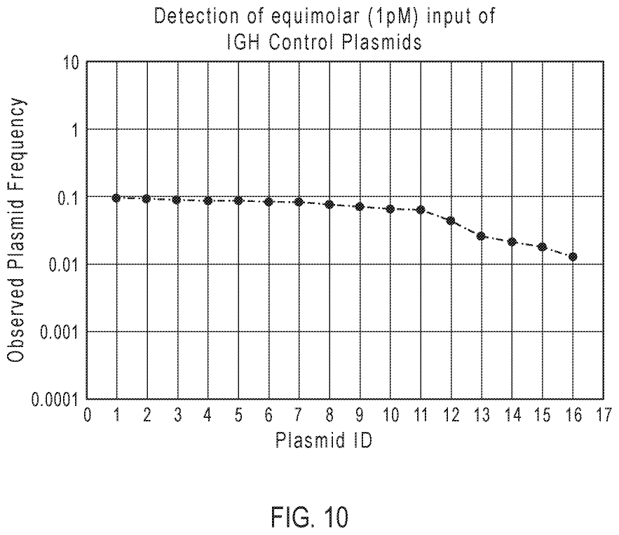

As shown in FIG. 10, performance of the assay on a pool of the 20 control plasmids at an equimolar concentration of 1 μM resulted in detected plasmid frequencies within one order of magnitude. Control plasmids 17-20, which were designed with sequence errors to not result in productive sequence reads, were not detected in the assay.

A synthetic oligo containing the IgH VDJ-C insert of control plasmid 2 (JX432218.1_IGHV3-9*01_96.3_IGHA1) was used to assess the limit of detection and show the capability of the assay and analysis workflows to identify and detect the frequency of a single clone of interest. The control synthetic oligo was added to a 25 ng PBL total RNA sample at varying concentration and the clonotype frequency of the control sequence was determined using multiplex reactions with an IgH V gene FR3-J multiplex primer set including forward primers SEQ ID NOs: 69-136 and reverse primers SEQ ID NOs: 443-446, with each primer at 200 nM, and amplification, sequencing, and analysis performed as described above and in Example 3. Control oligo input amounts ranged from 0.1 pg to 0.00001 pg. The clone detection frequency for replicate assays is shown in Table 16.

TABLE 16

| Control Oligo | | Clone Detection Frequency | Clone Detection Frequency |
|---|---|---|---|
| Input Amount | | Replicate 1 | Replicate 2 |
| 0.1 | pg | 0.517825 | 0.463189 |
| 0.01 | pg | 0.064037 | 0.053178 |
| 0.001 | pg | 0.005957 | 0.005111 |
| 0.0001 | pg | 0.000744 | 0.000590 |
| 0.00001 | pg | 0.000069 | 0.000049 |

In other clonotype frequency detection assays, the synthetic oligo containing the IgH VDJ-C insert of control plasmid 2 (JX432218.1_IGHV3-9*01_96.3_IGHA1) was added to 50 ng PBL total RNA, 100 ng PBL total RNA, and 100 ng bone marrow (BM) total RNA at input amounts from 0.1 pg to 0.0000001 pg. Multiplex amplification reactions were performed using an IgH V gene FR3-J multiplex primer set including forward primers SEQ ID NOs: 69-136 and reverse primers SEQ ID NOs: 443-446, and sequencing of the resulting amplicons and analysis was performed as described in Example 3. In a DNA assay, the synthetic oligo was added to 1 μg PBL gDNA at input amounts from 0.1 pg to 0.0000001 pg. Multiplex amplification reactions were performed using a multiplex primer set including forward primers SEQ ID NOs: 69-136 and reverse primers SEQ ID NOs: 443-446, and sequencing of the resulting amplicons and analysis was performed as described in Example 2. Exemplary limit of detection results for such clone frequency assays are shown in Table 17. The IgH FR3-J assays were able to identify the control CLL sequence at frequencies between 7.6×10⁻⁶ and 1.0×10⁻⁶ with a single library using only 100 ng of input PBL or bone marrow RNA. The IgH FR3-J assay was able to identify the control CLL sequence at about 5.2×10⁻⁶ using 1 pg of input PBL gDNA. The same assay was performed in a background of 1 μg bone marrow gDNA with a range of input amounts for the oligo and the resulting libraries were sequenced to a target depth of 10 M reads using the Ion S5™ System and Ion 540™ chip. The IgH FR3-J DNA assay with bone marrow background was able to detect the control CLL sequence to a frequency of 10-5 using a single library.

TABLE 17

| Input | | | | | Total | |
|---|---|---|---|---|---|---|
| RNA or | | | | | Productive | Clones |
| DNA | V gene | J gene | CDR3 NT sequence | Frequency | Reads | Detected |
| PBL | IGHV3-9 | IGHJ6 | GCAAAAGATGTTGGGGCTAC | 0.0000103 | 3,045,014 | 91,907 |
| RNA | | | GGGCTACTACGGTATGGACG | | | |
| 50 ng | | | TC (SEQ ID NO: 604) | | | |
| PBL | IGHV3-9 | IGHJ6 | GCAAAAGATGTTGGGGCTAC | 0.0000073 | 4,342,768 | 102,047 |
| RNA | | | GGGCTACTACGGTATGGACG | | | |
| 100 ng | | | TC (SEQ ID NO: 604) | | | |
| BM | IGHV3-9 | IGHJ6 | GCAAAAGATGTTGGGGCTAC | 0.0000010 | 5,943,175 | 53,785 |
| RNA | | | GGGCTACTACGGTATGGACG | | | |
| 100 ng | | | TC (SEQ ID NO: 604) | | | |
| PBL | IGHV3-9 | IGHJ6 | GCAAAAGATGTTGGGGCTAC | 0.0000052 | 10,839,583 | 2974 |
| DNA | | | GGGCTACTACGGTATGGACG | | | |
| 1 μg | | | TC (SEQ ID NO: 604) | | | |

For clonotype frequency detection assays with an IgH V gene FR1-C primer set, a synthetic oligo containing the IgH VDJ-C insert of control plasmid 13 (KC575862.1_IGHV4-59*01_96.9_IGHM*01) was added to 50 ng bone marrow (BM) total RNA at input amounts from 0.1 pg to 0.0000001 pg. Multiplex amplification reactions were performed using the multiplex primer set 9 of Table 13 and sequencing of the resulting amplicons and analysis was performed as described in Example 3. Exemplary limit of detection results for such clone frequency assays are shown in Table 18. The IgH FR1-C assay was able to identify the control bnAb sequence at frequencies between 1.1×10⁻⁴ and 8.7×10-5 with a single library using only 50 ng of input bone marrow RNA.

TABLE 18

| Input RNA | V gene | J gene | CDR3 NT sequence | Frequency |
|---|---|---|---|---|
| BM RNA | IGHV4-59 | IGHJ4 | GCGAGCGTGCGCAGGGGGCAGTTAGTCA | 0.0000868 |
| 50 ng | | | CTGCCCACTTTGACTCC (SEQ ID NO: 605) | |
| BM RNA | IGHV4-59 | IGHJ4 | GCGAGCGTGCGCAGGGGGCAGTTAGTCA | 0.0001175 |
| 50 ng | | | CTGCCCACTTTGACTCC (SEQ ID NO: 605) | |

The ability of the assays and workflows provided herein to quantify somatic hypermutation (SHM) and isotype of germline and mutated CLL IgH clones was evaluated using the control plasmids with CLL derived inserts. The selected plasmid constructs of Table 15 were added to a PBL total RNA background. Multiplex amplification reactions were performed using the multiplex primer set 9 of Table 13 and libraries were prepared, sequenced and sequences analyzed as described in Example 3, with the libraries sequenced to a target depth of 1.5 M reads using the Ion S5™ System and Ion 530™ chip. Exemplary results for such SHM quantifying assays are shown in Table 19. Table 19 shows the expected SHM status, SHM frequency and isotype for each plasmid based on the input into the assay and the observed SHM status, SHM frequency and isotype for each plasmid from the assay results. As shown, the assay accurately quantifies SHM and isotype of the control CLL panel.

TABLE 19

| | Expected | | | Observed | | |
|---|---|---|---|---|---|---|
| | | SHM | | | SHM | |
| Plasmid | SHM | Fre- | Iso- | SHM | Fre- | Iso- |
| ID | status | quency | type | status | quency | type |
| 2 | Mutated | .037 | IgA1 | Mutated | .048 | IgA1 |
| 6 | Mutated | .088 | IgG2 | Mutated | .102 | IgG2 |
| 3 | Mutated | .084 | IgG1 | Mutated | .088 | IgG1 |
| 5 | Mutated | .058 | IgA2 | Mutated | .057 | IgA2 |
| 10 | Germline | 0 | IgG3 | Germline | 0 | IgG3 |
| 12 | Germline | 0 | IgM | Germline | 0 | IgM |
| 14 | Germline | 0 | IgD | Germline | 0 | IgD |

Such results for limit of detection, clonotype frequency detection, and SHM and isotype quantification demonstrate the capability of the provided assays and analysis workflows to identify and detect rare BCR clones. This capability is well-suited for longitudinal studies where the frequency of a particular clone (or small groups of clones) is being tracked.

1-66. (canceled)

67. NEW A method for amplification of expression nucleic acid sequences of a B cell receptor (BCR) repertoire in a sample, comprising:
performing a single multiplex amplification reaction to amplify expressed target BCR nucleic acid template molecules using at least one set of:
i) (a) a plurality of V gene primers directed to a majority of different V genes of at least one BCR coding sequence comprising at least a portion of framework region 1 (FR1) within the V gene,
(b) a plurality of V gene primers directed to a majority of different V genes of at least one BCR coding sequence comprising at least a portion of framework region 2 (FR2) within the V gene, or
(c) a plurality of V gene primers directed to a majority of different V genes of at least one BCR coding sequence comprising at least a portion of framework region 3 (FR3) within the V gene; and
ii) (a) one or more C gene primers directed to at least a portion of a C gene of the at least one BCR coding sequence, or
(b) a plurality of J gene primers directed to at least a portion of a majority of different J genes of the at least one BCR coding sequence;
wherein each set of i) and ii) primers is directed to coding sequences of the same target BCR gene selected from an IgH, IgL, and IgK gene and wherein performing the amplification using the at least one set of i) and ii) primers results in amplicon molecules representing the target BCR repertoire in the sample;
thereby generating target BCR amplicon molecules comprising the expressed target BCR repertoire.

68. NEW The method of claim 67, wherein each of the plurality of V gene primers, the plurality of J gene primers and/or the one of more C gene primers has any one or more of the following criteria:
(1) includes two or more modified nucleotides within the primer, at least one of which is included near or at the termini of the primer and at least one of which is included at, or about the center nucleotide position of the primer;
(2) length is about 15 to about 40 bases in length;
(3) Tₘ of from above 60° C. to about 70° C.;
(4) has low cross-reactivity with non-target sequences present in the sample;
(5) at least the first four nucleotides (going from 3′ to 5′ direction) are non-complementary to any sequence within any other primer present in the same reaction; and
(6) are non-complementary to any consecutive stretch of at least 5 nucleotides within any other produced target amplicon.

69. NEW The method of claim 67, wherein each of the plurality of V gene primers, the plurality of J gene primers and/or the one or more C gene primers includes one or more cleavable groups, preferably located (i) near or at the termini of the primer or (ii) near or about the center nucleotide of the primer.

70. NEW The method of claim 67, wherein each of the plurality of V gene primers, the plurality of J gene primer and/or the one or more C gene primers includes two or more modified nucleotides having a cleavable group selected from a methylguanine, 8-oxo-guanine, xanthine, hypoxanthine, 5,6-dihydrouracil, uracil, 5-methylcytosine, thymine-dimer, 7-methylguanosine, 8-oxo-deoxyguanosine, xanthosine, inosine, dihydrouridine, bromodeoxyuridine, uridine or 5-methylcytidine.

71. NEW The method of any claim 67, wherein the at least one set of i) and ii) is i)(a) and ii)(a), wherein the plurality of V gene primers anneal to at least a portion of the FR1 portion of the template molecules, and wherein the one or more C gene primers comprises at least five primers that anneal to at least a portion of the C gene portion of the template molecules.

72. NEW The method of claim 71, wherein the at least one set of i) and ii) is selected from primers of Table 3 and Tables 6-10, respectively or at least one set of i) and ii) is selected from the primer sets of Table 11.

73. NEW The method of claim 67, wherein the at least one set of i) and ii) is i)(a) and ii)(b), wherein the plurality of V gene primers anneal to at least a portion of the FR1 portion of the template molecules, and wherein the plurality of J gene primers comprises at least two primers that anneal to at least a portion of the J gene portion of the template molecules.

74. NEW The method of claim 73, wherein the at least one set of i) and ii) is selected from primers of Table 3 and Table 5, respectively.

75. NEW The method of claim 67, wherein the at least one set of i) and ii) is i)(c) and ii)(a), wherein the plurality of V gene primers anneal to at least a portion of the FR3 portion of the template molecules, and wherein the one or more C gene primers comprises at least five primers that anneal to at least a portion of the C gene portion of the template molecules.

76. NEW The method of claim 75, wherein the at least one set of i) and ii) is selected from primers of Table 2 and Tables 6-10, respectively.

77. NEW The method of claim 67, wherein the at least one set of i) and ii) is i)(c) and ii)(b), wherein the plurality of V gene primers anneal to at least a portion of the FR3 portion of the template molecules, and wherein the plurality of J gene primers comprises at least two primers that anneal to at least a portion of the J gene portion of the template molecules.

78. NEW The method of claim 77, wherein the at least one set of i) and ii) is selected from primers of Table 2 and Table 5.

79. NEW A method for preparing an expressed BCR repertoire library, comprising:
i) treating the target BCR amplicon molecules according to claim 67 to form blunt-ended amplicon molecules; and
ii) ligating at least one adapter to at least one of the treated target BCR amplicon molecules, thereby producing a library of adapter-ligated target BCR amplicon molecules comprising the target BCR repertoire.

80. NEW A method for providing sequence of an expressed BCR repertoire in a sample, comprising:
i) performing sequencing of the target BCR library of claim 79;
ii) determining the sequence of the library molecules, wherein determining the sequence includes obtaining initial sequence reads, aligning the initial sequence read to a reference sequence, identifying productive reads, and correcting one or more indel errors to generate rescued productive sequence reads; and
iii) reporting the sequences determined for the library molecules, thereby providing sequence of the expressed BCR repertoire in the sample.

81. NEW A method for amplification of rearranged genomic DNA (gDNA) sequences of a B cell receptor (BCR) repertoire in a sample, comprising:
performing a single multiplex amplification reaction to amplify target BCR gDNA template molecules having a J gene portion and a V gene portion, the target BCR gDNA having rearranged VDJ or VJ gene segments, using at least one set of:
i) (a) a plurality of V gene primers directed to a majority of different V genes of at least one BCR coding sequence comprising at least a portion of FR1 within the V gene,
(b) a plurality of V gene primers directed to a majority of different V genes of at least one BCR coding sequence comprising at least a portion of FR2 within the V gene, or
(c) a plurality of V gene primers directed to a majority of different V genes of at least one BCR coding sequence comprising at least a portion of FR3 within the V gene; and
ii) a plurality of J gene primers directed to at least a portion of a majority of different J genes of the at least one BCR coding sequence;
wherein each set of i) and ii) primers is directed to coding sequences of the same target BCR gene selected from an IgH, IgL, and IgK gene and wherein performing the amplification using the at least one set of i) and ii) primers results in amplicon molecules representing the target BCR repertoire in the sample; thereby generating target BCR amplicon molecules comprising the target BCR repertoire.

82. NEW The method of claim 81, wherein each of the plurality of V gene primers and the plurality of J gene primers has any one or more of the following criteria:
(1) includes two or more modified nucleotides within the primer, at least one of which is included near or at the termini of the primer and at least one of which is included at, or about the center nucleotide position of the primer;
(2) length is about 15 to about 40 bases in length;
(3) Tₘ of from above 60° C. to about 70° C.;
(4) has low cross-reactivity with non-target sequences present in the sample;
(5) at least the first four nucleotides (going from 3′ to 5′ direction) are non-complementary to any sequence within any other primer present in the same reaction; and
(6) are non-complementary to any consecutive stretch of at least 5 nucleotides within any other produced target amplicon.

83. NEW The method of claim 81, wherein each of the plurality of V gene primers and/or the plurality of J gene primers includes two or more modified nucleotides having a cleavable group selected from a methylguanine, 8-oxo-guanine, xanthine, hypoxanthine, 5,6-dihydrouracil, uracil, 5-methylcytosine, thymine-dimer, 7-methylguanosine, 8-oxo-deoxyguanosine, xanthosine, inosine, dihydrouridine, bromodeoxyuridine, uridine or 5-methylcytidine.

84. NEW The method of any claim 81, wherein the at least one set of i) and ii) is i)(a) and ii), wherein the plurality of V gene primers anneal to at least a portion of the FR1 portion of the template molecules, and wherein the plurality of J gene primers comprises at least two primers that anneal to at least a portion of the J gene portion of the template molecules.

85. NEW The method of claim 84, wherein the at least one set of i) and ii) is selected from primers of Table 3 and Table 5, respectively.

86. NEW The method of claim 81, wherein the at least one set of i) and ii) is i)(c) and ii), wherein the plurality of V gene primers anneal to at least a portion of the FR3 portion of the template molecules, and wherein the plurality of J gene primers comprises at least two primers that anneal to at least a portion of the J gene portion of the template molecules.

87. NEW The method of claim 86, wherein the at least one set of i) and ii) is selected from primers of Table 2 and Table 5, respectively.

88. NEW A method for preparing a rearranged gDNA BCR repertoire library, comprising:
i) treating the target BCR amplicon molecules of claim 81 to form blunt-ended amplicon molecules; and
ii) ligating at least one adapter to at least one of the treated target BCR amplicon molecules, thereby producing a library of adapter-ligated target BCR amplicon molecules comprising the target BCR repertoire.

89. A method for providing sequence of a rearranged gDNA BCR repertoire in a sample, comprising:
i) performing sequencing of the target BCR library of claim 88;
ii) determining the sequence of the library molecules, wherein determining the sequence includes obtaining initial sequence reads, aligning the initial sequence read to a reference sequence, identifying productive reads, and correcting one or more indel errors to generate rescued productive sequence reads; and
iii) reporting the sequences determined for the library molecules, thereby providing sequence of the rearranged gDNA BCR repertoire in the sample.

90. NEW A method for screening for a biomarker for a disease or condition in a subject, comprising:
performing a single multiplex amplification reaction to amplify target BCR nucleic acid template molecules from a sample from the subject according to claim 67 or claim 81;
performing sequencing of the target BCR amplicon molecules and determining the sequence of the molecules, wherein determining the sequence includes obtaining initial sequence reads, aligning the initial sequence read to a reference sequence, identifying productive reads, and correcting one or more indel errors to generate rescued productive sequence reads;
identifying BCR repertoire clonal populations from the determined target BCR sequences; and
identifying the sequence of at least one BCR clone for use as a biomarker for the disease or condition in the subject.

91. NEW The method of according to claim 67 or claim 81, wherein the nucleic acid is cDNA produced by reverse transcribing RNA molecules extracted from a biological sample.

92. NEW The method according to claim 67 or claim 81, wherein the sample comprises hematopoietic cells, lymphocytes, tumor cells, or cell-free DNA (cfDNA) and is selected from the group consisting of peripheral blood mononuclear cells (PBMCs), B cells, circulating tumor cells, tumor infiltrating lymphocytes, formalin-fixed paraffin-embedded (FFPE) tissue, fresh tissue, frozen tissue, blood, and plasma.

93. NEW A composition for analysis of a B cell receptor (BCR) repertoire in a sample, comprising at least one set of:
i) (a) a plurality of V gene primers directed to a majority of different V genes of at least one BCR coding sequence comprising at least a portion of framework region 1 (FR1) within the V gene, or
(b) a plurality of V gene primers directed to a majority of different V genes of at least one BCR coding sequence comprising at least a portion of framework region 3 (FR3) within the V gene; and
ii) (a) one or more C gene primers directed to at least a portion of a C gene of the at least one BCR coding sequence, or
(b) a plurality of J gene primers directed to at least a portion of a majority of different J genes of the at least one BCR coding sequence;
wherein each set of i) and ii) primers directed to coding sequences of the same target BCR gene selected from IgH, IgL, and IgK; and
wherein each set of i) and ii) primers directed to the same target BCR gene is configured to amplify the target BCR repertoire.

94. NEW The composition of claim 93, wherein the at least one set of i) and ii) is selected from (i) primers of Table 3 and Table 5, respectively;
(ii) primers of Table 2 and Table 5, respectively;
(iii) primers of Table 2 and Tables 6-10, respectively; and
(iv) primers of Table 3 and Tables 6-10, respectively.


SEQUENCE LISTING (605 sequences)

SEQ ID NO: 1 - DNA, 17 bp, Artificial Sequence
  source: /note=“Description of Artificial Sequence Synthetic oligonucleotide”
catgcagctgagcagcc

SEQ ID NO: 2 - DNA, 17 bp, Artificial Sequence
  source: /note=“Description of Artificial Sequence Synthetic oligonucleotide”
catggagctgagcaggc

SEQ ID NO: 3 - DNA, 17 bp, Artificial Sequence
  source: /note=“Description of Artificial Sequence Synthetic oligonucleotide”
ggagctgagcagcctga

SEQ ID NO: 4 - DNA, 17 bp, Artificial Sequence
  source: /note=“Description of Artificial Sequence Synthetic oligonucleotide”
agagctgagcagcctga

SEQ ID NO: 5 - DNA, 17 bp, Artificial Sequence
  source: /note=“Description of Artificial Sequence Synthetic oligonucleotide”
ggagctgaggagcctga

SEQ ID NO: 6 - DNA, 18 bp, Artificial Sequence
  source: /note=“Description of Artificial Sequence Synthetic oligonucleotide”
cacagacctgagcagcct

SEQ ID NO: 7 - DNA, 18 bp, Artificial Sequence
  source: /note=“Description of Artificial Sequence Synthetic oligonucleotide”
tggagctaagcagcctga

SEQ ID NO: 8 - DNA, 19 bp, Artificial Sequence
  source: /note=“Description of Artificial Sequence Synthetic oligonucleotide”
catggagctgaggagccta

SEQ ID NO: 9 - DNA, 23 bp, Artificial Sequence
  source: /note=“Description of Artificial Sequence Synthetic oligonucleotide”
atggagctaagcagtctgagatc

SEQ ID NO: 10 - DNA, 23 bp, Artificial Sequence
  source: /note=“Description of Artificial Sequence Synthetic oligonucleotide”
tccttaccatgaccaacatggac

SEQ ID NO: 11 - DNA, 24 bp, Artificial Sequence
  source: /note=“Description of Artificial Sequence Synthetic oligonucleotide”
gtggtccttacaatgaccaacatg

SEQ ID NO: 12 - DNA, 25 bp, Artificial Sequence
  source: /note=“Description of Artificial Sequence Synthetic oligonucleotide”
ggttctaacagtgatcaacatggac

SEQ ID NO: 13 - DNA, 22 bp, Artificial Sequence
  source: /note=“Description of Artificial Sequence Synthetic oligonucleotide”
acacggcgtatctgcaaatgaa

SEQ ID NO: 14 - DNA, 22 bp, Artificial Sequence
  source: /note=“Description of Artificial Sequence Synthetic oligonucleotide”
cacgctgtatgtccaaatgagc

SEQ ID NO: 15 - DNA, 23 bp, Artificial Sequence
  source: /note=“Description of Artificial Sequence Synthetic oligonucleotide”
aacacgctctacctgcaaatgaa

SEQ ID NO: 16 - DNA, 24 bp, Artificial Sequence
  source: /note=“Description of Artificial Sequence Synthetic oligonucleotide”
gaactcaccgtatctgcaaacgaa

SEQ ID NO: 17 - DNA, 24 bp, Artificial Sequence
  source: /note=“Description of Artificial Sequence Synthetic oligonucleotide”
gaacacgctgtatgttcaaatgag

SEQ ID NO: 18 - DNA, 24 bp, Artificial Sequence
  source: /note=“Description of Artificial Sequence Synthetic oligonucleotide”
gaacacgctgtttctgcaaatgaa

SEQ ID NO: 19 - DNA, 24 bp, Artificial Sequence
  source: /note=“Description of Artificial Sequence Synthetic oligonucleotide”
aagcatcgcctatctgcaaatgaa

SEQ ID NO: 20 - DNA, 24 bp, Artificial Sequence
  source: /note=“Description of Artificial Sequence Synthetic oligonucleotide”
gaacaccctgtatctgcaaacgaa

SEQ ID NO: 21 - DNA, 25 bp, Artificial Sequence
  source: /note=“Description of Artificial Sequence Synthetic oligonucleotide”
agaacacgctgtatctgcaaatgag

SEQ ID NO: 22 - DNA, 25 bp, Artificial Sequence
  source: /note=“Description of Artificial Sequence Synthetic oligonucleotide”
ggaactccctgtatctgcaaaagaa

SEQ ID NO: 23 - DNA, 25 bp, Artificial Sequence
  source: /note=“Description of Artificial Sequence Synthetic oligonucleotide”
gcaagtccctgtatctgcaaaagaa

SEQ ID NO: 24 - DNA, 25 bp, Artificial Sequence
  source: /note=“Description of Artificial Sequence Synthetic oligonucleotide”
gaacacgctgcatcttcaaatgaac

SEQ ID NO: 25 - DNA, 25 bp, Artificial Sequence
  source: /note=“Description of Artificial Sequence Synthetic oligonucleotide”
gaactcactccgtttgcaaatgaac

SEQ ID NO: 26 - DNA, 25 bp, Artificial Sequence
  source: /note=“Description of Artificial Sequence Synthetic oligonucleotide”
gaacacgctgtatctgcaaatgaac

SEQ ID NO: 27 - DNA, 26 bp, Artificial Sequence
  source: /note=“Description of Artificial Sequence Synthetic oligonucleotide”
aagaacacgctgtatcttcaaatggg

SEQ ID NO: 28 - DNA, 26 bp, Artificial Sequence
  source: /note=“Description of Artificial Sequence Synthetic oligonucleotide”
aagaactccctctatctgcaagtgaa

SEQ ID NO: 29 - DNA, 26 bp, Artificial Sequence
  source: /note=“Description of Artificial Sequence Synthetic oligonucleotide”
aagaacaggctgtatctgcaaatgaa

SEQ ID NO: 30 - DNA, 27 bp, Artificial Sequence
  source: /note=“Description of Artificial Sequence Synthetic oligonucleotide”
caaaaactccctgtatctgcaaatgaa

SEQ ID NO: 31 - DNA, 27 bp, Artificial Sequence
  source: /note=“Description of Artificial Sequence Synthetic oligonucleotide”
taagaactcaccgtatctccaaacgaa

SEQ ID NO: 32 - DNA, 27 bp, Artificial Sequence
  source: /note=“Description of Artificial Sequence Synthetic oligonucleotide”
ccagaattcactgtctctgcaaatgaa

SEQ ID NO: 33 - DNA, 27 bp, Artificial Sequence
  source: /note=“Description of Artificial Sequence Synthetic oligonucleotide”
caggaacttcctgtatcagcaaatgaa

SEQ ID NO: 34 - DNA, 27 bp, Artificial Sequence
  source: /note=“Description of Artificial Sequence Synthetic oligonucleotide”
caagaacacgctgtatcttcaaatgag

SEQ ID NO: 35 - DNA, 27 bp, Artificial Sequence
  source: /note=“Description of Artificial Sequence Synthetic oligonucleotide”
caagaactccctgtatctgcaaatgaa

SEQ ID NO: 36 - DNA, 27 bp, Artificial Sequence
  source: /note=“Description of Artificial Sequence Synthetic oligonucleotide”
caagaactcactctgtttgcaaatgaa

SEQ ID NO: 37 - DNA, 28 bp, Artificial Sequence
  source: /note=“Description of Artificial Sequence Synthetic oligonucleotide”
caagaactcactgtatctgcaaatgaac

SEQ ID NO: 38 - DNA, 28 bp, Artificial Sequence
  source: /note=“Description of Artificial Sequence Synthetic oligonucleotide”
caaaaaacacgctgtatctgcaaatgat

SEQ ID NO: 39 - DNA, 28 bp, Artificial Sequence
  source: /note=“Description of Artificial Sequence Synthetic oligonucleotide”
caaagaacacgatgtatctgcaaatgag

SEQ ID NO: 40 - DNA, 28 bp, Artificial Sequence
  source: /note=“Description of Artificial Sequence Synthetic oligonucleotide”
ccaagaactcactgtatttgctaatgaa

SEQ ID NO: 41 - DNA, 28 bp, Artificial Sequence
  source: /note=“Description of Artificial Sequence Synthetic oligonucleotide”
ccaagaactcactgtatttgcaaatgaa

SEQ ID NO: 42 - DNA, 28 bp, Artificial Sequence
  source: /note=“Description of Artificial Sequence Synthetic oligonucleotide”
ccaataactcaccgtatctgcaaatgaa

SEQ ID NO: 43 - DNA, 28 bp, Artificial Sequence
  source: /note=“Description of Artificial Sequence Synthetic oligonucleotide”
caaaaaacacgctgtatctgcaaatgaa

SEQ ID NO: 44 - DNA, 28 bp, Artificial Sequence
  source: /note=“Description of Artificial Sequence Synthetic oligonucleotide”
caagaacacgctgtatcttcaaatgaac

SEQ ID NO: 45 - DNA, 28 bp, Artificial Sequence
  source: /note=“Description of Artificial Sequence Synthetic oligonucleotide”
caaaagcatcacctatctgcaaatgaac

SEQ ID NO: 46 - DNA, 28 bp, Artificial Sequence
  source: /note=“Description of Artificial Sequence Synthetic oligonucleotide”
caagaacacacttcatctgcaaatgaac

SEQ ID NO: 47 - DNA, 28 bp, Artificial Sequence
  source: /note=“Description of Artificial Sequence Synthetic oligonucleotide”
caaaagcatcacctatctgcaaatgaag

SEQ ID NO: 48 - DNA, 29 bp, Artificial Sequence
  source: /note=“Description of Artificial Sequence Synthetic oligonucleotide”
tcaaagaactcactgtatctgcaaatgaa

SEQ ID NO: 49 - DNA, 29 bp, Artificial Sequence
  source: /note=“Description of Artificial Sequence Synthetic oligonucleotide”
gctaagaactctctgtatctgcaaatgaa

SEQ ID NO: 50 - DNA, 29 bp, Artificial Sequence
  source: /note=“Description of Artificial Sequence Synthetic oligonucleotide”
ccaagaactccttgtatcttcaaatgaac

SEQ ID NO: 51 - DNA, 29 bp, Artificial Sequence
  source: /note=“Description of Artificial Sequence Synthetic oligonucleotide”
ccaagaagtccttgtatcttcaaatgaac

SEQ ID NO: 52 - DNA, 30 bp, Artificial Sequence
  source: /note=“Description of Artificial Sequence Synthetic oligonucleotide”
gccaagaagtccttgtatcttcatatgaac

SEQ ID NO: 53 - DNA, 19 bp, Artificial Sequence
  source: /note=“Description of Artificial Sequence Synthetic oligonucleotide”
cagttccccctgaagctga

SEQ ID NO: 54 - DNA, 20 bp, Artificial Sequence
  source: /note=“Description of Artificial Sequence Synthetic oligonucleotide”
cagttctccctgaagctggg

SEQ ID NO: 55 - DNA, 20 bp, Artificial Sequence
  source: /note=“Description of Artificial Sequence Synthetic oligonucleotide”
cagttctccctgaagccgag

SEQ ID NO: 56 - DNA, 21 bp, Artificial Sequence
  source: /note=“Description of Artificial Sequence Synthetic oligonucleotide”
ccagttctccctgaagctgag

SEQ ID NO: 57 - DNA, 22 bp, Artificial Sequence
  source: /note=“Description of Artificial Sequence Synthetic oligonucleotide”
aaccagttctccctgaacctga

SEQ ID NO: 58 - DNA, 22 bp, Artificial Sequence
  source: /note=“Description of Artificial Sequence Synthetic oligonucleotide”
aaccacttctccctgaagctga

SEQ ID NO: 59 - DNA, 23 bp, Artificial Sequence
  source: /note=“Description of Artificial Sequence Synthetic oligonucleotide”
gaaccaattctccctgaagctga

SEQ ID NO: 60 - DNA, 24 bp, Artificial Sequence
  source: /note=“Description of Artificial Sequence Synthetic oligonucleotide”
agaaccagttctacctgaagctga

SEQ ID NO: 61 - DNA, 24 bp, Artificial Sequence
  source: /note=“Description of Artificial Sequence Synthetic oligonucleotide”
agaagcagttctacctgaagctga

SEQ ID NO: 62 - DNA, 17 bp, Artificial Sequence
  source: /note=“Description of Artificial Sequence Synthetic oligonucleotide”
gcagtggagcagcctga

SEQ ID NO: 63 - DNA, 19 bp, Artificial Sequence
  source: /note=“Description of Artificial Sequence Synthetic oligonucleotide”
cagttctccctgcagctga

SEQ ID NO: 64 - DNA, 21 bp, Artificial Sequence
  source: /note=“Description of Artificial Sequence Synthetic oligonucleotide”
atctgcagatcagcacgctaa

SEQ ID NO: 65 - DNA, 22 bp, Artificial Sequence
  source: /note=“Description of Artificial Sequence Synthetic oligonucleotide”
tatctgcagatcagcagcctaa

SEQ ID NO: 66 - DNA, 22 bp, Artificial Sequence
  source: /note=“Description of Artificial Sequence Synthetic oligonucleotide”
tgtcttcagatcagcagcctaa

SEQ ID NO: 67 - DNA, 22 bp, Artificial Sequence
  source: /note=“Description of Artificial Sequence Synthetic oligonucleotide”
tacctgcagatcagcagcctaa

SEQ ID NO: 68 - DNA, 22 bp, Artificial Sequence
  source: /note=“Description of Artificial Sequence Synthetic oligonucleotide”
tatctgcagatctgcagcctaa

SEQ ID NO: 69 - RNA, 17 nt, Artificial Sequence
  source: /note=“Description of Artificial Sequence Synthetic oligonucleotide”
caugcagcugagcagcc

SEQ ID NO: 70 - RNA, 17 nt, Artificial Sequence
  source: /note=“Description of Artificial Sequence Synthetic oligonucleotide”
cauggagcugagcaggc

SEQ ID NO: 71 - RNA, 17 nt, Artificial Sequence
  source: /note=“Description of Artificial Sequence Synthetic oligonucleotide”
ggagcugagcagccuga

SEQ ID NO: 72 - RNA, 17 nt, Artificial Sequence
  source: /note=“Description of Artificial Sequence Synthetic oligonucleotide”
agagcugagcagccuga

SEQ ID NO: 73 - RNA, 17 nt, Artificial Sequence
  source: /note=“Description of Artificial Sequence Synthetic oligonucleotide”
ggagcugaggagccuga

SEQ ID NO: 74 - DNA, 18 bp, Artificial Sequence
  source: /note=“Description of Artificial Sequence Synthetic oligonucleotide”
cacagacctgagcagcct

SEQ ID NO: 75 - DNA, 18 bp, Artificial Sequence
  source: /note=“Description of Artificial Sequence Synthetic oligonucleotide”
  source: /note=“Description of Combined DNA/RNA Molecule Synthetic oligonucleotide”
tggagcuaagcagccuga

SEQ ID NO: 76 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
catggagcugaggagccua

SEQ ID NO: 77 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
atggagcuaagcagtctgagauc

SEQ ID NO: 78 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
tccttaccaugaccaacauggac

SEQ ID NO: 79 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gtggtcctuacaatgaccaacaug

SEQ ID NO: 80 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
ggttctaacagugatcaacauggac

SEQ ID NO: 81 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
acacggcguatctgcaaaugaa

SEQ ID NO: 82 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
cacgctguatgtccaaaugagc

SEQ ID NO: 83 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
aacacgctcuacctgcaaaugaa

SEQ ID NO: 84 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gaacucaccgtatcugcaaacgaa

SEQ ID NO: 85 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gaacacgcugtatgttcaaaugag

SEQ ID NO: 86 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gaacacgcugtttctgcaaaugaa

SEQ ID NO: 87 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
aagcatcgccuatctgcaaaugaa

SEQ ID NO: 88 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gaacacccugtatcugcaaacgaa

SEQ ID NO: 89 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
agaacacgcugtatctgcaaaugag

SEQ ID NO: 90 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
ggaacuccctgtatcugcaaaagaa

SEQ ID NO: 91 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
gcaaguccctgtatcugcaaaagaa

SEQ ID NO: 92 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
gaacacgcugcatcttcaaaugaac

SEQ ID NO: 93 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
gaactcacuccgtttgcaaaugaac

SEQ ID NO: 94 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
gaacacgcugtatctgcaaaugaac

SEQ ID NO: 95 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
aagaacacgcugtatcttcaaauggg

SEQ ID NO: 96 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
aagaactcccuctatctgcaagugaa

SEQ ID NO: 97 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
aagaacaggcugtatctgcaaaugaa

SEQ ID NO: 98 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
caaaaactcccugtatctgcaaaugaa

SEQ ID NO: 99 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
taagaacucaccgtatcuccaaacgaa

SEQ ID NO: 100 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
ccagaattcacugtctctgcaaaugaa

SEQ ID NO: 101 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
caggaacttccugtatcagcaaaugaa

SEQ ID NO: 102 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
caagaacacgcugtatcttcaaaugag

SEQ ID NO: 103 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
caagaactcccugtatctgcaaaugaa

SEQ ID NO: 104 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
caagaactcacuctgtttgcaaaugaa

SEQ ID NO: 105 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
caagaactcacugtatctgcaaaugaac

SEQ ID NO: 106 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
caaaaaacacgcugtatctgcaaaugat

SEQ ID NO: 107 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
caaagaacacgaugtatctgcaaaugag

SEQ ID NO: 108 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
ccaagaactcacugtatttgctaaugaa

SEQ ID NO: 109 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
ccaagaactcacugtatttgcaaaugaa

SEQ ID NO: 110 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
ccaataactcaccguatctgcaaaugaa

SEQ ID NO: 111 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
caaaaaacacgcugtatctgcaaaugaa

SEQ ID NO: 112 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
caagaacacgcugtatcttcaaaugaac

SEQ ID NO: 113 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
caaaagcatcaccuatctgcaaaugaac

SEQ ID NO: 114 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
caagaacacacutcatctgcaaaugaac

SEQ ID NO: 115 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
caaaagcatcaccuatctgcaaaugaag

SEQ ID NO: 116 - DNA, 29 bp, Artificial Sequence
  source: /note=“Description of Artificial Sequence Synthetic oligonucleotide”
  source: /note=“Description of Combined DNA/RNA Molecule Synthetic oligonucleotide”
tcaaagaactcacugtatctgcaaaugaa

SEQ ID NO: 117 - DNA, 29 bp, Artificial Sequence
  source: /note=“Description of Artificial Sequence Synthetic oligonucleotide”
  source: /note=“Description of Combined DNA/RNA Molecule Synthetic oligonucleotide”
gctaagaactcuctgtatctgcaaaugaa

SEQ ID NO: 118 - DNA, 29 bp, Artificial Sequence
  source: /note=“Description of Artificial Sequence Synthetic oligonucleotide”
  source: /note=“Description of Combined DNA/RNA Molecule Synthetic oligonucleotide”
ccaagaactccutgtatcttcaaaugaac

SEQ ID NO: 119 - DNA, 29 bp, Artificial Sequence
  source: /note=“Description of Artificial Sequence Synthetic oligonucleotide”
  source: /note=“Description of Combined DNA/RNA Molecule Synthetic oligonucleotide”
ccaagaagtccutgtatcttcaaaugaac

SEQ ID NO: 120 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
gccaagaagtccutgtatcttcataugaac

SEQ ID NO: 121 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
cagttcccccugaagcuga

SEQ ID NO: 122 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
cagttcuccctgaagcuggg

SEQ ID NO: 123 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
cagutctcccugaagccgag

SEQ ID NO: 124 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ccagttcuccctgaagcugag

SEQ ID NO: 125 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
aaccagttcuccctgaaccuga

SEQ ID NO: 126 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
aaccacttcuccctgaagcuga

SEQ ID NO: 127 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
gaaccaattcuccctgaagcuga

SEQ ID NO: 128 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
agaaccagttcuacctgaagcuga

SEQ ID NO: 129 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
agaagcagttcuacctgaagcuga

SEQ ID NO: 130 - RNA, 17 nt, Artificial Sequence
  source: /note=“Description of Artificial Sequence Synthetic oligonucleotide”
gcaguggagcagccuga

SEQ ID NO: 131 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
cagttcuccctgcagcuga

SEQ ID NO: 132 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
atctgcagaucagcacgcuaa

SEQ ID NO: 133 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
tatctgcagaucagcagccuaa

SEQ ID NO: 134 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
tgtcttcagaucagcagccuaa

SEQ ID NO: 135 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
tacctgcagaucagcagccuaa

SEQ ID NO: 136 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
tatctgcagauctgcagccuaa

SEQ ID NO: 137 - DNA, 23 bp, Artificial Sequence
  source: /note=“Description of Artificial Sequence Synthetic oligonucleotide”
agtgaaggtttcctgcaaggcat

SEQ ID NO: 138 - DNA, 23 bp, Artificial Sequence
  source: /note=“Description of Artificial Sequence Synthetic oligonucleotide”
agtgaaggtctcctgcaaggttt

SEQ ID NO: 139 - DNA, 23 bp, Artificial Sequence
  source: /note=“Description of Artificial Sequence Synthetic oligonucleotide”
agtgaaggtttcctgcaaggctt

SEQ ID NO: 140 - DNA, 23 bp, Artificial Sequence
  source: /note=“Description of Artificial Sequence Synthetic oligonucleotide”
agtgaaggtctcctgcaaggctt

SEQ ID NO: 141 - DNA, 23 bp, Artificial Sequence
  source: /note=“Description of Artificial Sequence Synthetic oligonucleotide”
agtgaaaatctcctgcaaggttt

SEQ ID NO: 142 - DNA, 23 bp, Artificial Sequence
  source: /note=“Description of Artificial Sequence Synthetic oligonucleotide”
ggtgaaggtctcctgcaaggctt

SEQ ID NO: 143 - DNA, 19 bp, Artificial Sequence
  source: /note=“Description of Artificial Sequence Synthetic oligonucleotide”
acgctgacctgcaccttct

SEQ ID NO: 144 - DNA, 19 bp, Artificial Sequence
  source: /note=“Description of Artificial Sequence Synthetic oligonucleotide”
acgctgacccgcaccttct

SEQ ID NO: 145 - DNA, 19 bp, Artificial Sequence
  source: /note=“Description of Artificial Sequence Synthetic oligonucleotide”
acactgacctgcgccttct

SEQ ID NO: 146 - DNA, 19 bp, Artificial Sequence
  source: /note=“Description of Artificial Sequence Synthetic oligonucleotide”
acgctgacctgcaccgtct

SEQ ID NO: 147 - DNA, 19 bp, Artificial Sequence
  source: /note=“Description of Artificial Sequence Synthetic oligonucleotide”
acactgacctgcaccttct

SEQ ID NO: 148 - DNA, 22 bp, Artificial Sequence
  source: /note=“Description of Artificial Sequence Synthetic oligonucleotide”
ctgaaactctcctgtgcagcct

SEQ ID NO: 149 - DNA, 22 bp, Artificial Sequence
  source: /note=“Description of Artificial Sequence Synthetic oligonucleotide”
ctgagactctcctttgcagcct

SEQ ID NO: 150 - DNA, 22 bp, Artificial Sequence
  source: /note=“Description of Artificial Sequence Synthetic oligonucleotide”
ctgagactctcctgttcagcct

SEQ ID NO: 151 - DNA, 22 bp, Artificial Sequence
  source: /note=“Description of Artificial Sequence Synthetic oligonucleotide”
cttagactctcctgtgcagcct

SEQ ID NO: 152 - DNA, 22 bp, Artificial Sequence
  source: /note=“Description of Artificial Sequence Synthetic oligonucleotide”
ctgagactctcctgtgcagcct

SEQ ID NO: 153 - DNA, 22 bp, Artificial Sequence
  source: /note=“Description of Artificial Sequence Synthetic oligonucleotide”
ctgagactctcctgtgcagcgt

SEQ ID NO: 154 - DNA, 22 bp, Artificial Sequence
  source: /note=“Description of Artificial Sequence Synthetic oligonucleotide”
ctgagactctcctgtacagctt

SEQ ID NO: 155 - DNA, 20 bp, Artificial Sequence
  source: /note=“Description of Artificial Sequence Synthetic oligonucleotide”
gtccctcacctgcgctgtct

SEQ ID NO: 156 - DNA, 20 bp, Artificial Sequence
  source: /note=“Description of Artificial Sequence Synthetic oligonucleotide”
gtccctcacctgtactgtct

SEQ ID NO: 157 - DNA, 20 bp, Artificial Sequence
  source: /note=“Description of Artificial Sequence Synthetic oligonucleotide”
gtccctcacctgcactgtct

SEQ ID NO: 158 - DNA, 20 bp, Artificial Sequence
  source: /note=“Description of Artificial Sequence Synthetic oligonucleotide”
gtccctcacctgcgctatct

SEQ ID NO: 159 - DNA, 20 bp, Artificial Sequence
  source: /note=“Description of Artificial Sequence Synthetic oligonucleotide”
gtccctcacctgcactgtca

SEQ ID NO: 160 - DNA, 26 bp, Artificial Sequence
  source: /note=“Description of Artificial Sequence Synthetic oligonucleotide”
gtctctgaagatctcctgtaagggtt

SEQ ID NO: 161 - DNA, 26 bp, Artificial Sequence
  source: /note=“Description of Artificial Sequence Synthetic oligonucleotide”
gtctctgaggatctcctgtaagggtt

SEQ ID NO: 162 - DNA, 23 bp, Artificial Sequence
  source: /note=“Description of Artificial Sequence Synthetic oligonucleotide”
cctctcactcacctgtgccatct

SEQ ID NO: 163 - DNA, 23 bp, Artificial Sequence
  source: /note=“Description of Artificial Sequence Synthetic oligonucleotide”
agtgaaggtttcctgcaaggctt

SEQ ID NO: 164 - DNA, 21 bp, Artificial Sequence
  source: /note=“Description of Artificial Sequence Synthetic oligonucleotide”
gggctgaggtgaagaagcttg

SEQ ID NO: 165 - DNA, 21 bp, Artificial Sequence
  source: /note=“Description of Artificial Sequence Synthetic oligonucleotide”
gagctgaggtgaagaagcctg

SEQ ID NO: 166 - DNA, 21 bp, Artificial Sequence
  source: /note=“Description of Artificial Sequence Synthetic oligonucleotide”
ggtctgagttgaagaagcctg

SEQ ID NO: 167 - DNA, 21 bp, Artificial Sequence
  source: /note=“Description of Artificial Sequence Synthetic oligonucleotide”
gggctgaggtgaagaagactg

SEQ ID NO: 168 - DNA, 21 bp, Artificial Sequence
  source: /note=“Description of Artificial Sequence Synthetic oligonucleotide”
ggcctgaggtgaagaagcctg

SEQ ID NO: 169 - DNA, 21 bp, Artificial Sequence
  source: /note=“Description of Artificial Sequence Synthetic oligonucleotide”
gggctgaggtgaagaagcctg

SEQ ID NO: 170 - DNA, 22 bp, Artificial Sequence
  source: /note=“Description of Artificial Sequence Synthetic oligonucleotide”
gtctggtcctacgctggtaaaa

SEQ ID NO: 171 - DNA, 22 bp, Artificial Sequence
  source: /note=“Description of Artificial Sequence Synthetic oligonucleotide”
gtctggtcctgcgctggtgaaa

SEQ ID NO: 172 - DNA, 22 bp, Artificial Sequence
  source: /note=“Description of Artificial Sequence Synthetic oligonucleotide”
gtctggtcctgtgctggtgaaa

SEQ ID NO: 173 - DNA, 22 bp, Artificial Sequence
  source: /note=“Description of Artificial Sequence Synthetic oligonucleotide”
gtctggtcctacgctggtgaaa

SEQ ID NO: 174 - DNA, 21 bp, Artificial Sequence
  source: /note=“Description of Artificial Sequence Synthetic oligonucleotide”
ggtcccttagactctcctgtg

SEQ ID NO: 175 - DNA, 21 bp, Artificial Sequence
  source: /note=“Description of Artificial Sequence Synthetic oligonucleotide”
ggtccctgagactctcctgta

SEQ ID NO: 176 - DNA, 21 bp, Artificial Sequence
  source: /note=“Description of Artificial Sequence Synthetic oligonucleotide”
cgtccctgagactctcctgta

SEQ ID NO: 177 - DNA, 21 bp, Artificial Sequence
  source: /note=“Description of Artificial Sequence Synthetic oligonucleotide”
ggtccctgaaactctcctgtg

SEQ ID NO: 178 - DNA, 21 bp, Artificial Sequence
  source: /note=“Description of Artificial Sequence Synthetic oligonucleotide”
ggtccctgagactctcctttg

SEQ ID NO: 179 - DNA, 21 bp, Artificial Sequence
  source: /note=“Description of Artificial Sequence Synthetic oligonucleotide”
gggccctgagactctcctgtg

SEQ ID NO: 180 - DNA, 21 bp, Artificial Sequence
  source: /note=“Description of Artificial Sequence Synthetic oligonucleotide”
ggtccctgagactctcctgtt

SEQ ID NO: 181 - DNA, 21 bp, Artificial Sequence
  source: /note=“Description of Artificial Sequence Synthetic oligonucleotide”
ggtccctgagactctcctgtg

SEQ ID NO: 182 - DNA, 19 bp, Artificial Sequence
  source: /note=“Description of Artificial Sequence Synthetic oligonucleotide”
gggcgcaggactgttgaag

SEQ ID NO: 183 - DNA, 19 bp, Artificial Sequence
  source: /note=“Description of Artificial Sequence Synthetic oligonucleotide”
aggtccaggactggtgaag

SEQ ID NO: 184 - DNA, 19 bp, Artificial Sequence
  source: /note=“Description of Artificial Sequence Synthetic oligonucleotide”
cggctcaggactggtgaag

SEQ ID NO: 185 - DNA, 19 bp, Artificial Sequence
  source: /note=“Description of Artificial Sequence Synthetic oligonucleotide”
gggcccaggactgttgaag

SEQ ID NO: 186 - DNA, 19 bp, Artificial Sequence
  source: /note=“Description of Artificial Sequence Synthetic oligonucleotide”
gggcccaggactggtgaag

SEQ ID NO: 187 - DNA, 19 bp, Artificial Sequence
  source: /note=“Description of Artificial Sequence Synthetic oligonucleotide”
aggtccgggactggtgaag

SEQ ID NO: 188 - DNA, 22 bp, Artificial Sequence
  source: /note=“Description of Artificial Sequence Synthetic oligonucleotide”
gcagtctggagcagaggtgaaa

SEQ ID NO: 189 - DNA, 22 bp, Artificial Sequence
  source: /note=“Description of Artificial Sequence Synthetic oligonucleotide”
gcagtccggagcagaggtgaaa

SEQ ID NO: 190 - DNA, 21 bp, Artificial Sequence
  source: /note=“Description of Artificial Sequence Synthetic oligonucleotide”
ggctgaggtgaagaagcctgg

SEQ ID NO: 191 - DNA, 21 bp, Artificial Sequence
  source: /note=“Description of Artificial Sequence Synthetic oligonucleotide”
agctgaggtgaagaagcctgg

SEQ ID NO: 192 - DNA, 21 bp, Artificial Sequence
  source: /note=“Description of Artificial Sequence Synthetic oligonucleotide”
gcctgaggtgaagaagcctgg

SEQ ID NO: 193 - DNA, 21 bp, Artificial Sequence
  source: /note=“Description of Artificial Sequence Synthetic oligonucleotide”
ggctgaggtgaagaagcttgg

SEQ ID NO: 194 - DNA, 21 bp, Artificial Sequence
  source: /note=“Description of Artificial Sequence Synthetic oligonucleotide”
ggctgaggtgaagaagactgg

SEQ ID NO: 195 - DNA, 21 bp, Artificial Sequence
  source: /note=“Description of Artificial Sequence Synthetic oligonucleotide”
gtctgagttgaagaagcctgg

SEQ ID NO: 196 - DNA, 20 bp, Artificial Sequence
  source: /note=“Description of Artificial Sequence Synthetic oligonucleotide”
gtcctacgctggtgaaaccc

SEQ ID NO: 197 - DNA, 20 bp, Artificial Sequence
  source: /note=“Description of Artificial Sequence Synthetic oligonucleotide”
gtcctgtgctggtgaaaccc

SEQ ID NO: 198 - DNA, 20 bp, Artificial Sequence
  source: /note=“Description of Artificial Sequence Synthetic oligonucleotide”
gtcctgcgctggtgaaaccc

SEQ ID NO: 199 - DNA, 20 bp, Artificial Sequence
  source: /note=“Description of Artificial Sequence Synthetic oligonucleotide”
gtcctacgctggtaaaaccc

SEQ ID NO: 200 - DNA, 20 bp, Artificial Sequence
  source: /note=“Description of Artificial Sequence Synthetic oligonucleotide”
gtccaggactggtgaagccc

SEQ ID NO: 201 - DNA, 20 bp, Artificial Sequence
  source: /note=“Description of Artificial Sequence Synthetic oligonucleotide”
gcccaggactgttgaagcct

SEQ ID NO: 202 - DNA, 20 bp, Artificial Sequence
  source: /note=“Description of Artificial Sequence Synthetic oligonucleotide”
gtccgggactggtgaagccc

SEQ ID NO: 203 - DNA, 20 bp, Artificial Sequence
  source: /note=“Description of Artificial Sequence Synthetic oligonucleotide”
gctcaggactggtgaagcct

SEQ ID NO: 204 - DNA, 20 bp, Artificial Sequence
  source: /note=“Description of Artificial Sequence Synthetic oligonucleotide”
gcccaggactggtgaagcct

SEQ ID NO: 205 - DNA, 20 bp, Artificial Sequence
  source: /note=“Description of Artificial Sequence Synthetic oligonucleotide”
gcgcaggactgttgaagcct

SEQ ID NO: 206 - DNA, 23 bp, Artificial Sequence
  source: /note=“Description of Artificial Sequence Synthetic oligonucleotide”
gcagtccggagcagaggtgaaaa

SEQ ID NO: 207 - DNA, 23 bp, Artificial Sequence
  source: /note=“Description of Artificial Sequence Synthetic oligonucleotide”
gcagtctggagcagaggtgaaaa

SEQ ID NO: 208 - DNA, 22 bp, Artificial Sequence
  source: /note=“Description of Artificial Sequence Synthetic oligonucleotide”
ggtctgagttgaagaagcctgg

SEQ ID NO: 209 - DNA, 22 bp, Artificial Sequence
  source: /note=“Description of Artificial Sequence Synthetic oligonucleotide”
ggcctgaggtgaagaagcctgg

SEQ ID NO: 210 - DNA, 22 bp, Artificial Sequence
  source: /note=“Description of Artificial Sequence Synthetic oligonucleotide”
gggctgaggtgaagaagactgg

SEQ ID NO: 211 - DNA, 22 bp, Artificial Sequence
  source: /note=“Description of Artificial Sequence Synthetic oligonucleotide”
gggctgaggtgaagaagcttgg

SEQ ID NO: 212 - DNA, 22 bp, Artificial Sequence
  source: /note=“Description of Artificial Sequence Synthetic oligonucleotide”
gagctgaggtgaagaagcctgg

SEQ ID NO: 213 - DNA, 22 bp, Artificial Sequence
  source: /note=“Description of Artificial Sequence Synthetic oligonucleotide”
gggctgaggtgaagaagcctgg

SEQ ID NO: 214 - DNA, 20 bp, Artificial Sequence
  source: /note=“Description of Artificial Sequence Synthetic oligonucleotide”
cctgcgctggtgaaacccac

SEQ ID NO: 215 - DNA, 20 bp, Artificial Sequence
  source: /note=“Description of Artificial Sequence Synthetic oligonucleotide”
cctgtgctggtgaaacccac

SEQ ID NO: 216 - DNA, 20 bp, Artificial Sequence
  source: /note=“Description of Artificial Sequence Synthetic oligonucleotide”
cctacgctggtgaaacccac

SEQ ID NO: 217 - DNA, 20 bp, Artificial Sequence
  source: /note=“Description of Artificial Sequence Synthetic oligonucleotide”
cctacgctggtaaaacccac

SEQ ID NO: 218 - DNA, 22 bp, Artificial Sequence
  source: /note=“Description of Artificial Sequence Synthetic oligonucleotide”
ggctgaggtgaagaagcctggg

SEQ ID NO: 219 - DNA, 22 bp, Artificial Sequence
  source: /note=“Description of Artificial Sequence Synthetic oligonucleotide”
gcctgaggtgaagaagcctggg

SEQ ID NO: 220 - DNA, 22 bp, Artificial Sequence
  source: /note=“Description of Artificial Sequence Synthetic oligonucleotide”
ggctgaggtgaagaagcttggg

SEQ ID NO: 221 - DNA, 22 bp, Artificial Sequence
  source: /note=“Description of Artificial Sequence Synthetic oligonucleotide”
agctgaggtgaagaagcctggg

SEQ ID NO: 222 - DNA, 22 bp, Artificial Sequence
  source: /note=“Description of Artificial Sequence Synthetic oligonucleotide”
gtctgagttgaagaagcctggg

SEQ ID NO: 223 - DNA, 22 bp, Artificial Sequence
  source: /note=“Description of Artificial Sequence Synthetic oligonucleotide”
ggctgaggtgaagaagactggg

SEQ ID NO: 224 - DNA, 20 bp, Artificial Sequence
  source: /note=“Description of Artificial Sequence Synthetic oligonucleotide”
tgcgctggtgaaacccacac

SEQ ID NO: 225 - DNA, 20 bp, Artificial Sequence
  source: /note=“Description of Artificial Sequence Synthetic oligonucleotide”
tacgctggtaaaacccacac

SEQ ID NO: 226 - DNA, 20 bp, Artificial Sequence
  source: /note=“Description of Artificial Sequence Synthetic oligonucleotide”
tgtgctggtgaaacccacag

SEQ ID NO: 227 - DNA, 20 bp, Artificial Sequence
  source: /note=“Description of Artificial Sequence Synthetic oligonucleotide”
tacgctggtgaaacccacac

SEQ ID NO: 228 - DNA, 22 bp, Artificial Sequence
  source: /note=“Description of Artificial Sequence Synthetic oligonucleotide”
tcctcggtgaaggtctcctgca

SEQ ID NO: 229 - DNA, 22 bp, Artificial Sequence
  source: /note=“Description of Artificial Sequence Synthetic oligonucleotide”
gcctcagtgaaggtctcctgca

SEQ ID NO: 230 - DNA, 22 bp, Artificial Sequence
  source: /note=“Description of Artificial Sequence Synthetic oligonucleotide”
tcctcagtgaaggtctcctgca

SEQ ID NO: 231 - DNA, 22 bp, Artificial Sequence
  source: /note=“Description of Artificial Sequence Synthetic oligonucleotide”
acctcagtgaaggtctcctgca

SEQ ID NO: 232 - DNA, 22 bp, Artificial Sequence
  source: /note=“Description of Artificial Sequence Synthetic oligonucleotide”
gcctcagtgaaggtttcctgca

SEQ ID NO: 233 - DNA, 22 bp, Artificial Sequence
  source: /note=“Description of Artificial Sequence Synthetic oligonucleotide”
tcctcagtgaaggtttcctgca

SEQ ID NO: 234 - DNA, 22 bp, Artificial Sequence
  source: /note=“Description of Artificial Sequence Synthetic oligonucleotide”
gctacagtgaaaatctcctgca

SEQ ID NO: 235 - DNA, 20 bp, Artificial Sequence
  source: /note=“Description of Artificial Sequence Synthetic oligonucleotide”
cagaccctcacactgacctg

SEQ ID NO: 236 - DNA, 20 bp, Artificial Sequence
  source: /note=“Description of Artificial Sequence Synthetic oligonucleotide”
gagaccctcacgctgacctg

SEQ ID NO: 237 - DNA, 20 bp, Artificial Sequence
  source: /note=“Description of Artificial Sequence Synthetic oligonucleotide”
cagaccctcacgctgacctg

SEQ ID NO: 238 - DNA, 20 bp, Artificial Sequence
  source: /note=“Description of Artificial Sequence Synthetic oligonucleotide”
cagaccctcacgctgacccg

SEQ ID NO: 239 - DNA, 20 bp, Artificial Sequence
  source: /note=“Description of Artificial Sequence Synthetic oligonucleotide”
ctgtccctcacctgtactgt

SEQ ID NO: 240 - DNA, 20 bp, Artificial Sequence
  source: /note=“Description of Artificial Sequence Synthetic oligonucleotide”
ccgtccctcacctgcactgt

SEQ ID NO: 241 - DNA, 20 bp, Artificial Sequence
  source: /note=“Description of Artificial Sequence Synthetic oligonucleotide”
ctctcactcacctgtgccat

SEQ ID NO: 242 - DNA, 20 bp, Artificial Sequence
  source: /note=“Description of Artificial Sequence Synthetic oligonucleotide”
ctgtccctcacctgcactgt

SEQ ID NO: 243 - DNA, 20 bp, Artificial Sequence
  source: /note=“Description of Artificial Sequence Synthetic oligonucleotide”
ctgtccctcacctgcgctat

SEQ ID NO: 244 - DNA, 20 bp, Artificial Sequence
  source: /note=“Description of Artificial Sequence Synthetic oligonucleotide”
ctgtccctcacctgcgctgt

SEQ ID NO: 245 - DNA, 24 bp, Artificial Sequence
  source: /note=“Description of Artificial Sequence Synthetic oligonucleotide”
gggagtctctgaggatctcctgta

SEQ ID NO: 246 - DNA, 24 bp, Artificial Sequence
  source: /note=“Description of Artificial Sequence Synthetic oligonucleotide”
gggagtctctgaagatctcctgta

SEQ ID NO: 247 - DNA, 22 bp, Artificial Sequence
  source: /note=“Description of Artificial Sequence Synthetic oligonucleotide”
tcggtgaaggtctcctgcaagg

SEQ ID NO: 248 - DNA, 22 bp, Artificial Sequence
  source: /note=“Description of Artificial Sequence Synthetic oligonucleotide”
tcagtgaaggtttcctgcaagg

SEQ ID NO: 249 - DNA, 22 bp, Artificial Sequence
  source: /note=“Description of Artificial Sequence Synthetic oligonucleotide”
acagtgaaaatctcctgcaagg

SEQ ID NO: 250 - DNA, 22 bp, Artificial Sequence
  source: /note=“Description of Artificial Sequence Synthetic oligonucleotide”
tcagtgaaggtctcctgcaagg

SEQ ID NO: 251 - DNA, 19 bp, Artificial Sequence
  source: /note=“Description of Artificial Sequence Synthetic oligonucleotide”
tcacgctgacctgcaccgt

SEQ ID NO: 252 - DNA, 19 bp, Artificial Sequence
  source: /note=“Description of Artificial Sequence Synthetic oligonucleotide”
tcacgctgacccgcacctt

SEQ ID NO: 253 - DNA, 19 bp, Artificial Sequence
  source: /note=“Description of Artificial Sequence Synthetic oligonucleotide”
tcacactgacctgcgcctt

SEQ ID NO: 254 - DNA, 19 bp, Artificial Sequence
  source: /note=“Description of Artificial Sequence Synthetic oligonucleotide”
tcacactgacctgcacctt

SEQ ID NO: 255 - DNA, 19 bp, Artificial Sequence
  source: /note=“Description of Artificial Sequence Synthetic oligonucleotide”
tcacgctgacctgcacctt

SEQ ID NO: 256 - DNA, 22 bp, Artificial Sequence
  source: /note=“Description of Artificial Sequence Synthetic oligonucleotide”
ccctgagactctcctgtacagc

SEQ ID NO: 257 - DNA, 22 bp, Artificial Sequence
  source: /note=“Description of Artificial Sequence Synthetic oligonucleotide”
ccctgagactctcctgttcagc

SEQ ID NO: 258 - DNA, 22 bp, Artificial Sequence
  source: /note=“Description of Artificial Sequence Synthetic oligonucleotide”
ccctgagactctcctttgcagc

SEQ ID NO: 259 - DNA, 22 bp, Artificial Sequence
  source: /note=“Description of Artificial Sequence Synthetic oligonucleotide”
ccctgaaactctcctgtgcagc

SEQ ID NO: 260 - DNA, 22 bp, Artificial Sequence
  source: /note=“Description of Artificial Sequence Synthetic oligonucleotide”
ccctgagactctcctgtgcagc

SEQ ID NO: 261 - DNA, 22 bp, Artificial Sequence
  source: /note=“Description of Artificial Sequence Synthetic oligonucleotide”
cccttagactctcctgtgcagc

SEQ ID NO: 262 - DNA, 26 bp, Artificial Sequence
  source: /note=“Description of Artificial Sequence Synthetic oligonucleotide”
gagtctctgaagatctcctgtaaggg

SEQ ID NO: 263 - DNA, 26 bp, Artificial Sequence
  source: /note=“Description of Artificial Sequence Synthetic oligonucleotide”
gagtctctgaggatctcctgtaaggg

SEQ ID NO: 264 - DNA, 24 bp, Artificial Sequence
  source: /note=“Description of Artificial Sequence Synthetic oligonucleotide”
gtgaaggtctcctgcaaggcttct

SEQ ID NO: 265 - DNA, 24 bp, Artificial Sequence
  source: /note=“Description of Artificial Sequence Synthetic oligonucleotide”
gtgaaaatctcctgcaaggtttct

SEQ ID NO: 266 - DNA, 24 bp, Artificial Sequence
  source: /note=“Description of Artificial Sequence Synthetic oligonucleotide”
gtgaaggtttcctgcaaggcatct

SEQ ID NO: 267 - DNA, 24 bp, Artificial Sequence
  source: /note=“Description of Artificial Sequence Synthetic oligonucleotide”
gtgaaggtttcctgcaaggcttct

SEQ ID NO: 268 - DNA, 24 bp, Artificial Sequence
  source: /note=“Description of Artificial Sequence Synthetic oligonucleotide”
gtgaaggtttcctgcaaggcttcc

SEQ ID NO: 269 - DNA, 24 bp, Artificial Sequence
  source: /note=“Description of Artificial Sequence Synthetic oligonucleotide”
gtgaaggtctcctgcaaggtttcc

SEQ ID NO: 270 - DNA, 20 bp, Artificial Sequence
  source: /note=“Description of Artificial Sequence Synthetic oligonucleotide”
acgctgacccgcaccttctc

SEQ ID NO: 271 - DNA, 20 bp, Artificial Sequence
  source: /note=“Description of Artificial Sequence Synthetic oligonucleotide”
acgctgacctgcaccttctc

SEQ ID NO: 272 - DNA, 20 bp, Artificial Sequence
  source: /note=“Description of Artificial Sequence Synthetic oligonucleotide”
acgctgacctgcaccgtctc

SEQ ID NO: 273 - DNA, 20 bp, Artificial Sequence
  source: /note=“Description of Artificial Sequence Synthetic oligonucleotide”
acactgacctgcgccttctc

SEQ ID NO: 274 - DNA, 20 bp, Artificial Sequence
  source: /note=“Description of Artificial Sequence Synthetic oligonucleotide”
acactgacctgcaccttctc

SEQ ID NO: 275 - DNA, 21 bp, Artificial Sequence
  source: /note=“Description of Artificial Sequence Synthetic oligonucleotide”
gtccctcacctgcactgtctc

SEQ ID NO: 276 - DNA, 21 bp, Artificial Sequence
  source: /note=“Description of Artificial Sequence Synthetic oligonucleotide”
gtccctcacctgtactgtctc

SEQ ID NO: 277 - DNA, 21 bp, Artificial Sequence
  source: /note=“Description of Artificial Sequence Synthetic oligonucleotide”
gtccctcacctgcgctatctc

SEQ ID NO: 278 - DNA, 21 bp, Artificial Sequence
  source: /note=“Description of Artificial Sequence Synthetic oligonucleotide”
gtccctcacctgcactgtcac

SEQ ID NO: 279 - DNA, 21 bp, Artificial Sequence
  source: /note=“Description of Artificial Sequence Synthetic oligonucleotide”
ctcactcacctgtgccatctc

SEQ ID NO: 280 - DNA, 21 bp, Artificial Sequence
  source: /note=“Description of Artificial Sequence Synthetic oligonucleotide”
gtccctcacctgcgctgtcta

SEQ ID NO: 281 - DNA, 21 bp, Artificial Sequence
  source: /note=“Description of Artificial Sequence Synthetic oligonucleotide”
gtccctcacctgcgctgtctc

SEQ ID NO: 282 - DNA, 27 bp, Artificial Sequence
  source: /note=“Description of Artificial Sequence Synthetic oligonucleotide”
gtctctgaggatctcctgtaagggttc

SEQ ID NO: 283 - DNA, 27 bp, Artificial Sequence
  source: /note=“Description of Artificial Sequence Synthetic oligonucleotide”
gtctctgaagatctcctgtaagggttc

SEQ ID NO: 284 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
agtgaagguttccugcaaggcat

SEQ ID NO: 285 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
agtgaaggtcucctgcaaggtut

SEQ ID NO: 286 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
agtgaaggttucctgcaaggcut

SEQ ID NO: 287 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
agtgaaggtcucctgcaaggcut

SEQ ID NO: 288 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
agtgaaaatcucctgcaaggtut

SEQ ID NO: 289 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
ggtgaaggtcucctgcaaggcut

SEQ ID NO: 290 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
acgctgaccugcacctuct

SEQ ID NO: 291 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
acgcugacccgcacctuct

SEQ ID NO: 292 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
acactgaccugcgcctuct

SEQ ID NO: 293 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
acgctgaccugcaccguct

SEQ ID NO: 294 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
acactgaccugcacctuct

SEQ ID NO: 295 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ctgaaacuctcctgugcagcct

SEQ ID NO: 296 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ctgagacuctccttugcagcct

SEQ ID NO: 297 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ctgagacuctcctgtucagcct

SEQ ID NO: 298 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
cttagacuctcctgugcagcct

SEQ ID NO: 299 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ctgagacuctcctgugcagcct

SEQ ID NO: 300 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ctgagacuctcctgugcagcgt

SEQ ID NO: 301 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ctgagactcucctgtacagcut

SEQ ID NO: 302 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gtccctcaccugcgctguct

SEQ ID NO: 303 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gtcccucacctgtactguct

SEQ ID NO: 304 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gtccctcaccugcactguct

SEQ ID NO: 305 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gtccctcaccugcgctauct

SEQ ID NO: 306 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gtccctcaccugcactguca

SEQ ID NO: 307 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
gtctctgaagauctcctgtaagggut

SEQ ID NO: 308 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
gtctctgaggauctcctgtaagggut

SEQ ID NO: 309 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
cctctcacucacctgtgccauct

SEQ ID NO: 310 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
agtgaaggttucctgcaaggcut

SEQ ID NO: 311 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gggctgaggugaagaagctug

SEQ ID NO: 312 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gagctgaggugaagaagccug

SEQ ID NO: 313 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggtctgagtugaagaagccug

SEQ ID NO: 314 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gggctgaggugaagaagacug

SEQ ID NO: 315 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggcctgaggugaagaagccug

SEQ ID NO: 316 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gggctgaggugaagaagccug

SEQ ID NO: 317 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gtctggtccuacgctgguaaaa

SEQ ID NO: 318 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gtctggtccugcgctggugaaa

SEQ ID NO: 319 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gtctggtccugtgctggugaaa

SEQ ID NO: 320 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gtctggtccuacgctggugaaa

SEQ ID NO: 321 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggtccctuagactctcctgug

SEQ ID NO: 322 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggtcccugagactctcctgua

SEQ ID NO: 323 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
cgtcccugagactctcctgua

SEQ ID NO: 324 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggtcccugaaactctcctgug

SEQ ID NO: 325 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggtcccugagactctccttug

SEQ ID NO: 326 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gggcccugagactctcctgug

SEQ ID NO: 327 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggtcccugagactctcctgut

SEQ ID NO: 328 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggtcccugagactctcctgug

SEQ ID NO: 329 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
gggcgcaggacugtugaag

SEQ ID NO: 330 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
agguccaggactggugaag

SEQ ID NO: 331 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
cggcucaggactggugaag

SEQ ID NO: 332 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
gggcccaggacugtugaag

SEQ ID NO: 333 - RNA, 19 nt, Artificial Sequence
  source: /note=“Description of Artificial Sequence Synthetic oligonucleotide”
gggcccaggacuggugaag

SEQ ID NO: 334 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
agguccgggactggugaag

SEQ ID NO: 335 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gcagtcuggagcagaggugaaa

SEQ ID NO: 336 - RNA, 22 nt, Artificial Sequence
  source: /note=“Description of Artificial Sequence Synthetic oligonucleotide”
gcaguccggagcagaggugaaa

SEQ ID NO: 337 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggctgaggugaagaagccugg

SEQ ID NO: 338 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
agctgaggugaagaagccugg

SEQ ID NO: 339 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gcctgaggugaagaagccugg

SEQ ID NO: 340 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggctgaggugaagaagctugg

SEQ ID NO: 341 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggctgaggugaagaagacugg

SEQ ID NO: 342 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gtctgagtugaagaagccugg

SEQ ID NO: 343 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gtccuacgctggugaaaccc

SEQ ID NO: 344 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gtcctgugctggugaaaccc

SEQ ID NO: 345 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gtccugcgctggugaaaccc

SEQ ID NO: 346 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gtccuacgctgguaaaaccc

SEQ ID NO: 347 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gtccaggacuggugaagccc

SEQ ID NO: 348 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gcccaggacugtugaagcct

SEQ ID NO: 349 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gtccgggacuggugaagccc

SEQ ID NO: 350 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gcucaggactggugaagcct

SEQ ID NO: 351 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gcccaggacuggugaagcct

SEQ ID NO: 352 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gcgcaggacugtugaagcct

SEQ ID NO: 353 - RNA, 23 nt, Artificial Sequence
  source: /note=“Description of Artificial Sequence Synthetic oligonucleotide”
gcaguccggagcagaggugaaaa

SEQ ID NO: 354 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
gcagtcuggagcagaggugaaaa

SEQ ID NO: 355 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ggtctgagtugaagaagccugg

SEQ ID NO: 356 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ggcctgaggugaagaagccugg

SEQ ID NO: 357 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gggctgaggugaagaagacugg

SEQ ID NO: 358 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gggctgaggugaagaagctugg

SEQ ID NO: 359 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gagctgaggugaagaagccugg

SEQ ID NO: 360 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gggctgaggugaagaagccugg

SEQ ID NO: 361 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
ccugcgctggugaaacccac

SEQ ID NO: 362 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
ccugtgctggugaaacccac

SEQ ID NO: 363 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
ccuacgctggugaaacccac

SEQ ID NO: 364 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
ccuacgctgguaaaacccac

SEQ ID NO: 365 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ggctgaggugaagaagccuggg

SEQ ID NO: 366 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gcctgaggugaagaagccuggg

SEQ ID NO: 367 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ggctgaggugaagaagctuggg

SEQ ID NO: 368 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
agctgaggugaagaagccuggg

SEQ ID NO: 369 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gtctgagtugaagaagccuggg

SEQ ID NO: 370 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ggctgaggugaagaagacuggg

SEQ ID NO: 371 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
tgcgcuggugaaacccacac

SEQ ID NO: 372 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
tacgcugguaaaacccacac

SEQ ID NO: 373 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
tgugctggugaaacccacag

SEQ ID NO: 374 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
tacgcuggugaaacccacac

SEQ ID NO: 375 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
tcctcggugaaggtctccugca

SEQ ID NO: 376 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gcctcagugaaggtctccugca

SEQ ID NO: 377 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
tcctcagugaaggtctccugca

SEQ ID NO: 378 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
acctcagugaaggtctccugca

SEQ ID NO: 379 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gcctcagugaaggtttccugca

SEQ ID NO: 380 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
tcctcagugaaggtttccugca

SEQ ID NO: 381 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gctacagugaaaatctccugca

SEQ ID NO: 382 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
cagacccucacactgaccug

SEQ ID NO: 383 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gagacccucacgctgaccug

SEQ ID NO: 384 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
cagacccucacgctgaccug

SEQ ID NO: 385 - RNA, 20 nt, Artificial Sequence
  source: /note=“Description of Artificial Sequence Synthetic oligonucleotide”
cagacccucacgcugacccg

SEQ ID NO: 386 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
ctgtcccucacctgtacugt

SEQ ID NO: 387 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
ccgtcccucacctgcacugt

SEQ ID NO: 388 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
ctctcacucacctgugccat

SEQ ID NO: 389 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
ctgtcccucacctgcacugt

SEQ ID NO: 390 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
ctgtcccucacctgcgcuat

SEQ ID NO: 391 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
ctgtcccucacctgcgcugt

SEQ ID NO: 392 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gggagtctcugaggatctcctgua

SEQ ID NO: 393 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gggagtctcugaagatctcctgua

SEQ ID NO: 394 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
tcggugaaggtctccugcaagg

SEQ ID NO: 395 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
tcagugaaggtttccugcaagg

SEQ ID NO: 396 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
acagtgaaaauctccugcaagg

SEQ ID NO: 397 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
tcagtgaagguctccugcaagg

SEQ ID NO: 398 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
tcacgcugaccugcaccgt

SEQ ID NO: 399 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
tcacgcugacccgcaccut

SEQ ID NO: 400 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
tcacactgaccugcgccut

SEQ ID NO: 401 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
tcacacugacctgcaccut

SEQ ID NO: 402 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
tcacgcugacctgcaccut

SEQ ID NO: 403 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ccctgagacuctcctguacagc

SEQ ID NO: 404 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ccctgagacuctcctgtucagc

SEQ ID NO: 405 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ccctgagacuctccttugcagc

SEQ ID NO: 406 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ccctgaaacuctcctgugcagc

SEQ ID NO: 407 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ccctgagacuctcctgugcagc

SEQ ID NO: 408 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
cccttagacuctcctgugcagc

SEQ ID NO: 409 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
gagtctcugaagatctcctguaaggg

SEQ ID NO: 410 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
gagtctcugaggatctcctguaaggg

SEQ ID NO: 411 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gtgaaggtcucctgcaaggctuct

SEQ ID NO: 412 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gtgaaaatcucctgcaaggttuct

SEQ ID NO: 413 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gtgaaggttucctgcaaggcauct

SEQ ID NO: 414 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gtgaaggttucctgcaaggctuct

SEQ ID NO: 415 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gtgaaggttucctgcaaggctucc

SEQ ID NO: 416 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gtgaaggtcucctgcaaggttucc

SEQ ID NO: 417 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
acgcugacccgcaccttcuc

SEQ ID NO: 418 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
acgctgaccugcaccttcuc

SEQ ID NO: 419 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
acgctgaccugcaccgtcuc

SEQ ID NO: 420 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
acactgaccugcgccttcuc

SEQ ID NO: 421 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
acactgaccugcaccttcuc

SEQ ID NO: 422 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gtccctcaccugcactgtcuc

SEQ ID NO: 423 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gtccctcaccugtactgtcuc

SEQ ID NO: 424 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gtccctcaccugcgctatcuc

SEQ ID NO: 425 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gtccctcaccugcactgucac

SEQ ID NO: 426 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ctcactcaccugtgccatcuc

SEQ ID NO: 427 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gtccctcaccugcgctgtcua

SEQ ID NO: 428 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gtccctcaccugcgctgtcuc

SEQ ID NO: 429 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
gtctctgaggauctcctgtaagggtuc

SEQ ID NO: 430 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
gtctctgaagatcucctgtaagggtuc

SEQ ID NO: 431 - DNA, 25 bp, Artificial Sequence
  source: /note=“Description of Artificial Sequence Synthetic oligonucleotide”
ctgggtgcgacaggcccctggacaa

SEQ ID NO: 432 - DNA, 25 bp, Artificial Sequence
  source: /note=“Description of Artificial Sequence Synthetic oligonucleotide”
tggatccgtcagcccccagggaagg

SEQ ID NO: 433 - DNA, 21 bp, Artificial Sequence
  source: /note=“Description of Artificial Sequence Synthetic oligonucleotide”
ggtccgccaggctccagggaa

SEQ ID NO: 434 - DNA, 25 bp, Artificial Sequence
  source: /note=“Description of Artificial Sequence Synthetic oligonucleotide”
tggatccgccagcccccagggaagg

SEQ ID NO: 435 - DNA, 25 bp, Artificial Sequence
  source: /note=“Description of Artificial Sequence Synthetic oligonucleotide”
gggtgcgccagatgcccgggaaagg

SEQ ID NO: 436 - DNA, 25 bp, Artificial Sequence
  source: /note=“Description of Artificial Sequence Synthetic oligonucleotide”
tggatcaggcagtccccatcgagag

SEQ ID NO: 437 - DNA, 25 bp, Artificial Sequence
  source: /note=“Description of Artificial Sequence Synthetic oligonucleotide”
ttgggtgcgacaggcccctggacaa

SEQ ID NO: 438 - DNA, 18 bp, Artificial Sequence
  source: /note=“Description of Artificial Sequence Synthetic oligonucleotide”
gaggagacggtgaccgtg

SEQ ID NO: 439 - DNA, 19 bp, Artificial Sequence
  source: /note=“Description of Artificial Sequence Synthetic oligonucleotide”
gagacagtgaccagggtgc

SEQ ID NO: 440 - DNA, 19 bp, Artificial Sequence
  source: /note=“Description of Artificial Sequence Synthetic oligonucleotide”
gagacggtgaccattgtcc

SEQ ID NO: 441 - DNA, 19 bp, Artificial Sequence
  source: /note=“Description of Artificial Sequence Synthetic oligonucleotide”
tgaggagacggtgaccagg

SEQ ID NO: 442 - DNA, 22 bp, Artificial Sequence
  source: /note=“Description of Artificial Sequence Synthetic oligonucleotide”
ccagtggcagaggagtccattc

SEQ ID NO: 443 - RNA, 18 nt, Artificial Sequence
  source: /note=“Description of Artificial Sequence Synthetic oligonucleotide”
gaggagacggugaccgug

SEQ ID NO: 444 - RNA, 19 nt, Artificial Sequence
  source: /note=“Description of Artificial Sequence Synthetic oligonucleotide”
gagacagugaccagggugc

SEQ ID NO: 445 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
gagacggugaccattgucc

SEQ ID NO: 446 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
tgaggagacggugaccagg

SEQ ID NO: 447 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ccaguggcagaggagtccatuc

SEQ ID NO: 448 - DNA, 20 bp, Artificial Sequence
  source: /note=“Description of Artificial Sequence Synthetic oligonucleotide”
aggtcacactgagtggctcc

SEQ ID NO: 449 - DNA, 20 bp, Artificial Sequence
  source: /note=“Description of Artificial Sequence Synthetic oligonucleotide”
acgaccacgttcccatcttg

SEQ ID NO: 450 - DNA, 20 bp, Artificial Sequence
  source: /note=“Description of Artificial Sequence Synthetic oligonucleotide”
atgaccacgttcccatctgg

SEQ ID NO: 451 - DNA, 22 bp, Artificial Sequence
  source: /note=“Description of Artificial Sequence Synthetic oligonucleotide”
gcgatgaccacgttcccatctg

SEQ ID NO: 452 - DNA, 22 bp, Artificial Sequence
  source: /note=“Description of Artificial Sequence Synthetic oligonucleotide”
gcgacgaccacgttcccatctt

SEQ ID NO: 453 - DNA, 22 bp, Artificial Sequence
  source: /note=“Description of Artificial Sequence Synthetic oligonucleotide”
aggcgatgaccacgttcccatc

SEQ ID NO: 454 - DNA, 22 bp, Artificial Sequence
  source: /note=“Description of Artificial Sequence Synthetic oligonucleotide”
atgcgacgaccacgttcccatc

SEQ ID NO: 455 - DNA, 21 bp, Artificial Sequence
  source: /note=“Description of Artificial Sequence Synthetic oligonucleotide”
atgcgacgaccacgttcccat

SEQ ID NO: 456 - DNA, 21 bp, Artificial Sequence
  source: /note=“Description of Artificial Sequence Synthetic oligonucleotide”
aggcgatgaccacgttcccat

SEQ ID NO: 457 - DNA, 21 bp, Artificial Sequence
  source: /note=“Description of Artificial Sequence Synthetic oligonucleotide”
gctttcgctccaggtcacact

SEQ ID NO: 458 - DNA, 21 bp, Artificial Sequence
  source: /note=“Description of Artificial Sequence Synthetic oligonucleotide”
cgctttcgctccaggtcacac

SEQ ID NO: 459 - DNA, 20 bp, Artificial Sequence
  source: /note=“Description of Artificial Sequence Synthetic oligonucleotide”
cgctttcgctccaggtcaca

SEQ ID NO: 460 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
aggtcacacugagtggcucc

SEQ ID NO: 461 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
acgaccacgutcccatctug

SEQ ID NO: 462 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
atgaccacgutcccatcugg

SEQ ID NO: 463 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gcgatgaccacgutcccatcug

SEQ ID NO: 464 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
gcgacgaccacgutcccatcut

SEQ ID NO: 465 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
aggcgaugaccacgttcccauc

SEQ ID NO: 466 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
atgcgacgaccacgutcccauc

SEQ ID NO: 467 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
augcgacgaccacgtucccat

SEQ ID NO: 468 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
aggcgaugaccacgtucccat

SEQ ID NO: 469 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gctttcgcuccaggucacact

SEQ ID NO: 470 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
cgctttcgcuccaggucacac

SEQ ID NO: 471 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
cgctttcgcuccaggucaca

SEQ ID NO: 472 - DNA, 19 bp, Artificial Sequence
  source: /note=“Description of Artificial Sequence Synthetic oligonucleotide”
gacagtcacggacgttggg

SEQ ID NO: 473 - DNA, 23 bp, Artificial Sequence
  source: /note=“Description of Artificial Sequence Synthetic oligonucleotide”
accacagggctgttatcctttgg

SEQ ID NO: 474 - DNA, 22 bp, Artificial Sequence
  source: /note=“Description of Artificial Sequence Synthetic oligonucleotide”
accacagggctgttatcctttg

SEQ ID NO: 475 - DNA, 24 bp, Artificial Sequence
  source: /note=“Description of Artificial Sequence Synthetic oligonucleotide”
caggaccacagggctgttatcctt

SEQ ID NO: 476 - DNA, 23 bp, Artificial Sequence
  source: /note=“Description of Artificial Sequence Synthetic oligonucleotide”
caggaccacagggctgttatcct

SEQ ID NO: 477 - DNA, 23 bp, Artificial Sequence
  source: /note=“Description of Artificial Sequence Synthetic oligonucleotide”
cccatgtaccaggtgacagtcac

SEQ ID NO: 478 - DNA, 22 bp, Artificial Sequence
  source: /note=“Description of Artificial Sequence Synthetic oligonucleotide”
cccatgtaccaggtgacagtca

SEQ ID NO: 479 - DNA, 21 bp, Artificial Sequence
  source: /note=“Description of Artificial Sequence Synthetic oligonucleotide”
cccatgtaccaggtgacagtc

SEQ ID NO: 480 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
gacagucacggacgtuggg

SEQ ID NO: 481 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
accacagggcugttatccttugg

SEQ ID NO: 482 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
accacagggcugttatccttug

SEQ ID NO: 483 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
caggaccacagggcugttatccut

SEQ ID NO: 484 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
caggaccacagggcugttaucct

SEQ ID NO: 485 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
cccatguaccaggtgacagucac

SEQ ID NO: 486 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
cccatguaccaggtgacaguca

SEQ ID NO: 487 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
cccatguaccaggtgacaguc

SEQ ID NO: 488 - DNA, 18 bp, Artificial Sequence
  source: /note=“Description of Artificial Sequence Synthetic oligonucleotide”
cgacaccgtcaccggttc

SEQ ID NO: 489 - DNA, 22 bp, Artificial Sequence
  source: /note=“Description of Artificial Sequence Synthetic oligonucleotide”
ggccgctgtgctctcggaggtg

SEQ ID NO: 490 - DNA, 22 bp, Artificial Sequence
  source: /note=“Description of Artificial Sequence Synthetic oligonucleotide”
ggccgctgtgcccccagaggtg

SEQ ID NO: 491 - DNA, 22 bp, Artificial Sequence
  source: /note=“Description of Artificial Sequence Synthetic oligonucleotide”
ggcggctgtgctctcggaggtg

SEQ ID NO: 492 - DNA, 22 bp, Artificial Sequence
  source: /note=“Description of Artificial Sequence Synthetic oligonucleotide”
ggccgctgtgccctcagagacg

SEQ ID NO: 493 - DNA, 22 bp, Artificial Sequence
  source: /note=“Description of Artificial Sequence Synthetic oligonucleotide”
ggctgctgtgcccccagaggtg

SEQ ID NO: 494 - DNA, 21 bp, Artificial Sequence
  source: /note=“Description of Artificial Sequence Synthetic oligonucleotide”
ggccgctgtgccctcagagac

SEQ ID NO: 495 - DNA, 21 bp, Artificial Sequence
  source: /note=“Description of Artificial Sequence Synthetic oligonucleotide”
ggctgctgtgcccccagaggt

SEQ ID NO: 496 - DNA, 21 bp, Artificial Sequence
  source: /note=“Description of Artificial Sequence Synthetic oligonucleotide”
ggcggctgtgctctcggaggt

SEQ ID NO: 497 - DNA, 21 bp, Artificial Sequence
  source: /note=“Description of Artificial Sequence Synthetic oligonucleotide”
ggccgctgtgctctcggaggt

SEQ ID NO: 498 - DNA, 21 bp, Artificial Sequence
  source: /note=“Description of Artificial Sequence Synthetic oligonucleotide”
ggccgctgtgcccccagaggt

SEQ ID NO: 499 - DNA, 20 bp, Artificial Sequence
  source: /note=“Description of Artificial Sequence Synthetic oligonucleotide”
gggctgctgtgcccccagag

SEQ ID NO: 500 - DNA, 20 bp, Artificial Sequence
  source: /note=“Description of Artificial Sequence Synthetic oligonucleotide”
gggcggctgtgctctcggag

SEQ ID NO: 501 - DNA, 20 bp, Artificial Sequence
  source: /note=“Description of Artificial Sequence Synthetic oligonucleotide”
gggccgctgtgcccccagag

SEQ ID NO: 502 - DNA, 20 bp, Artificial Sequence
  source: /note=“Description of Artificial Sequence Synthetic oligonucleotide”
gggccgctgtgccctcagag

SEQ ID NO: 503 - DNA, 20 bp, Artificial Sequence
  source: /note=“Description of Artificial Sequence Synthetic oligonucleotide”
gggccgctgtgctctcggag

SEQ ID NO: 504 - DNA, 21 bp, Artificial Sequence
  source: /note=“Description of Artificial Sequence Synthetic oligonucleotide”
cagggcggctgtgctctcgga

SEQ ID NO: 505 - DNA, 21 bp, Artificial Sequence
  source: /note=“Description of Artificial Sequence Synthetic oligonucleotide”
cagggccgctgtgccctcaga

SEQ ID NO: 506 - DNA, 21 bp, Artificial Sequence
  source: /note=“Description of Artificial Sequence Synthetic oligonucleotide”
cagggctgctgtgcccccaga

SEQ ID NO: 507 - DNA, 21 bp, Artificial Sequence
  source: /note=“Description of Artificial Sequence Synthetic oligonucleotide”
cagggccgctgtgcccccaga

SEQ ID NO: 508 - DNA, 21 bp, Artificial Sequence
  source: /note=“Description of Artificial Sequence Synthetic oligonucleotide”
cagggccgctgtgctctcgga

SEQ ID NO: 509 - DNA, 20 bp, Artificial Sequence
  source: /note=“Description of Artificial Sequence Synthetic oligonucleotide”
ctgagttccacgacaccgtc

SEQ ID NO: 510 - DNA, 21 bp, Artificial Sequence
  source: /note=“Description of Artificial Sequence Synthetic oligonucleotide”
ccctgagttccacgacaccgt

SEQ ID NO: 511 - DNA, 21 bp, Artificial Sequence
  source: /note=“Description of Artificial Sequence Synthetic oligonucleotide”
gcctgagttccacgacaccgt

SEQ ID NO: 512 - DNA, 20 bp, Artificial Sequence
  source: /note=“Description of Artificial Sequence Synthetic oligonucleotide”
ccctgagttccacgacaccg

SEQ ID NO: 513 - DNA, 20 bp, Artificial Sequence
  source: /note=“Description of Artificial Sequence Synthetic oligonucleotide”
gcctgagttccacgacaccg

SEQ ID NO: 514 - DNA, 18 bp, Artificial Sequence
  source: /note=“Description of Artificial Sequence Synthetic oligonucleotide”
  source: /note=“Description of Combined DNA/RNA Molecule Synthetic oligonucleotide”
cgacaccgucaccggtuc

SEQ ID NO: 515 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ggccgctgugctctcggaggug

SEQ ID NO: 516 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ggccgctgugcccccagaggug

SEQ ID NO: 517 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ggcggctgugctctcggaggug

SEQ ID NO: 518 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ggccgcugtgcccucagagacg

SEQ ID NO: 519 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ggctgctgugcccccagaggug

SEQ ID NO: 520 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggccgcugtgcccucagagac

SEQ ID NO: 521 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggcugctgugcccccagaggt

SEQ ID NO: 522 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggcggcugtgctcucggaggt

SEQ ID NO: 523 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggccgcugtgctcucggaggt

SEQ ID NO: 524 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ggccgcugugcccccagaggt

SEQ ID NO: 525 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gggcugctgugcccccagag

SEQ ID NO: 526 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gggcggcugtgctcucggag

SEQ ID NO: 527 - RNA, 20 nt, Artificial Sequence
  source: /note=“Description of Artificial Sequence Synthetic oligonucleotide”
gggccgcugugcccccagag

SEQ ID NO: 528 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gggccgcugtgcccucagag

SEQ ID NO: 529 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gggccgcugtgctcucggag

SEQ ID NO: 530 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
cagggcggcugtgctcucgga

SEQ ID NO: 531 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
cagggccgcugtgcccucaga

SEQ ID NO: 532 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
cagggcugctgugcccccaga

SEQ ID NO: 533 - RNA, 21 nt, Artificial Sequence
  source: /note=“Description of Artificial Sequence Synthetic oligonucleotide”
cagggccgcugugcccccaga

SEQ ID NO: 534 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
cagggccgcugtgctcucgga

SEQ ID NO: 535 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
ctgagtuccacgacaccguc

SEQ ID NO: 536 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
cccugagtuccacgacaccgt

SEQ ID NO: 537 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gccugagtuccacgacaccgt

SEQ ID NO: 538 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
cccugagtuccacgacaccg

SEQ ID NO: 539 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gccugagtuccacgacaccg

SEQ ID NO: 540 - DNA, 22 bp, Artificial Sequence
  source: /note=“Description of Artificial Sequence Synthetic oligonucleotide”
ggagaaagtgatggagtcggga

SEQ ID NO: 541 - DNA, 22 bp, Artificial Sequence
  source: /note=“Description of Artificial Sequence Synthetic oligonucleotide”
ggacaaagtgatggagtcggga

SEQ ID NO: 542 - DNA, 21 bp, Artificial Sequence
  source: /note=“Description of Artificial Sequence Synthetic oligonucleotide”
gtcctgtgcgaggcagccaac

SEQ ID NO: 543 - DNA, 20 bp, Artificial Sequence
  source: /note=“Description of Artificial Sequence Synthetic oligonucleotide”
gtcctgtgcgaggcagccaa

SEQ ID NO: 544 - DNA, 24 bp, Artificial Sequence
  source: /note=“Description of Artificial Sequence Synthetic oligonucleotide”
aggaagtcctgtgcgaggcagcca

SEQ ID NO: 545 - DNA, 23 bp, Artificial Sequence
  source: /note=“Description of Artificial Sequence Synthetic oligonucleotide”
aggaagtcctgtgcgaggcagcc

SEQ ID NO: 546 - DNA, 21 bp, Artificial Sequence
  source: /note=“Description of Artificial Sequence Synthetic oligonucleotide”
gtatttccaggagaaagtgat

SEQ ID NO: 547 - DNA, 21 bp, Artificial Sequence
  source: /note=“Description of Artificial Sequence Synthetic oligonucleotide”
gtatttccaggacaaagtgat

SEQ ID NO: 548 - DNA, 21 bp, Artificial Sequence
  source: /note=“Description of Artificial Sequence Synthetic oligonucleotide”
tgtatttccaggacaaagtga

SEQ ID NO: 549 - DNA, 21 bp, Artificial Sequence
  source: /note=“Description of Artificial Sequence Synthetic oligonucleotide”
tgtatttccaggagaaagtga

SEQ ID NO: 550 - DNA, 20 bp, Artificial Sequence
  source: /note=“Description of Artificial Sequence Synthetic oligonucleotide”
tgtatttccaggacaaagtg

SEQ ID NO: 551 - DNA, 20 bp, Artificial Sequence
  source: /note=“Description of Artificial Sequence Synthetic oligonucleotide”
tgtatttccaggagaaagtg

SEQ ID NO: 552 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ggagaaagugatggagucggga

SEQ ID NO: 553 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
ggacaaagugatggagucggga

SEQ ID NO: 554 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gucctgugcgaggcagccaac

SEQ ID NO: 555 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gucctgugcgaggcagccaa

SEQ ID NO: 556 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
aggaagucctgugcgaggcagcca

SEQ ID NO: 557 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
aggaagucctgugcgaggcagcc

SEQ ID NO: 558 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gtattuccaggagaaagugat

SEQ ID NO: 559 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
gtattuccaggacaaagugat

SEQ ID NO: 560 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
tgtattuccaggacaaaguga

SEQ ID NO: 561 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
tgtattuccaggagaaaguga

SEQ ID NO: 562 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
tgtattuccaggacaaagug

SEQ ID NO: 563 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
tgtattuccaggagaaagug

SEQ ID NO: 564 - DNA, 19 bp, Artificial Sequence
  source: /note=“Description of Artificial Sequence Synthetic oligonucleotide”
ggaagtagcctgtggccag

SEQ ID NO: 565 - DNA, 19 bp, Artificial Sequence
  source: /note=“Description of Artificial Sequence Synthetic oligonucleotide”
ggaagtagcccgtggccag

SEQ ID NO: 566 - DNA, 20 bp, Artificial Sequence
  source: /note=“Description of Artificial Sequence Synthetic oligonucleotide”
gaggtggcatcagaggcaat

SEQ ID NO: 567 - DNA, 20 bp, Artificial Sequence
  source: /note=“Description of Artificial Sequence Synthetic oligonucleotide”
gaggtggcattggagggaat

SEQ ID NO: 568 - DNA, 19 bp, Artificial Sequence
  source: /note=“Description of Artificial Sequence Synthetic oligonucleotide”
gaggtggcattggagggaa

SEQ ID NO: 569 - DNA, 19 bp, Artificial Sequence
  source: /note=“Description of Artificial Sequence Synthetic oligonucleotide”
gaggtggcatcagaggcaa

SEQ ID NO: 570 - DNA, 19 bp, Artificial Sequence
  source: /note=“Description of Artificial Sequence Synthetic oligonucleotide”
tggaggtggcatcagaggc

SEQ ID NO: 571 - DNA, 19 bp, Artificial Sequence
  source: /note=“Description of Artificial Sequence Synthetic oligonucleotide”
cggaggtggcattggaggg

SEQ ID NO: 572 - DNA, 18 bp, Artificial Sequence
  source: /note=“Description of Artificial Sequence Synthetic oligonucleotide”
tggaggtggcatcagagg

SEQ ID NO: 573 - DNA, 18 bp, Artificial Sequence
  source: /note=“Description of Artificial Sequence Synthetic oligonucleotide”
cggaggtggcattggagg

SEQ ID NO: 574 - DNA, 27 bp, Artificial Sequence
  source: /note=“Description of Artificial Sequence Synthetic oligonucleotide”
gcctgtgtcccaggtcaccatcaccgg

SEQ ID NO: 575 - DNA, 27 bp, Artificial Sequence
  source: /note=“Description of Artificial Sequence Synthetic oligonucleotide”
gcctgtgtcccaggtcacagtcactga

SEQ ID NO: 576 - DNA, 27 bp, Artificial Sequence
  source: /note=“Description of Artificial Sequence Synthetic oligonucleotide”
gcctgtgtcgcaggtcaccatcaccgg

SEQ ID NO: 577 - DNA, 26 bp, Artificial Sequence
  source: /note=“Description of Artificial Sequence Synthetic oligonucleotide”
gcctgtgtcccaggtcaccatcaccg

SEQ ID NO: 578 - DNA, 26 bp, Artificial Sequence
  source: /note=“Description of Artificial Sequence Synthetic oligonucleotide”
gcctgtgtcgcaggtcaccatcaccg

SEQ ID NO: 579 - DNA, 26 bp, Artificial Sequence
  source: /note=“Description of Artificial Sequence Synthetic oligonucleotide”
gcctgtgtcccaggtcacagtcactg

SEQ ID NO: 580 - DNA, 26 bp, Artificial Sequence
  source: /note=“Description of Artificial Sequence Synthetic oligonucleotide”
agcctgtgtcccaggtcaccatcacc

SEQ ID NO: 581 - DNA, 26 bp, Artificial Sequence
  source: /note=“Description of Artificial Sequence Synthetic oligonucleotide”
agcctgtgtcgcaggtcaccatcacc

SEQ ID NO: 582 - DNA, 26 bp, Artificial Sequence
  source: /note=“Description of Artificial Sequence Synthetic oligonucleotide”
agcctgtgtcccaggtcacagtcact

SEQ ID NO: 583 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
ggaaguagcctguggccag

SEQ ID NO: 584 - RNA, 19 nt, Artificial Sequence
  source: /note=“Description of Artificial Sequence Synthetic oligonucleotide”
ggaaguagcccguggccag

SEQ ID NO: 585 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gagguggcaucagaggcaat

SEQ ID NO: 586 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
gagguggcatuggagggaat

SEQ ID NO: 587 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
gagguggcatuggagggaa

SEQ ID NO: 588 - RNA, 19 nt, Artificial Sequence
  source: /note=“Description of Artificial Sequence Synthetic oligonucleotide”
gagguggcaucagaggcaa

SEQ ID NO: 589 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
tggagguggcaucagaggc

SEQ ID NO: 590 - DNA, 19 bp, Artificial Sequence
  source: /note=“Description of Artificial Sequence Synthetic oligonucleotide”
  source: /note=“Description of Combined DNA/RNA Molecule Synthetic oligonucleotide”
cggagguggcatuggaggg

SEQ ID NO: 591 - DNA, 18 bp, Artificial Sequence
  source: /note=“Description of Artificial Sequence Synthetic oligonucleotide”
  source: /note=“Description of Combined DNA/RNA Molecule Synthetic oligonucleotide”
tggagguggcaucagagg

SEQ ID NO: 592 - DNA, 18 bp, Artificial Sequence
  source: /note=“Description of Artificial Sequence Synthetic oligonucleotide”
  source: /note=“Description of Combined DNA/RNA Molecule Synthetic oligonucleotide”
cggagguggcatuggagg

SEQ ID NO: 593 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
gcctgtgucccaggtcaccaucaccgg

SEQ ID NO: 594 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
gcctgtgtcccaggucacagtcacuga

SEQ ID NO: 595 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
gcctgtgucgcaggtcaccaucaccgg

SEQ ID NO: 596 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
gcctgtgucccaggtcaccaucaccg

SEQ ID NO: 597 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
gcctgtgucgcaggtcaccaucaccg

SEQ ID NO: 598 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
gcctgtgtcccaggucacagtcacug

SEQ ID NO: 599 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
agcctgtgucccaggtcaccaucacc

SEQ ID NO: 600 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
agcctgtgucgcaggtcaccaucacc

SEQ ID NO: 601 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
agcctgtgucccaggtcacagucact

SEQ ID NO: 602 - DNA, 10 bp, Artificial Sequence
  source: /note=“Description of Artificial Sequence Synthetic oligonucleotide”
aaaatttggt

SEQ ID NO: 603 - DNA, 22 bp, Artificial Sequence
  source: /note=“Description of Artificial Sequence Synthetic oligonucleotide”
aaaatttttatccccccccggg

SEQ ID NO: 604 - DNA, 42 bp, Artificial Sequence
  source: /note=“Description of Artificial Sequence Synthetic oligonucleotide”
gcaaaagatgttggggctacgggctactacggtatggacgtc

SEQ ID NO: 605 - DNA, 45 bp, Artificial Sequence
  source: /note=“Description of Artificial Sequence Synthetic oligonucleotide”
gcgagcgtgcgcagggggcagttagtcactgcccactttgactcc